(19) United States
(12) Patent Application Publication
(10) Pub. No.: US 2026/0282740 A1
(43) Pub. Date: Sep. 17, 2026

(54) HETEROCYCLIC COMPOUND, ORGANIC LIGHT-EMITTING ELEMENT, AND COMPOSITION FOR ORGANIC MATERIAL LAYER OF ORGANIC LIGHT-EMITTING ELEMENT

(71) Applicant: LT MATERIALS CO., LTD., Yongin-si, Gyeonggi-do (KR)

(72) Inventors: Kyu-Tae SEO, Yongin-si (KR); Hyun-Joo LEE, Yongin-si (KR); Geon-Yu PARK, Yongin-si (KR); Young-Seok NO, Yongin-si (KR); Dong-Jun KIM, Yongin-si (KR); Dae-Hyuk CHOI, Yongin-si (KR)

(73) Assignee: LT MATERIALS CO., LTD., Yongin-si, Gyeonggi-do (KR)

(21) Appl. No.: 18/877,734

(22) PCT Filed: Jul. 27, 2023
(86) PCT No.: PCT/KR2023/010941

§ 371 Date: Dec. 20, 2024

(30) Foreign Application Priority Data
Jul. 27, 2022 (KR) 10-2022-0093434

(51) Int. Cl. H10K 85/60; C07D 405/14; C07D 409/14; C09K 11/02; H10K 50/11

(52) U.S. Cl. CPC H10K 85/654; C07D 405/14; C07D 409/14; C09K 11/02; H10K 85/615; H10K 85/622; H10K 85/6572; H10K 85/6574; H10K 85/6576; H10K 50/11

(57) ABSTRACT

The present specification relates to a heterocyclic compound, an organic light emitting device, and a composition for an organic material layer of an organic light emitting device.

### TECHNICAL FIELD

This application claims priority to and the benefit of Korean Patent Application No. 10-2022-0093434 filed in the Korean Intellectual Property Office on Jul. 27, 2022, the entire contents of which are incorporated herein by reference.

The present specification relates to a heterocyclic compound, an organic light emitting device, and a composition for an organic material layer of an organic light emitting device.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### CITATION LIST

(Patent Document 1) U.S. Pat. No. 4,356,429

### DETAILED DESCRIPTION OF THE INVENTION

### Technical Problem

The present specification has been made in an effort to provide a heterocyclic compound, an organic light emitting device, and a composition for an organic material layer of an organic light emitting device.

### Technical Solution

In an exemplary embodiment of the present specification, provided is a heterocyclic compound of the following Chemical Formula 1.

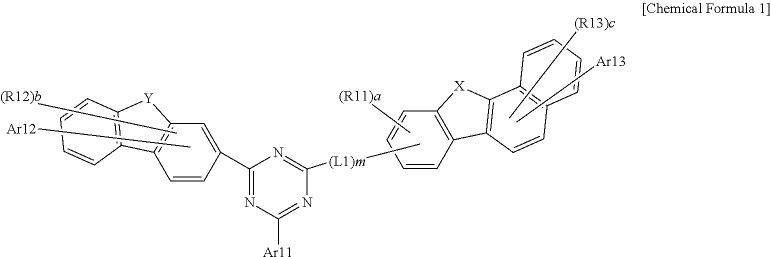

In Chemical Formula 1,
X and Y are the same as or different from each other, and are each independently O; S; or CRR′,
Ar11 and Ar12 are the same as or different from each other, and are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar13 is a substituted or unsubstituted C6 to C10 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group,
R11 to R13 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R and R′ are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
m and c are the same as or different from each other, and are each independently an integer from 0 to 5,
a is an integer from 0 to 3,
b is an integer from 0 to 6, and
when a to c and m are 2 or higher, substituents in the parenthesis are the same as or different from each other.

In another exemplary embodiment of the present specification, provided is an organic light emitting device including: a first electrode; a second electrode disposed to face the first electrode; and an organic material layer having one or more layers disposed between the first electrode and the second electrode, in which one or more layers of the organic material layer include the heterocyclic compound.

In still another exemplary embodiment of the present specification, provided is a composition for an organic material layer of an organic light emitting device, including the heterocyclic compound.

### Advantageous Effects

When used in an organic light emitting device, the heterocyclic compound described in the present specification can lower the driving voltage of the device, improve the light efficiency, and improve the service life characteristics of the device. In particular, the heterocyclic compound of Chemical Formula 1 necessarily has a substituent of Ar13 in the core structure. Specifically, by having a substituent of a substituted or unsubstituted C6 to C10 aryl group; or a substituted or unsubstituted C2 to C60 heteroaryl group, the driving voltage of the device can be lowered, the light efficiency can be improved, and the service life characteristics of the device can be improved due to the thermal stability of the compound.

In particular, both the heterocyclic compound represented by Chemical Formula 1 and the compound represented by Chemical Formula 2 can be used as a material for a light emitting layer of an organic light emitting device. In this case, it is possible to lower a driving voltage of the device, improve the light efficiency, and particularly improve the service life characteristics of the device by the thermal stability of the compound.

### BRIEF DESCRIPTION OF DRAWINGS

FIGS. 1 to 3 are views each exemplarily illustrating a stacking structure of an organic light emitting device according to an exemplary embodiment of the present specification.

### EXPLANATION OF REFERENCE NUMERALS AND SYMBOLS

100: Substrate
200: Positive electrode
300: Organic material layer
301: Hole injection layer
302: Hole transport layer
303: Light Emitting Layer
304: Hole blocking layer
305: Electron transport layer
306: Electron injection layer
400: Negative electrode

### BEST MODE

Hereinafter, the present specification will be described in more detail.

When one part “includes” one constituent element in the present specification, unless otherwise specifically described, this does not mean that another constituent element is excluded, but means that another constituent element may be further included.

In the present specification,

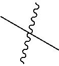

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or with a substituent to which two or more substituents selected among the exemplified substituents are bonded.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

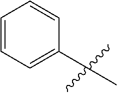 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

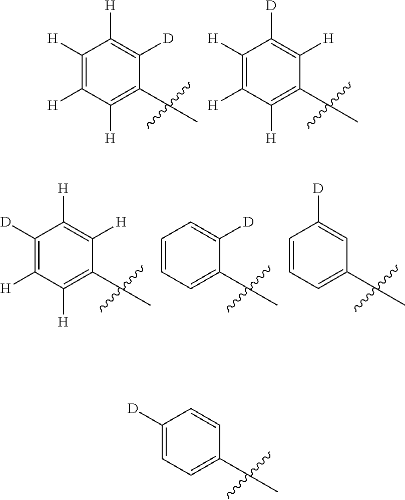

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

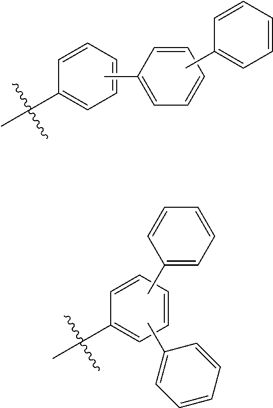

In the present specification, the fluorenyl group may be substituted, and adjacent substituents may be bonded to each other to form a ring.

When the fluorenyl group is substituted, the substituent may be the following structure, but is not limited thereto.

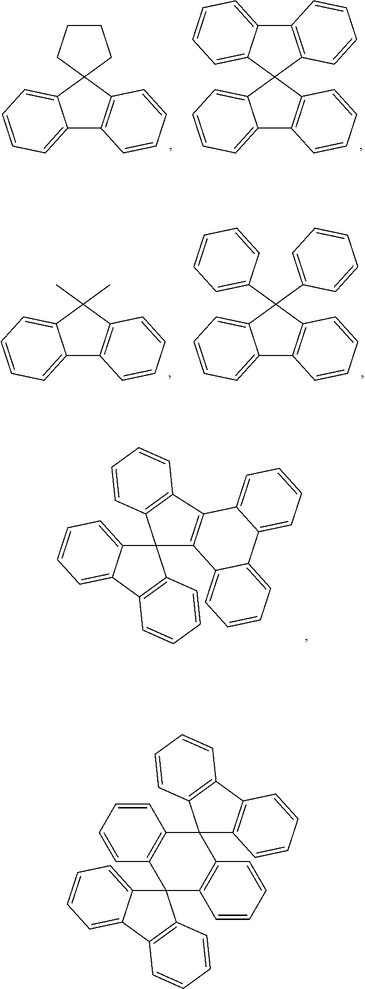

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 2,3-dihydrobenzo[b]thiophene, 2,3-dihydrobenzofuran, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, a silyl group includes Si and is a substituent to which the Si atom is directly linked as a radical, and is represented by —Si(R101)(R102)(R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

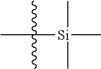 [SMILES: *[Si](C)(C)C]

(a trimethyl silyl group),

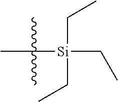 [SMILES: *[Si](CC)(CC)CC]

(a triethylsilyl group),

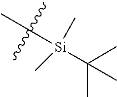 [SMILES: *[Si](C)(C)C(C)(C)C]

(a t-butyldimethylsilyl group),

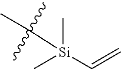 [SMILES: *[Si](C)(C)C=C]

(a vinyldimethylsilyl group),

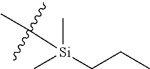 [SMILES: *[Si](C)(C)CCC]

(a propyldimethylsilyl group),

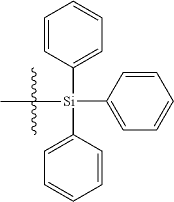 [SMILES: *[Si](c1ccccc1)(c1ccccc1)c1ccccc1]

(a triphenylsilyl group),

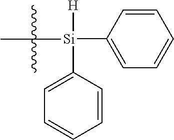 [SMILES: *[SiH](c1ccccc1)c1ccccc1]

(a diphenylsilyl group),

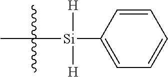 [SMILES: *[SiH2]c1ccccc1]

(a phenylsilyl group) and the like, but are not limited thereto.

In the present specification, the phosphine oxide group is represented by —P(═O)(R104)(R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, the amine group is represented by —N(R106)(R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

An exemplary embodiment of the present specification provides the heterocyclic compound represented by Chemical Formula 1.

In an exemplary embodiment of the present application, X and Y are the same as or different from each other, and are each independently O; S; or CRR′.

In an exemplary embodiment of the present application, X may be O.

In an exemplary embodiment of the present application, X may be S.

In an exemplary embodiment of the present application, Y may be O.

In an exemplary embodiment of the present application, Y may be S.

In an exemplary embodiment of the present application, X is O, and Y may be O.

In an exemplary embodiment of the present application, X is O, and Y may be S.

In an exemplary embodiment of the present application, X is S, and Y may be S.

In an exemplary embodiment of the present application, X is S, and Y may be O.

In an exemplary embodiment of the present application, X and Y may be the same as each other.

In an exemplary embodiment of the present application, X and Y may be different from each other.

In an exemplary embodiment of the present application, Ar11 and Ar12 are the same as or different from each other, and may be each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, Ar11 and Ar12 are the same as or different from each other, and may be each independently a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, Ar11 and Ar12 are the same as or different from each other, and may be each independently a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, Ar11 and Ar12 are the same as or different from each other, and may be each independently a substituted or unsubstituted C6 to C20 aryl group.

In still yet another exemplary embodiment, Ar11 and Ar12 are the same as or different from each other, and may be each independently a C6 to C20 aryl group.

In a further exemplary embodiment, Ar11 and Ar12 are the same as or different from each other, and may be each independently a substituted or unsubstituted phenyl group; or a substituted or unsubstituted biphenyl group.

In another further exemplary embodiment, Ar11 and Ar12 are the same as or different from each other, and may be each independently a phenyl group; or a biphenyl group.

In an exemplary embodiment of the present application, Ar13 may be a substituted or unsubstituted C6 to C10 aryl group; or a substituted or unsubstituted C2 to C60 heteroaryl group.

In another exemplary embodiment, Ar13 may be a substituted or unsubstituted C6 to C10 aryl group; or a substituted or unsubstituted C2 to C40 heteroaryl group.

In still another exemplary embodiment, Ar13 may be a substituted or unsubstituted C6 to C10 aryl group.

In yet another exemplary embodiment, Ar13 may be a substituted or unsubstituted monocyclic C6 to C10 aryl group; or a substituted or unsubstituted polycyclic C6 to C10 aryl group.

In still yet another exemplary embodiment, Ar13 may be a substituted or unsubstituted phenyl group; or a substituted or unsubstituted naphthyl group.

In a further exemplary embodiment, Ar13 may be a phenyl group; or a naphthyl group.

In another further exemplary embodiment, Ar13 may be a phenyl group.

In an exemplary embodiment of the present application, Ar11 may be represented by any one of the following structural formulae.

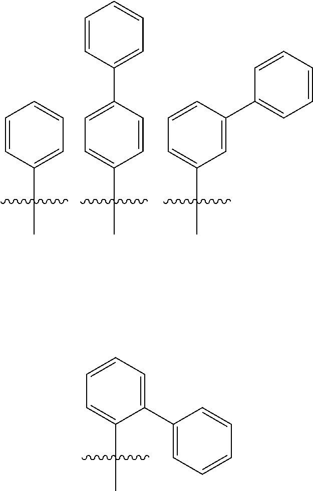

In an exemplary embodiment of the present application, L1 is a direct bond; a substituted or unsubstituted C6 to C60 arylene group; or a substituted or unsubstituted C2 to C60 heteroarylene group.

In another exemplary embodiment, L1 is a direct bond; a substituted or unsubstituted C6 to C40 arylene group; or a substituted or unsubstituted C2 to C40 heteroarylene group.

In still another exemplary embodiment, L1 is a direct bond; or a substituted or unsubstituted C6 to C40 arylene group.

In yet another exemplary embodiment, L1 is a direct bond; or a C6 to C40 arylene group.

In still yet another exemplary embodiment, L1 is a direct bond; or a C6 to C20 arylene group.

In a further exemplary embodiment, L1 may be a direct bond; a substituted or unsubstituted phenylene group; or a substituted or unsubstituted biphenylene group.

In another further exemplary embodiment, L1 may be a direct bond; a phenylene group; or a biphenylene group.

In an exemplary embodiment of the present application, R11 to R13 are the same as or different from each other, and may be each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, R11 to R13 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, R11 to R13 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C40 heterocycloalkyl group; a substituted or unsubstituted C6 to C20 aryl group; or a substituted or unsubstituted C2 to C20 heteroaryl group.

In yet another exemplary embodiment, R11 to R13 are the same as or different from each other, and may be each independently hydrogen; deuterium; a halogen group; a cyano group; a C1 to C20 alkyl group; a C3 to C20 cycloalkyl group; a C2 to C40 heterocycloalkyl group; a C6 to C20 aryl group; or a C2 to C20 heteroaryl group.

In still yet another exemplary embodiment, R11 to R13 are the same as or different from each other, and may be each independently hydrogen; or deuterium.

In an exemplary embodiment of the present application, provided is a heterocyclic compound in which Chemical Formula 1 is represented by any one of the following Chemical Formulae 3 to 6.

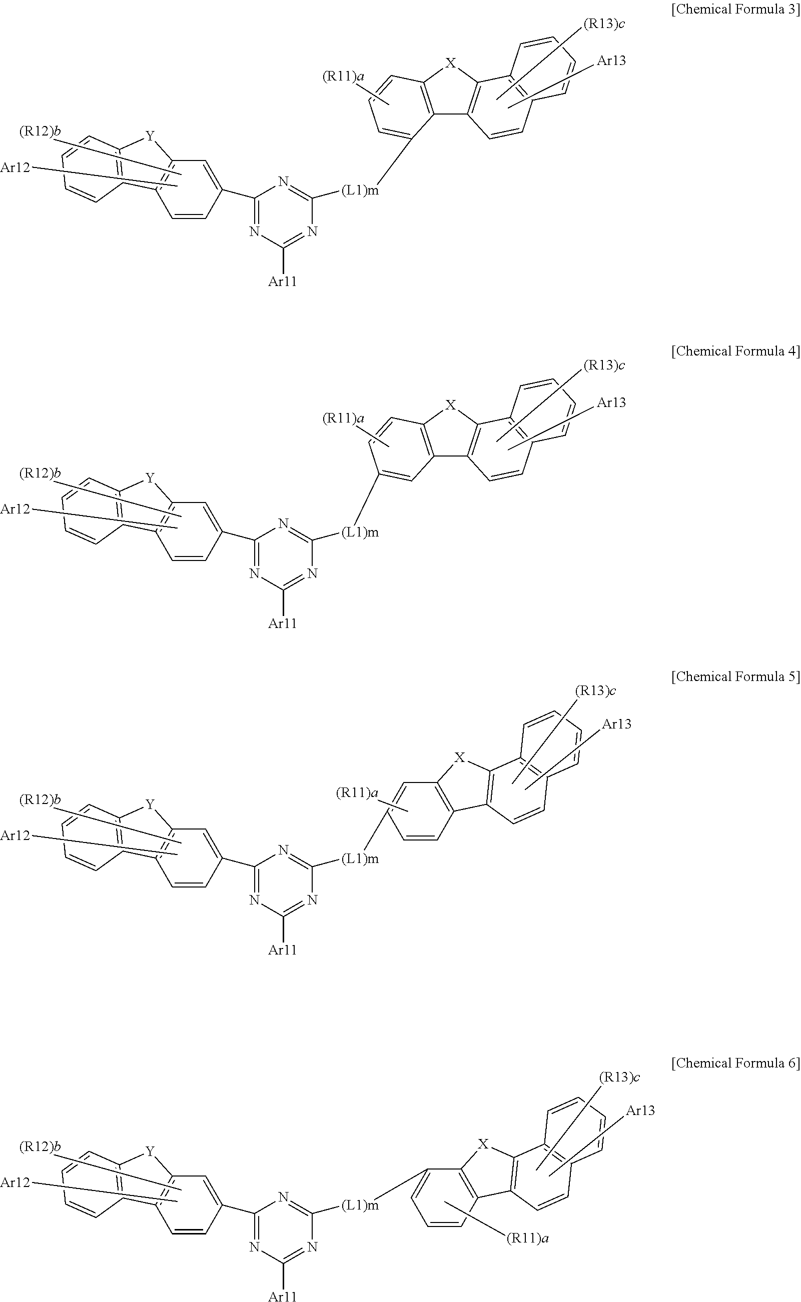

In Chemical Formulae 3 to 6,
the definition of each substituent is the same as the definition in Chemical Formula 1.

In an exemplary embodiment of the present application, provided is a heterocyclic compound in which the

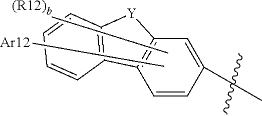

of Chemical Formula 1 is represented by any one of the following Chemical Formulae 1-1 to 1-5.

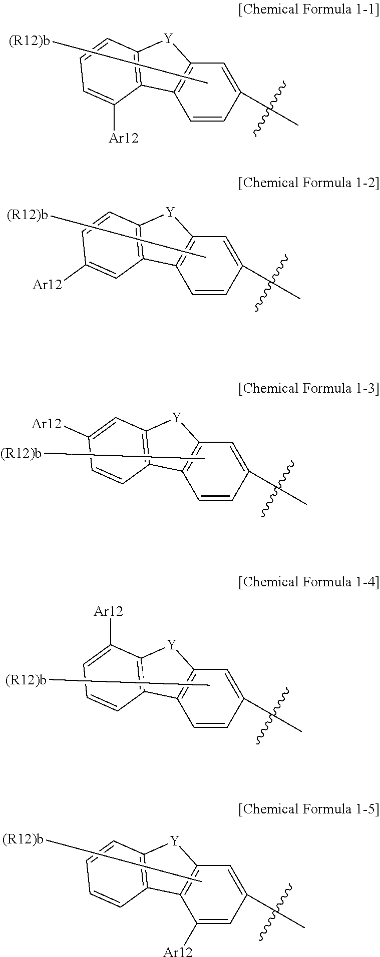

In Chemical Formulae 1-1 to 1-5,

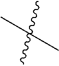

means a position linked to Chemical Formula 1, and
the definition of each substituent is the same as the definition in Chemical Formula 1.

In an exemplary embodiment of the present application, provided is a heterocyclic compound in which the

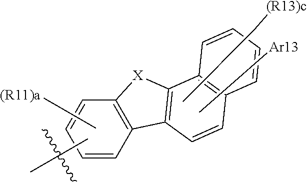

of Chemical Formula 1 is represented by any one of the following Chemical Formulae 2-1 to 2-5.

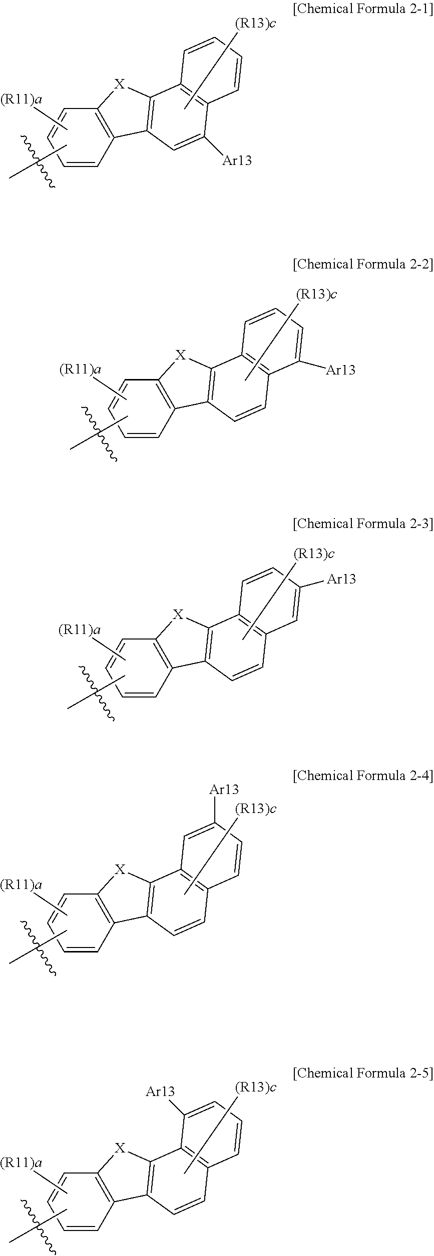

In Chemical Formulae 2-1 to 2-5,

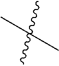

means a position linked to Chemical Formula 1, and
the definition of each substituent is the same as the definition in Chemical Formula 1.

In an exemplary embodiment of the present specification, Chemical Formula 1 may be represented by any one of the following compounds.

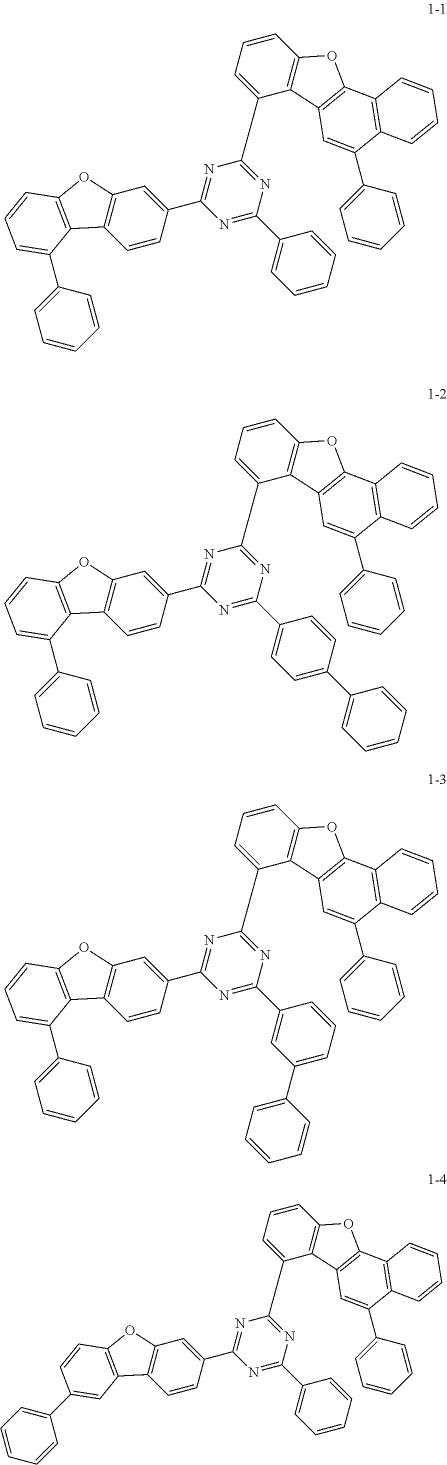 [SMILES: c1ccc(-c2nc(-c3ccc4c(c3)oc3cccc(-c5ccccc5)c34)nc(-c3cccc4oc5c6ccccc6c(-c6ccccc6)cc5c34)n2)cc1 ; c1ccc(-c2ccc(-c3nc(-c4ccc5c(c4)oc4cccc(-c6ccccc6)c45)nc(-c4cccc5oc6c7ccccc7c(-c7ccccc7)cc6c45)n3)cc2)cc1 ; c1ccc(-c2cccc(-c3nc(-c4ccc5c(c4)oc4cccc(-c6ccccc6)c45)nc(-c4cccc5oc6c7ccccc7c(-c7ccccc7)cc6c45)n3)c2)cc1 ; c1ccc(-c2ccc3oc4cc(-c5nc(-c6ccccc6)nc(-c6cccc7oc8c9ccccc9c(-c9ccccc9)cc8c67)n5)ccc4c3c2)cc1]

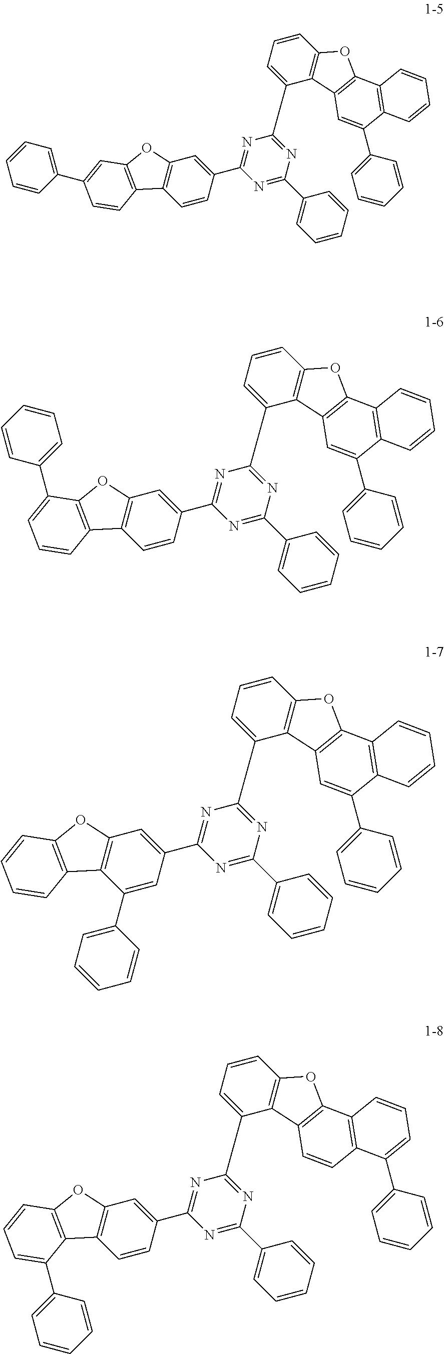 [SMILES: c1ccc(-c2ccc3c(c2)oc2cc(-c4nc(-c5ccccc5)nc(-c5cccc6oc7c8ccccc8c(-c8ccccc8)cc7c56)n4)ccc23)cc1 ; c1ccc(-c2nc(-c3ccc4c(c3)oc3c(-c5ccccc5)cccc34)nc(-c3cccc4oc5c6ccccc6c(-c6ccccc6)cc5c34)n2)cc1 ; c1ccc(-c2nc(-c3cc(-c4ccccc4)c4c(c3)oc3ccccc34)nc(-c3cccc4oc5c6ccccc6c(-c6ccccc6)cc5c34)n2)cc1 ; c1ccc(-c2nc(-c3ccc4c(c3)oc3cccc(-c5ccccc5)c34)nc(-c3cccc4oc5c6cccc(-c7ccccc7)c6ccc5c34)n2)cc1]

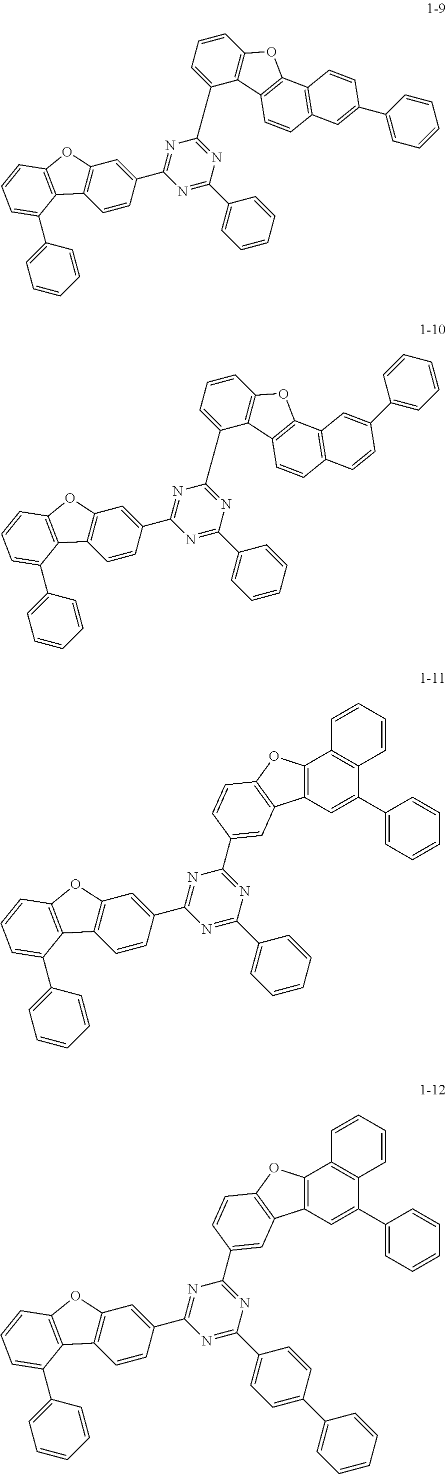 [SMILES: c1ccc(-c2ccc3c(ccc4c3oc3cccc(-c5nc(-c6ccccc6)nc(-c6ccc7c(c6)oc6cccc(-c8ccccc8)c67)n5)c34)c2)cc1 ; c1ccc(-c2ccc3ccc4c(oc5cccc(-c6nc(-c7ccccc7)nc(-c7ccc8c(c7)oc7cccc(-c9ccccc9)c78)n6)c54)c3c2)cc1 ; c1ccc(-c2nc(-c3ccc4c(c3)oc3cccc(-c5ccccc5)c34)nc(-c3ccc4oc5c6ccccc6c(-c6ccccc6)cc5c4c3)n2)cc1 ; c1ccc(-c2ccc(-c3nc(-c4ccc5c(c4)oc4cccc(-c6ccccc6)c45)nc(-c4ccc5oc6c7ccccc7c(-c7ccccc7)cc6c5c4)n3)cc2)cc1]

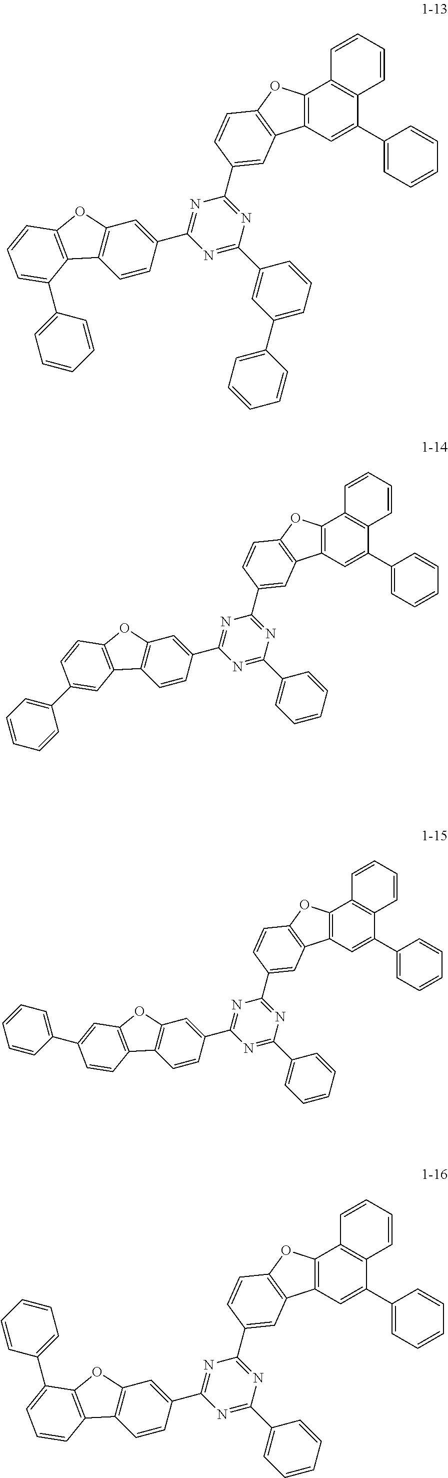 [SMILES: c1ccc(-c2cccc(-c3nc(-c4ccc5c(c4)oc4cccc(-c6ccccc6)c45)nc(-c4ccc5oc6c7ccccc7c(-c7ccccc7)cc6c5c4)n3)c2)cc1 ; c1ccc(-c2ccc3oc4cc(-c5nc(-c6ccccc6)nc(-c6ccc7oc8c9ccccc9c(-c9ccccc9)cc8c7c6)n5)ccc4c3c2)cc1 ; c1ccc(-c2ccc3c(c2)oc2cc(-c4nc(-c5ccccc5)nc(-c5ccc6oc7c8ccccc8c(-c8ccccc8)cc7c6c5)n4)ccc23)cc1 ; c1ccc(-c2nc(-c3ccc4c(c3)oc3c(-c5ccccc5)cccc34)nc(-c3ccc4oc5c6ccccc6c(-c6ccccc6)cc5c4c3)n2)cc1]

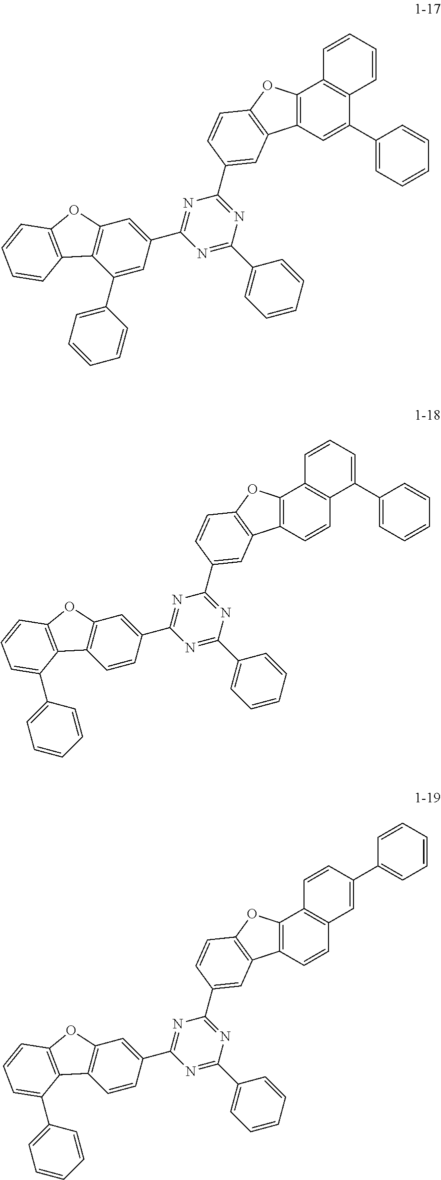 [SMILES: c1ccc(-c2nc(-c3cc(-c4ccccc4)c4c(c3)oc3ccccc34)nc(-c3ccc4oc5c6ccccc6c(-c6ccccc6)cc5c4c3)n2)cc1 ; c1ccc(-c2nc(-c3ccc4c(c3)oc3cccc(-c5ccccc5)c34)nc(-c3ccc4oc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)oc7cccc(-c9ccccc9)c78)n6)ccc5oc34)c2)cc1]

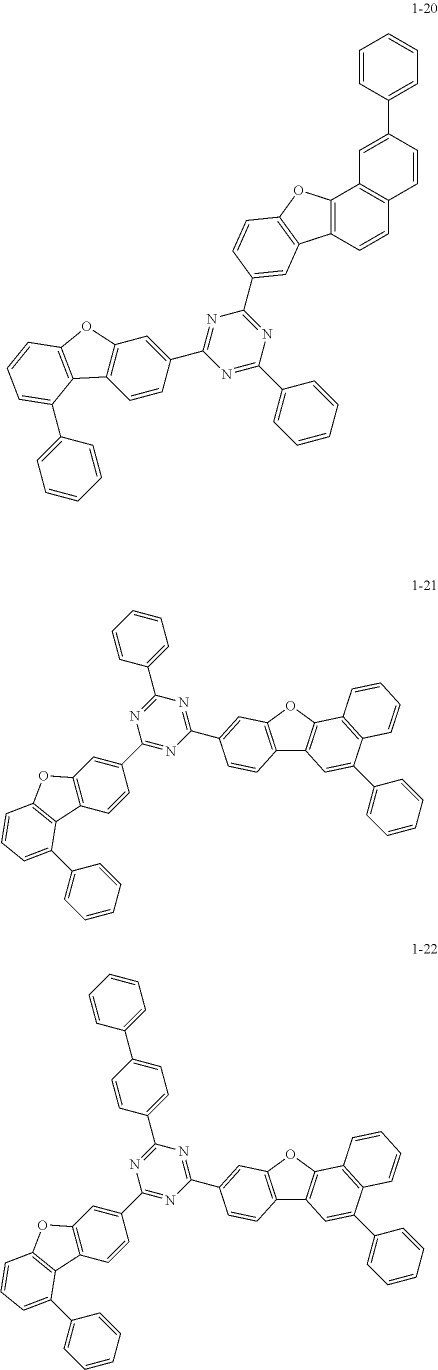 [SMILES: c1ccc(-c2ccc3ccc4c5cc(-c6nc(-c7ccccc7)nc(-c7ccc8c(c7)oc7cccc(-c9ccccc9)c78)n6)ccc5oc4c3c2)cc1 ; c1ccc(-c2nc(-c3ccc4c(c3)oc3c5ccccc5c(-c5ccccc5)cc43)nc(-c3ccc4c(c3)oc3cccc(-c5ccccc5)c34)n2)cc1 ; c1ccc(-c2ccc(-c3nc(-c4ccc5c(c4)oc4c6ccccc6c(-c6ccccc6)cc54)nc(-c4ccc5c(c4)oc4cccc(-c6ccccc6)c45)n3)cc2)cc1]

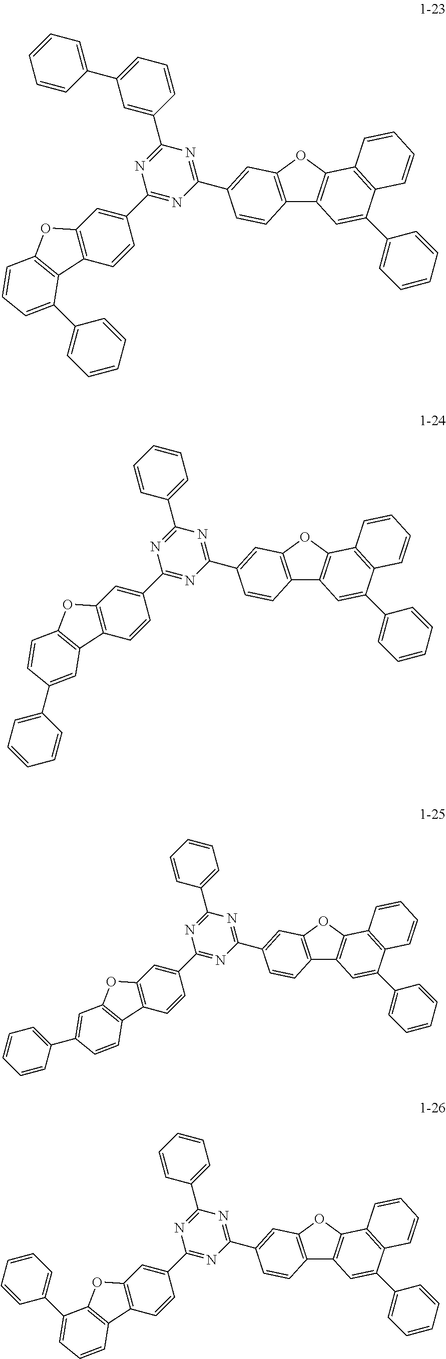 [SMILES: c1ccc(-c2cccc(-c3nc(-c4ccc5c(c4)oc4c6ccccc6c(-c6ccccc6)cc54)nc(-c4ccc5c(c4)oc4cccc(-c6ccccc6)c45)n3)c2)cc1 ; c1ccc(-c2ccc3oc4cc(-c5nc(-c6ccccc6)nc(-c6ccc7c(c6)oc6c8ccccc8c(-c8ccccc8)cc76)n5)ccc4c3c2)cc1 ; c1ccc(-c2ccc3c(c2)oc2cc(-c4nc(-c5ccccc5)nc(-c5ccc6c(c5)oc5c7ccccc7c(-c7ccccc7)cc65)n4)ccc23)cc1 ; c1ccc(-c2nc(-c3ccc4c(c3)oc3c(-c5ccccc5)cccc34)nc(-c3ccc4c(c3)oc3c5ccccc5c(-c5ccccc5)cc43)n2)cc1]

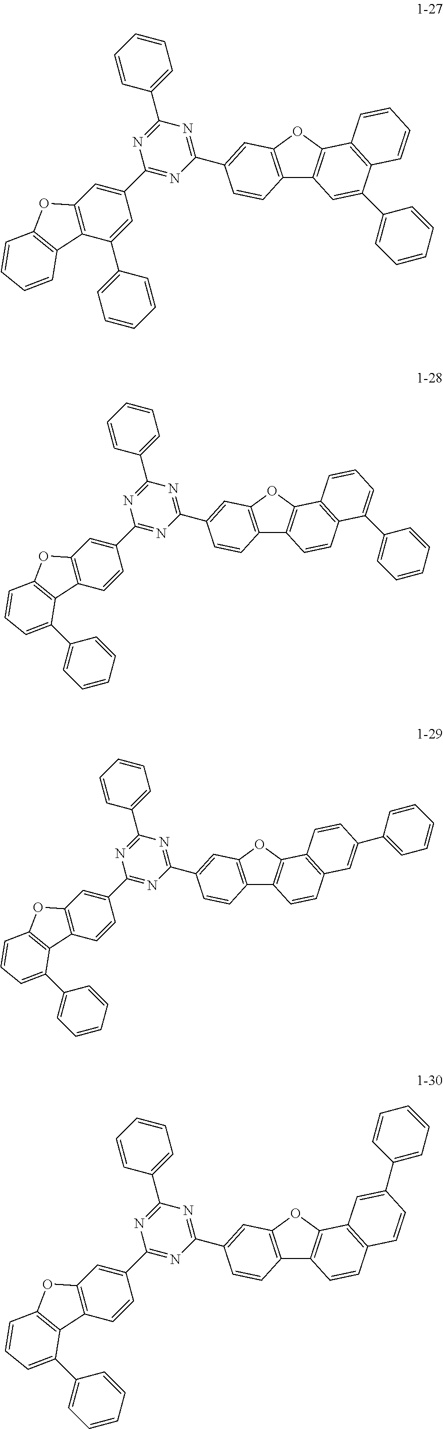 [SMILES: c1ccc(-c2nc(-c3ccc4c(c3)oc3c5ccccc5c(-c5ccccc5)cc43)nc(-c3cc(-c4ccccc4)c4c(c3)oc3ccccc34)n2)cc1 ; c1ccc(-c2nc(-c3ccc4c(c3)oc3c5cccc(-c6ccccc6)c5ccc43)nc(-c3ccc4c(c3)oc3cccc(-c5ccccc5)c34)n2)cc1 ; c1ccc(-c2ccc3c(ccc4c5ccc(-c6nc(-c7ccccc7)nc(-c7ccc8c(c7)oc7cccc(-c9ccccc9)c78)n6)cc5oc34)c2)cc1 ; c1ccc(-c2ccc3ccc4c5ccc(-c6nc(-c7ccccc7)nc(-c7ccc8c(c7)oc7cccc(-c9ccccc9)c78)n6)cc5oc4c3c2)cc1]

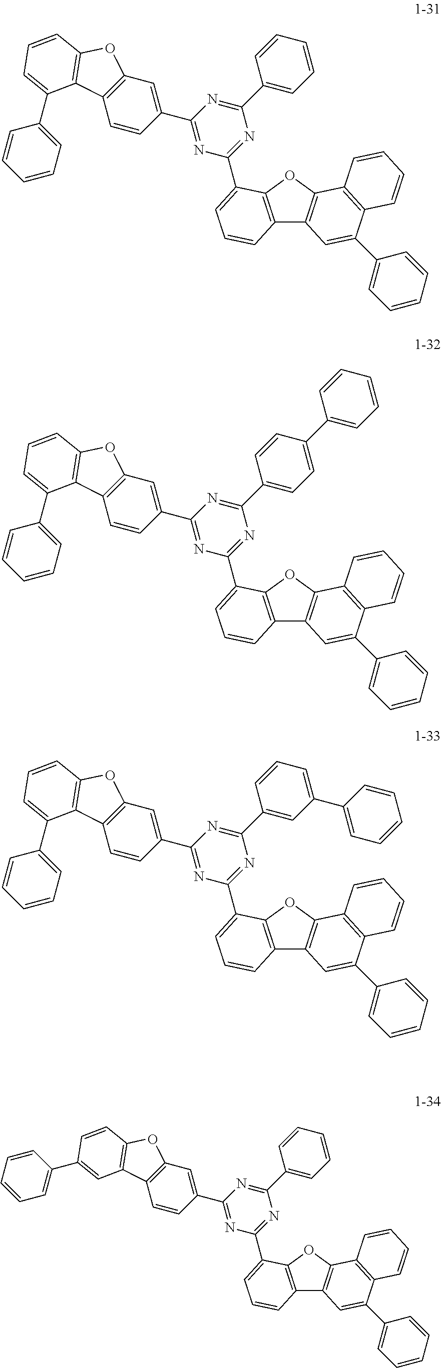 [SMILES: c1ccc(-c2nc(-c3ccc4c(c3)oc3cccc(-c5ccccc5)c34)nc(-c3cccc4c3oc3c5ccccc5c(-c5ccccc5)cc43)n2)cc1 ; c1ccc(-c2ccc(-c3nc(-c4ccc5c(c4)oc4cccc(-c6ccccc6)c45)nc(-c4cccc5c4oc4c6ccccc6c(-c6ccccc6)cc54)n3)cc2)cc1 ; c1ccc(-c2cccc(-c3nc(-c4ccc5c(c4)oc4cccc(-c6ccccc6)c45)nc(-c4cccc5c4oc4c6ccccc6c(-c6ccccc6)cc54)n3)c2)cc1 ; c1ccc(-c2ccc3oc4cc(-c5nc(-c6ccccc6)nc(-c6cccc7c6oc6c8ccccc8c(-c8ccccc8)cc76)n5)ccc4c3c2)cc1]

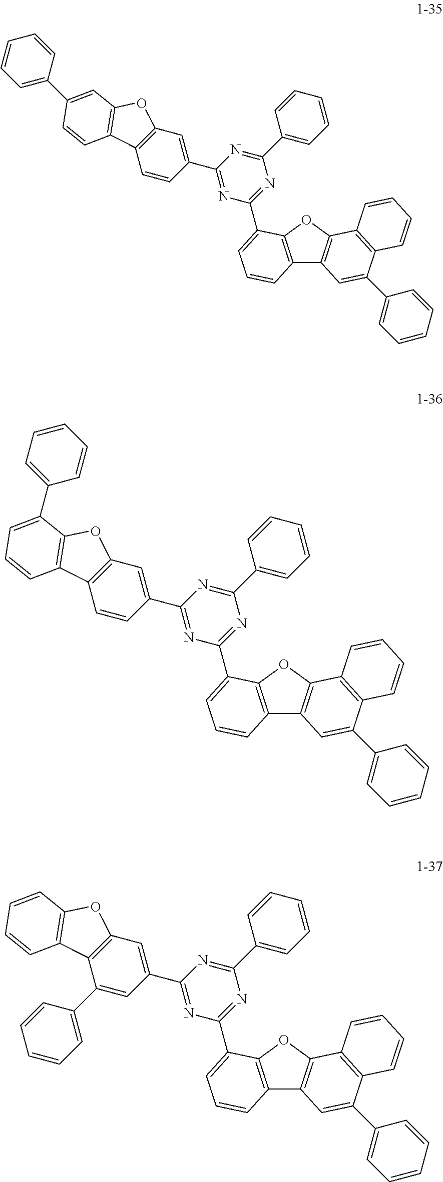 [SMILES: c1ccc(-c2ccc3c(c2)oc2cc(-c4nc(-c5ccccc5)nc(-c5cccc6c5oc5c7ccccc7c(-c7ccccc7)cc65)n4)ccc23)cc1 ; c1ccc(-c2nc(-c3ccc4c(c3)oc3c(-c5ccccc5)cccc34)nc(-c3cccc4c3oc3c5ccccc5c(-c5ccccc5)cc43)n2)cc1 ; c1ccc(-c2nc(-c3cc(-c4ccccc4)c4c(c3)oc3ccccc34)nc(-c3cccc4c3oc3c5ccccc5c(-c5ccccc5)cc43)n2)cc1]

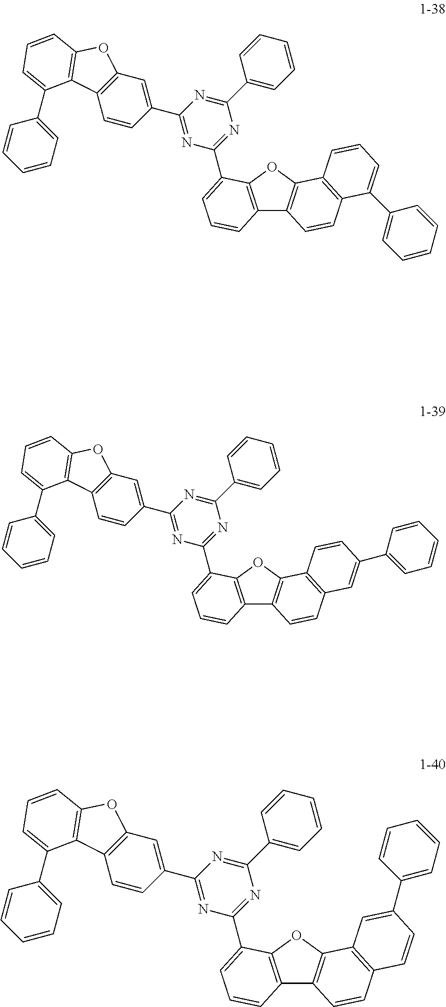 [SMILES: c1ccc(-c2nc(-c3ccc4c(c3)oc3cccc(-c5ccccc5)c34)nc(-c3cccc4c3oc3c5cccc(-c6ccccc6)c5ccc43)n2)cc1 ; c1ccc(-c2ccc3c(ccc4c5cccc(-c6nc(-c7ccccc7)nc(-c7ccc8c(c7)oc7cccc(-c9ccccc9)c78)n6)c5oc34)c2)cc1 ; c1ccc(-c2ccc3ccc4c5cccc(-c6nc(-c7ccccc7)nc(-c7ccc8c(c7)oc7cccc(-c9ccccc9)c78)n6)c5oc4c3c2)cc1]

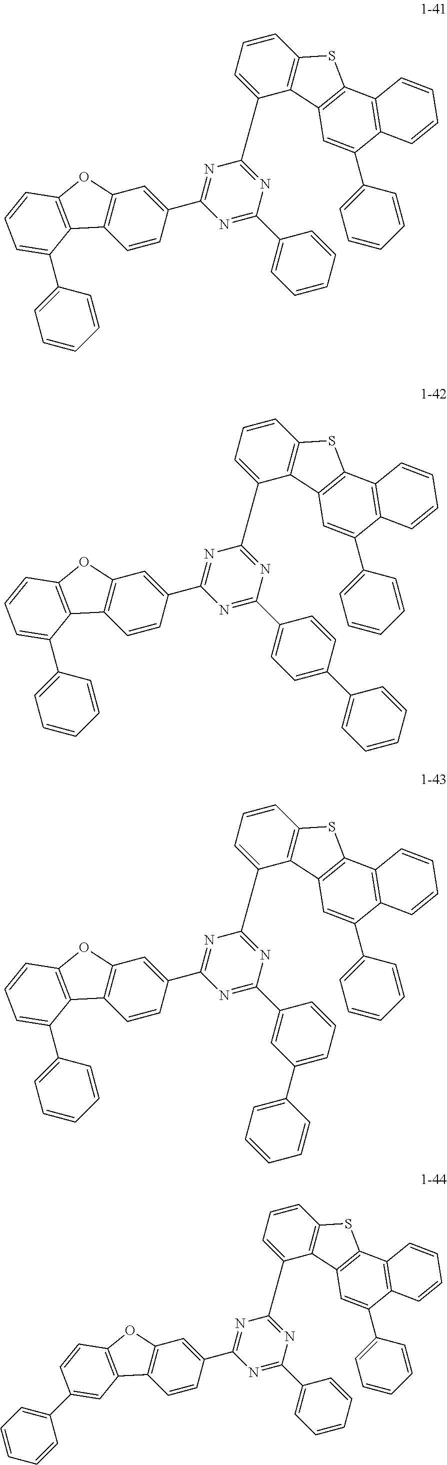 [SMILES: c1ccc(-c2nc(-c3ccc4c(c3)oc3cccc(-c5ccccc5)c34)nc(-c3cccc4sc5c6ccccc6c(-c6ccccc6)cc5c34)n2)cc1 ; c1ccc(-c2ccc(-c3nc(-c4ccc5c(c4)oc4cccc(-c6ccccc6)c45)nc(-c4cccc5sc6c7ccccc7c(-c7ccccc7)cc6c45)n3)cc2)cc1 ; c1ccc(-c2cccc(-c3nc(-c4ccc5c(c4)oc4cccc(-c6ccccc6)c45)nc(-c4cccc5sc6c7ccccc7c(-c7ccccc7)cc6c45)n3)c2)cc1 ; c1ccc(-c2ccc3oc4cc(-c5nc(-c6ccccc6)nc(-c6cccc7sc8c9ccccc9c(-c9ccccc9)cc8c67)n5)ccc4c3c2)cc1]

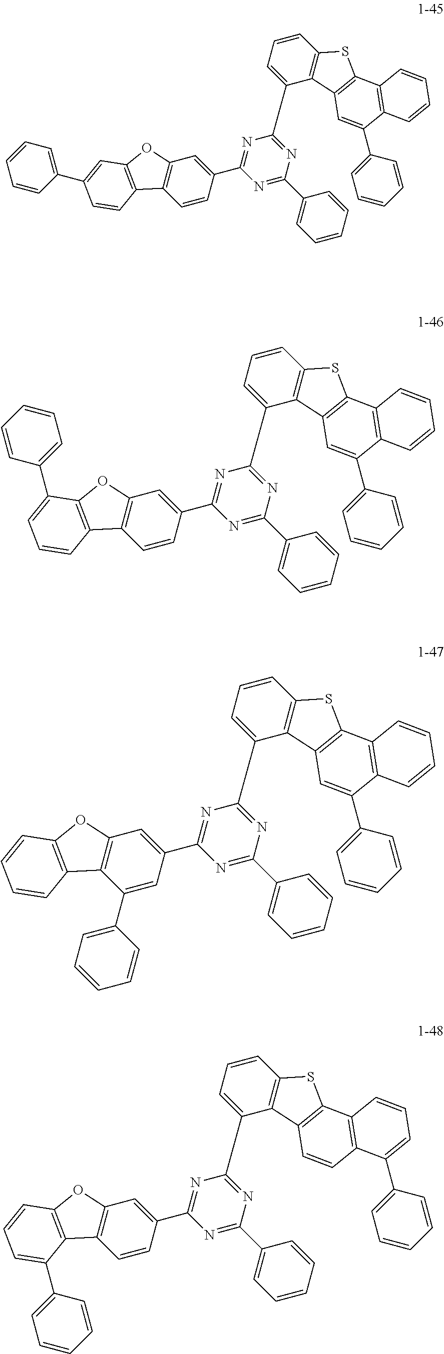 [SMILES: c1ccc(-c2ccc3c(c2)oc2cc(-c4nc(-c5ccccc5)nc(-c5cccc6sc7c8ccccc8c(-c8ccccc8)cc7c56)n4)ccc23)cc1 ; c1ccc(-c2nc(-c3ccc4c(c3)oc3c(-c5ccccc5)cccc34)nc(-c3cccc4sc5c6ccccc6c(-c6ccccc6)cc5c34)n2)cc1 ; c1ccc(-c2nc(-c3cc(-c4ccccc4)c4c(c3)oc3ccccc34)nc(-c3cccc4sc5c6ccccc6c(-c6ccccc6)cc5c34)n2)cc1 ; c1ccc(-c2nc(-c3ccc4c(c3)oc3cccc(-c5ccccc5)c34)nc(-c3cccc4sc5c6cccc(-c7ccccc7)c6ccc5c34)n2)cc1]

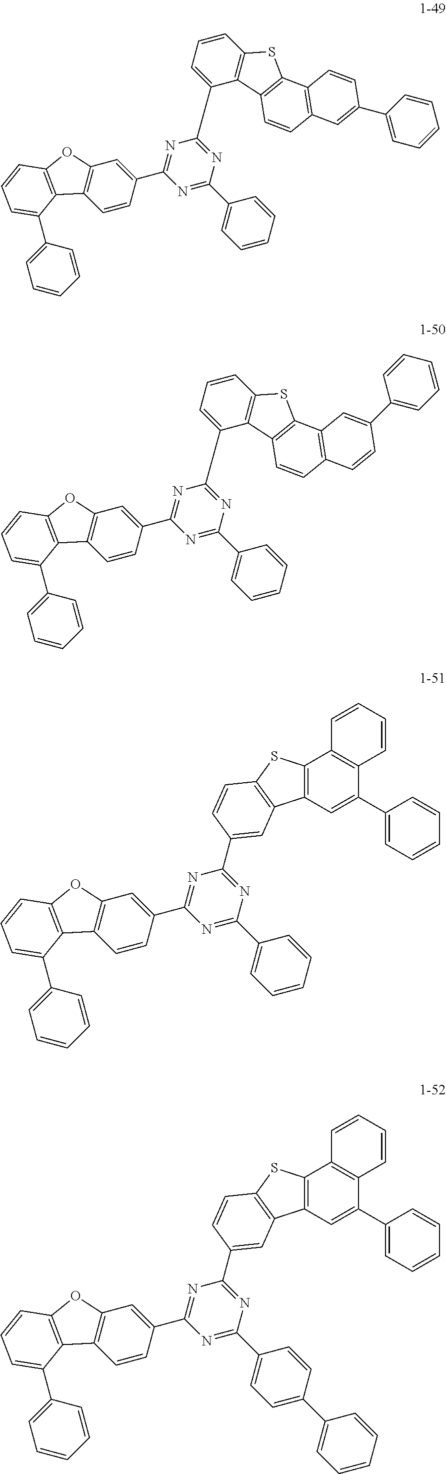 [SMILES: c1ccc(-c2ccc3c(ccc4c3sc3cccc(-c5nc(-c6ccccc6)nc(-c6ccc7c(c6)oc6cccc(-c8ccccc8)c67)n5)c34)c2)cc1 ; c1ccc(-c2ccc3ccc4c(sc5cccc(-c6nc(-c7ccccc7)nc(-c7ccc8c(c7)oc7cccc(-c9ccccc9)c78)n6)c54)c3c2)cc1 ; c1ccc(-c2nc(-c3ccc4c(c3)oc3cccc(-c5ccccc5)c34)nc(-c3ccc4sc5c6ccccc6c(-c6ccccc6)cc5c4c3)n2)cc1 ; c1ccc(-c2ccc(-c3nc(-c4ccc5c(c4)oc4cccc(-c6ccccc6)c45)nc(-c4ccc5sc6c7ccccc7c(-c7ccccc7)cc6c5c4)n3)cc2)cc1]

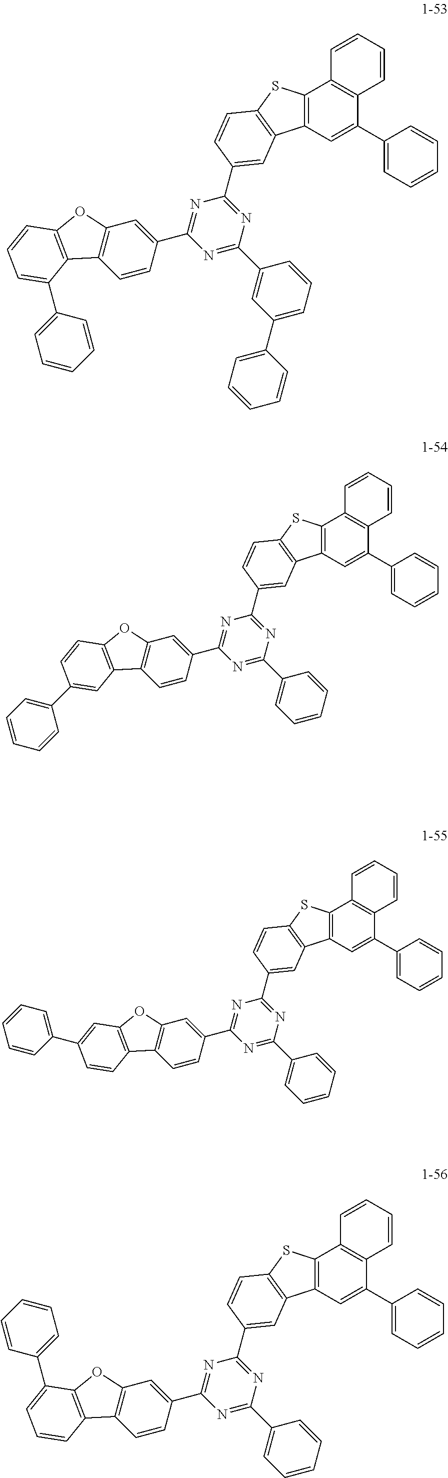 [SMILES: c1ccc(-c2cccc(-c3nc(-c4ccc5c(c4)oc4cccc(-c6ccccc6)c45)nc(-c4ccc5sc6c7ccccc7c(-c7ccccc7)cc6c5c4)n3)c2)cc1 ; c1ccc(-c2ccc3oc4cc(-c5nc(-c6ccccc6)nc(-c6ccc7sc8c9ccccc9c(-c9ccccc9)cc8c7c6)n5)ccc4c3c2)cc1 ; c1ccc(-c2ccc3c(c2)oc2cc(-c4nc(-c5ccccc5)nc(-c5ccc6sc7c8ccccc8c(-c8ccccc8)cc7c6c5)n4)ccc23)cc1 ; c1ccc(-c2nc(-c3ccc4c(c3)oc3c(-c5ccccc5)cccc34)nc(-c3ccc4sc5c6ccccc6c(-c6ccccc6)cc5c4c3)n2)cc1]

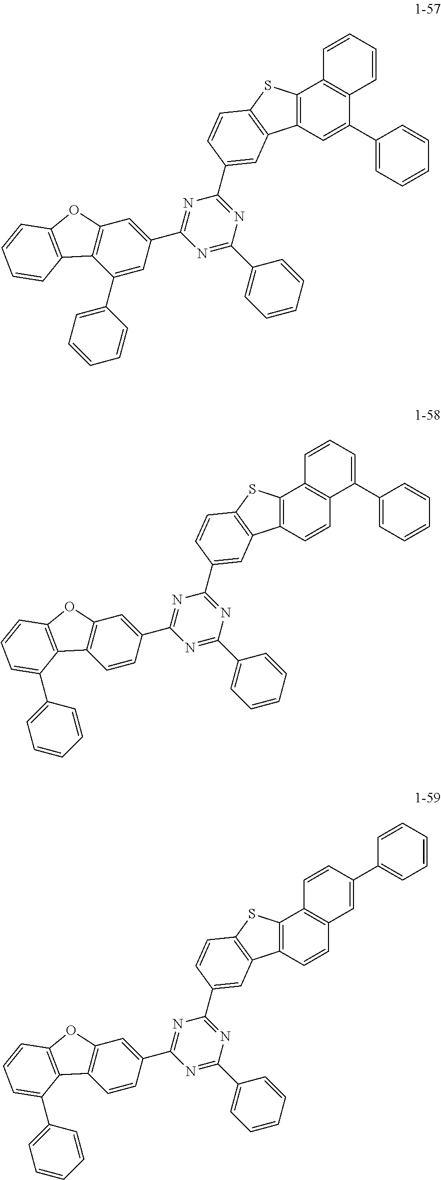 [SMILES: c1ccc(-c2nc(-c3cc(-c4ccccc4)c4c(c3)oc3ccccc34)nc(-c3ccc4sc5c6ccccc6c(-c6ccccc6)cc5c4c3)n2)cc1 ; c1ccc(-c2nc(-c3ccc4c(c3)oc3cccc(-c5ccccc5)c34)nc(-c3ccc4sc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)oc7cccc(-c9ccccc9)c78)n6)ccc5sc34)c2)cc1]

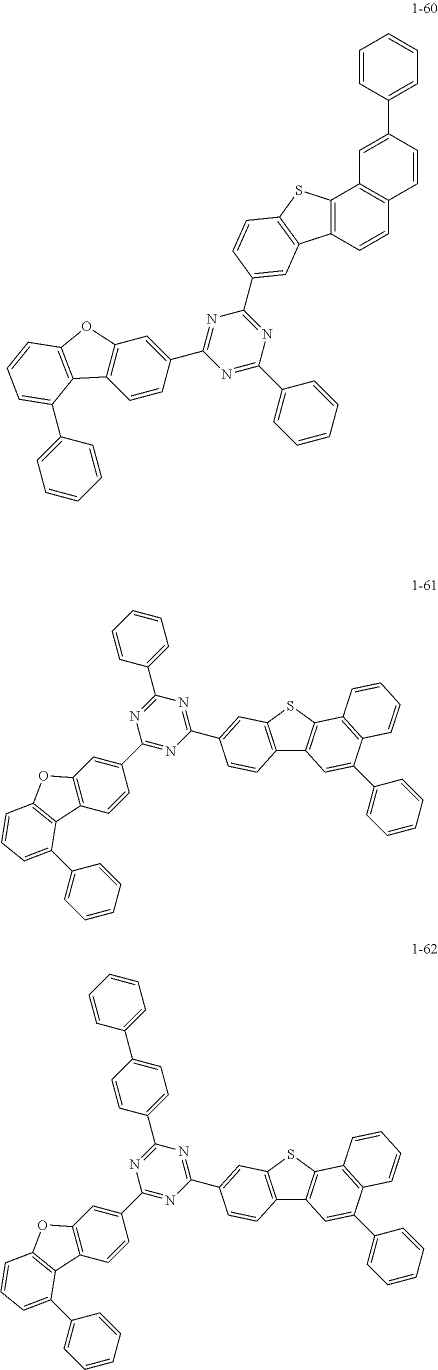 [SMILES: c1ccc(-c2ccc3ccc4c5cc(-c6nc(-c7ccccc7)nc(-c7ccc8c(c7)oc7cccc(-c9ccccc9)c78)n6)ccc5sc4c3c2)cc1 ; c1ccc(-c2nc(-c3ccc4c(c3)oc3cccc(-c5ccccc5)c34)nc(-c3ccc4c(c3)sc3c5ccccc5c(-c5ccccc5)cc43)n2)cc1 ; c1ccc(-c2ccc(-c3nc(-c4ccc5c(c4)oc4cccc(-c6ccccc6)c45)nc(-c4ccc5c(c4)sc4c6ccccc6c(-c6ccccc6)cc54)n3)cc2)cc1]

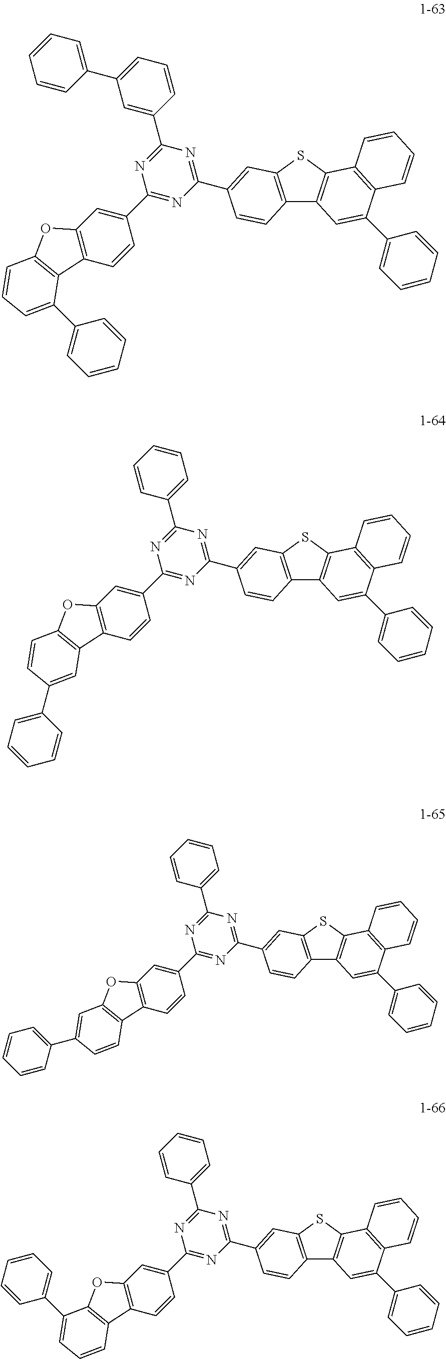 [SMILES: c1ccc(-c2cccc(-c3nc(-c4ccc5c(c4)oc4cccc(-c6ccccc6)c45)nc(-c4ccc5c(c4)sc4c6ccccc6c(-c6ccccc6)cc54)n3)c2)cc1 ; c1ccc(-c2ccc3oc4cc(-c5nc(-c6ccccc6)nc(-c6ccc7c(c6)sc6c8ccccc8c(-c8ccccc8)cc76)n5)ccc4c3c2)cc1 ; c1ccc(-c2ccc3c(c2)oc2cc(-c4nc(-c5ccccc5)nc(-c5ccc6c(c5)sc5c7ccccc7c(-c7ccccc7)cc65)n4)ccc23)cc1 ; c1ccc(-c2nc(-c3ccc4c(c3)oc3c(-c5ccccc5)cccc34)nc(-c3ccc4c(c3)sc3c5ccccc5c(-c5ccccc5)cc43)n2)cc1]

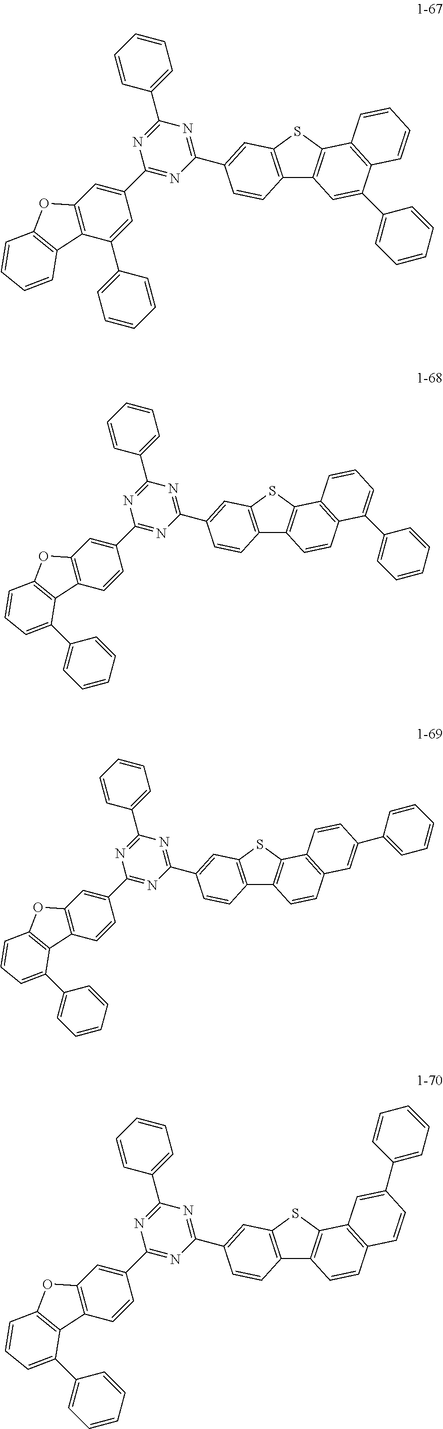 [SMILES: c1ccc(-c2nc(-c3cc(-c4ccccc4)c4c(c3)oc3ccccc34)nc(-c3ccc4c(c3)sc3c5ccccc5c(-c5ccccc5)cc43)n2)cc1 ; c1ccc(-c2nc(-c3ccc4c(c3)oc3cccc(-c5ccccc5)c34)nc(-c3ccc4c(c3)sc3c5cccc(-c6ccccc6)c5ccc43)n2)cc1 ; c1ccc(-c2ccc3c(ccc4c5ccc(-c6nc(-c7ccccc7)nc(-c7ccc8c(c7)oc7cccc(-c9ccccc9)c78)n6)cc5sc34)c2)cc1 ; c1ccc(-c2ccc3ccc4c5ccc(-c6nc(-c7ccccc7)nc(-c7ccc8c(c7)oc7cccc(-c9ccccc9)c78)n6)cc5sc4c3c2)cc1]

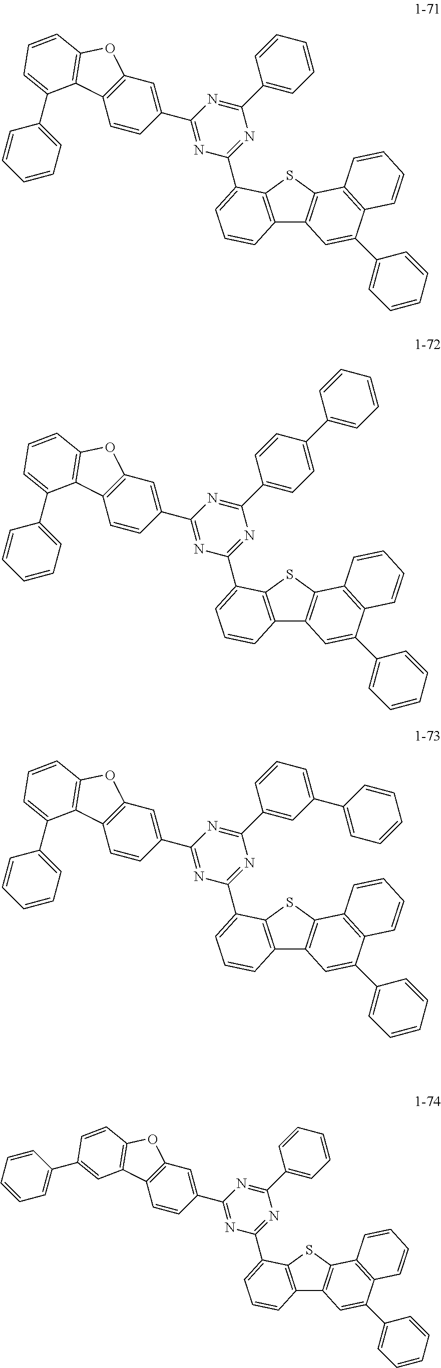 [SMILES: c1ccc(-c2nc(-c3ccc4c(c3)oc3cccc(-c5ccccc5)c34)nc(-c3cccc4c3sc3c5ccccc5c(-c5ccccc5)cc43)n2)cc1 ; c1ccc(-c2ccc(-c3nc(-c4ccc5c(c4)oc4cccc(-c6ccccc6)c45)nc(-c4cccc5c4sc4c6ccccc6c(-c6ccccc6)cc54)n3)cc2)cc1 ; c1ccc(-c2cccc(-c3nc(-c4ccc5c(c4)oc4cccc(-c6ccccc6)c45)nc(-c4cccc5c4sc4c6ccccc6c(-c6ccccc6)cc54)n3)c2)cc1 ; c1ccc(-c2ccc3oc4cc(-c5nc(-c6ccccc6)nc(-c6cccc7c6sc6c8ccccc8c(-c8ccccc8)cc76)n5)ccc4c3c2)cc1]

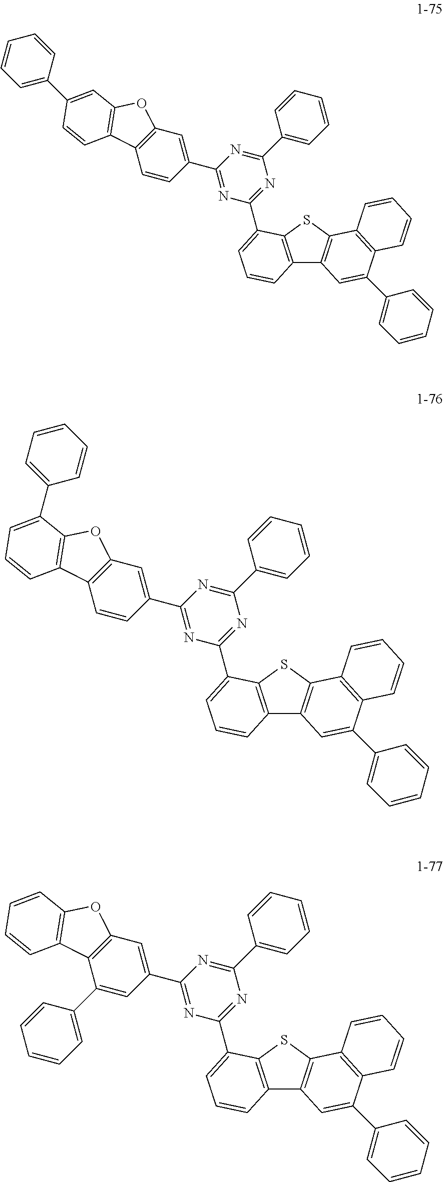 [SMILES: c1ccc(-c2ccc3c(c2)oc2cc(-c4nc(-c5ccccc5)nc(-c5cccc6c5sc5c7ccccc7c(-c7ccccc7)cc65)n4)ccc23)cc1 ; c1ccc(-c2nc(-c3ccc4c(c3)oc3c(-c5ccccc5)cccc34)nc(-c3cccc4c3sc3c5ccccc5c(-c5ccccc5)cc43)n2)cc1 ; c1ccc(-c2nc(-c3cc(-c4ccccc4)c4c(c3)oc3ccccc34)nc(-c3cccc4c3sc3c5ccccc5c(-c5ccccc5)cc43)n2)cc1]

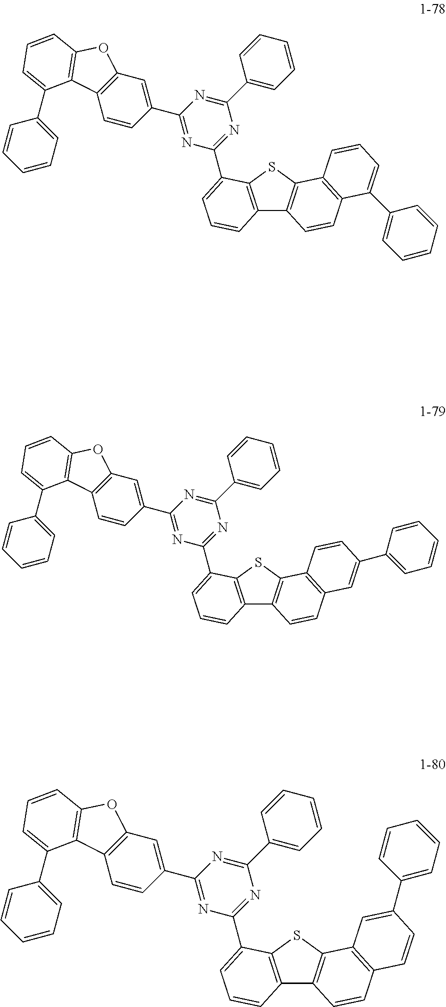 [SMILES: c1ccc(-c2nc(-c3ccc4c(c3)oc3cccc(-c5ccccc5)c34)nc(-c3cccc4c3sc3c5cccc(-c6ccccc6)c5ccc43)n2)cc1 ; c1ccc(-c2ccc3c(ccc4c5cccc(-c6nc(-c7ccccc7)nc(-c7ccc8c(c7)oc7cccc(-c9ccccc9)c78)n6)c5sc34)c2)cc1 ; c1ccc(-c2ccc3ccc4c5cccc(-c6nc(-c7ccccc7)nc(-c7ccc8c(c7)oc7cccc(-c9ccccc9)c78)n6)c5sc4c3c2)cc1]

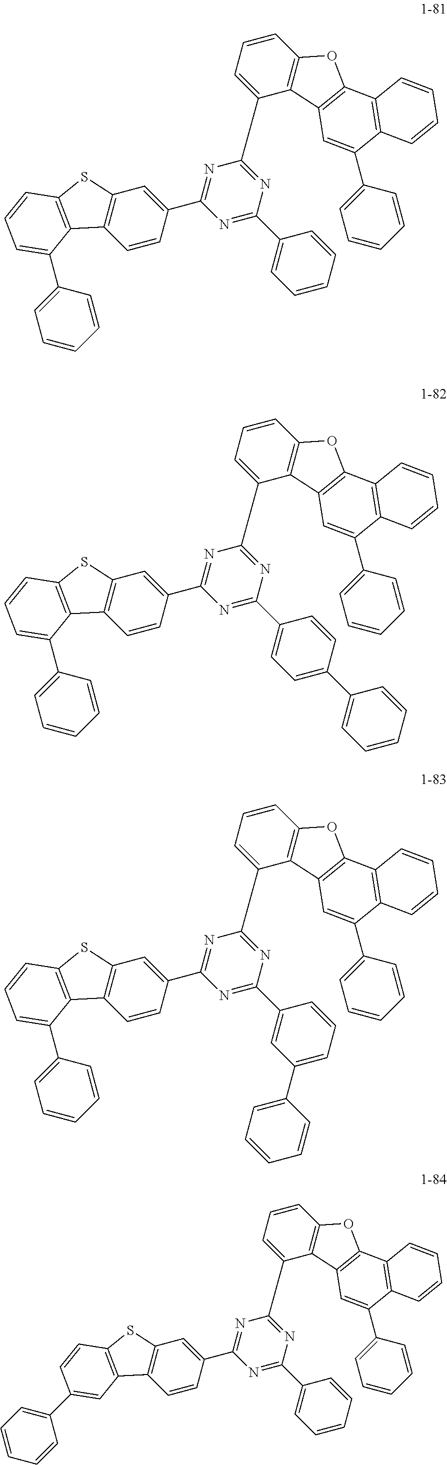 [SMILES: c1ccc(-c2nc(-c3ccc4c(c3)sc3cccc(-c5ccccc5)c34)nc(-c3cccc4oc5c6ccccc6c(-c6ccccc6)cc5c34)n2)cc1 ; c1ccc(-c2ccc(-c3nc(-c4ccc5c(c4)sc4cccc(-c6ccccc6)c45)nc(-c4cccc5oc6c7ccccc7c(-c7ccccc7)cc6c45)n3)cc2)cc1 ; c1ccc(-c2cccc(-c3nc(-c4ccc5c(c4)sc4cccc(-c6ccccc6)c45)nc(-c4cccc5oc6c7ccccc7c(-c7ccccc7)cc6c45)n3)c2)cc1 ; c1ccc(-c2ccc3sc4cc(-c5nc(-c6ccccc6)nc(-c6cccc7oc8c9ccccc9c(-c9ccccc9)cc8c67)n5)ccc4c3c2)cc1]

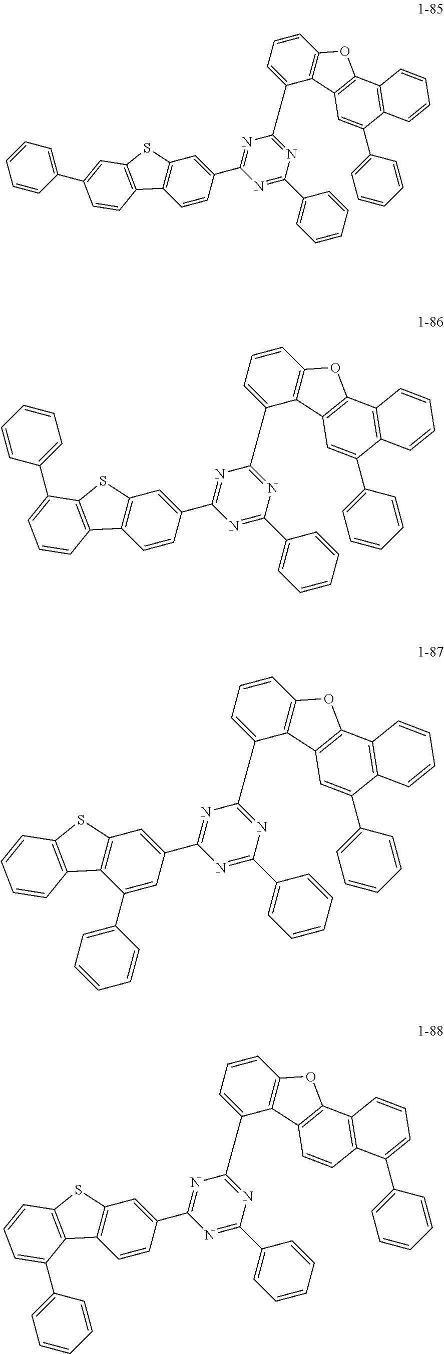 [SMILES: c1ccc(-c2ccc3c(c2)sc2cc(-c4nc(-c5ccccc5)nc(-c5cccc6oc7c8ccccc8c(-c8ccccc8)cc7c56)n4)ccc23)cc1 ; c1ccc(-c2nc(-c3ccc4c(c3)sc3c(-c5ccccc5)cccc34)nc(-c3cccc4oc5c6ccccc6c(-c6ccccc6)cc5c34)n2)cc1 ; c1ccc(-c2nc(-c3cc(-c4ccccc4)c4c(c3)sc3ccccc34)nc(-c3cccc4oc5c6ccccc6c(-c6ccccc6)cc5c34)n2)cc1 ; c1ccc(-c2nc(-c3ccc4c(c3)sc3cccc(-c5ccccc5)c34)nc(-c3cccc4oc5c6cccc(-c7ccccc7)c6ccc5c34)n2)cc1]

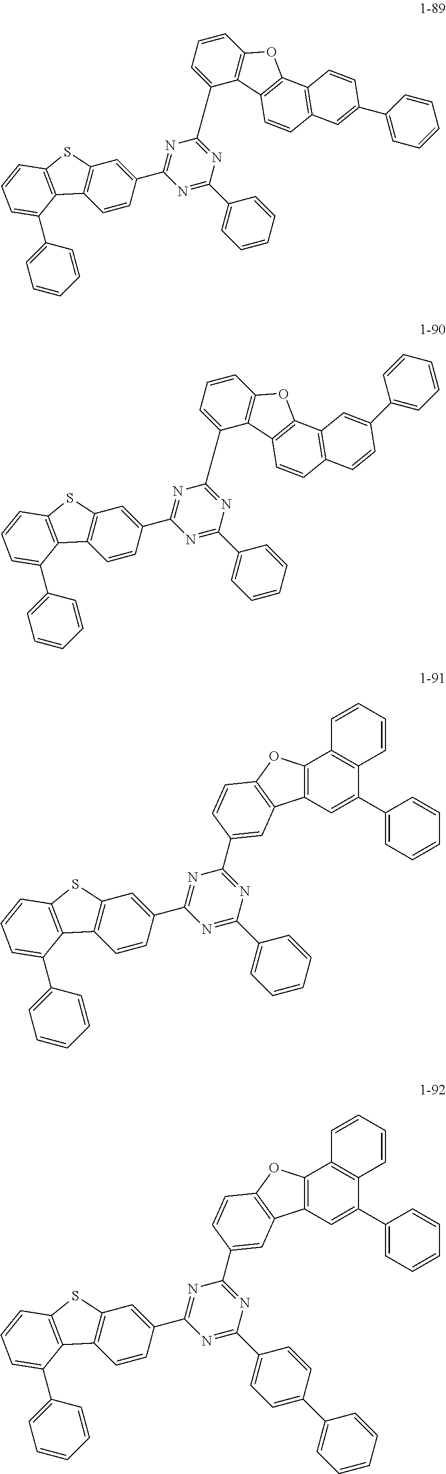 [SMILES: c1ccc(-c2ccc3c(ccc4c3oc3cccc(-c5nc(-c6ccccc6)nc(-c6ccc7c(c6)sc6cccc(-c8ccccc8)c67)n5)c34)c2)cc1 ; c1ccc(-c2ccc3ccc4c(oc5cccc(-c6nc(-c7ccccc7)nc(-c7ccc8c(c7)sc7cccc(-c9ccccc9)c78)n6)c54)c3c2)cc1 ; c1ccc(-c2nc(-c3ccc4c(c3)sc3cccc(-c5ccccc5)c34)nc(-c3ccc4oc5c6ccccc6c(-c6ccccc6)cc5c4c3)n2)cc1 ; c1ccc(-c2ccc(-c3nc(-c4ccc5c(c4)sc4cccc(-c6ccccc6)c45)nc(-c4ccc5oc6c7ccccc7c(-c7ccccc7)cc6c5c4)n3)cc2)cc1]

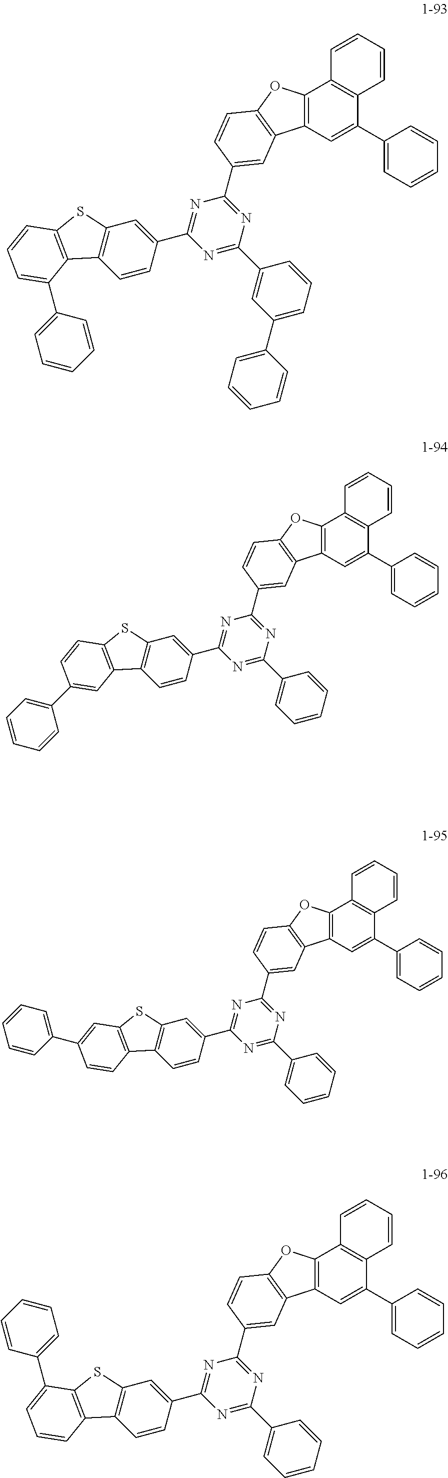 [SMILES: c1ccc(-c2cccc(-c3nc(-c4ccc5c(c4)sc4cccc(-c6ccccc6)c45)nc(-c4ccc5oc6c7ccccc7c(-c7ccccc7)cc6c5c4)n3)c2)cc1 ; c1ccc(-c2ccc3sc4cc(-c5nc(-c6ccccc6)nc(-c6ccc7oc8c9ccccc9c(-c9ccccc9)cc8c7c6)n5)ccc4c3c2)cc1 ; c1ccc(-c2ccc3c(c2)sc2cc(-c4nc(-c5ccccc5)nc(-c5ccc6oc7c8ccccc8c(-c8ccccc8)cc7c6c5)n4)ccc23)cc1 ; c1ccc(-c2nc(-c3ccc4c(c3)sc3c(-c5ccccc5)cccc34)nc(-c3ccc4oc5c6ccccc6c(-c6ccccc6)cc5c4c3)n2)cc1]

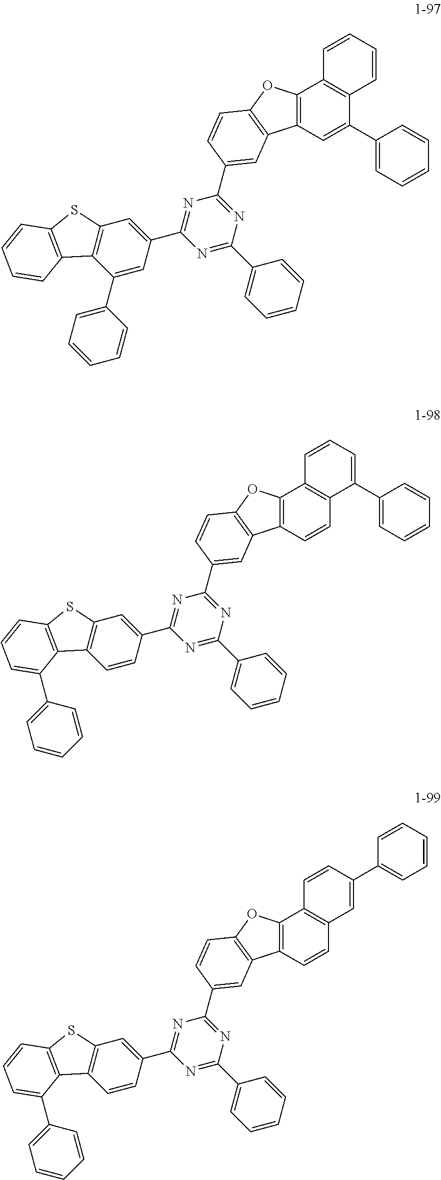 [SMILES: c1ccc(-c2nc(-c3cc(-c4ccccc4)c4c(c3)sc3ccccc34)nc(-c3ccc4oc5c6ccccc6c(-c6ccccc6)cc5c4c3)n2)cc1 ; c1ccc(-c2nc(-c3ccc4c(c3)sc3cccc(-c5ccccc5)c34)nc(-c3ccc4oc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)sc7cccc(-c9ccccc9)c78)n6)ccc5oc34)c2)cc1]

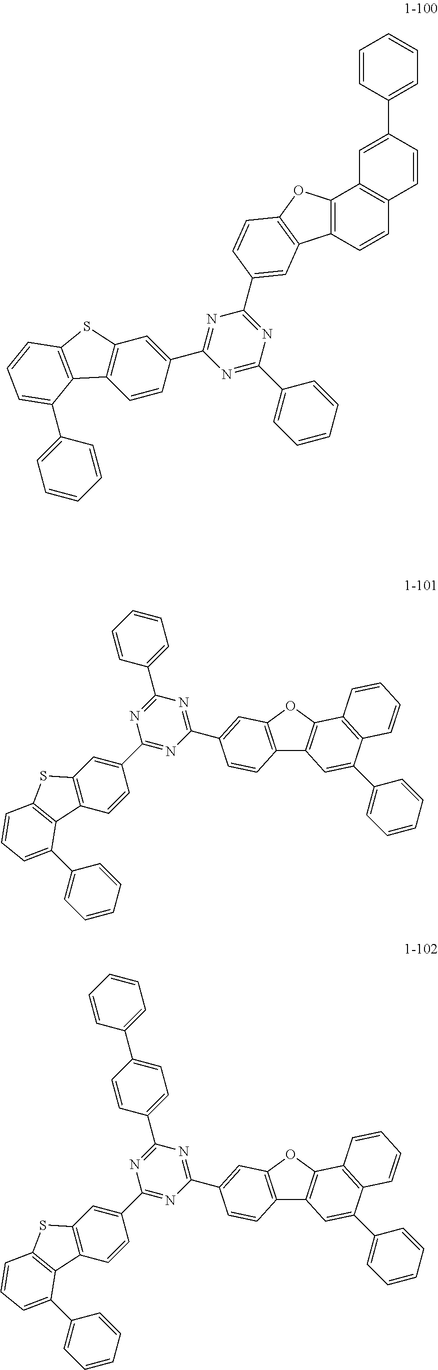 [SMILES: c1ccc(-c2ccc3ccc4c5cc(-c6nc(-c7ccccc7)nc(-c7ccc8c(c7)sc7cccc(-c9ccccc9)c78)n6)ccc5oc4c3c2)cc1 ; c1ccc(-c2nc(-c3ccc4c(c3)oc3c5ccccc5c(-c5ccccc5)cc43)nc(-c3ccc4c(c3)sc3cccc(-c5ccccc5)c34)n2)cc1 ; c1ccc(-c2ccc(-c3nc(-c4ccc5c(c4)oc4c6ccccc6c(-c6ccccc6)cc54)nc(-c4ccc5c(c4)sc4cccc(-c6ccccc6)c45)n3)cc2)cc1]

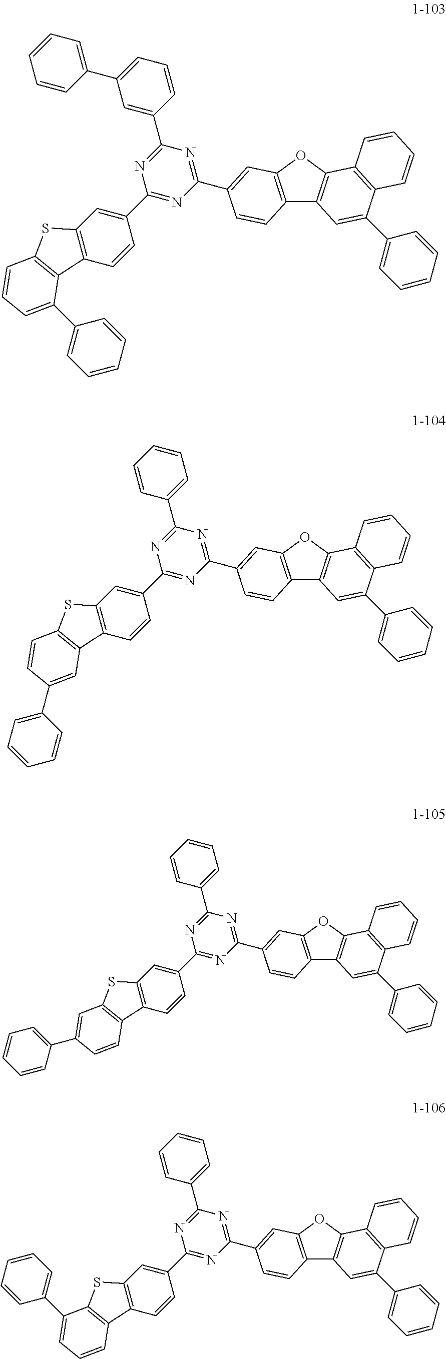 [SMILES: c1ccc(-c2cccc(-c3nc(-c4ccc5c(c4)oc4c6ccccc6c(-c6ccccc6)cc54)nc(-c4ccc5c(c4)sc4cccc(-c6ccccc6)c45)n3)c2)cc1 ; c1ccc(-c2ccc3sc4cc(-c5nc(-c6ccccc6)nc(-c6ccc7c(c6)oc6c8ccccc8c(-c8ccccc8)cc76)n5)ccc4c3c2)cc1 ; c1ccc(-c2ccc3c(c2)sc2cc(-c4nc(-c5ccccc5)nc(-c5ccc6c(c5)oc5c7ccccc7c(-c7ccccc7)cc65)n4)ccc23)cc1 ; c1ccc(-c2nc(-c3ccc4c(c3)oc3c5ccccc5c(-c5ccccc5)cc43)nc(-c3ccc4c(c3)sc3c(-c5ccccc5)cccc34)n2)cc1]

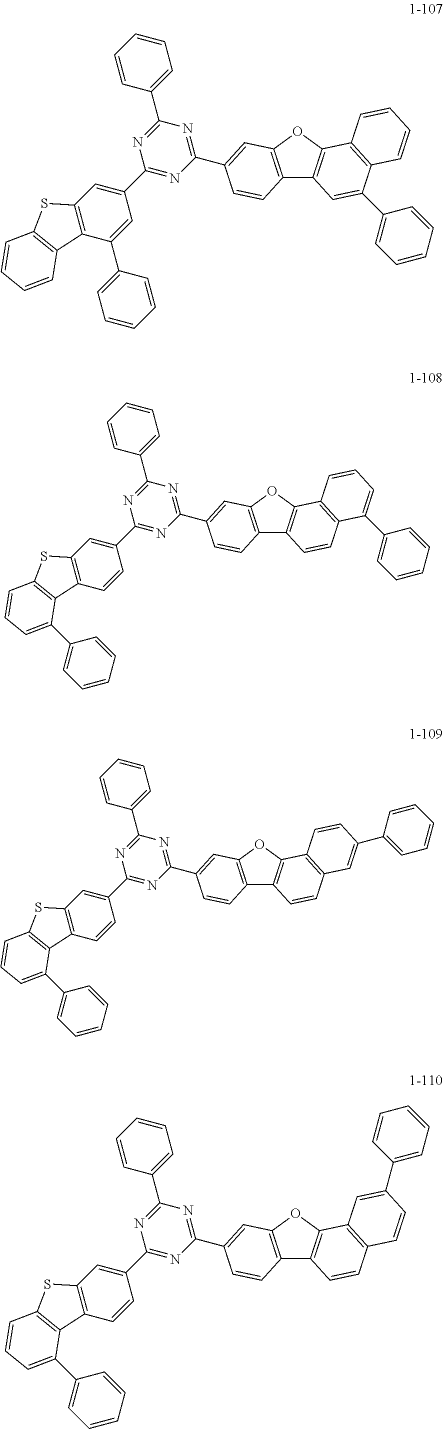 [SMILES: c1ccc(-c2nc(-c3ccc4c(c3)oc3c5ccccc5c(-c5ccccc5)cc43)nc(-c3cc(-c4ccccc4)c4c(c3)sc3ccccc34)n2)cc1 ; c1ccc(-c2nc(-c3ccc4c(c3)oc3c5cccc(-c6ccccc6)c5ccc43)nc(-c3ccc4c(c3)sc3cccc(-c5ccccc5)c34)n2)cc1 ; c1ccc(-c2ccc3c(ccc4c5ccc(-c6nc(-c7ccccc7)nc(-c7ccc8c(c7)sc7cccc(-c9ccccc9)c78)n6)cc5oc34)c2)cc1 ; c1ccc(-c2ccc3ccc4c5ccc(-c6nc(-c7ccccc7)nc(-c7ccc8c(c7)sc7cccc(-c9ccccc9)c78)n6)cc5oc4c3c2)cc1]

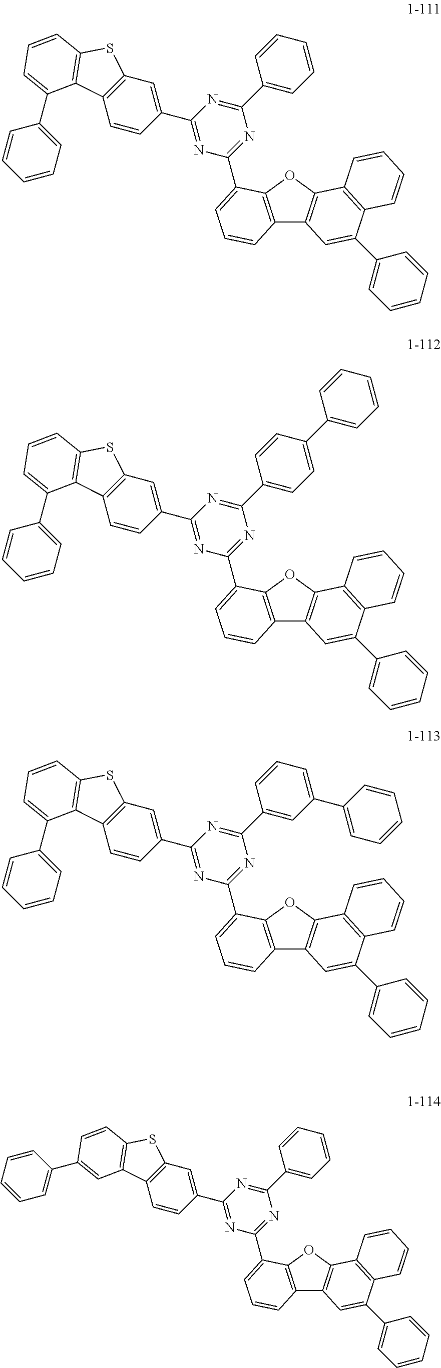 [SMILES: c1ccc(-c2nc(-c3ccc4c(c3)sc3cccc(-c5ccccc5)c34)nc(-c3cccc4c3oc3c5ccccc5c(-c5ccccc5)cc43)n2)cc1 ; c1ccc(-c2ccc(-c3nc(-c4ccc5c(c4)sc4cccc(-c6ccccc6)c45)nc(-c4cccc5c4oc4c6ccccc6c(-c6ccccc6)cc54)n3)cc2)cc1 ; c1ccc(-c2cccc(-c3nc(-c4ccc5c(c4)sc4cccc(-c6ccccc6)c45)nc(-c4cccc5c4oc4c6ccccc6c(-c6ccccc6)cc54)n3)c2)cc1 ; c1ccc(-c2ccc3sc4cc(-c5nc(-c6ccccc6)nc(-c6cccc7c6oc6c8ccccc8c(-c8ccccc8)cc76)n5)ccc4c3c2)cc1]

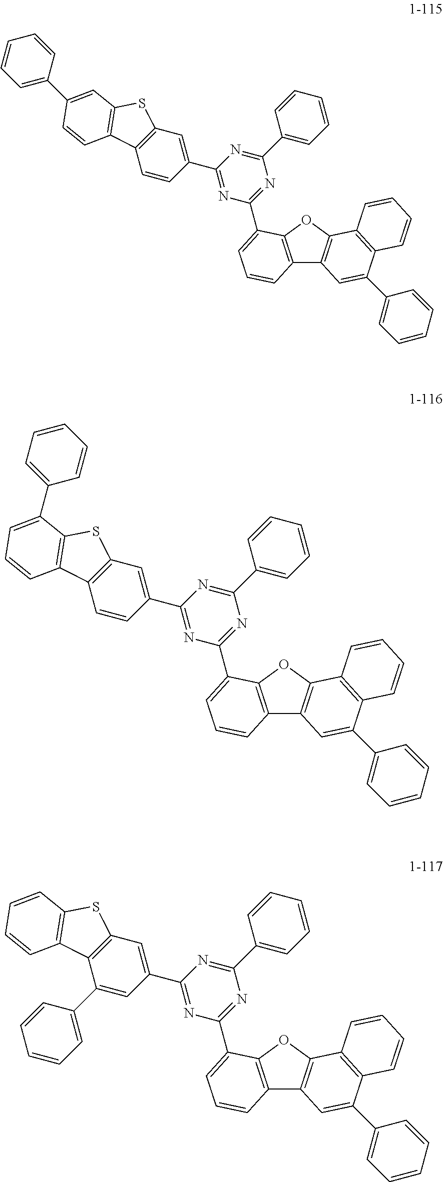 [SMILES: c1ccc(-c2ccc3c(c2)sc2cc(-c4nc(-c5ccccc5)nc(-c5cccc6c5oc5c7ccccc7c(-c7ccccc7)cc65)n4)ccc23)cc1 ; c1ccc(-c2nc(-c3ccc4c(c3)sc3c(-c5ccccc5)cccc34)nc(-c3cccc4c3oc3c5ccccc5c(-c5ccccc5)cc43)n2)cc1 ; c1ccc(-c2nc(-c3cc(-c4ccccc4)c4c(c3)sc3ccccc34)nc(-c3cccc4c3oc3c5ccccc5c(-c5ccccc5)cc43)n2)cc1]

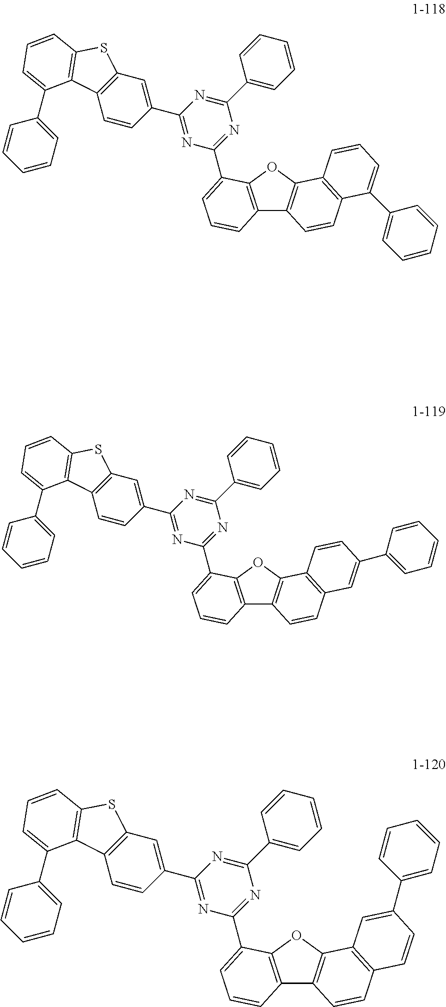 [SMILES: c1ccc(-c2nc(-c3ccc4c(c3)sc3cccc(-c5ccccc5)c34)nc(-c3cccc4c3oc3c5cccc(-c6ccccc6)c5ccc43)n2)cc1 ; c1ccc(-c2ccc3c(ccc4c5cccc(-c6nc(-c7ccccc7)nc(-c7ccc8c(c7)sc7cccc(-c9ccccc9)c78)n6)c5oc34)c2)cc1 ; c1ccc(-c2ccc3ccc4c5cccc(-c6nc(-c7ccccc7)nc(-c7ccc8c(c7)sc7cccc(-c9ccccc9)c78)n6)c5oc4c3c2)cc1]

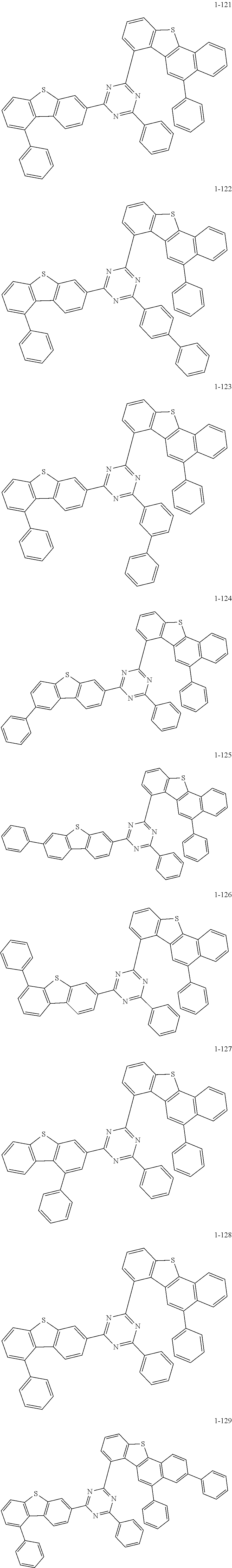

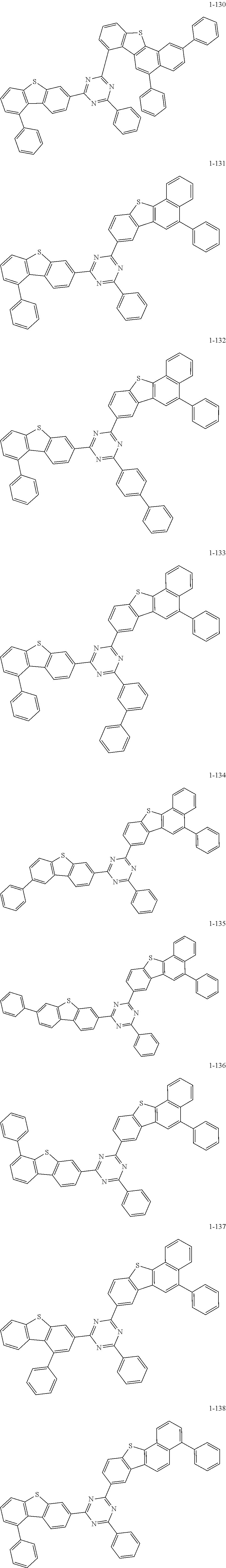

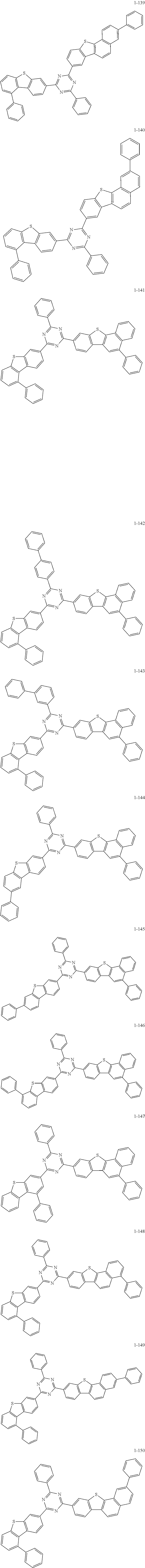

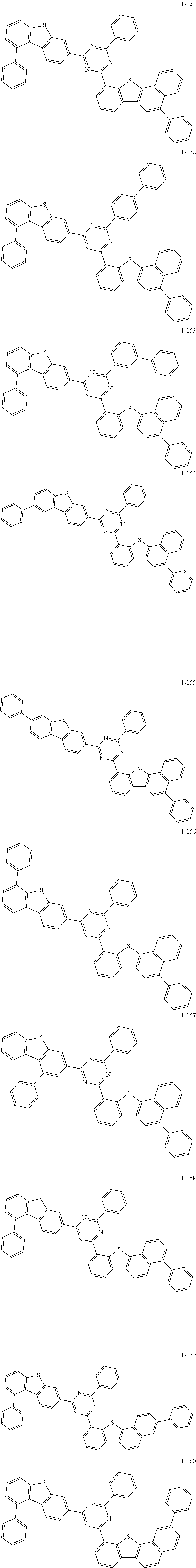

In another exemplary embodiment of the present specification, a first electrode; a second electrode; and an organic material layer having one or more layers provided between the first electrode and the second electrode are included, and one or more layers of the organic material layer include the heterocyclic compound of Chemical Formula 1.

In an exemplary embodiment of the present specification, the organic material layer including the heterocyclic compound may further include a compound of the following Chemical Formula 2.

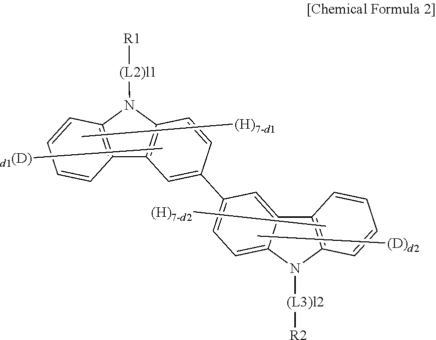

in Chemical Formula 2,
L2 and L3 are each independently a direct bond; or a substituted or unsubstituted arylene group having 6 to 60 carbon atoms,
l1 and l2 are each an integer from 1 to 3, and when l1 and l2 are each 2 or higher, substituents in the parenthesis are the same as or different from each other,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms,
H is hydrogen and D is deuterium, and
d1 and d2 are each independently an integer from 0 to 7.

In the case of an organic light emitting device simultaneously including the heterocyclic compound of Chemical Formula 1 and the compound of Chemical Formula 2, better efficiency and service life effects are exhibited. From this result, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, the compound of Chemical Formula 2 serves as a donor and the compound of Chemical Formula 1 serves as an acceptor, so that it could be confirmed that when the compounds were used as a host of a light emitting layer, excellent device characteristics were exhibited.

In an exemplary embodiment of the present specification, L2 and L3 are each independently a direct bond; or a substituted or unsubstituted C6 to C30 arylene group.

In an exemplary embodiment of the present specification, L2 and L3 are each independently a direct bond; or a C6 to C30 arylene group which is unsubstituted or substituted with deuterium.

In an exemplary embodiment of the present specification, L2 and L3 are each independently a direct bond; or a C6 to C12 arylene group which is unsubstituted or substituted with deuterium.

In an exemplary embodiment of the present specification, L2 and L3 are each independently a direct bond; a phenylene group which is unsubstituted or substituted with deuterium; or a biphenylene group which is unsubstituted or substituted with deuterium.

In an exemplary embodiment of the present specification, R1 and R2 are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R1 and R2 are each independently a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, R1 and R2 are each independently a substituted or unsubstituted C1 to C30 alkyl group; a substituted or unsubstituted C3 to C30 cycloalkyl group; a substituted or unsubstituted C6 to C30 aryl group; or a substituted or unsubstituted C2 to C30 heteroaryl group.

In an exemplary embodiment of the present specification, R1 and R2 are each independently a substituted or unsubstituted C6 to C30 aryl group; or a substituted or unsubstituted C2 to C30 heteroaryl group.

In an exemplary embodiment of the present specification, R1 and R2 are each independently a substituted or unsubstituted C6 to C30 aryl group; or a C2 to C30 heteroaryl group which is substituted or unsubstituted and includes O or S.

In an exemplary embodiment of the present specification, R1 and R2 are each independently a substituted or unsubstituted phenyl group; a substituted or unsubstituted biphenyl group; a substituted or unsubstituted terphenyl group; a substituted or unsubstituted naphthyl group; a substituted or unsubstituted triphenylene group; a substituted or unsubstituted fluorene group; a substituted or unsubstituted dibenzofuran group; or a substituted or unsubstituted dibenzothiophene group.

In an exemplary embodiment of the present specification, R1 and R2 are each independently a phenyl group which is unsubstituted or substituted with deuterium; a biphenyl group which is unsubstituted or substituted with deuterium; a terphenyl group which is unsubstituted or substituted with deuterium; a naphthyl group which is unsubstituted or substituted with deuterium; a triphenylene group which is unsubstituted or substituted with deuterium; a fluorene group which is unsubstituted or substituted with one or more substituents selected among deuterium and an alkyl group; a dibenzofuran group which is unsubstituted or substituted with deuterium; or a dibenzothiophene group which is unsubstituted or substituted with deuterium.

In an exemplary embodiment of the present specification, R1 and R2 are each independently a C6 to C30 aryl group which is unsubstituted or substituted with one or more substituents selected among deuterium and an alkyl group; or a C2 to C30 heteroaryl group which is unsubstituted or substituted with deuterium.

In an exemplary embodiment of the present specification, d1 and d2 may be each independently 0 or 7.

In an exemplary embodiment of the present specification, Chemical Formula 2 may be represented by the following Chemical Formula 2-1 or 2-2.

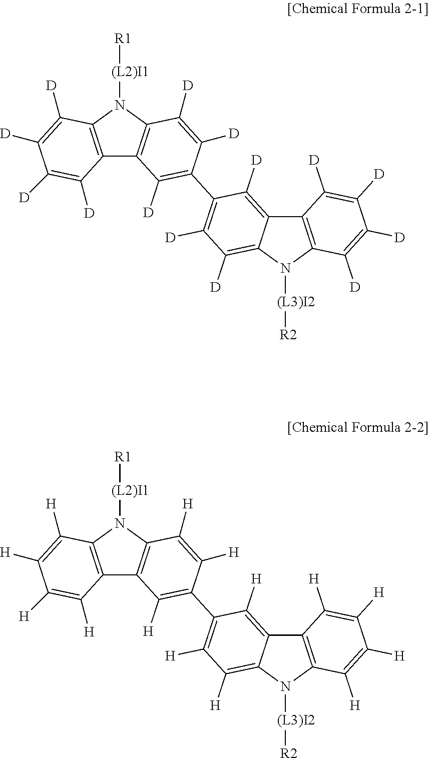

In Chemical Formulae 2-1 and 2-2, the definition of each substituent is the same as the definition in Chemical Formula 2.

In an exemplary embodiment of the present specification, the deuterium content of Chemical Formula 2 is 0% to 100%.

In an exemplary embodiment of the present specification, the deuterium content of Chemical 2 is 0% or 10% to 100%.

In an exemplary embodiment of the present specification, the deuterium content of Chemical 2 is 0% or 30% to 100%.

In an exemplary embodiment of the present specification, the deuterium content of Chemical Formula 2 may be 0%.

In an exemplary embodiment of the present specification, the deuterium content of Chemical Formula 2 may be more than 0% and 100% or less.

In an exemplary embodiment of the present specification, the deuterium content of Chemical Formula 2 may be 10% to 100%.

An organic light emitting device according to an exemplary embodiment of the present specification may include a heterocyclic compound of Chemical Formula 1 with a deuterium content of more than 0% and a compound of Chemical Formula 2 with a deuterium content of 0% in the organic material layer.

An organic light emitting device according to another exemplary embodiment may include a heterocyclic compound of Chemical Formula 1 with a deuterium content of more than 0% and a compound of Chemical Formula 2 with a deuterium content of more than 0% in the organic material layer.

In an exemplary embodiment of the present specification, when Chemical Formula 2 includes deuterium, there are effects of lowering the driving voltage and increasing the light emitting efficiency and service life compared to the case where Chemical Formula 2 does not include deuterium.

Likewise, in the case of the compound of Chemical Formula 2, in an electron transport moiety and a hole transport moiety, which directly exchange electrons, the vibrational frequency of interatomic bond in molecules changes continuously as electrons move, affecting the stability of interatomic bond in molecules and the stability of the molecular structure. By substituting the compound with deuterium, which has a higher molecular weight than hydrogen, the energy of the molecule is lowered by reducing the change of the vibrational frequency, and thus the stability of molecules is enhanced.

In addition, since the single bond dissociation energy of carbon and deuterium is higher than the single bond dissociation energy of carbon and hydrogen, the thermal stability of molecule is increased, so that there is an effect of improving the service life of the device.

In an exemplary embodiment of the present specification, Chemical Formula 2 may be represented by any one of the following compounds.

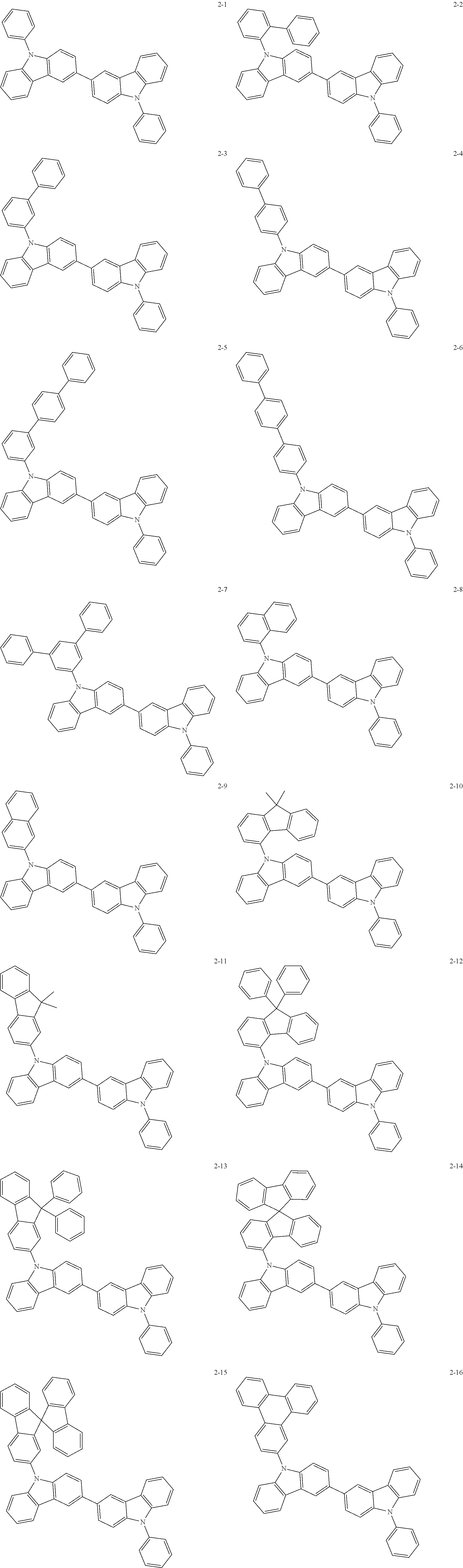

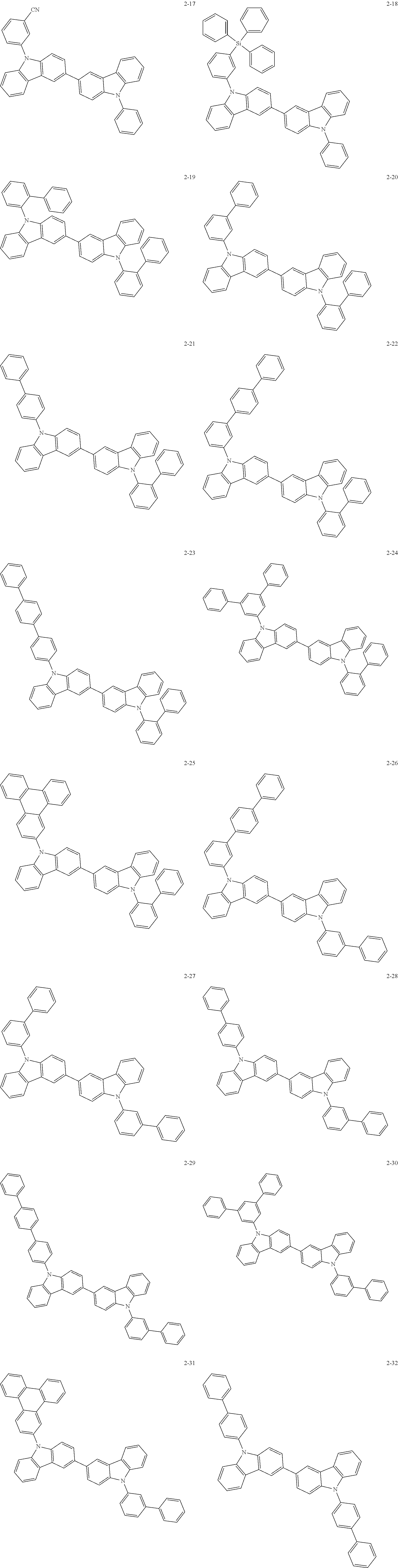

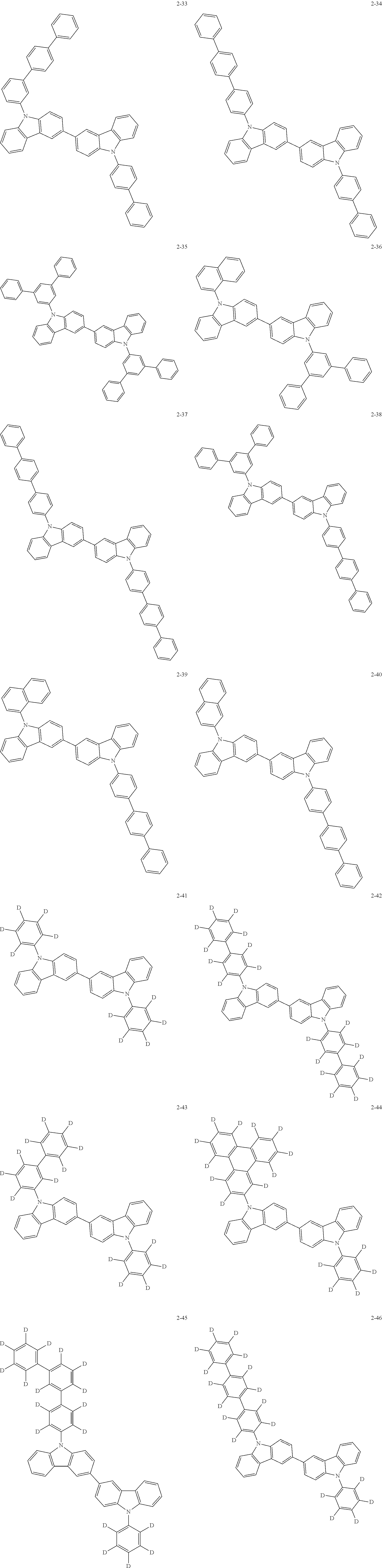

[chemical structure]

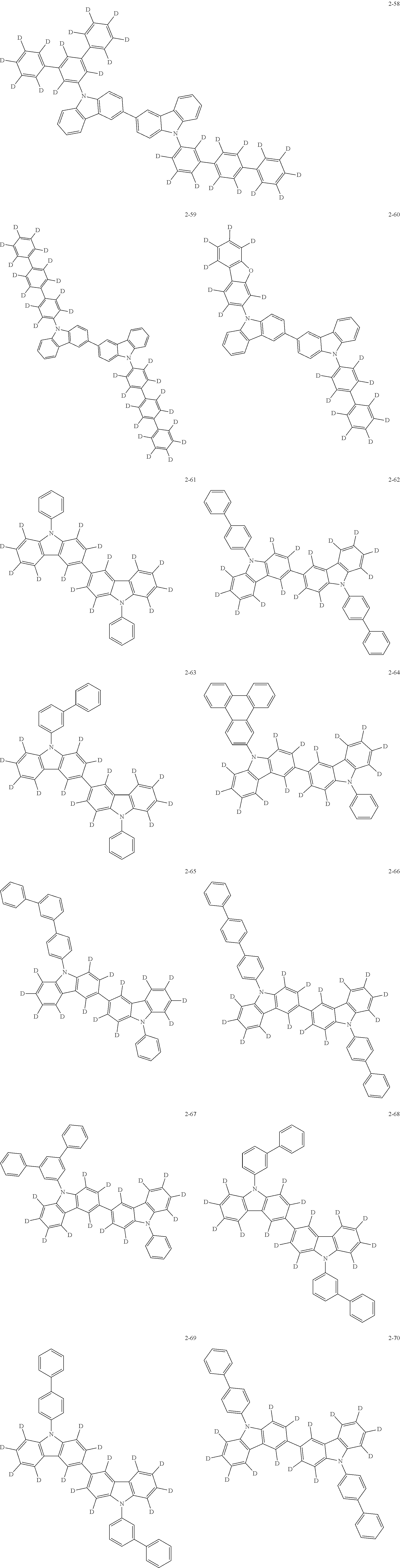

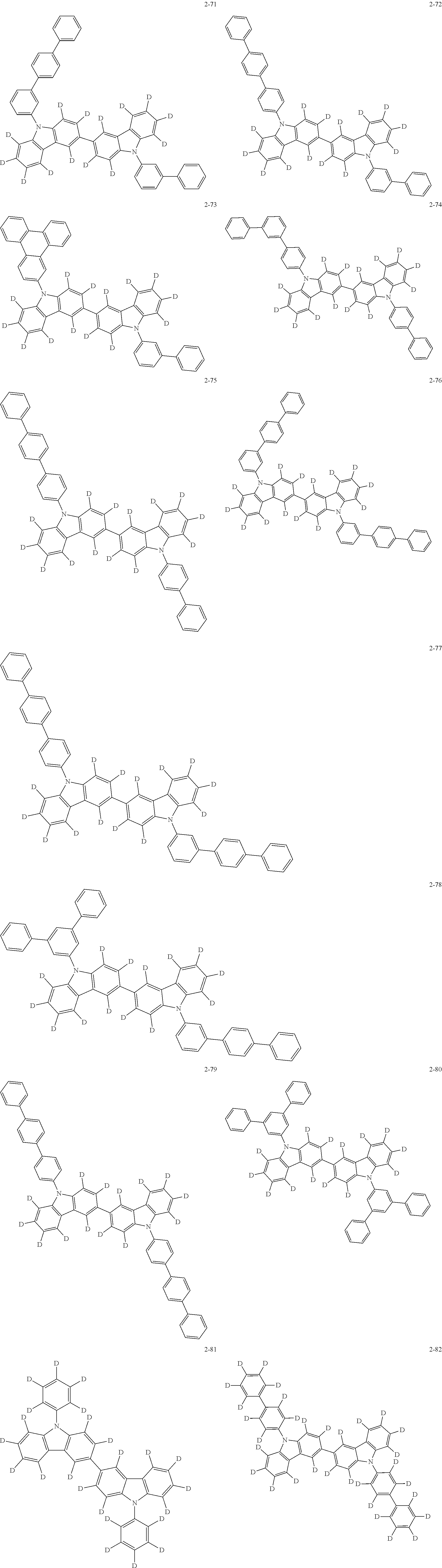

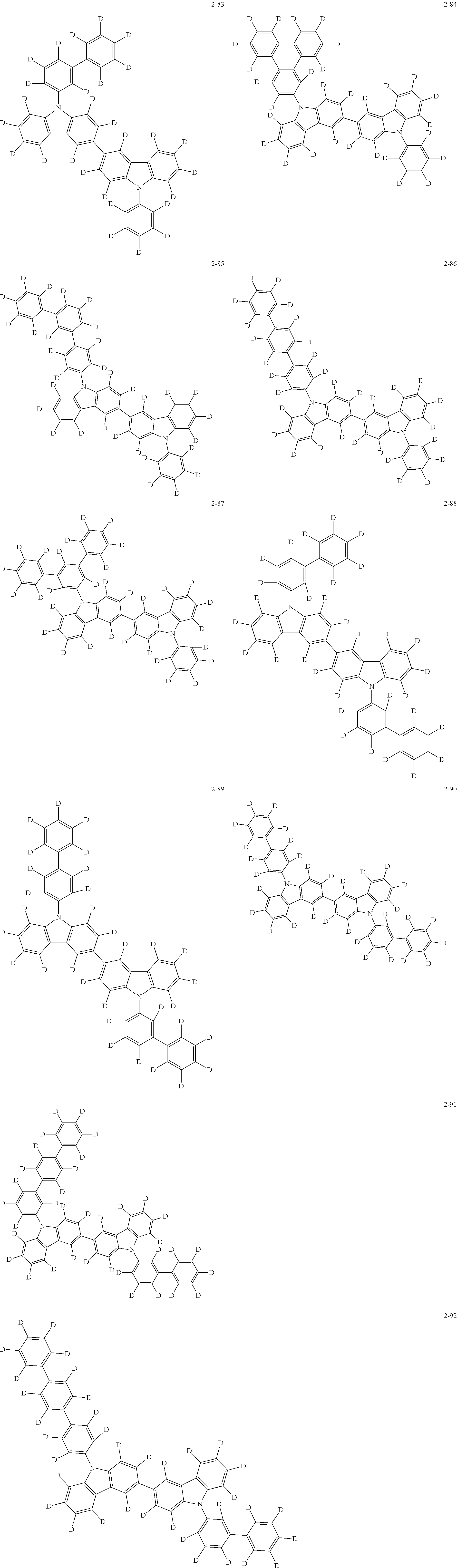

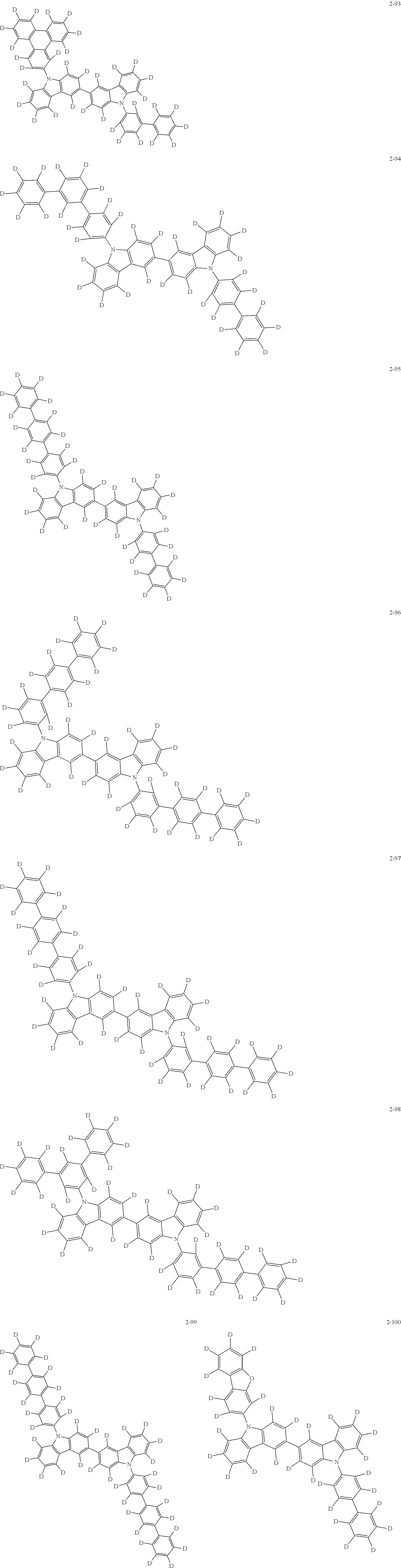

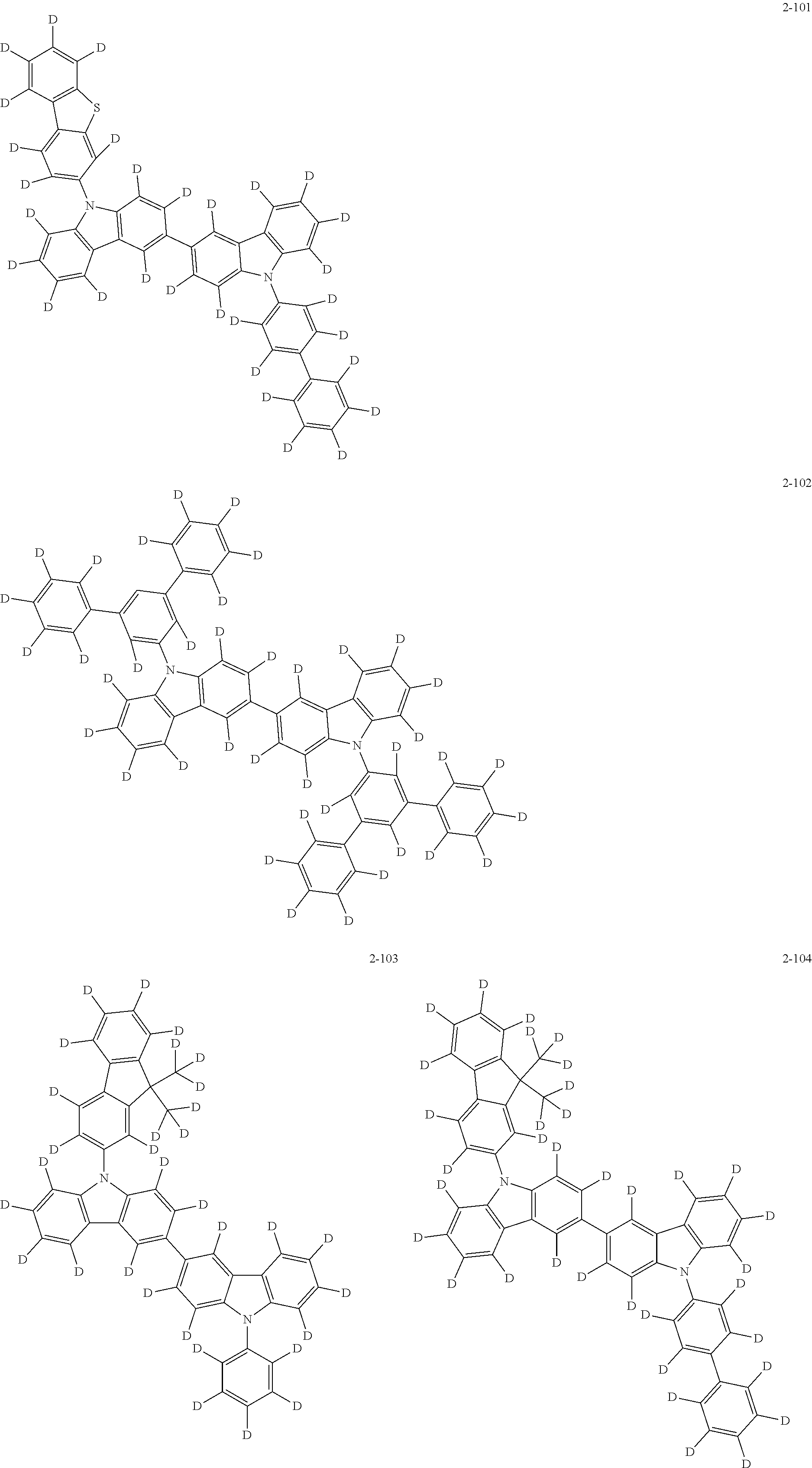

In an exemplary embodiment of the present specification, the organic material layer may include the heterocyclic compound of Chemical Formula 1 and the compound of Chemical Formula 2 at a weight ratio of 1:10 to 10:1, preferably at a weight ratio of 1:8 to 8:1, 1:5 to 5:1 and 1:3 to 3:1.

In an exemplary embodiment of the present specification, the organic material layer may include the heterocyclic compound of Chemical Formula 1 and the compound of Chemical Formula 2 at a weight ratio of 1:1 to 1:3.

In an exemplary embodiment of the present specification, the organic material layer may further include a phosphorescent dopant.

In an exemplary embodiment of the present specification, the phosphorescent dopant may be a green, blue or red phosphorescent dopant.

In an exemplary embodiment of the present specification, the phosphorescent dopant may be a green phosphorescent dopant.

In another exemplary embodiment, the phosphorescent dopant may be a red phosphorescent dopant.

In an exemplary embodiment of the present specification, the phosphorescent dopant may be an iridium-based dopant.

As a material for the phosphorescent dopant, those known in the art may be used. For example, phosphorescent dopant materials represented by LL′MX′, LL′L″M, LMX′X″, L₂MX′ and L₃M may be used, but the scope of the present invention is not limited by these examples.

Here, L, L′, L″, X′ and X″ are bidentate ligands different from each other, and M is a metal forming an octahedral complex.

M may be iridium, platinum, osmium, and the like.

L, L′ and L″ are anionic bidentate ligands coordinated to M with the iridium-based dopant by sp2 carbon and heteroatoms, and non-limiting examples of L, L′ and L″ include 1-phenylisoquinoline, 2-(1-naphthyl)benzoxazole, 2-phenylbenzoxazole, 2-phenylbenzothiazole, 2-phenylbenzothiazole, 7,8-benzoquinoline, thiophene group pyrizine, phenylpyridine, benzothiophene group pyrizine, 3-methoxy-2-phenylpyridine, thiophene group pyrizine, tolylpyridine, and the like.

X′ and X″ may function to trap electrons or holes, and non-limiting examples of X′ and X″ include acetylacetonate (acac), hexafluoroacetylacetonate, salicylidene, picolinate, 8-hydroxyquinolinate and the like.

In an exemplary embodiment of the present specification, as the iridium-based dopant, Ir(ppy)₃ as a green phosphorescent dopant may be used.

In an exemplary embodiment of the present specification, as the iridium-based dopant, Ir(piq)₂(acac) as a red phosphorescent dopant may be used.

In an exemplary embodiment of the present specification, the content of the dopant may be 1% to 15%, preferably 1% to 10% based on the entire light emitting layer.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1 alone or using the heterocyclic compound of Chemical Formula 1 together with the compound of Chemical Formula 2.

The heterocyclic compound of Chemical Formula 1 and the compound of Chemical Formula 2 may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

In the organic light emitting device of the present invention, the organic material layer includes a light emitting layer, and the light emitting layer may include the heterocyclic compound of Chemical Formula 1.

In the organic light emitting device of the present specification, the organic material layer includes a light emitting layer, and the light emitting layer may include the heterocyclic compound of Chemical Formula 1 and the compound of Chemical Formula 2.

In the organic light emitting device of the present specification, the organic material layer includes a light emitting layer, the light emitting layer includes a host, and the host may include the heterocyclic compound of Chemical Formula 1.

In the organic light emitting device of the present specification, the heterocyclic compound of Chemical Formula 1 may be used as a green host or a red host.

In the organic light emitting device of the present specification, the organic material layer includes a light emitting layer, the light emitting layer includes a host, and the host may include the heterocyclic compound of Chemical Formula 1 and the compound of Chemical Formula 2.

In the organic light emitting device of the present specification, the compound of Chemical Formula 2 may be used as a green host or a red host.

When the heterocyclic compound of Chemical Formula 1 and the compound of Chemical Formula 2 are combined to form a host, better efficiency and service life are exhibited than when one host is used.

In an exemplary embodiment of the present specification, the heterocyclic compound of Chemical Formula 1 may be used as an N-type host material, and the compound of Chemical Formula 2 may be used as a P-type host material.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a host material of a light emitting layer of a blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a host material of a light emitting layer of a green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a host material of a light emitting layer of a red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

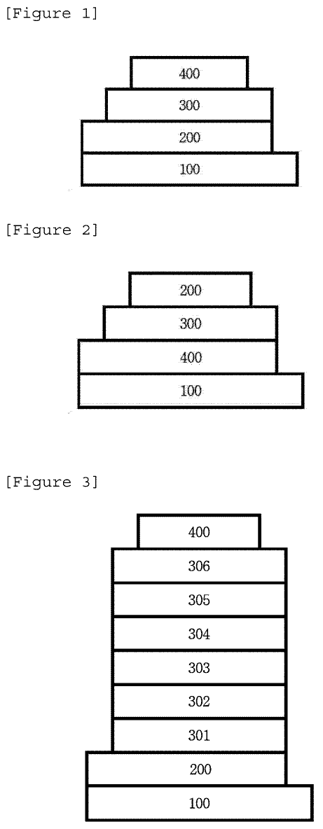

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented.

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 and the compound of Chemical Formula 2 will be exemplified below, but these materials are provided only for exemplification and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is a composition for an organic material layer of an organic light emitting device, including the heterocyclic compound of Chemical Formula 1.

Furthermore, another exemplary embodiment of the present specification provides a composition for an organic material layer of an organic light emitting device, which includes the heterocyclic compound of Chemical Formula 1 and the compound of Chemical Formula 2.

The specific contents on the heterocyclic compound of Chemical Formula 1 and the compound of Chemical Formula 2 are the same as those described above.

The weight ratio of the heterocyclic compound represented by Chemical Formula 1 and the compound of Chemical Formula 2 in the composition may be 1:10 to 10:1, 1:8 to 8:1, 1:5 to 5:1, and 1:3 to 3:1, but is not limited thereto.

In an exemplary embodiment of the present specification, the composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

In an exemplary embodiment of the present specification, the composition may be in a form in which the heterocyclic compound of Chemical Formula 1 and the compound of Chemical Formula 2 are simply mixed.

The composition may additionally include materials publicly known in the art such as solvents and additives.

The composition may be used when an organic material for an organic light emitting device is formed, and particularly, may be more preferably used when a host of a light emitting layer is formed.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer, including heterocyclic compound of Chemical Formula 1.

The method for manufacturing an organic light emitting device according to another exemplary embodiment includes: preparing a substrate; forming a first electrode on the substrate; forming an organic material layer having one or more layers on the first electrode; and forming a second electrode on the organic material layer, in which the forming of the organic material layer includes forming an organic material layer having one or more layers by using a composition for an organic material layer, which includes the heterocyclic compound of Chemical Formula 1 and the compound of Chemical Formula 2, and the forming of the organic material layer may form the organic material layer by pre-mixing the heterocyclic compound of Chemical Formula 1; and the compound of Chemical Formula 2 and using a thermal vacuum deposition method.

The pre-mixing means that before the heterocyclic compound of Chemical Formula 1 and the compound of Chemical Formula 2 are deposited onto an organic material layer, the materials are first mixed and the mixture is contained in one common container and mixed.

The pre-mixed material may be referred to as a composition for an organic material layer according to an exemplary embodiment of the present specification.

### Mode for Invention

Hereinafter, the present specification will be described in more detail through Examples, but these Examples are provided only for exemplifying the present application, and are not intended to limit the scope of the present application.

### PREPARATION EXAMPLES

### [Preparation Example 1] Preparation of Compound 1-1[D]

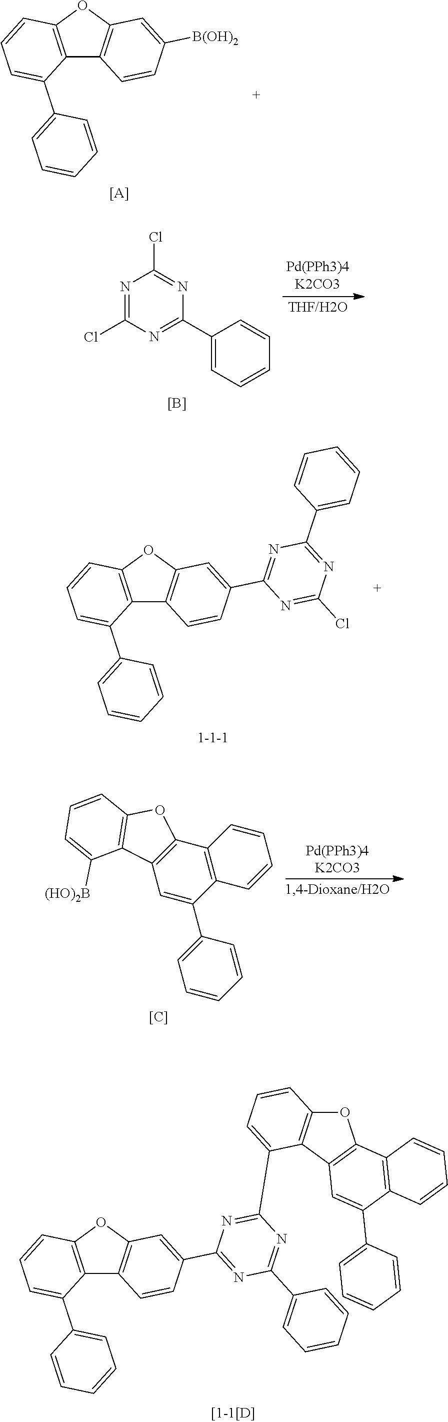

### Preparation of Compound 1-1-1

9-Phenyldibenzo[b,d]furan-3-yl)boronic acid (10 g, 0.035 mol), 2,4-dichloro-6-phenyl-1,3,5-triazine (7.85 g, 0.035 mol), Pd(PPh₃)₄ (2.01 g, 0.002 mol), potassium carbonate (8.85 g, 0.069 mol), and a mixture of tetrahydrofuran) (100 mL)/water (H₂O) (30 mL) were refluxed at 120° C. in a one neck round bottom flask. The mixture was subjected to extraction with dichloromethane, and the extract was concentrated, and then filtered through silica gel. The filtered product was concentrated, and then treated with dichloromethane/methanol to obtain Compound 1-1-1. (12.8 g, 85%)

### Preparation of Compound 1-1[D]

2-Chloro-4-phenyl-6-(9-phenyldibenzo[b,d]furan-3-yl)-1,3,5-triazine (12.8 g, 0.030 mol), (5-phenylnaphtho[1,2-b]benzofuran-7-yl)boronic acid (9.98 g, 0.030 mol), Pd(PPh₃)₄ (17 g, 0.0015 mol), potassium carbonate (6.25 g, 0.059 mol), and a mixture of 1,4-dioxane (80 mL)/H₂O (24 mL) were refluxed at 125° C. in a one neck round bottom flask. After the reaction was completed, the precipitated solid was filtered to obtain the resulting product. The solid thus obtained was dissolved in dichlorobenzene, and the resulting solution was filtered through silica gel. The filtered product was concentrated, and then treated with methanol to obtain Compound 1-1[D]. (18.5 g, 91%)

A target compound (D) was synthesized in the same manner as the preparation in Preparation Example 1, except that Intermediates A, B, and C in the following Table 1 were used in Preparation Example 1.

TABLE 1

| Com- | | | | | |
|---|---|---|---|---|---|
| pound | A | B | C | D | Yield |
| 1-1 | 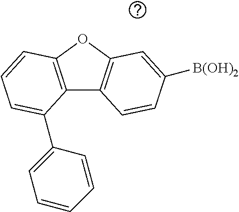 | 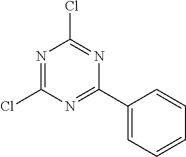 | 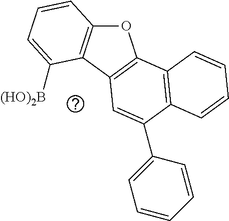 | 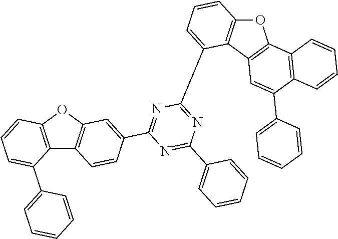 | 77 |
| 1-4 | 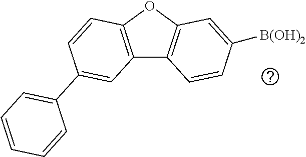 | 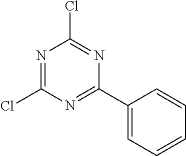 | 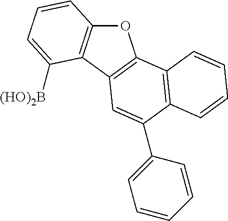 | 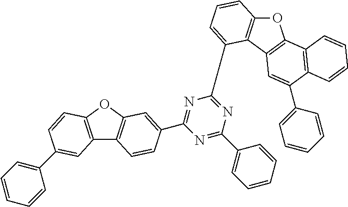 | 76 |
| 1-5 | 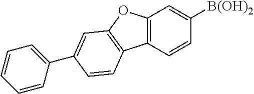 | 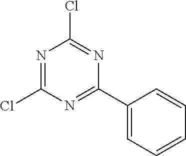 | 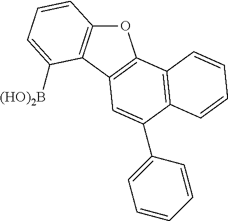 | 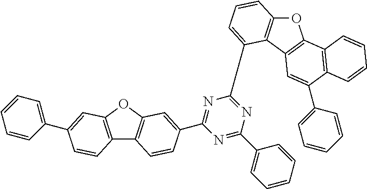 | 85 |
| 1-6 | 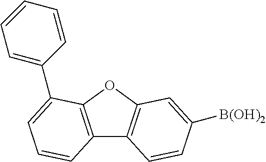 | 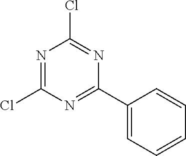 | 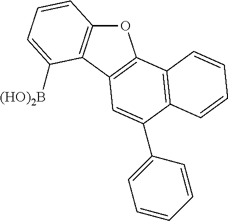 | 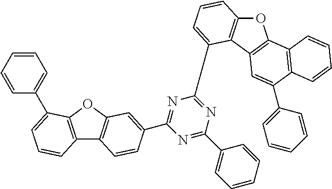 | 73 |
| 1-12 | 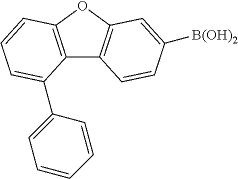 | 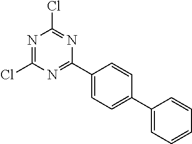 | 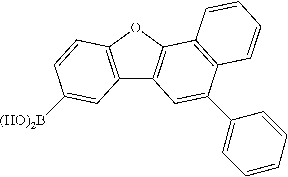 | 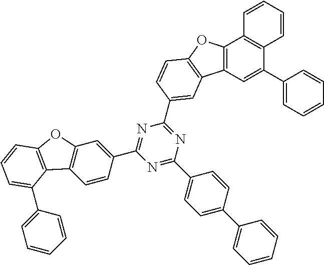 | 74 |
| 1-14 | 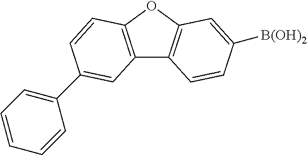 | 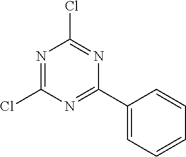 | 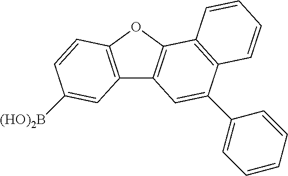 | 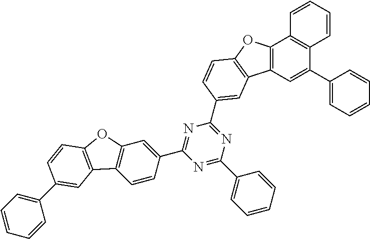 | 71 |
| 1-15 | 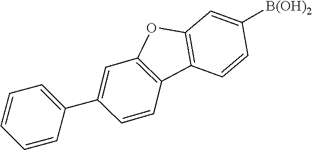 | 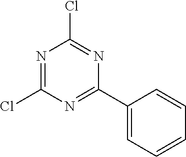 | 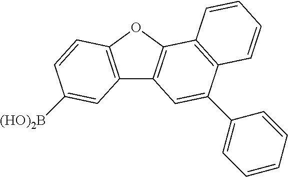 | 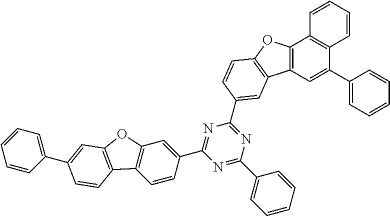 | 85 |
| 1-17 | 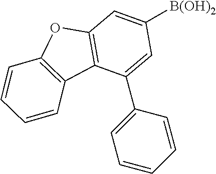 | 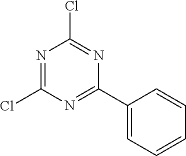 | 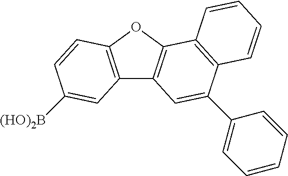 | 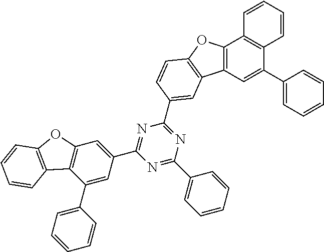 | 68 |
| 1-21 | 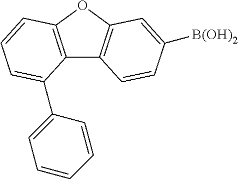 | 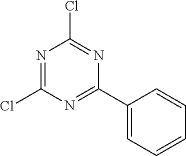 | 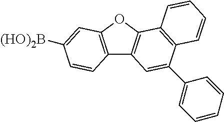 | 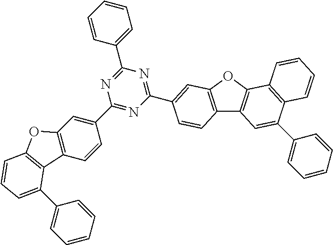 | 66 |
| 1-23 | 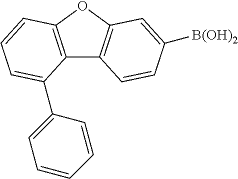 | 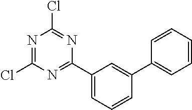 | 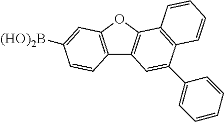 | 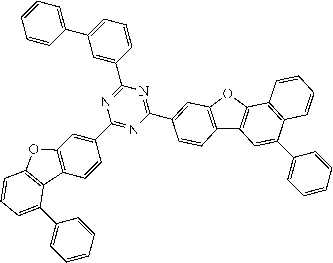 | 71 |
| 1-25 | 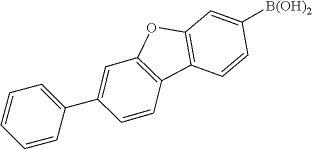 | 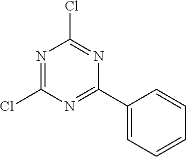 | 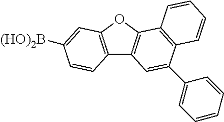 | 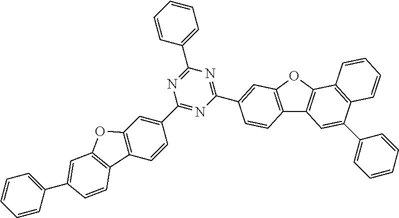 | 70 |
| 1-26 | 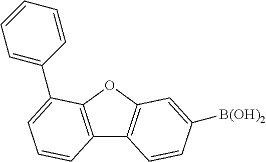 | 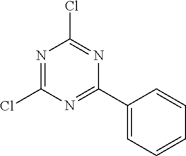 | 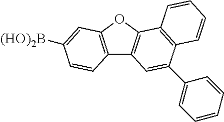 | 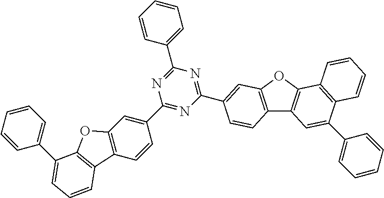 | 77 |
| 1-33 | 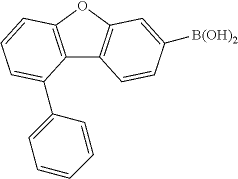 | 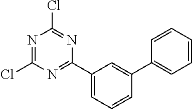 | 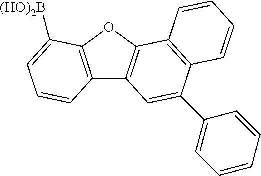 | 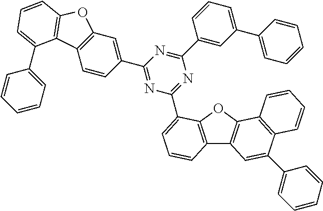 | 65 |
| 1-35 | 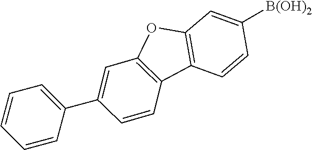 | 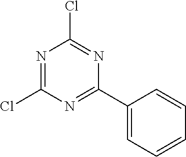 | 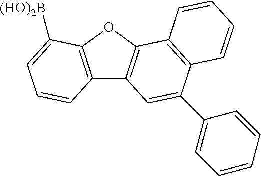 | 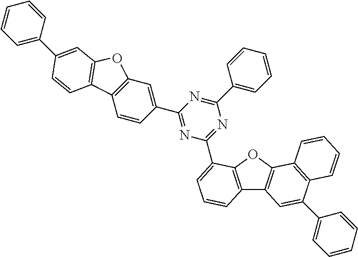 | 70 |
| 1-36 | 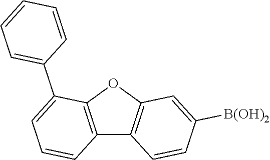 | 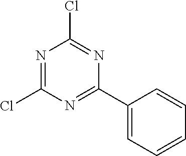 | 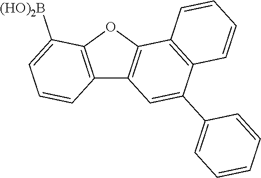 | 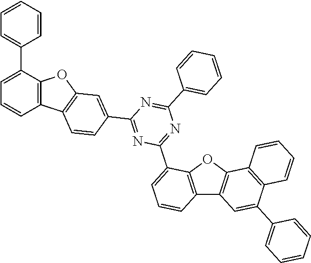 | 74 |
| 1-37 | 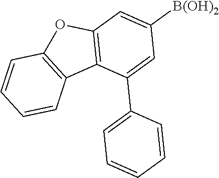 | 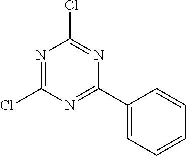 | 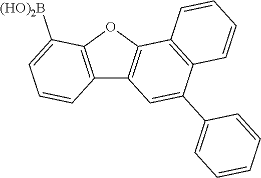 | 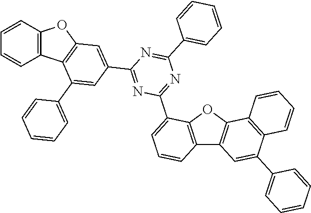 | 74 |
| 1-41 | 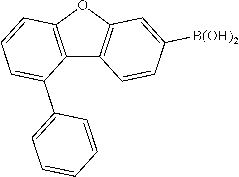 | 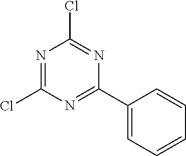 | 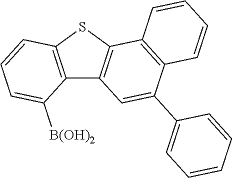 | 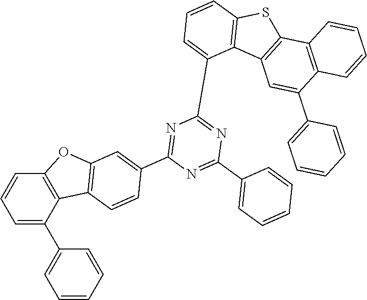 | 72 |
| 1-44 | 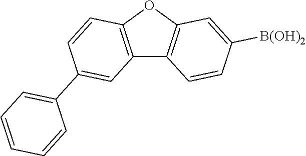 | 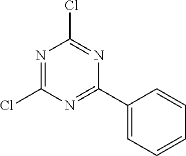 | 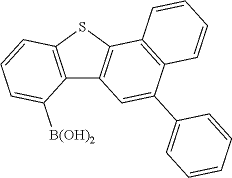 | 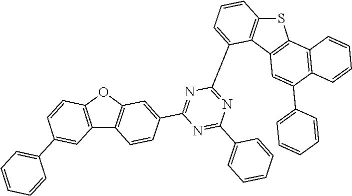 | 81 |
| 1-45 | 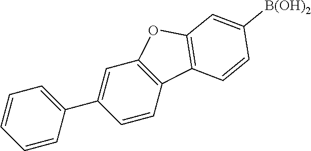 | 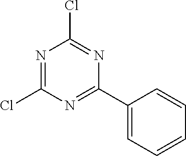 | 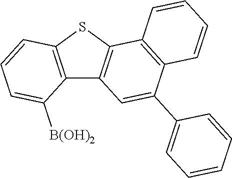 | 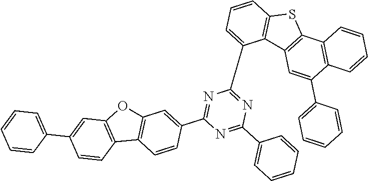 | 76 |
| 1-46 | 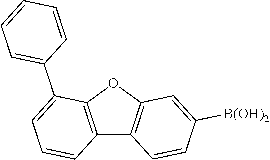 | 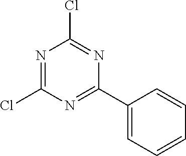 | 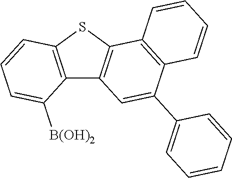 | 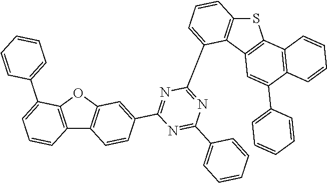 | 82 |
| 1-52 | 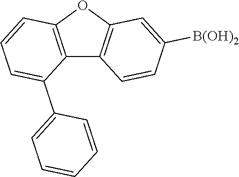 | 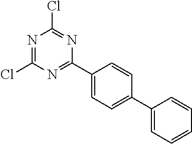 | 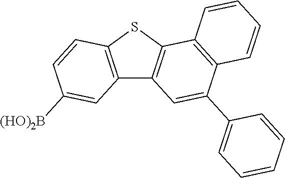 | 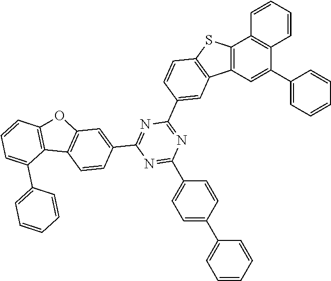 | 69 |
| 1-54 | 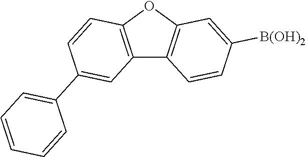 | 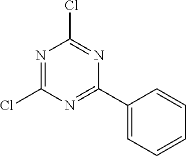 | 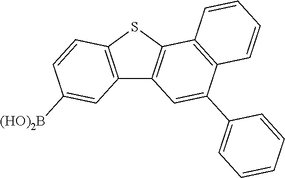 | 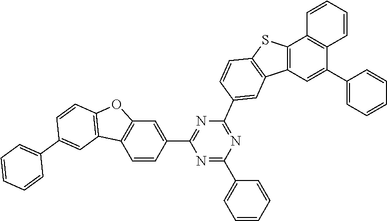 | 66 |
| 1-56 | 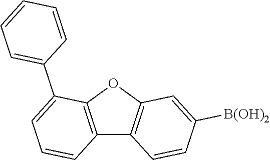 | 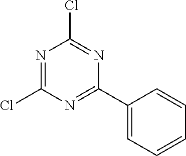 | 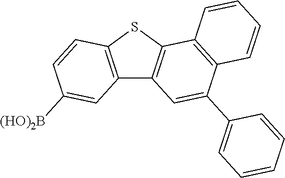 | 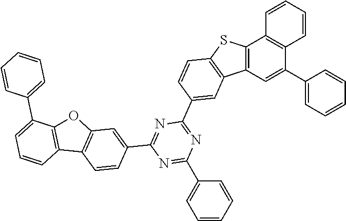 | 68 |
| 1-58 | 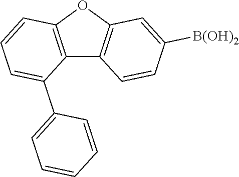 | 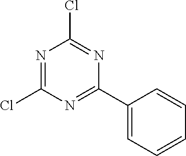 | 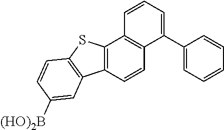 | 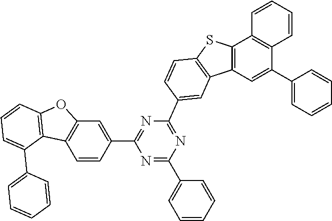 | 78 |
| 1-62 | 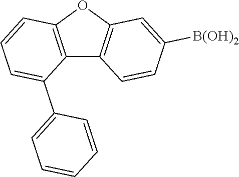 | 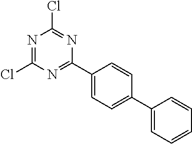 | 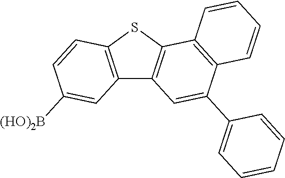 | 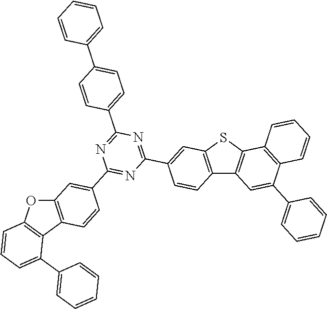 | 79 |
| 1-63 | 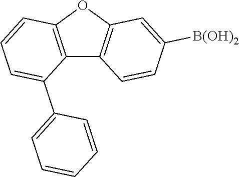 | 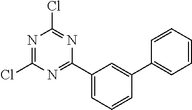 | 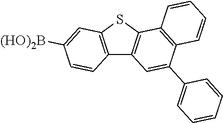 | 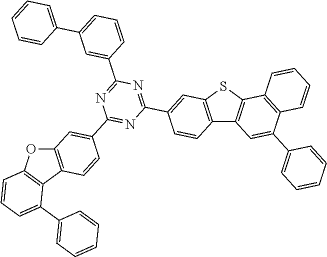 | 65 |
| 1-65 | 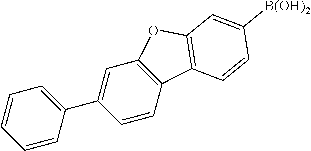 | 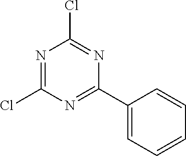 | 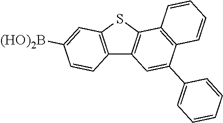 | 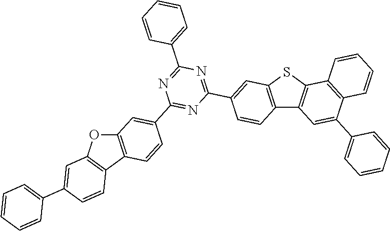 | 71 |
| 1-67 | 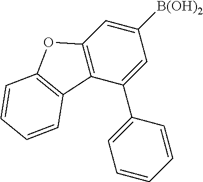 | 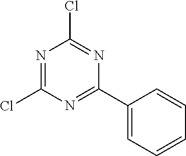 | 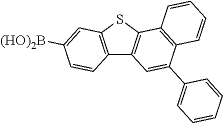 | 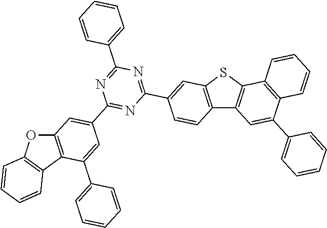 | 71 |
| 1-71 | 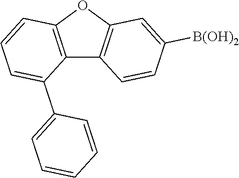 | 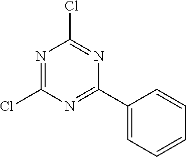 | 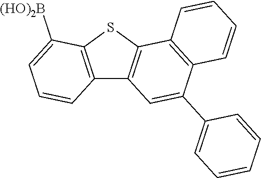 | 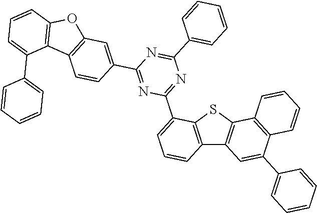 | 77 |
| 1-74 | 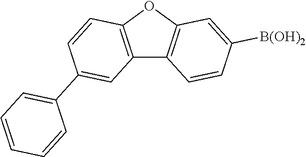 | 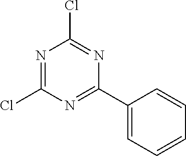 | 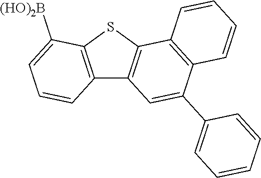 | 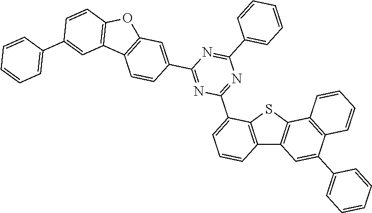 | 72 |
| 1-75 | 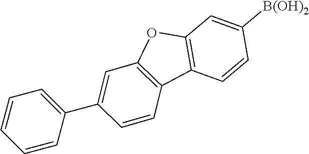 | 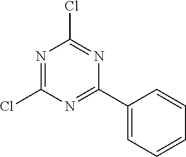 | 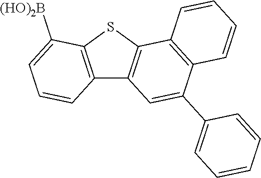 | 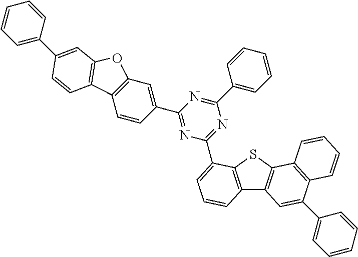 | 80 |
| 1-77 | 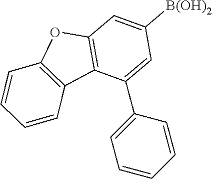 | 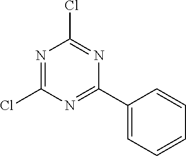 | 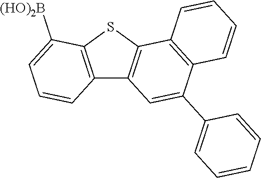 | 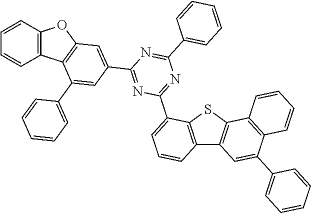 | 66 |
| 1-81 | 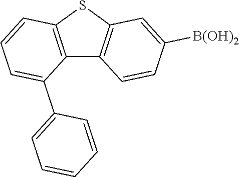 | 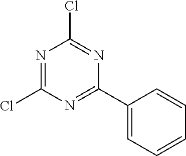 | 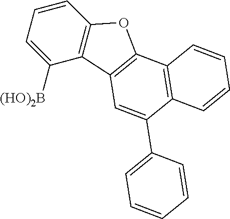 | 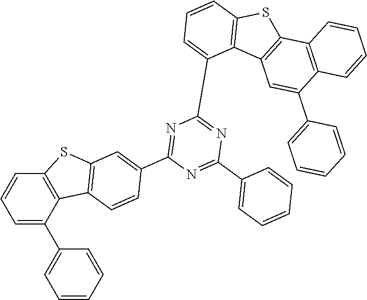 | 72 |
| 1-85 | 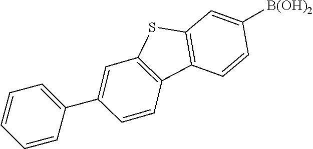 | 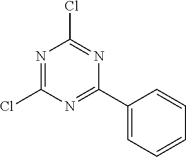 | 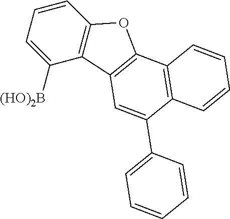 | 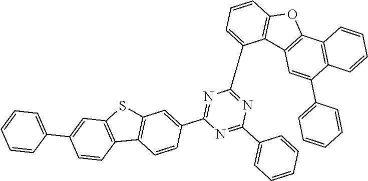 | 84 |
| 1-87 | 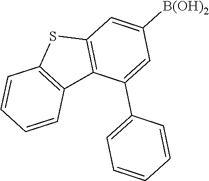 | 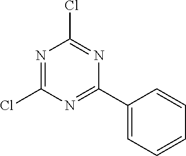 | 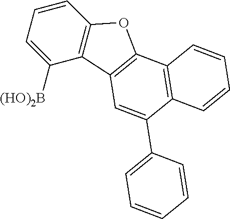 | 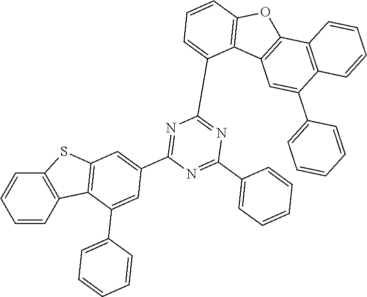 | 78 |
| 1-90 | 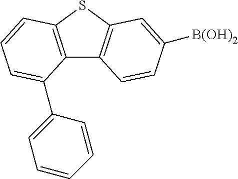 | 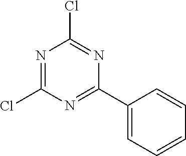 | 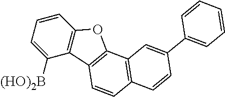 | 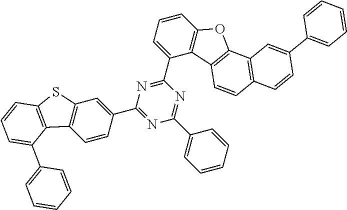 | 75 |
| 1-95 | 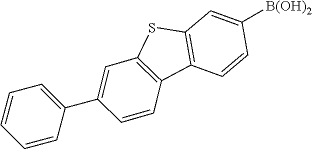 | 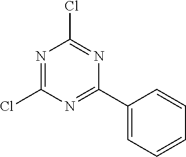 | 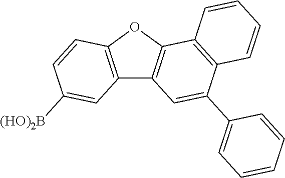 | 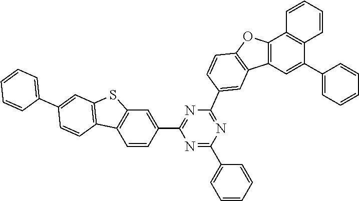 | 75 |
| 1-96 | 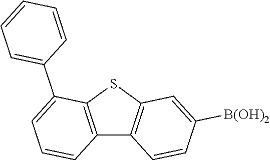 | 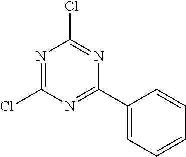 | 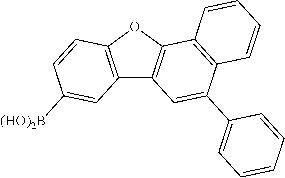 | 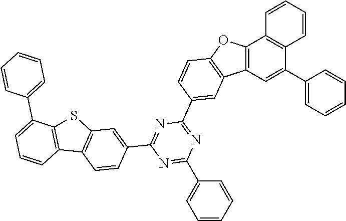 | 71 |
| 1-97 | 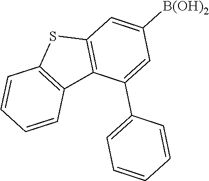 | 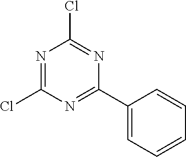 | 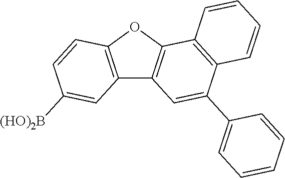 | 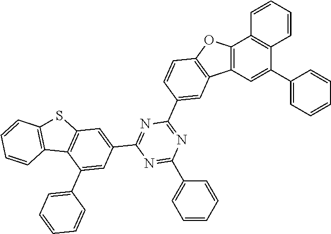 | 70 |
| 1-100 | 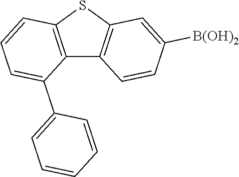 | 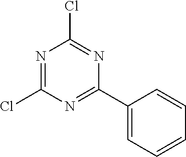 | 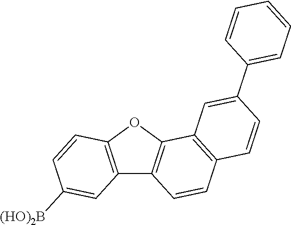 | 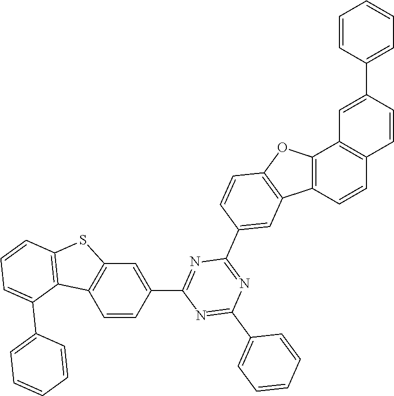 | 84 |
| 1-101 | 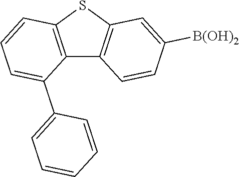 | 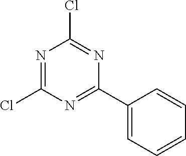 | 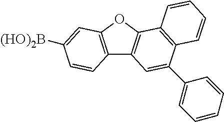 | 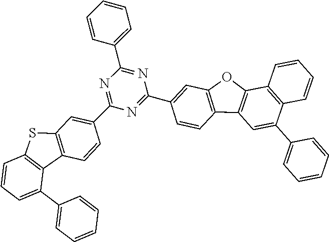 | 78 |
| 1-104 | 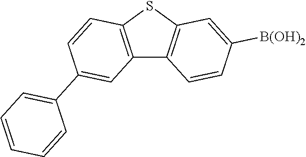 | 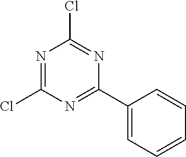 | 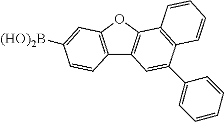 | 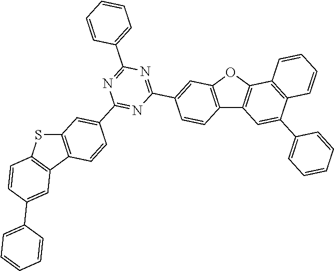 | 83 |
| 1-106 | 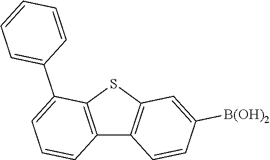 | 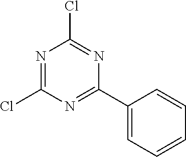 | 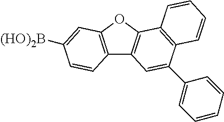 | 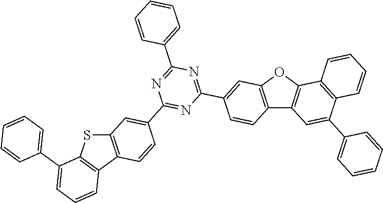 | 66 |
| 1-107 | 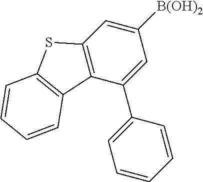 | 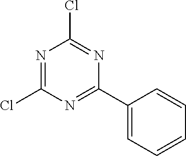 | 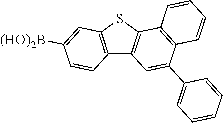 | 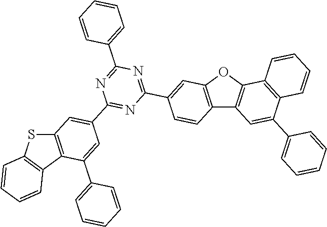 | 65 |
| 1-113 | 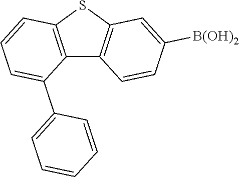 | 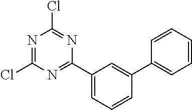 | 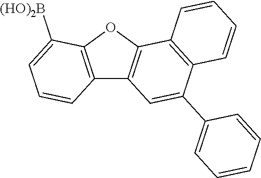 | 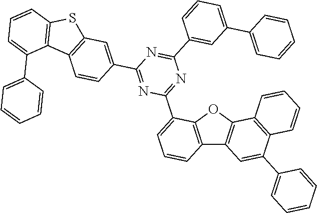 | 85 |
| 1-115 | 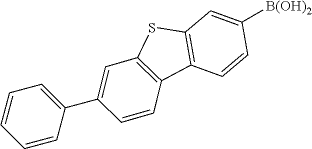 | 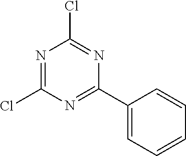 | 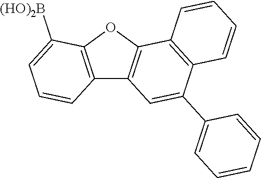 | 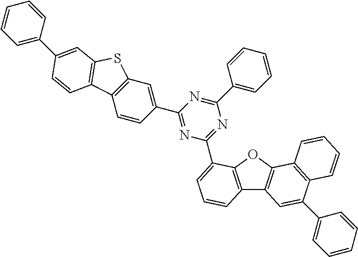 | 79 |
| 1-116 | 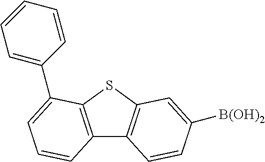 | 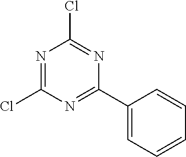 | 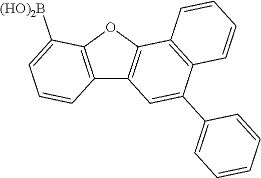 | 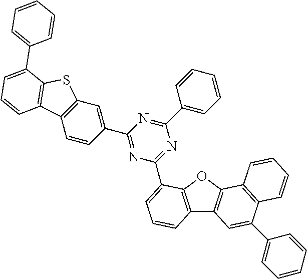 | 69 |
| 1-117 | 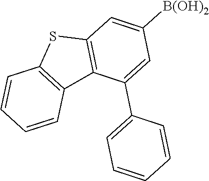 | 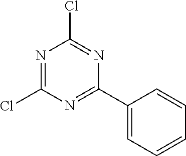 | 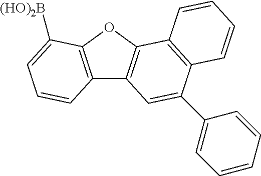 | 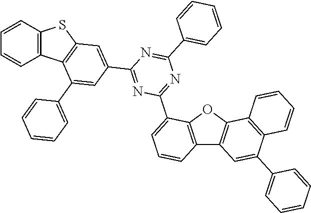 | 67 |
| 1-123 | 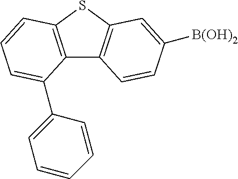 | 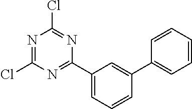 | 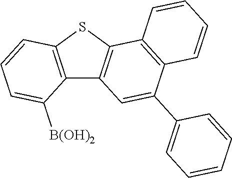 | 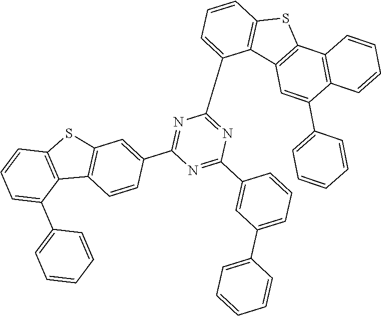 | 65 |
| 1-125 | 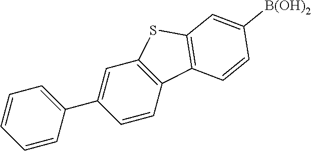 | 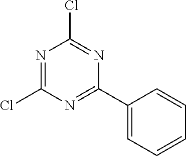 | 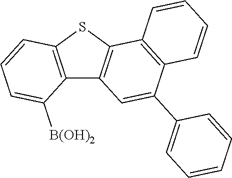 | 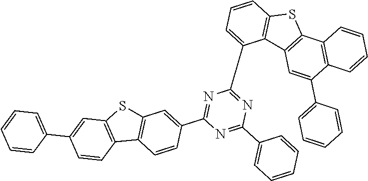 | 67 |
| 1-126 | 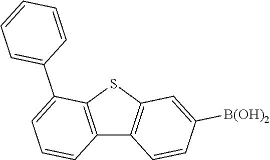 | 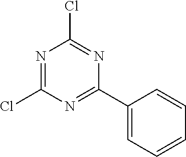 | 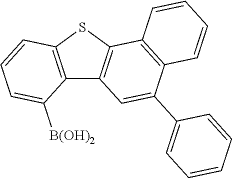 | 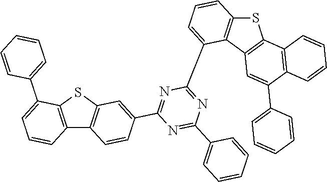 | 68 |
| 1-127 | 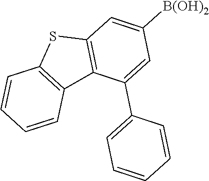 | 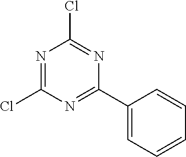 | 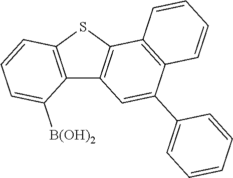 | 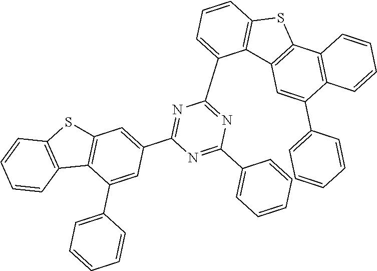 | 82 |
| 1-131 | 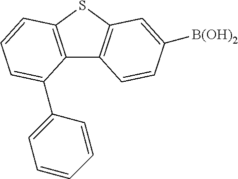 | 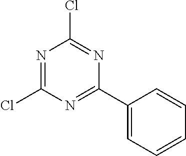 | 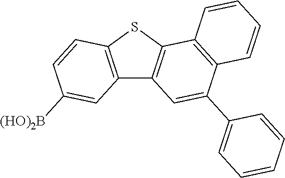 | 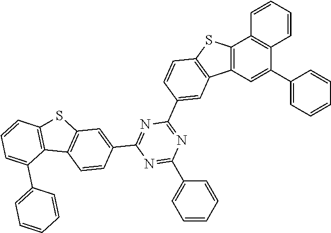 | 81 |
| 1-135 | 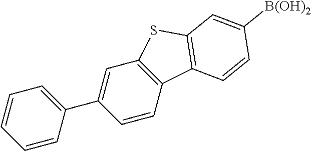 | 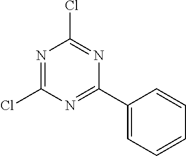 | 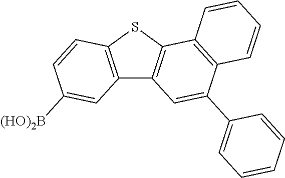 | 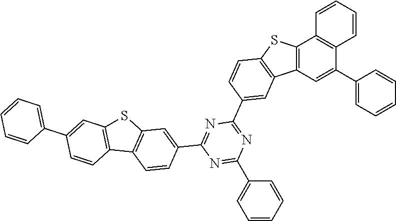 | 70 |
| 1-137 | 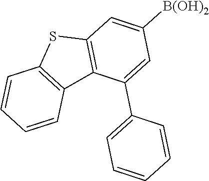 | 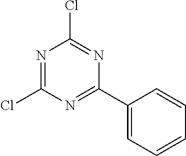 | 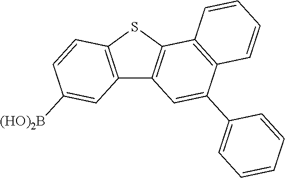 | 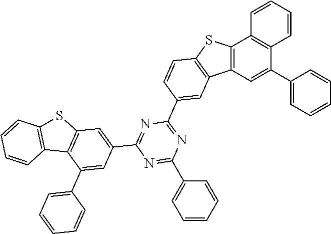 | 75 |
| 1-139 | 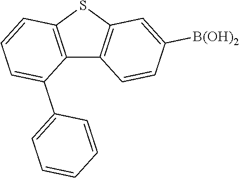 | 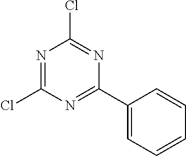 | 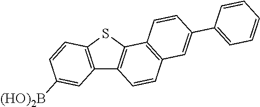 | 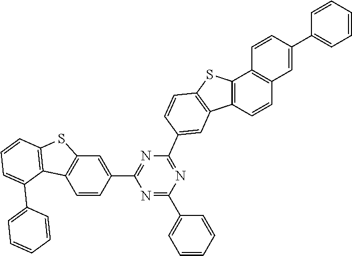 | 83 |
| 1-143 | 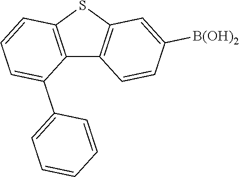 | 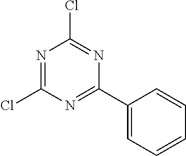 | 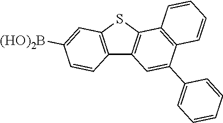 | 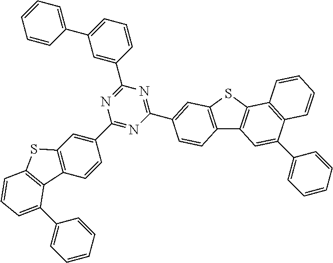 | 82 |
| 1-145 | 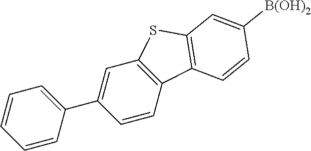 | 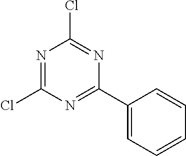 | 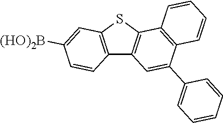 | 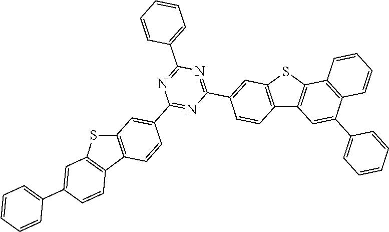 | 81 |
| 1-146 | 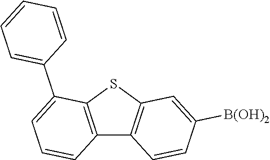 | 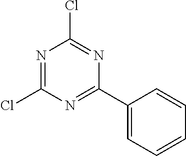 | 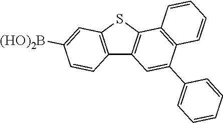 | 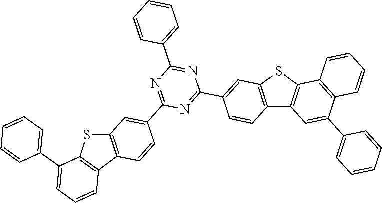 | 68 |
| 1-147 | 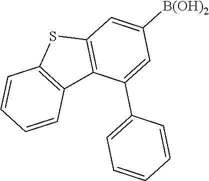 | 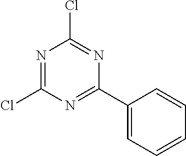 | 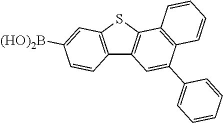 | 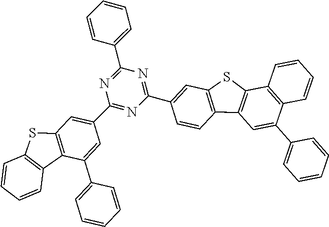 | 83 |
| 1-154 | 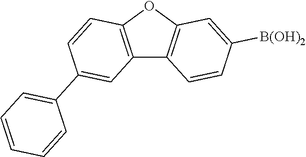 | 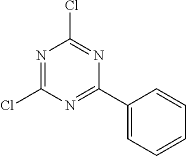 | 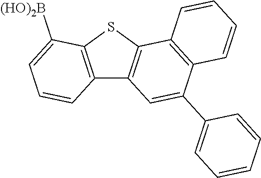 | 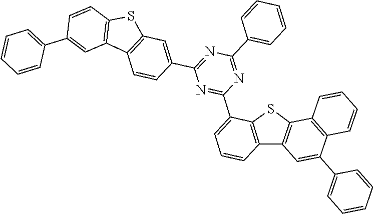 | 75 |
| 1-155 | 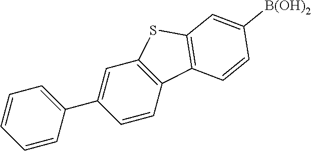 | 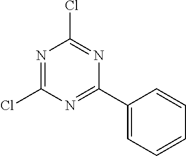 | 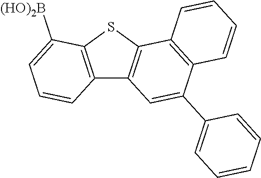 | 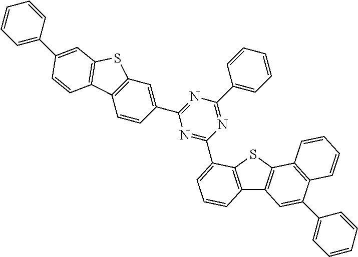 | 75 |
| 1-156 | 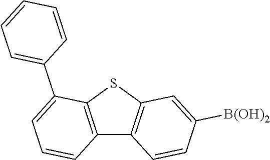 | 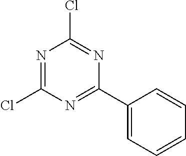 | 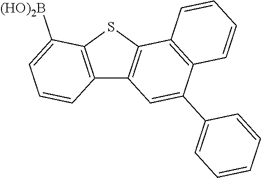 | 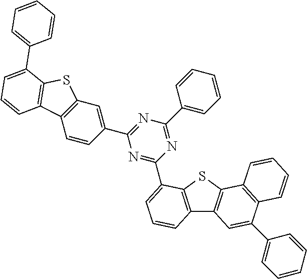 | 65 |
| 1-157 | 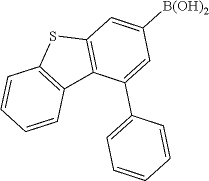 | 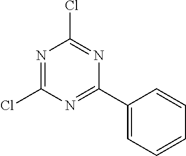 | 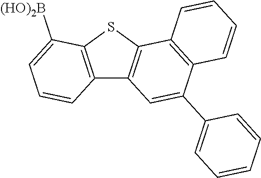 | 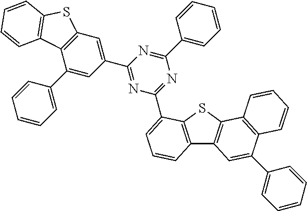 | 65 |

### [Preparation Example 2] Preparation of Compound 2-[C]-1

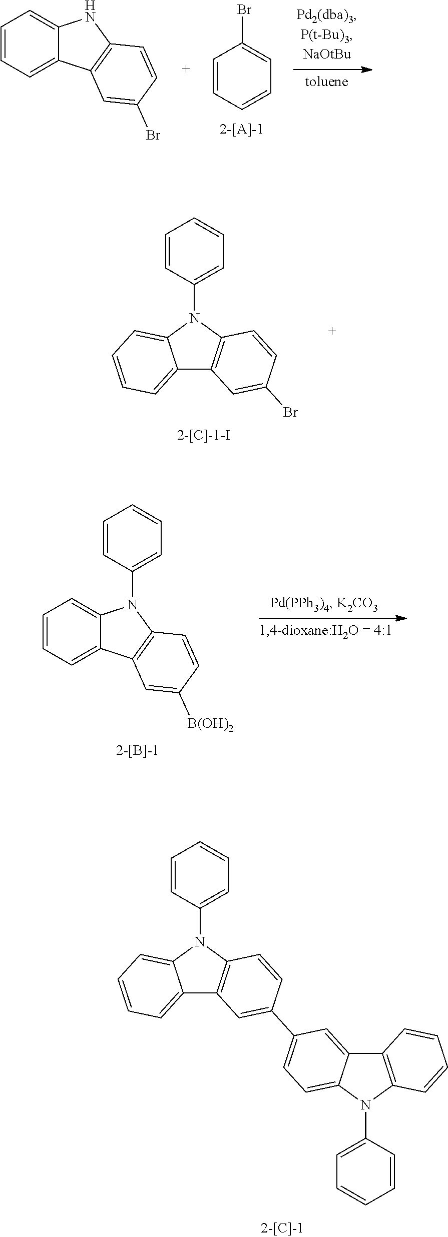 [SMILES: Brc1ccc2[nH]c3ccccc3c2c1 ; Brc1ccccc1 ; Brc1ccc2c(c1)c1ccccc1n2-c1ccccc1 ; OB(O)c1ccc2c(c1)c1ccccc1n2-c1ccccc1 ; c1ccc(-n2c3ccccc3c3cc(-c4ccc5c(c4)c4ccccc4n5-c4ccccc4)ccc32)cc1]

### Preparation of Compound 2-[C]-1-i

3-Bromo-9H-carbazole (10 g, 49.59 mmol), 2-bromobenzene-1-ylium (A) (24.2 g, 148.77 mmol), Pd₂(dba)₃ (2.27 g, 2.48 mmol), P(t-Bu)₃ (2.42 mL, 9.92 mmol), and NaOtBu (9.53 g, 99.18 mmol) were put into a one neck round bottom flask, and then toluene (100 mL) was added thereto, and the resulting mixture was heated at 135° C. for 15 hours. When the reaction was completed, extraction was performed with MC and H₂O, and then column purification was performed to obtain Compound 2-[C]-1-i. (14 g, 98%)

### Preparation of Compound 2-[C]-1

After 2-[C]-1-i (14 g, 43.4 mmol), (9-phenyl-9H-carbazol-3-yl)boronic acid (B) (14.9 g, 52 mmol), Pd(PPh₃)₄ (2.5 g, 2.17 mmol), and K₂CO₃ (17.9 g, 130 mmol) were put into a one neck round bottom flask, a mixture of 1,4-dioxane/water (140 ml/35 ml) was heated at 120° C. for 4 hours. After the reaction was completed, the temperature was lowered to room temperature, and then the resulting solid was washed with distilled water and MeOH to obtain Compound 2-[C]-1. (17 g, 80%)

The following Compound 2-C was synthesized in the same manner as in Preparation Example 2, except that another Compound 2-A in the following Table 2 was used instead of Compound 2-[A]-1, and Compound 2-B in the following Table 2 was used instead of Compound 2-f[B]-1.

TABLE 2

| | | | | Yield |
|---|---|---|---|---|
| Compound | 2-A | 2-B | 2-C | (%) |
| 2-2 | 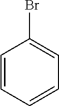 | 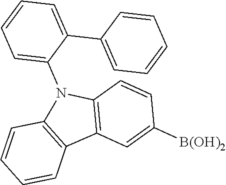 [SMILES: OB(O)c1ccc2c(c1)c1ccccc1n2-c1ccccc1-c1ccccc1] | 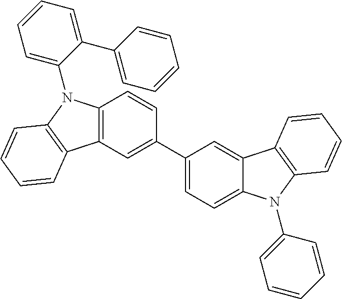 [SMILES: c1ccc(-c2ccccc2-n2c3ccccc3c3cc(-c4ccc5c(c4)c4ccccc4n5-c4ccccc4)ccc32)cc1] | 80% |
| 2-3 | 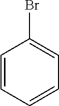 | 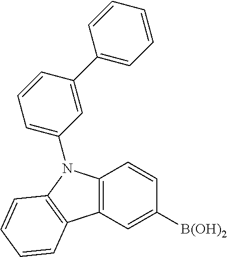 [SMILES: OB(O)c1ccc2c(c1)c1ccccc1n2-c1cccc(-c2ccccc2)c1] | 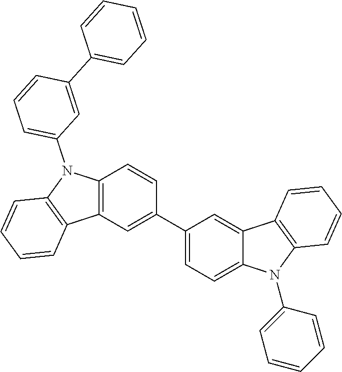 [SMILES: c1ccc(-c2cccc(-n3c4ccccc4c4cc(-c5ccc6c(c5)c5ccccc5n6-c5ccccc5)ccc43)c2)cc1] | 76% |
| 2-4 | 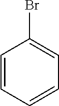 | 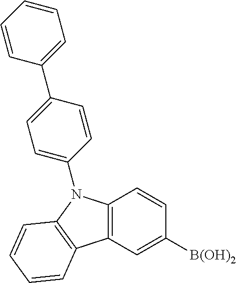 [SMILES: OB(O)c1ccc2c(c1)c1ccccc1n2-c1ccc(-c2ccccc2)cc1] | 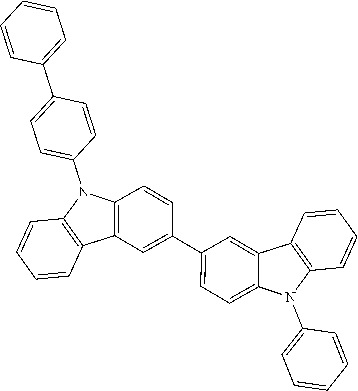 [SMILES: c1ccc(-c2ccc(-n3c4ccccc4c4cc(-c5ccc6c(c5)c5ccccc5n6-c5ccccc5)ccc43)cc2)cc1] | 69% |
| 2-5 | 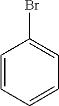 | 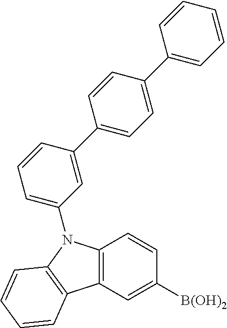 [SMILES: OB(O)c1ccc2c(c1)c1ccccc1n2-c1cccc(-c2ccc(-c3ccccc3)cc2)c1] | 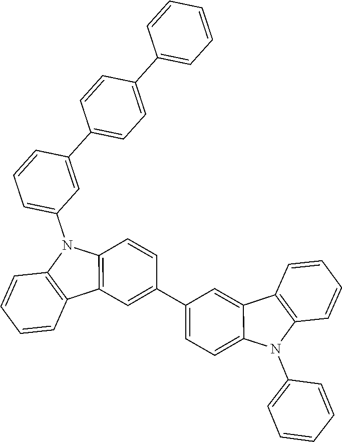 [SMILES: c1ccc(-c2ccc(-c3cccc(-n4c5ccccc5c5cc(-c6ccc7c(c6)c6ccccc6n7-c6ccccc6)ccc54)c3)cc2)cc1] | 68% |
| 2-6 | 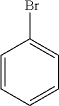 | 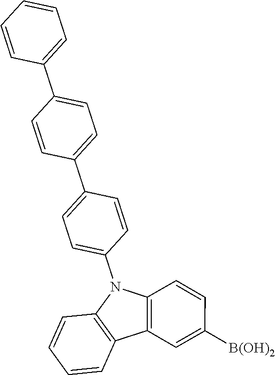 [SMILES: OB(O)c1ccc2c(c1)c1ccccc1n2-c1ccc(-c2ccc(-c3ccccc3)cc2)cc1] | 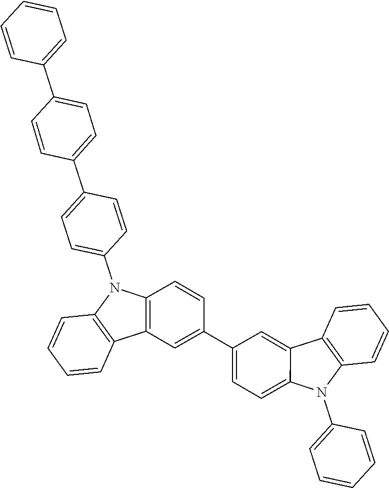 [SMILES: c1ccc(-c2ccc(-c3ccc(-n4c5ccccc5c5cc(-c6ccc7c(c6)c6ccccc6n7-c6ccccc6)ccc54)cc3)cc2)cc1] | 70% |
| 2-7 | 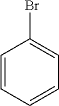 | 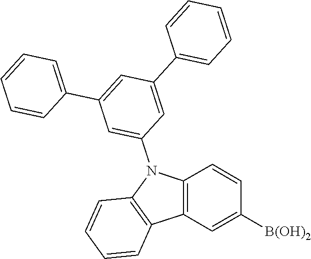 [SMILES: OB(O)c1ccc2c(c1)c1ccccc1n2-c1cc(-c2ccccc2)cc(-c2ccccc2)c1] | 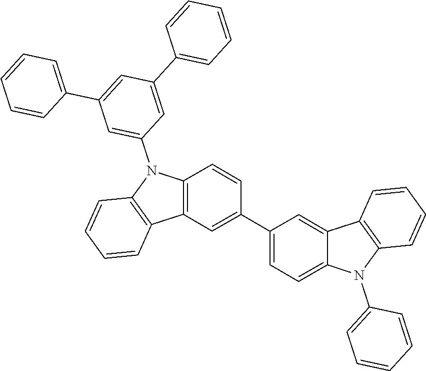 [SMILES: c1ccc(-c2cc(-c3ccccc3)cc(-n3c4ccccc4c4cc(-c5ccc6c(c5)c5ccccc5n6-c5ccccc5)ccc43)c2)cc1] | 73% |
| 2-11 | 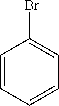 | 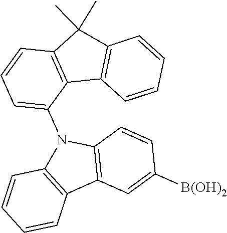 [SMILES: CC1(C)c2ccccc2-c2c(-n3c4ccccc4c4cc(B(O)O)ccc43)cccc21] | 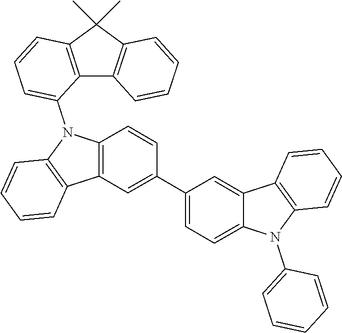 [SMILES: CC1(C)c2ccccc2-c2c(-n3c4ccccc4c4cc(-c5ccc6c(c5)c5ccccc5n6-c5ccccc5)ccc43)cccc21] | 69% |
| 2-16 | 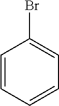 | 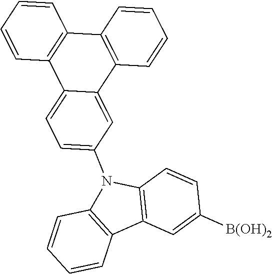 [SMILES: OB(O)c1ccc2c(c1)c1ccccc1n2-c1ccc2c3ccccc3c3ccccc3c2c1] | 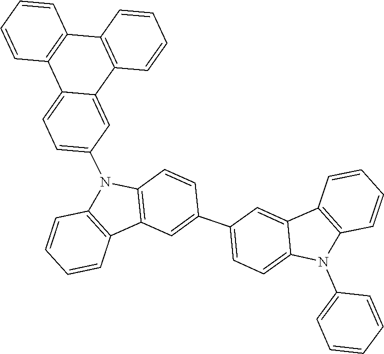 [SMILES: c1ccc(-n2c3ccccc3c3cc(-c4ccc5c(c4)c4ccccc4n5-c4ccc5c6ccccc6c6ccccc6c5c4)ccc32)cc1] | 70% |
| 2-19 | 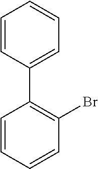 [SMILES: Brc1ccccc1-c1ccccc1] | 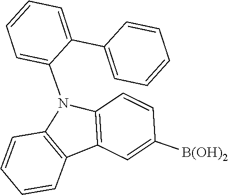 [SMILES: OB(O)c1ccc2c(c1)c1ccccc1n2-c1ccccc1-c1ccccc1] | 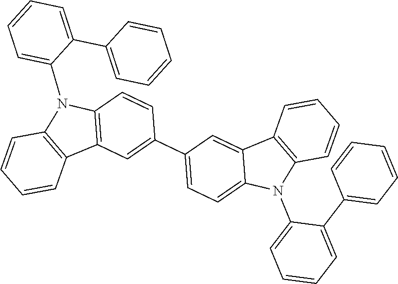 [SMILES: c1ccc(-c2ccccc2-n2c3ccccc3c3cc(-c4ccc5c(c4)c4ccccc4n5-c4ccccc4-c4ccccc4)ccc32)cc1] | 76% |
| 2-20 | 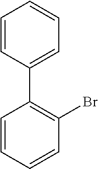 [SMILES: Brc1ccccc1-c1ccccc1] | 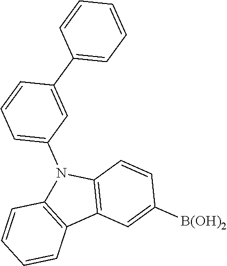 [SMILES: OB(O)c1ccc2c(c1)c1ccccc1n2-c1cccc(-c2ccccc2)c1] | 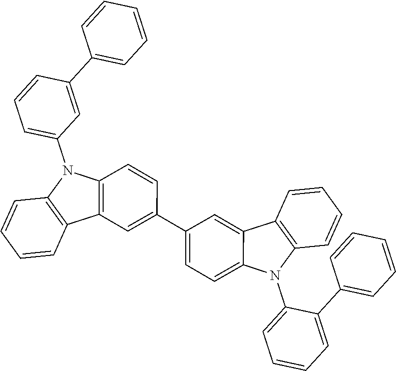 [SMILES: c1ccc(-c2cccc(-n3c4ccccc4c4cc(-c5ccc6c(c5)c5ccccc5n6-c5ccccc5-c5ccccc5)ccc43)c2)cc1] | 84% |
| 2-21 | 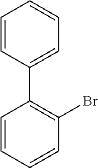 [SMILES: Brc1ccccc1-c1ccccc1] | 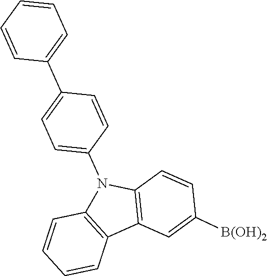 [SMILES: OB(O)c1ccc2c(c1)c1ccccc1n2-c1ccc(-c2ccccc2)cc1] | 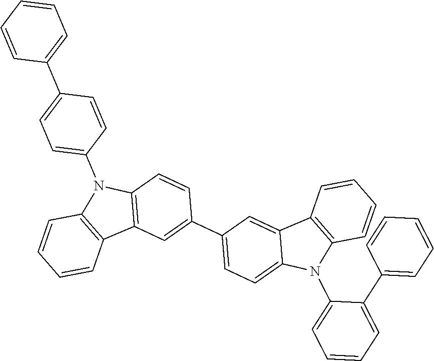 [SMILES: c1ccc(-c2ccc(-n3c4ccccc4c4cc(-c5ccc6c(c5)c5ccccc5n6-c5ccccc5-c5ccccc5)ccc43)cc2)cc1] | 80% |
| 2-22 | 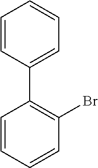 [SMILES: Brc1ccccc1-c1ccccc1] | 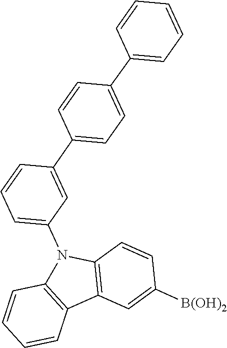 [SMILES: OB(O)c1ccc2c(c1)c1ccccc1n2-c1cccc(-c2ccc(-c3ccccc3)cc2)c1] | 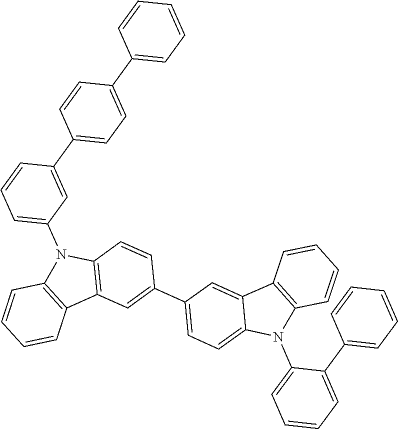 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1] | 83% |
| 2-23 | 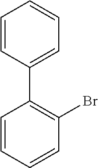 [SMILES: Brc1ccccc1-c1ccccc1] | 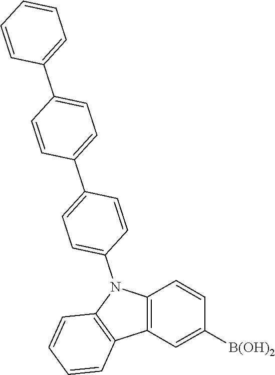 [SMILES: OB(O)c1ccc2c(c1)c1ccccc1n2-c1ccc(-c2ccc(-c3ccccc3)cc2)cc1] | 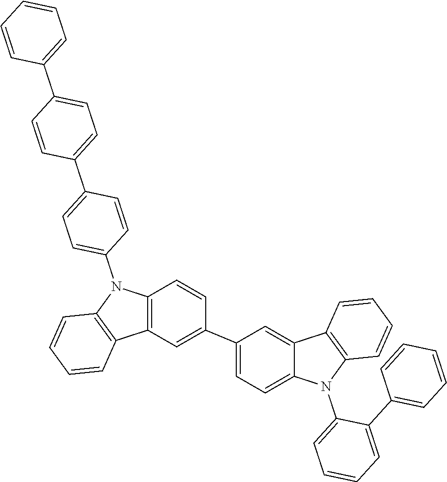 [SMILES: c1ccc(-c2ccc(-c3ccc(-n4c5ccccc5c5cc(-c6ccc7c(c6)c6ccccc6n7-c6ccccc6-c6ccccc6)ccc54)cc3)cc2)cc1] | 70% |
| 2-26 | 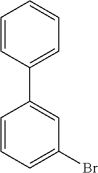 [SMILES: Brc1cccc(-c2ccccc2)c1] | 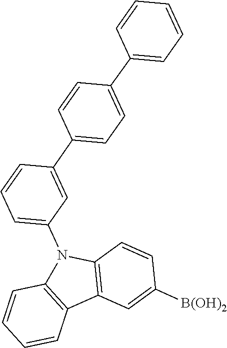 [SMILES: OB(O)c1ccc2c(c1)c1ccccc1n2-c1cccc(-c2ccc(-c3ccccc3)cc2)c1] | 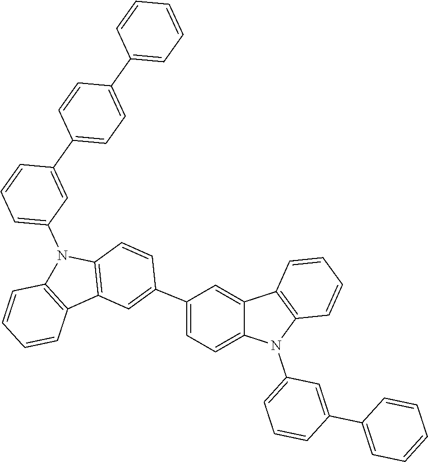 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] | 73% |
| 2-27 | 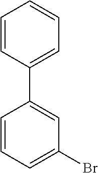 [SMILES: Brc1cccc(-c2ccccc2)c1] | 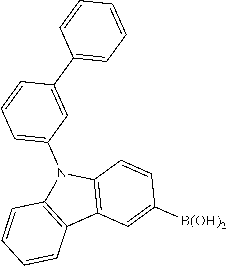 [SMILES: OB(O)c1ccc2c(c1)c1ccccc1n2-c1cccc(-c2ccccc2)c1] | 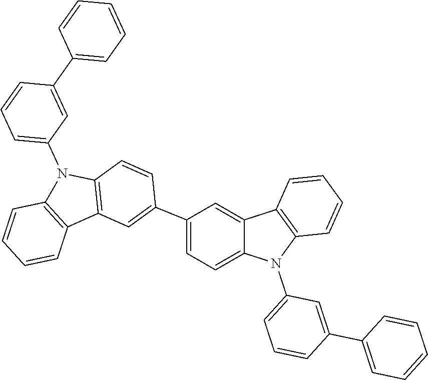 [SMILES: c1ccc(-c2cccc(-n3c4ccccc4c4cc(-c5ccc6c(c5)c5ccccc5n6-c5cccc(-c6ccccc6)c5)ccc43)c2)cc1] | 84% |
| 2-28 | 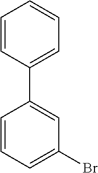 [SMILES: Brc1cccc(-c2ccccc2)c1] | 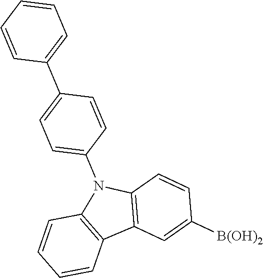 [SMILES: OB(O)c1ccc2c(c1)c1ccccc1n2-c1ccc(-c2ccccc2)cc1] | 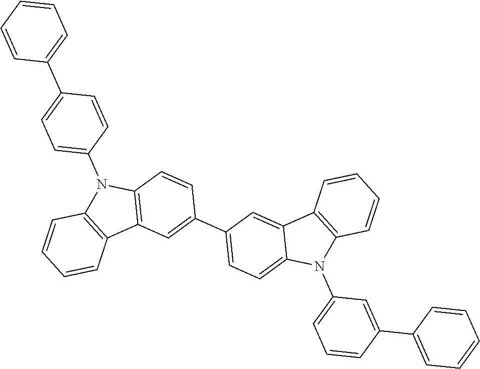 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 74% |
| 2-29 | 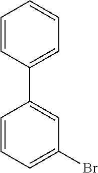 [SMILES: Brc1cccc(-c2ccccc2)c1] | 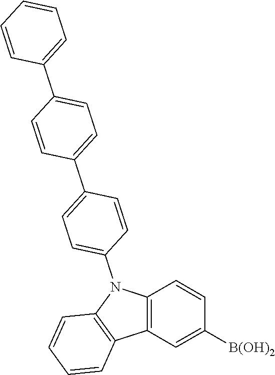 [SMILES: OB(O)c1ccc2c(c1)c1ccccc1n2-c1ccc(-c2ccc(-c3ccccc3)cc2)cc1] | 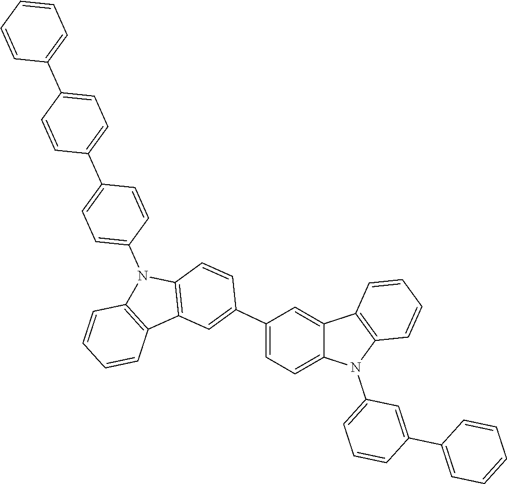 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1] | 69% |
| 2-30 | 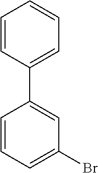 [SMILES: Brc1cccc(-c2ccccc2)c1] | 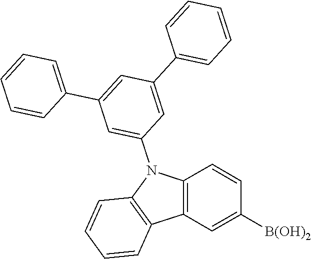 [SMILES: OB(O)c1ccc2c(c1)c1ccccc1n2-c1cc(-c2ccccc2)cc(-c2ccccc2)c1] | 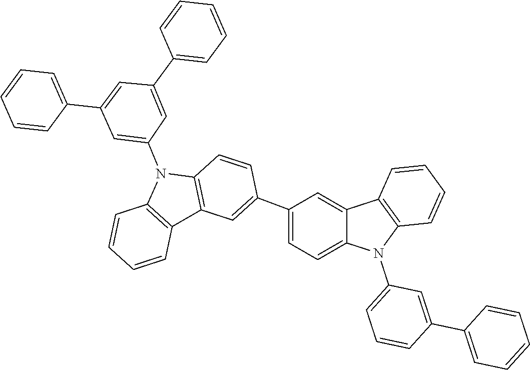 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] | 76% |
| 2-32 | 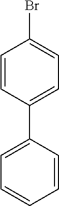 [SMILES: Brc1ccc(-c2ccccc2)cc1] | 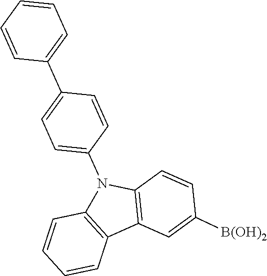 [SMILES: OB(O)c1ccc2c(c1)c1ccccc1n2-c1ccc(-c2ccccc2)cc1] | 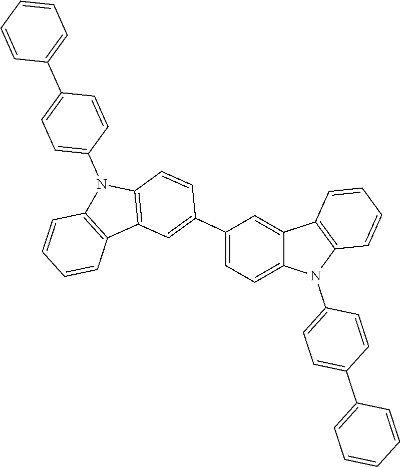 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 75% |
| 2-33 | 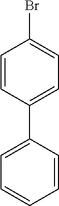 [SMILES: Brc1ccc(-c2ccccc2)cc1] | 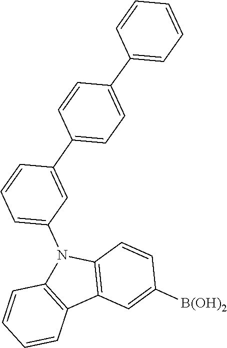 [SMILES: OB(O)c1ccc2c(c1)c1ccccc1n2-c1cccc(-c2ccc(-c3ccccc3)cc2)c1] | 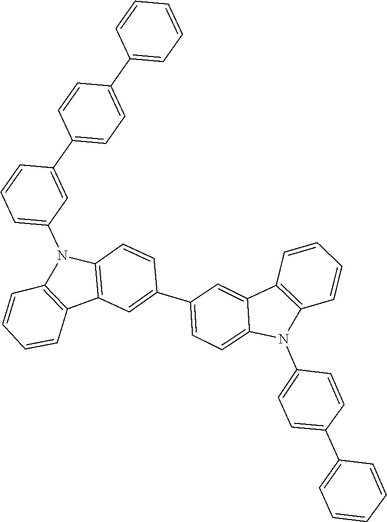 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1] | 72% |
| 2-34 | 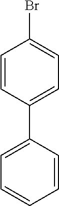 [SMILES: Brc1ccc(-c2ccccc2)cc1] | 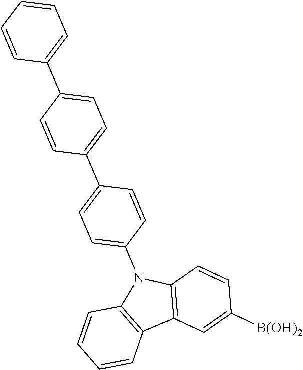 [SMILES: OB(O)c1ccc2c(c1)c1ccccc1n2-c1ccc(-c2ccc(-c3ccccc3)cc2)cc1] | 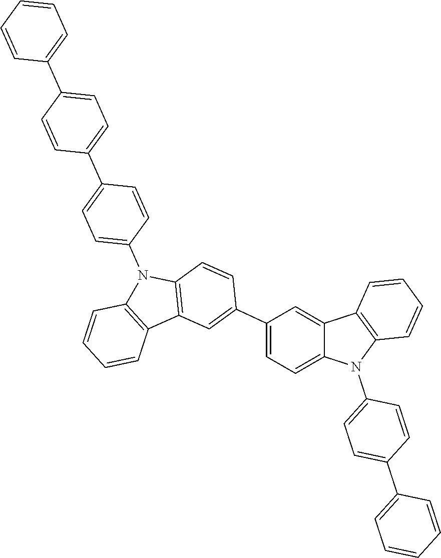 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1] | 84% |
| 2-38 | 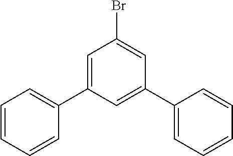 [SMILES: Brc1cc(-c2ccccc2)cc(-c2ccccc2)c1] | 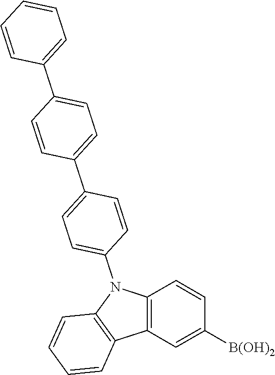 [SMILES: OB(O)c1ccc2c(c1)c1ccccc1n2-c1ccc(-c2ccc(-c3ccccc3)cc2)cc1] | 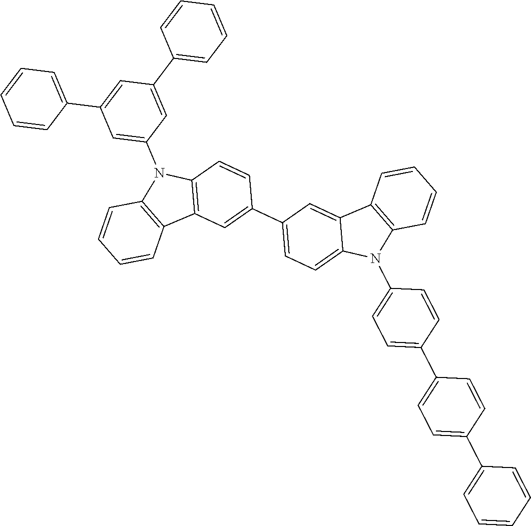 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1] | 74% |
| 2-40 | 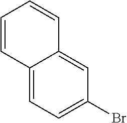 [SMILES: Brc1ccc2ccccc2c1] | 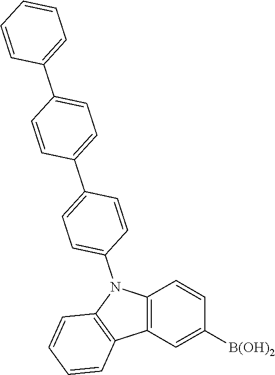 [SMILES: OB(O)c1ccc2c(c1)c1ccccc1n2-c1ccc(-c2ccc(-c3ccccc3)cc2)cc1] | 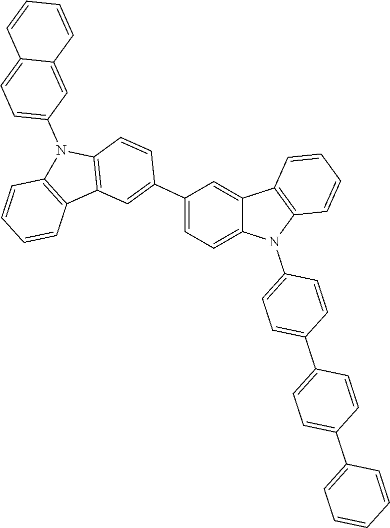 [SMILES: c1ccc(-c2ccc(-c3ccc(-n4c5ccccc5c5cc(-c6ccc7c(c6)c6ccccc6n7-c6ccc7ccccc7c6)ccc54)cc3)cc2)cc1] | 84% |
| 2-41 | 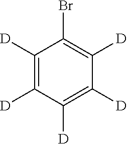 [SMILES: [2H]c1c([2H])c([2H])c(Br)c([2H])c1[2H]] | 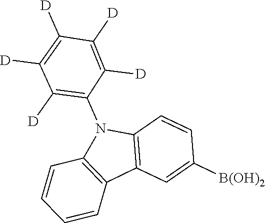 [SMILES: [2H]c1c([2H])c([2H])c(-n2c3ccccc3c3cc(B(O)O)ccc32)c([2H])c1[2H]] | 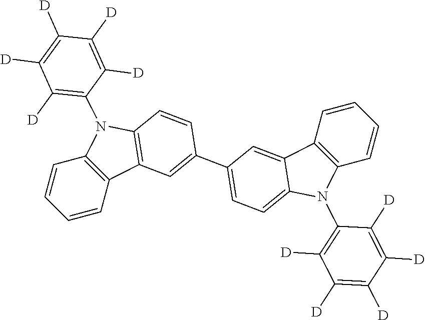 [SMILES: [2H]c1c([2H])c([2H])c(-n2c3ccccc3c3cc(-c4ccc5c(c4)c4ccccc4n5-c4c([2H])c([2H])c([2H])c([2H])c4[2H])ccc32)c([2H])c1[2H]] | 70% |
| 2-42 | 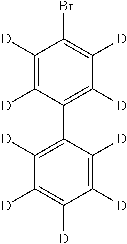 [SMILES: [2H]c1c([2H])c([2H])c(-c2c([2H])c([2H])c(Br)c([2H])c2[2H])c([2H])c1[2H]] | 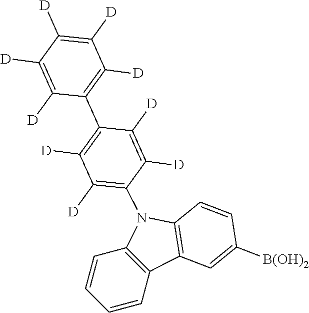 [SMILES: [2H]c1c([2H])c([2H])c(-c2c([2H])c([2H])c(-n3c4ccccc4c4cc(B(O)O)ccc43)c([2H])c2[2H])c([2H])c1[2H]] | 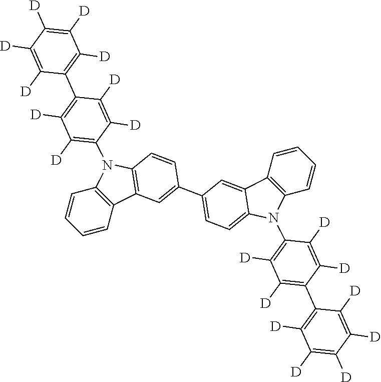 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]] | 84% |
| 2-43 | 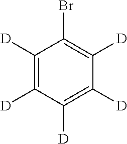 [SMILES: [2H]c1c([2H])c([2H])c(Br)c([2H])c1[2H]] | 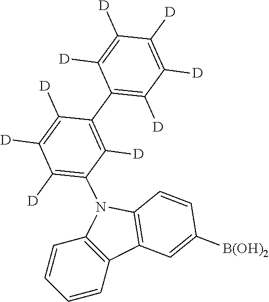 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 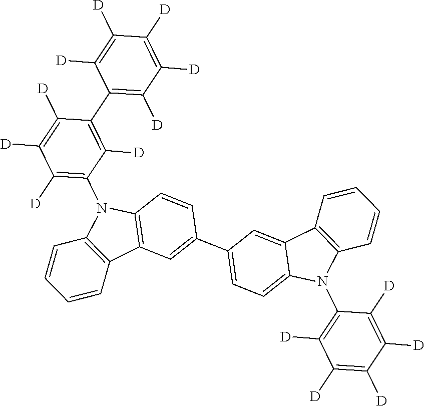 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c([2H])c5[2H])ccc43)c2[2H])c([2H])c1[2H]] | 81% |
| 2-45 | 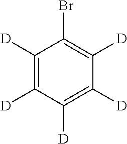 [SMILES: [2H]c1c([2H])c([2H])c(Br)c([2H])c1[2H]] | 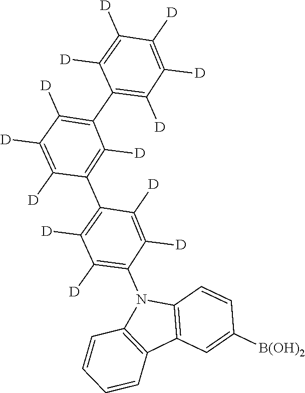 [SMILES: [2H]c1c([2H])c([2H])c(-c2c([2H])c([2H])c([2H])c(-c3c([2H])c([2H])c(-n4c5ccccc5c5cc(B(O)O)ccc54)c([2H])c3[2H])c2[2H])c([2H])c1[2H]] | 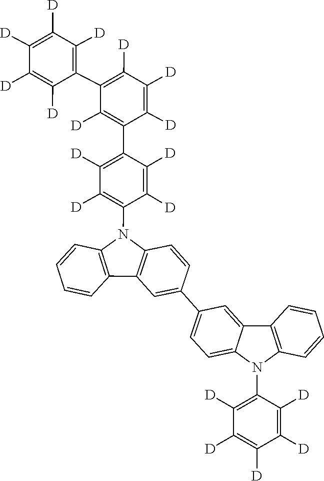 [SMILES: [2H]c1c([2H])c([2H])c(-c2c([2H])c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c2[2H])c([2H])c1[2H]] | 68% |
| 2-46 | 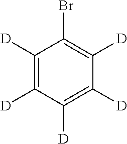 [SMILES: [2H]c1c([2H])c([2H])c(Br)c([2H])c1[2H]] | 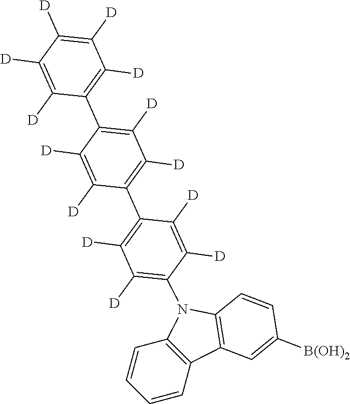 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 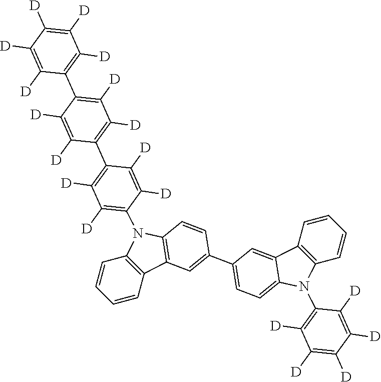 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c([2H])c6[2H])ccc54)c([2H])c3[2H])c([2H])c2[2H])c([2H])c1[2H]] | 81% |
| 2-48 | 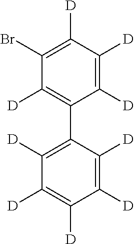 [SMILES: [2H]c1c([2H])c([2H])c(-c2c([2H])c([2H])c([2H])c(Br)c2[2H])c([2H])c1[2H]] | 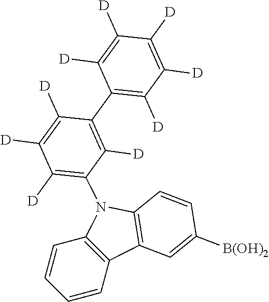 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 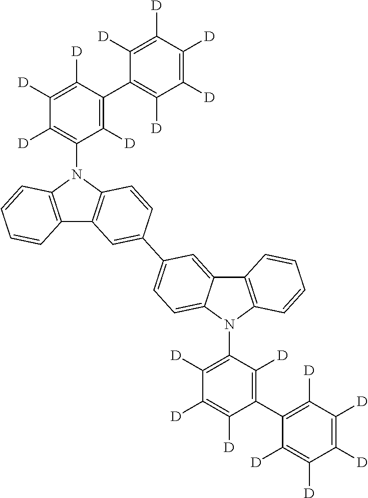 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]] | 70% |
| 2-49 | 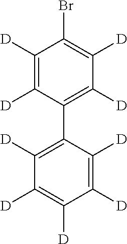 [SMILES: [2H]c1c([2H])c([2H])c(-c2c([2H])c([2H])c(Br)c([2H])c2[2H])c([2H])c1[2H]] | 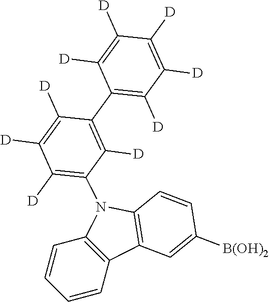 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 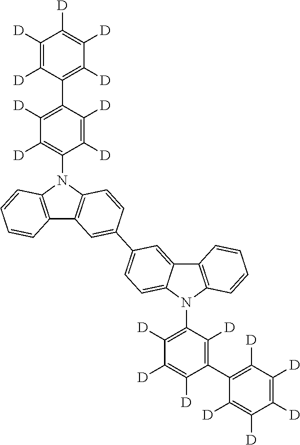 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c([2H])c2[2H])c([2H])c1[2H]] | 71% |
| 2-50 | 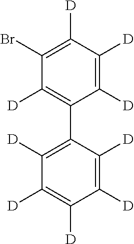 [SMILES: [2H]c1c([2H])c([2H])c(-c2c([2H])c([2H])c([2H])c(Br)c2[2H])c([2H])c1[2H]] | 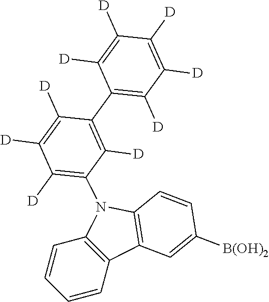 [SMILES: [2H]c1c([2H])c([2H])c(-c2c([2H])c([2H])c([2H])c(-n3c4ccccc4c4cc(B(O)O)ccc43)c2[2H])c([2H])c1[2H]] | 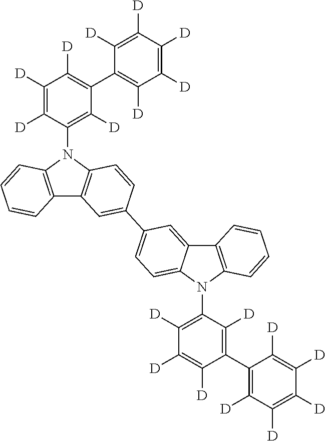 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c([2H])c(-c6c([2H])c([2H])c([2H])c([2H])c6[2H])c5[2H])ccc43)c2[2H])c([2H])c1[2H]] | 74% |
| 2-51 | 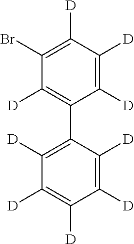 [SMILES: [2H]c1c([2H])c([2H])c(-c2c([2H])c([2H])c([2H])c(Br)c2[2H])c([2H])c1[2H]] | 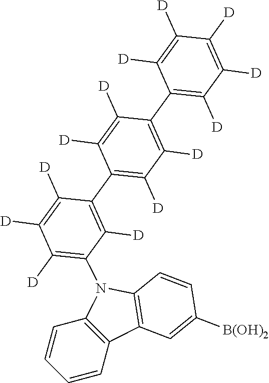 [SMILES: [2H]c1c([2H])c([2H])c(-c2c([2H])c([2H])c(-c3c([2H])c([2H])c([2H])c(-n4c5ccccc5c5cc(B(O)O)ccc54)c3[2H])c([2H])c2[2H])c([2H])c1[2H]] | 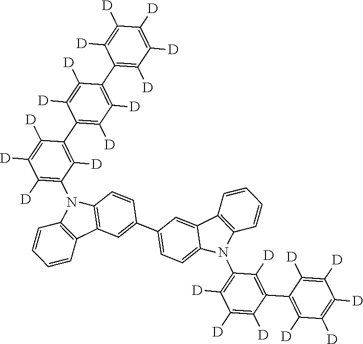 [SMILES: [2H]c1c([2H])c([2H])c(-c2c([2H])c([2H])c(-c3c([2H])c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c3[2H])c([2H])c2[2H])c([2H])c1[2H]] | 80% |
| 2-52 | 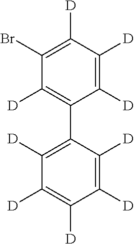 [SMILES: [2H]c1c([2H])c([2H])c(-c2c([2H])c([2H])c([2H])c(Br)c2[2H])c([2H])c1[2H]] | 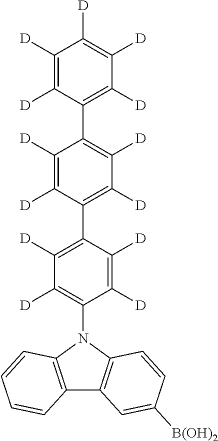 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 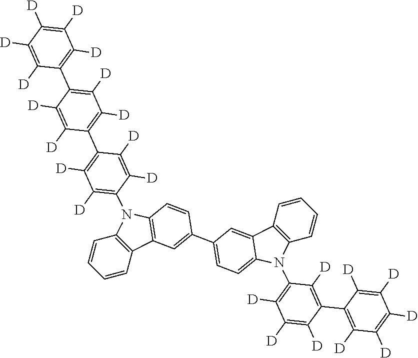 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c([2H])c([2H])c7[2H])c6[2H])ccc54)c([2H])c3[2H])c([2H])c2[2H])c([2H])c1[2H]] | 84% |
| 2-55 | 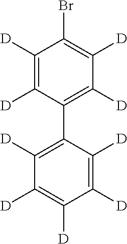 [SMILES: [2H]c1c([2H])c([2H])c(-c2c([2H])c([2H])c(Br)c([2H])c2[2H])c([2H])c1[2H]] | 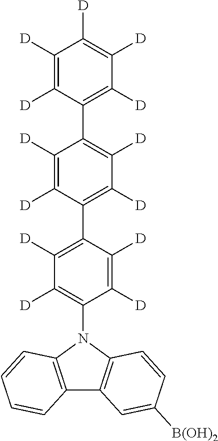 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 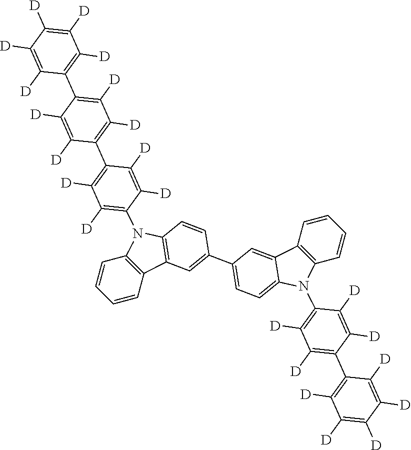 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c(-c7c([2H])c([2H])c([2H])c([2H])c7[2H])c([2H])c6[2H])ccc54)c([2H])c3[2H])c([2H])c2[2H])c([2H])c1[2H]] | 76% |
| 2-57 | 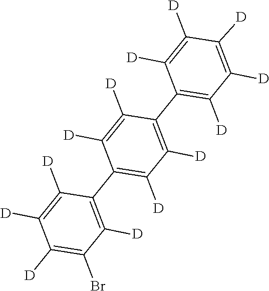 [SMILES: [2H]c1c([2H])c([2H])c(-c2c([2H])c([2H])c(-c3c([2H])c([2H])c([2H])c(Br)c3[2H])c([2H])c2[2H])c([2H])c1[2H]] | 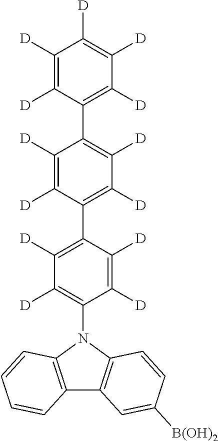 [SMILES: [2H]c1c([2H])c([2H])c(-c2c([2H])c([2H])c(-c3c([2H])c([2H])c(-n4c5ccccc5c5cc(B(O)O)ccc54)c([2H])c3[2H])c([2H])c2[2H])c([2H])c1[2H]] | 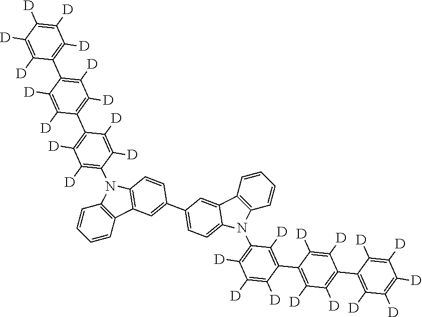 [SMILES: [2H]c1c([2H])c([2H])c(-c2c([2H])c([2H])c(-c3c([2H])c([2H])c(-n4c5ccccc5c5cc(-c6ccc7c(c6)c6ccccc6n7-c6c([2H])c([2H])c([2H])c(-c7c([2H])c([2H])c(-c8c([2H])c([2H])c([2H])c([2H])c8[2H])c([2H])c7[2H])c6[2H])ccc54)c([2H])c3[2H])c([2H])c2[2H])c([2H])c1[2H]] | 68% |
| 2-60 | 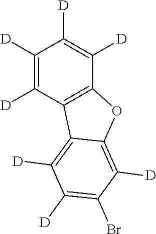 [SMILES: [2H]c1c([2H])c([2H])c2c(oc3c([2H])c(Br)c([2H])c([2H])c32)c1[2H]] | 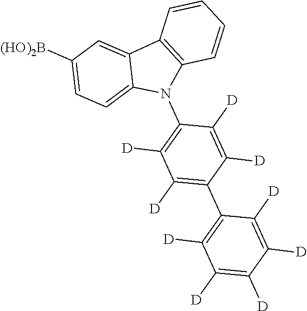 [SMILES: [2H]c1c([2H])c([2H])c(-c2c([2H])c([2H])c(-n3c4ccccc4c4cc(B(O)O)ccc43)c([2H])c2[2H])c([2H])c1[2H]] | 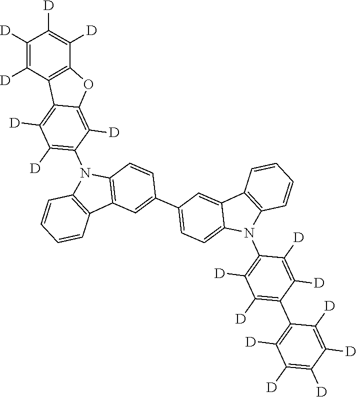 [SMILES: [2H]c1c([2H])c([2H])c(-c2c([2H])c([2H])c(-n3c4ccccc4c4cc(-c5ccc6c(c5)c5ccccc5n6-c5c([2H])c([2H])c6c(oc7c([2H])c([2H])c([2H])c([2H])c76)c5[2H])ccc43)c([2H])c2[2H])c([2H])c1[2H]] | 80% |

### [Preparation Example 3] Preparation of Compound 2-[F]-61

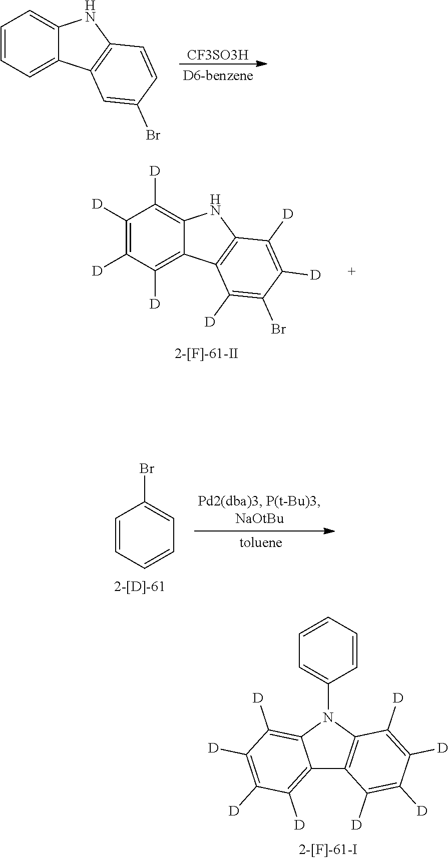

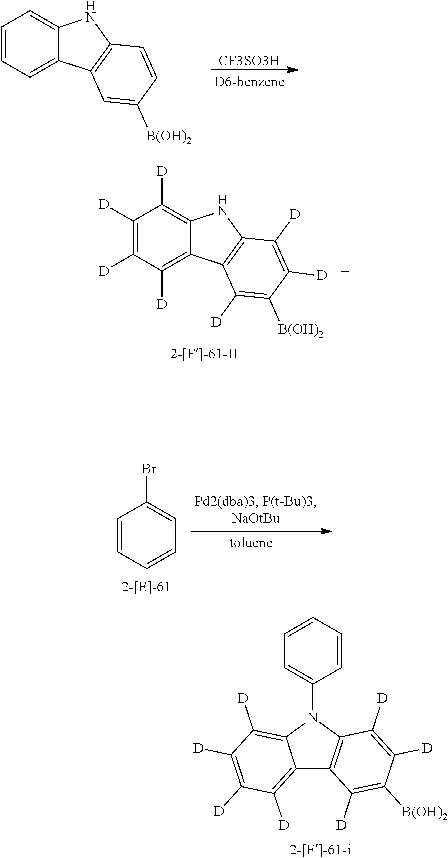

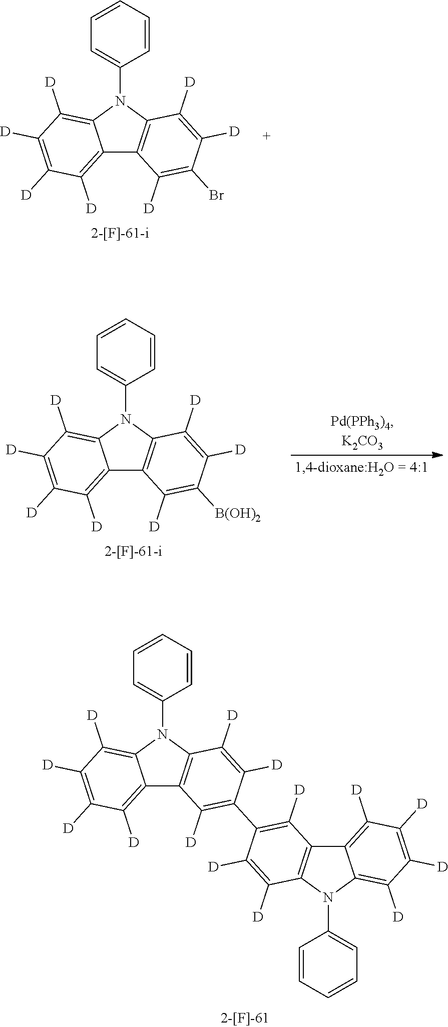 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c(B(O)O)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### Preparation of Compound 2-[F]-61-ii

After 3-bromo-9H-carbazole (10 g, 40.23 mmol) and 1000 ml of D₆-benzene were put into a one neck round bottom flask, CF₃SO₃H (170 g, 1075 mmol) was added thereto, and then the resulting mixture was stirred at 50° C. When the reaction was completed, the resulting product was neutralized with D₂O, then extracted with MC and an aqueous Na₂CO₃ solution, and then column purification was performed to obtain Compound 2-[F]-61-ii. (10 g, 98%)

### Preparation of Compound 2-[F]-61-i

After 2 2-[F]-61-ii (10 g, 39.5 mmol), bromobenzene (12.4 g, 79 mmol), Pd₂(dba)₃ (1.81 g, 1.98 mmol), P(t-Bu)₃ (1.93 mL, 7.9 mmol), and NaOtBu (11.4 g, 118.51 mmol) were put into a one neck round bottom flask, toluene (100 mL) was added thereto, and the resulting mixture was heated at 135° C. for 10 hours. When the reaction was completed, extraction was performed with MC and H₂O, and then column purification was performed to obtain Compound 2-[F]-61-i. (11 g, 84%)

### Preparation of Compound 2-[F′]-61-ii

After 9H-carbazol-3-ylboronic acid (10 g, 47.3 mmol) and 1000 ml of D₆-benzene were put into a one neck round bottom flask, CF₃SO₃H (170 g, 1075 mmol) was added thereto, and then the resulting mixture was stirred at 50° C. When the reaction was completed, the resulting product was neutralized with D₂O, then extracted with MC and an aqueous Na₂CO₃ solution, and then column purification was performed to obtain 2-[F′]-61-ii. (9 g, 87%)

### Preparation of Compound 2-[F]-61-i

After 2-[F′]-61-iI (9 g, 41.3 mmol), bromobenzene (12.9 g, 82.5 mmol), Pd₂(dba)₃ (1.89 g, 2.06 mmol), P(t-Bu)₃ (2 mL, 8.25 mmol), and NaOtBu (7.93 g, 82.54 mmol) were put into a one neck round bottom flask, toluene (100 mL) was added thereto, and the resulting mixture was heated at 135° C. for 10 hours. After the reaction was completed, extraction was performed with MC and H₂O, and then column purification was performed to obtain 2-[F]-61-i. (10 g, 82%)

### Preparation of Compound 2-[F]-61

After 2-[F]-61-i (10 g, 30.37 mmol), 2-[F′]-61-i (17.87 g, 60.75 mmol), Pd(PPh₃)₄ (1.39 g, 1.52 mmol), and K₂CO₃ (12.59 g, 91.13 mmol) were put into a one neck round bottom flask, a mixture of 1,4-dioxane/water (140 ml/35 ml) was heated at 120° C. for 4 hours. After the reaction was completed, the temperature was lowered to room temperature, and then the resulting solid was washed with distilled water and MeOH to obtain Compound 2-[F]-61. (13 g, 85%)

The following Compound (F) was synthesized in the same manner as in Preparation Example 3, except that another compound represented by D in the following Table 3 was used instead of 2-[D]-61 and another compound represented by E in the following Table 3 was used instead of 2-[E]-61.

TABLE 3

| | | | | Yield |
|---|---|---|---|---|
| Compound | D | E | F | (%) |
| 2-62 | 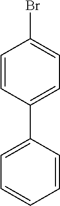 | 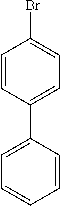 | 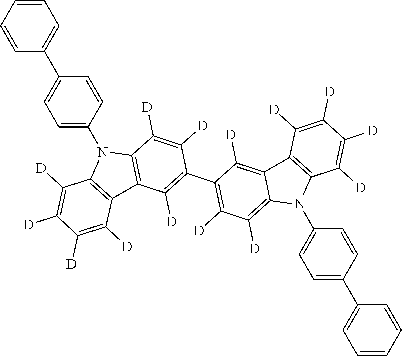 | 75% |
| 2-63 | 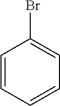 | 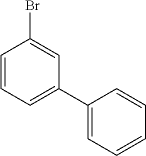 | 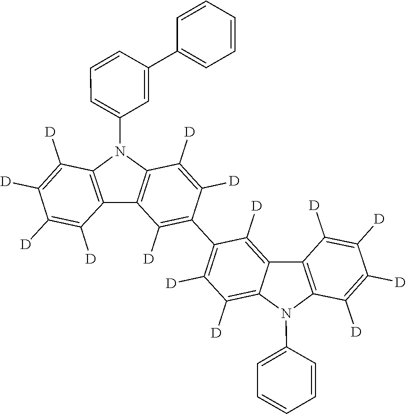 | 73% |
| 2-64 | 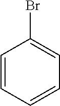 | 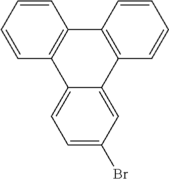 | 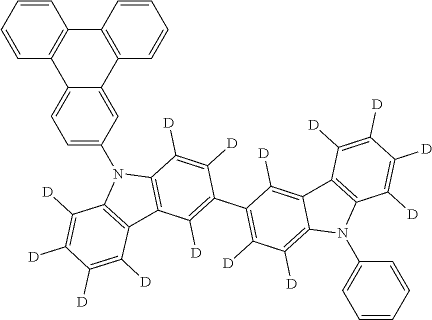 | 66% |
| 2-65 | 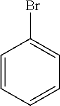 | 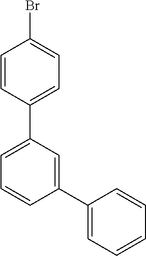 | 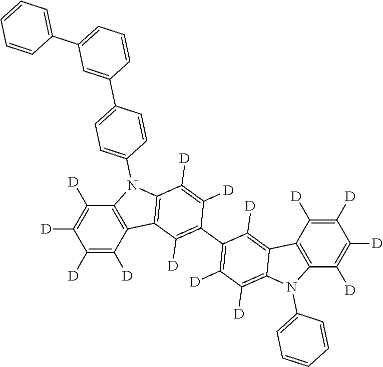 | 69% |
| 2-66 | 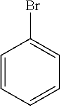 | 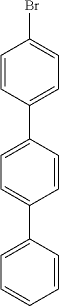 | 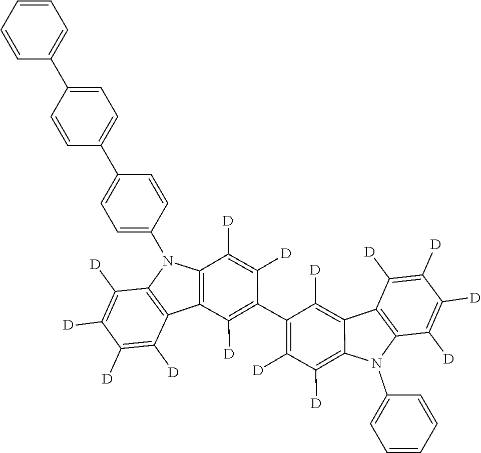 | 75% |
| 2-68 | 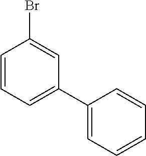 | 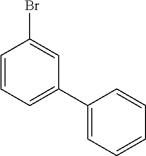 | 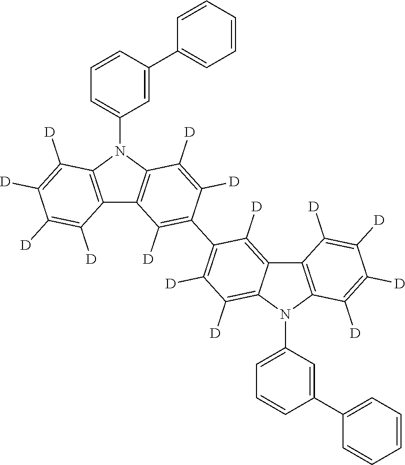 | 78% |
| 2-69 | 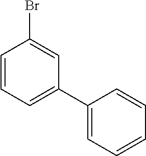 | 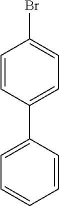 | 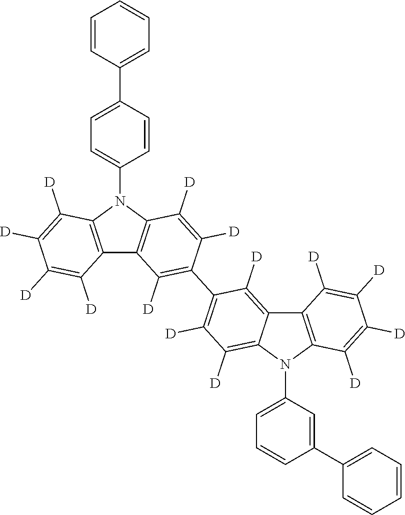 | 82% |
| 2-70 | 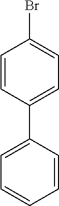 | 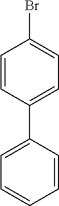 | 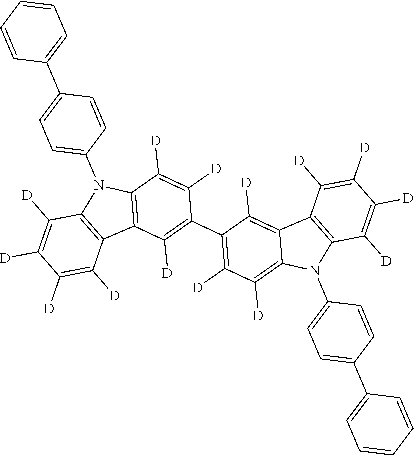 | 67% |
| 2-74 | 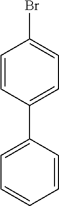 | 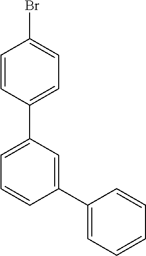 | 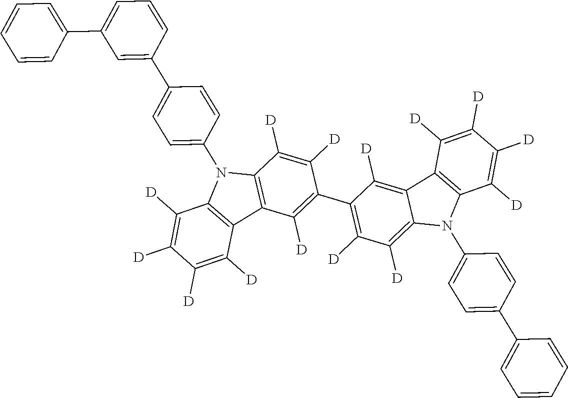 | 68% |
| 2-75 | 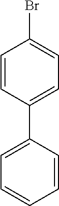 | 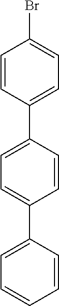 | 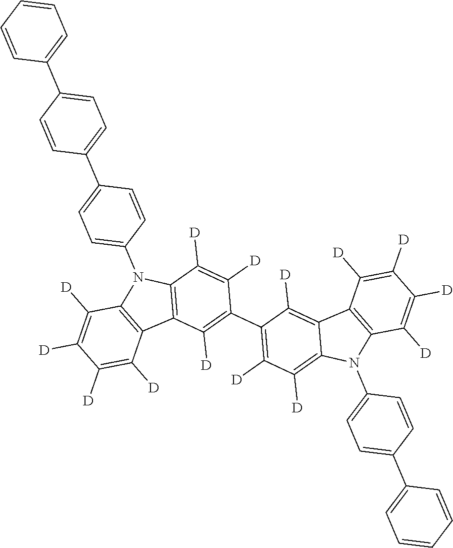 | 75% |
| 2-81 | 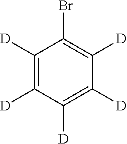 | 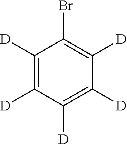 | 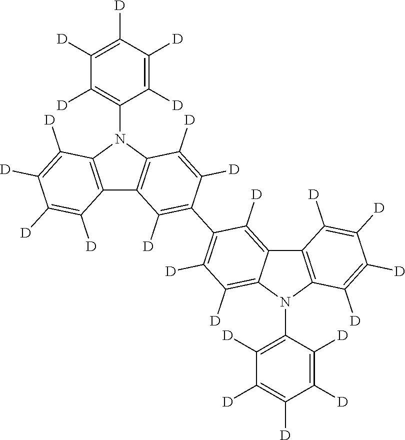 | 64% |
| 2-82 | 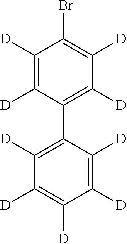 | 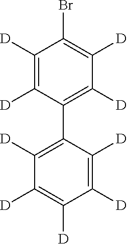 | 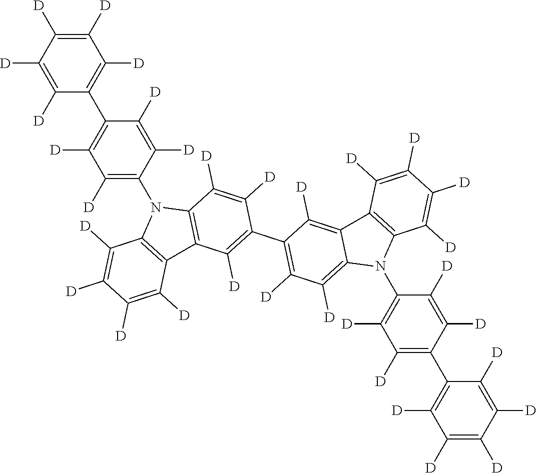 | 78% |
| 2-83 | 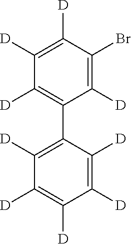 | 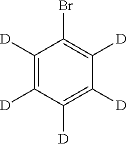 | 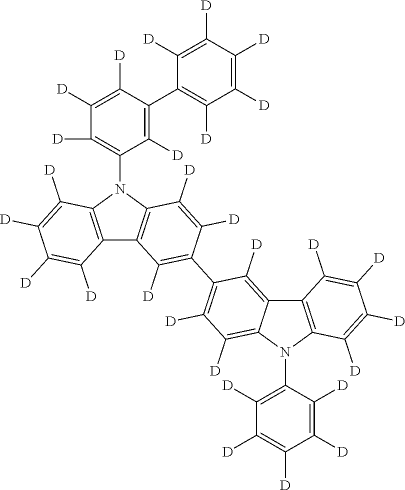 | 70% |
| 2-85 | 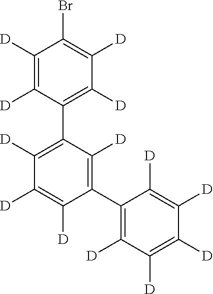 | 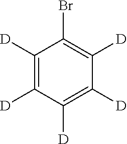 | 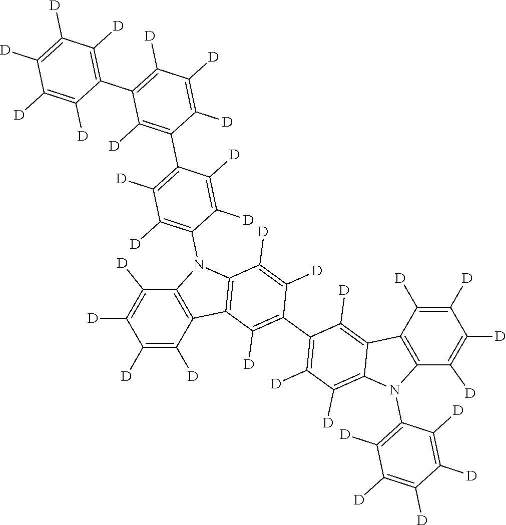 | 70% |
| 2-86 | 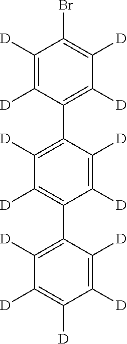 | 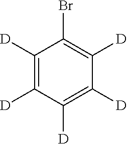 | 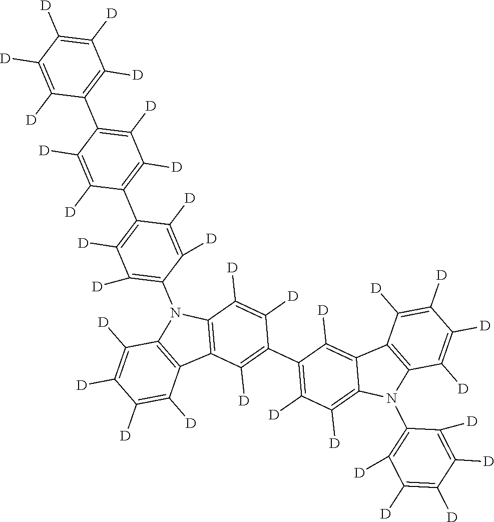 | 66% |
| 2-87 | 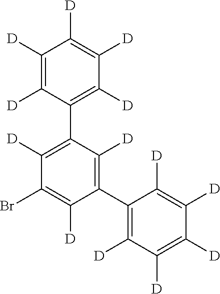 | 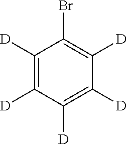 | 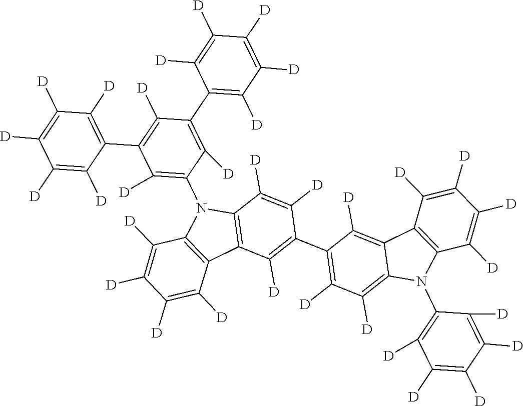 | 77% |
| 2-88 | 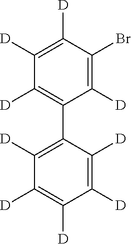 | 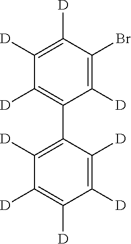 | 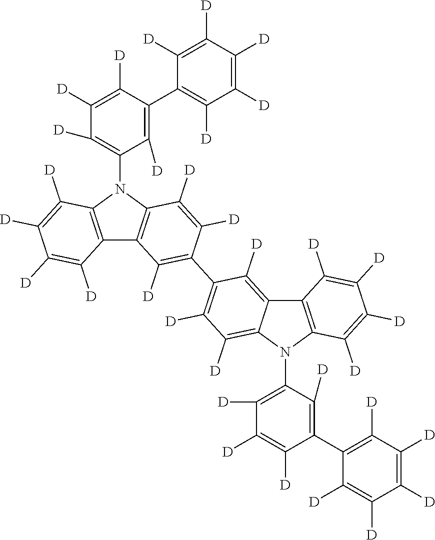 | 75% |
| 2-89 | 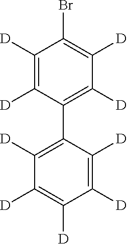 | 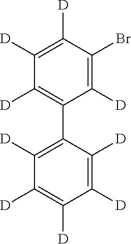 | 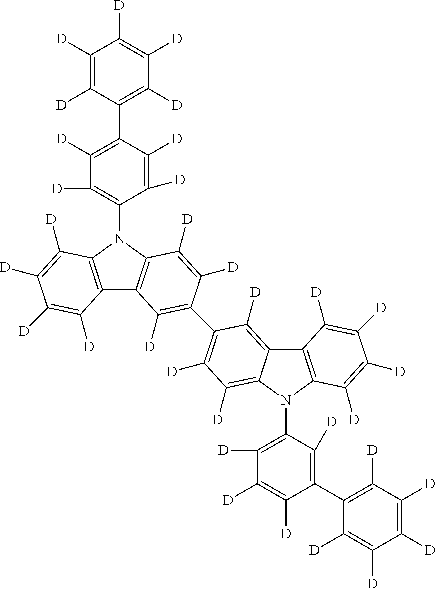 | 67% |
| 2-90 | 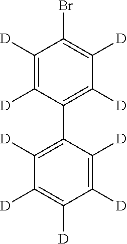 | 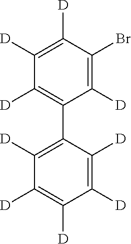 | 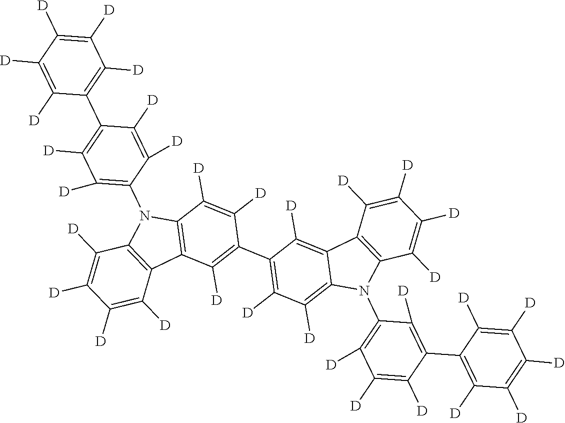 | 73% |
| 2-92 | 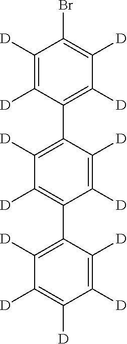 | 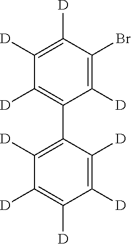 | 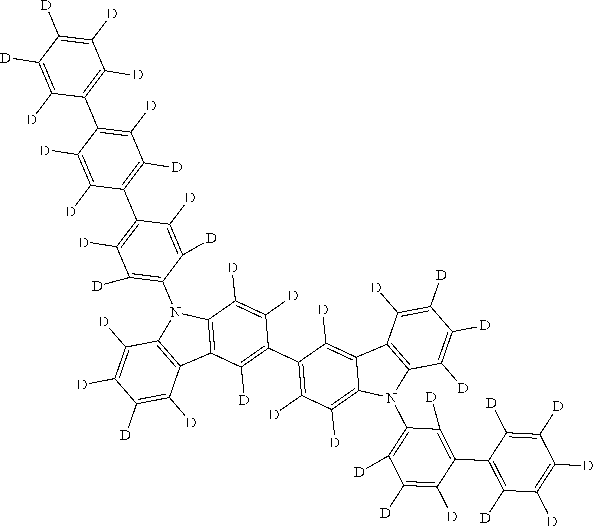 | 68% |
| 2-102 | 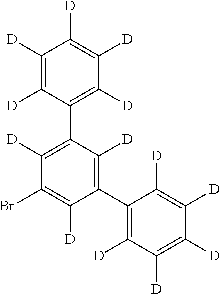 | 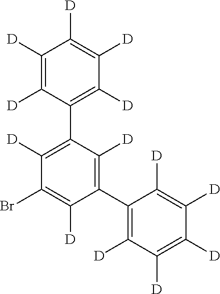 | 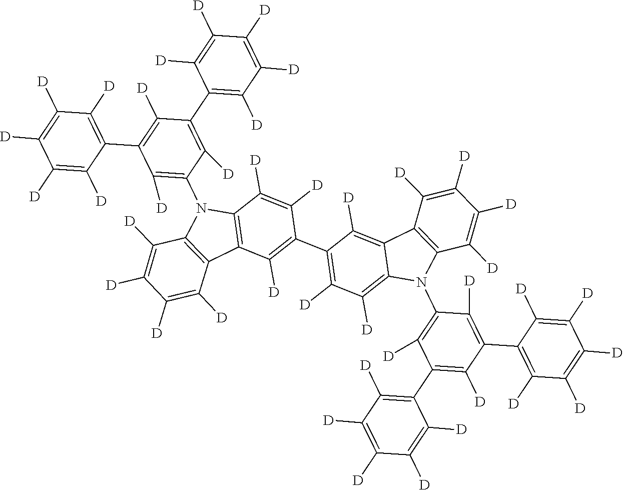 | 70% |

Compounds were prepared in the same manner as in the Preparation Examples, and the synthesis confirmation results thereof are shown in the following Tables 4 and 5. Table 4 shows measured values of field desorption mass spectrometry (FD-MS), and Table 5 shows NMR values.

TABLE 4

| Compound | FD-Mass | compound | FD-Mass |
|---|---|---|---|
| 1-1 | m/z = 691.77 (C49H29N3O2 = | 1-4 | m/z = 691.77 (C49H29N3O2 = |
| | 691.23) | | 691.23) |
| 1-5 | m/z = 691.77 (C49H29N3O2 = | 1-6 | m/z = 691.77 (C49H29N3O2 = |
| | 691.23) | | 691.23) |
| 1-12 | m/z = 767.87 (C55H33N3O2 = | 1-14 | m/z = 691.77 (C49H29N3O2 = |
| | 767.26) | | 691.23) |
| 1-15 | m/z = 691.77 (C49H29N3O2 = | 1-17 | m/z = 691.77 (C49H29N3O2 = |
| | 691.23) | | 691.23) |
| 1-21 | m/z = 691.77 (C49H29N3O2 = | 1-23 | m/z = 767.87 (C55H33N3O2 = |
| | 691.23) | | 767.26) |
| 1-25 | m/z = 691.77 (C49H29N3O2 = | 1-26 | m/z = 691.77 (C49H29N3O2 = |
| | 691.23) | | 691.23) |
| 1-33 | m/z = 767.87 (C55H33N3O2 = | 1-35 | m/z = 691.77 (C49H29N3O2 = |
| | 767.26) | | 691.23) |
| 1-36 | m/z = 691.77 (C49H29N3O2 = | 1-37 | m/z = 691.77 (C49H29N3O2 = |
| | 691.23) | | 691.23) |
| 1-41 | m/z = 707.84 (C49H29N3OS = | 1-44 | m/z = 707.84 (C49H29N3OS = |
| | 707.20) | | 707.20) |
| 1-45 | m/z = 707.84 (C49H29N3OS = | 1-46 | m/z = 707.84 (C49H29N3OS = |
| | 707.20) | | 707.20) |
| 1-52 | m/z = 783.94 (C55H33N3OS = | 1-54 | m/z = 707.84 (C49H29N3OS = |
| | 783.23) | | 707.20) |
| 1-56 | m/z = 707.84 (C49H29N3OS = | 1-58 | m/z = 707.84 (C49H29N3OS = |
| | 707.20) | | 707.20) |
| 1-62 | m/z = 783.94 (C55H33N3OS = | 1-63 | m/z = 783.94 (C55H33N3OS = |
| | 783.23) | | 783.23) |
| 1-65 | m/z = 707.84 (C49H29N3OS = | 1-67 | m/z = 707.84 (C49H29N3OS = |
| | 707.20) | | 707.20) |
| 1-71 | m/z = 707.84 (C49H29N3OS = | 1-74 | m/z = 707.84 (C49H29N3OS = |
| | 707.20) | | 707.20) |
| 1-75 | m/z = 707.84 (C49H29N3OS = | 1-77 | m/z = 707.84 (C49H29N3OS = |
| | 707.20) | | 707.20) |
| 1-81 | m/z = 707.84 (C49H29N3OS = | 1-85 | m/z = 707.84 (C49H29N3OS = |
| | 707.20) | | 707.20) |
| 1-87 | m/z = 707.84 (C49H29N3OS = | 1-90 | m/z = 707.84 (C49H29N3OS = |
| | 707.20) | | 707.20) |
| 1-95 | m/z = 707.84 (C49H29N3OS = | 1-96 | m/z = 707.84 (C49H29N3OS = |
| | 707.20) | | 707.20) |
| 1-97 | m/z = 707.84 (C49H29N3OS = | 1-100 | m/z = 707.84 (C49H29N3OS = |
| | 707.20) | | 707.20) |
| 1-101 | m/z = 707.84 (C49H29N3OS = | 1-104 | m/z = 707.84 (C49H29N3OS = |
| | 707.20) | | 707.20) |
| 1-106 | m/z = 707.84 (C49H29N3OS = | 1-107 | m/z = 707.84 (C49H29N3OS = |
| | 707.20) | | 707.20) |
| 1-113 | m/z = 783.94 (C55H33N3OS = | 1-115 | m/z = 707.84 (C49H29N3OS = |
| | 783.23) | | 707.20) |
| 1-116 | m/z = 707.84 (C49H29N3OS = | 1-117 | m/z = 707.84 (C49H29N3OS = |
| | 707.20) | | 707.20) |
| 1-123 | m/z = 800.00 (C55H33N3S2 = | 1-125 | m/z = 723.90 (C49H29N3S2 = |
| | 799.21) | | 723.18) |
| 1-126 | m/z = 723.90 (C49H29N3S2 = | 1-127 | m/z = 723.90 (C49H29N3S2 = |
| | 723.18) | | 723.18) |
| 1-131 | m/z = 723.90 (C49H29N3S2 = | 1-135 | m/z = 723.90 (C49H29N3S2 = |
| | 723.18) | | 723.18) |
| 1-137 | m/z = 723.90 (C49H29N3S2 = | 1-139 | m/z = 723.90 (C49H29N3S2 = |
| | 723.18) | | 723.18) |
| 1-143 | m/z = 800.00 (C55H33N3S2 = | 1-145 | m/z = 723.90 (C49H29N3S2 = |
| | 799.21) | | 723.18) |
| 1-146 | m/z = 723.90 (C49H29N3S2 = | 1-147 | m/z = 723.90 (C49H29N3S2 = |
| | 723.18) | | 723.18) |
| 1-154 | m/z = 723.90 (C49H29N3S2 = | 1-155 | m/z = 723.90 (C49H29N3S2 = |
| | 723.18) | | 723.18) |
| 2-3 | m/z = 560.69(C42H28N2 = | 2-4 | m/z = 560.69(C42H28N2 = |
| | 560.23) | | 560.23) |
| 2-7 | m/z = 636.78(C48H32N2 = | 2-27 | m/z = 712.88(C54H36N2 = |
| | 636.26) | | 712.29) |
| 2-28 | m/z = 712.88(C54H36N2 = | 2-29 | m/z = 712.88(C54H36N2 = |
| | 712.29) | | 712.29) |
| 2-32 | m/z = 636.78(C48H32N2 = | 2-34 | m/z = 712.88(C54H36N2 = |
| | 636.26) | | 712.29) |
| 2-38 | m/z = 788.97(C60H40N2 = | 2-46 | m/z = 654.89(C48H14D18N2 = |
| | 788.32) | | 654.37) |
| 2-49 | m/z = 654.89(C48H14D18N2 = | 2-62 | m/z = 650.87(C48H18D14N2 = |
| | 654.37) | | 650.34) |
| 2-65 | m/z = 650.87(C48H18D14N2 = | 2-70 | m/z = 650.87(C48H18D14N2 = |
| | 650.34) | | 650.34) |
| 2-82 | m/z = 668.98(C48D32N2 = | 2-83 | m/z = 588.86(C42D28N2 = |
| | 668.46) | | 588.40) |
| 2-85 | m/z = 668.98(C48D32N2 = | 2-86 | m/z = 668.98(C48D32N2 = |
| | 668.46) | | 668.46) |
| 2-89 | m/z = 668.98(C48D32N2 = | 2-90 | m/z = 668.98(C48D32N2 = |
| | 668.46) | | 668.46) |

TABLE 5

| Com- | |
|---|---|
| pound | ¹H NMR (DMSO, 300 Mz) |
| 1-1 | δ = 8.55 (1H, d), 8.28 (2H, d), 8.18 (1H, d), 7.95 (2H, d), |
| | 7.79-7.71 (6H, m), 7.64-7.41 (17H, m) |
| 1-4 | δ = 8.55 (1H, d), 8.28 (2H, d), 8.18 (1H, d), 7.95 (1H, d), |
| | 7.81-7.71 (8H, m), 7.64-7.41 (16H, m) |
| 1-5 | δ = 8.55 (1H, d), 8.28 (2H, d), 8.18 (1H, d), 7.95 (2H, d), |
| | 7.79-7.71 (6H, m), 7.64-7.41 (17H, m) |
| 1-6 | δ = 8.55 (1H, d), 8.28 (2H, d), 8.18 (1H, d), 7.95 (1H, d), |
| | 7.85-7.71 (7H, m), 7.64-7.38 (17H, m) |
| 1-12 | δ = 8.55 (1H, d), 8.18 (1H, d), 7.95 (1H, d), 7.85~7.71 |
| | (12H, m), 7.64~7.41 (16H, m), 7.25 (2H, d) |
| 1-14 | δ = 8.55 (1H, d), 8.28 (2H, d), 8.18 (1H, d), 7.95 (1H, d), |
| | 7.81~7.71 (10H, m), 7.64 (1H, s), 7.55~7.41 (13H, m) |
| 1-15 | δ = 8.55 (1H, d), 8.28 (2H, d), 8.18 (1H, d), 7.95 (2H, d), |
| | 7.81~7.71 (8H, m), 7.64~7.41 (15H, m) |
| 1-17 | δ = 8.55 (1H, d), 8.28 (2H, d), 8.18 (1H, d), 7.89 (1H, d), |
| | 7.81~7.32 (24H, m) |
| 1-21 | δ = 8.55 (1H, d), 8.28 (2H, d), 8.18 (1H, d), 7.95 (2H, d), |
| | 7.79~7.71 (8H, m), 7.64~7.41 (15H, m) |
| 1-23 | δ = 8.55 (1H, d), 8.24 (1H, d), 8.18 (1H, d), 7.95 (2H, d), |
| | 7.79~7.41 (28H, m) |
| 1-25 | δ = 8.55 (1H, d), 8.28 (2H, d), 8.18 (1H, d), 7.95 (3H, d), |
| | 7.79~7.71 (6H, m), 7.64 (3H, s), 7.55~7.41 (13H, m) |
| 1-26 | δ = 8.55 (1H, d), 8.28 (2H, d), 8.18 (1H, d), 7.95 (2H, d), |
| | 7.85~7.71 (7H, m), 7.64~7.38 (16H, d) |
| 1-33 | δ = 8.55 (1H, d), 8.24 (1H, d), 8.18 (1H, d), 7.95 (1H, d), |
| | 7.85~7.7 (10H, m), 7.64~7.38 (19H, m) |
| 1-35 | δ = 8.55 (1H, d), 8.28 (2H, d), 8.18 (1H, d), 7.95 (2H, d), |
| | 7.85~7.71 (7H, m), 7.64 (2H, s), 7.55~7.38 (14H, m) |
| 1-36 | δ = 8.55 (1H, d), 8.28 (2H, d), 8.18 (1H, d), 7.95 (1H, d), |
| | 7.85~7.71 (8H, m), 7.64 (1H, s), 7.55~7.38 (15H, m) |
| 1-37 | δ = 8.55 (1H, d), 8.28 (2H, d), 8.18 (1H, d), 7.89~7.79 |
| | (7H, m), 7.71~7.32 (18H, m) |
| 1-41 | δ = 8.55 (1H, d), 8.28 (2H, d), 8.08 (1H, d), 8.00~7.94 |
| | (3H, m), 7.82~7.75 (7H, m), 7.64~7.41 (15H, m) |
| 1-44 | δ = 8.55 (1H, d), 8.28 (2H, d), 8.08 (1H, d), 8.00~7.94 |
| | (3H, m), 7.82~7.71 (7H, m), 7.64 (1H, s), 7.56~7.41 |
| | (14H, m) |
| 1-45 | δ = 8.55 (1H, d), 8.28 (2H, d), 8.08 (1H, d), 8.00~7.94 |
| | (4H, m), 7.82~7.75 (5H, m), 7.64 (2H, s), 7.56~7.41 |
| | (14H, m) |
| 1-46 | δ = 8.55 (1H, d), 8.28 (2H, d), 8.28 (1H, d), 8.08 (1H, d), |
| | 8.00~7.94 (3H, m), 7.85~7.75 (6H, m), 7.64 (1H, s), |
| | 7.56~7.38 (15H, m) |
| 1-52 | δ = 8.55 (1H, d), 8.08 (1H, d), 8 (2H, d), 7.95 (1H, d), |
| | 7.86~7.75 (10H, m), 7.64~7.41 (16H, m), 7.25 (2H, d) |
| 1-54 | δ = 8.55 (1H, d), 8.28 (2H, d), 8.08 (1H, d), 8.00~7.95 |
| | (3H, m), 7.86 (1H, d), 7.81~7.71 (7H, d), 7.64 (1H, s), |
| | 7.55~7.41 (13H, m) |
| 1-56 | δ = 8.55 (1H, d), 8.28 (2H, d), 8.08 (1H, d), 8.00~7.95 |
| | (3H, m), 7.86~7.75 (7H, m), 7.64 (1H, s), 7.55~7.38 |
| | (14H, m) |
| 1-58 | δ = 8.42 (1H, d), 8.28 (2H, d), 8.28 (1H, d), 8.05~7.95 |
| | (4H, m), 7.86~7.75 (8H, m), 7.64~7.41 (14H, m) |
| 1-62 | δ = 8.55 (1H, d), 8.11~8.08 (3H, m), 8.00~7.95 (2H, m), |
| | 7.85~7.75 (9H, m), 7.64~7.41 (16H, m), 7.25 (2H, d) |
| 1-63 | δ = 8.55 (1H, d), 8.24 (1H, d), 8.11~8.08 (3H, m), 8.00 |
| | (1H, s), 7.95 (1H, d), 7.82~7.7 (8H, m), 7.64~7.41 |
| | (18H, m) |
| 1-65 | δ = 8.55 (1H, d), 8.28 (2H, d), 8.11 (1H, d), 8.08 (1H, s), |
| | 8.00 (1H, s), 7.95 (2H, d), 7.82~7.75 (5H, m), 7.64~7.41 |
| | (13H, m) |
| 1-67 | δ = 8.55 (1H, d), 8.28 (2H, d), 8.11-8.08 (3H, m), 8.00 |
| | (1H, s), 7.89-7.79 (6H, m), 7.66-7.32 (16H, m) |
| 1-71 | δ = 8.55 (1H, d), 8.41, (1H, d), 8.28 (2H, d), 8.08 (1H, d), |
| | 8.00 (1H, s), 7.95 (1H, d), 7.8-7.75 (7H, m), 7.64-7.41 |
| | (15H, m) |
| 1-74 | δ = 8.55 (1H, d), 8.41 (1H, d), 8.28 (2H, d), 8.08 (1H, d), |
| | 8.00 (1H, s), 7.95 (1H, d), 7.81-7.71 (7H, m), 7.58-7.41 |
| | (14H, m) |
| 1-75 | δ = 8.55 (1H, d), 8.41 (1H, d), 8.28 (2H, d), 8.08 (1H, d), |
| | 8 (1H, s), 7.95 (2H, d), 7.8-7.75 (5H, m), 7.64-7.41 |
| | (16H, m) |
| 1-77 | δ = 8.55 (1H, d), 8.41 (1H, d), 8.28 (2H, d), 8.08 (1H, d), |
| | 8.00 (1H, s), 7.89 (1H, d), 7.8 (1H, d), 7.79 (4H, d), |
| | 7.66-7.32 (16H, m) |
| 1-81 | δ = 8.55 (1H, d), 8.28 (2H, d), 8.18-8.08 (3H, m), 7.94 |
| | (1H, d), 7.82-7.71 (8H, m), 7.62-7.41 (14H, m) |
| 1-85 | δ = 8.55 (1H, d), 8.28 (2H, d), 8.18-8.05 (6H, m), |
| | 7.82-7.75 (4H, m), 7.71-7.41 (16H, m) |
| 1-87 | δ = 8.55 (1H, d), 8.45 (1H, d), 8.28 (2H, d), 8.18 (1H, d), |
| | 8.04 (1H, s), 7.98 (1H, d), 7.79-7.71 (7H, m), 7.62-7.41 |
| | (15H, m) |
| 1-90 | δ = 8.34 (1H, s), 8.28 (2H, d), 8.11 (1H, d), 8.08 (1H, s), |
| | 7.94-7.73 (9H, m), 7.62-7.41 (15H, m) |
| 1-95 | δ = 8.55 (1H, d), 8.28 (2H, d), 8.18-8.05 (6H, m), |
| | 7.82-7.71 (7H, m), 7.55-7.41 (13H, m) |
| 1-96 | δ = 8.55 (1H, d), 8.41 (1H, d), 8.28 (2H, d), 8.20-8.08 |
| | (4H, m), 7.82-7.71 (7H, m), 7.58-7.41 (14H, m) |
| 1-97 | δ = 8.55 (1H, d), 8.45 (1H, d), 8.28 (2H, d), 8.18 (1H, d), |
| | 8.04 (1H, s), 7.98 (1H, d), 7.81-7.71 (9H, m), 7.55-7.41 |
| | (13H, m) |
| 1-100 | δ = 8.34 (1H, s), 8.28 (2H, d), 8.11 (1H, d), 8.08 (1H, s), |
| | 7.94-7.71 (11H, m), 7.56-7.41 (13H, m) |
| 1-101 | δ = 8.55 (1H, d), 8.28 (2H, d), 8.18-8.08 (3H, m), |
| | 7.95-7.94 (2H, m), 7.82-7.71 (8H, m), 7.64 (1H, s), |
| | 7.56-7.41 (12H, m) |
| 1-104 | δ = 8.55 (1H, d), 8.28 (2H, d), 8.18-8.08 (3H, m), |
| | 8.00-7.95 (3H, m), 7.86-7.71 (6H, m), 7.64 (1H, s), |
| | 7.55-7.41 (13H, m) |
| 1-106 | δ = 8.55 (1H, d), 8.41 (1H, d), 8.28 (2H, d), 8.20-8.08 |
| | (4H, m), 7.95 (1H, d), 7.82-7.71 (5H, m), 7.64-7.41 |
| | (15H, m) |
| 1-107 | δ = 8.55 (1H, d), 8.45 (1H, d), 8.28 (2H, d), 8.18 (1H, d), |
| | 8.04-7.95 (3H, m), 7.79-7.71 (7H, m), 7.64 (1H, s), |
| | 7.55-7.41 (13H, m) |
| 1-113 | δ = 8.55 (1H, d), 8.24-8.08 (4H, m), 7.94 (1H, d), |
| | 7.85-7.71 (9H, m), 7.70 (1H, s), 7.57-7.38 (17H, m) |
| 1-115 | δ = 8.55 (1H, d), 8.28 (2H, d), 8.18-8.05 (6H, m), |
| | 7.85-7.79 (5H, m), 7.71 (1H, s), 7.55-7.38 (14H, m) |
| 1-116 | δ = 8.55 (1H, d), 8.41 (1H, d), 8.28 (2H, d), 8.20-8.08 |
| | (4H, m), 7.85-7.79 (5H, m), 7.71 (1H, s), 7.58-7.38 |
| | (15H, m) |
| 1-117 | δ = 8.55 (1H, d), 8.45 (1H, d), 8.28 (2H, d), 8.18 (1H, d), |
| | 8.04 (1H, s), 7.98 (1H, d), 7.85-7.77 (7H, m), 7.71 (1H, |
| | s), 7.55-7.38 (14H, m), |
| 1-123 | δ = 8.55 (1H, d), 8.24 (1H, d), 8.11-8.08 (3H, m), 8.00 |
| | (1H, s), 7.94 (2H, d), 7.82-7.79 (7H, m), 7.70 (1H, s), |
| | 7.57-7.41 (17H, m) |
| 1-125 | δ = 8.55 (1H, d), 8.28 (2H, d), 8.11-8.00 (7H, m), 7.94 |
| | (1H, s), 7.82-7.79 (4H, m), 7.56-7.41 (14H, m) |
| 1-126 | δ = 8.55 (1H, d), 8.41 (1H, d), 8.28 (2H, d), 8.20 (1H, d), |
| | 8.11-8.08 (3H, m), 8.00 (1H, s), 7.94 (1H, s), 7.82-7.79 |
| | (4H, m), 7.58-7.41 (15H, m) |
| 1-127 | δ = 8.55 (1H, d), 8.45 (1H, d), 8.28 (2H, d), 8.08-7.94 |
| | (5H, m), 7.82-7.77 (6H, m), 7.56-7.41 (14H, m) |
| 1-131 | δ = 8.55 (1H, d), 8.28 (2H, d), 8.11-8.08 (3H, m), 8.00 |
| | (2H, s), 7.94 (1H, d), 7.86-7.79 (8H, m), 7.56-7.41 |
| | (12H, m), |
| 1-135 | δ = 8.55 (1H, d), 8.28 (2H, d), 8.11-8.05 (6H, m), 8.00 |
| | (2H, s), 7.86-7.79 (5H, m) 7.55-7.41 (13H, m) |
| 1-137 | δ = 8.55 (1H, d), 8.45 (1H, d), 8.28 (2H, d), 8.08-7.98 |
| | (5H, m), 7.86-7.77 (7H, m), 7.55-7.41 (12H, m) |
| 1-139 | δ = 8.28 (2H, d), 8.11-7.94 (6H, m), 7.86-7.73 (8H, m), |
| | 7.58-7.41 (13H, m) |
| 1-143 | δ = 8.55 (1H, d), 8.24 (1H, d), 8.11-8.08 (5H, m), 8.00 |
| | (1H, s), 7.82-7.79 (7H, m), 7.70 (1H, s), 7.57-7.41 |
| | (16H, m) |
| 1-145 | δ = 8.55 (1H, d), 8.28 (2H, d), 8.11-8.00 (9H, m), |
| | 7.82-7.79 (4H, m), 7.55-7.41 (13H, m) |
| 1-146 | δ = 8.55 (1H, d), 8.41 (1H, d), 8.28 (2H, d), 8.20 (1H, d), |
| | 8.11-8.08 (5H, m), 8.00 (1H, s), 7.82-7.79 (4H, m) 7.58-7.41 |
| | (14H, m) |
| 1-147 | δ = 8.55 (1H, d), 8.45 (1H, d), 8.28 (2H, d), 8.11-7.98 |
| | (6H, m), 7.82-7.77 (6H, m), 7.55-7.41 (13H, m) |
| 1-154 | δ = 8.55 (1H, d), 8.41 (1H, d), 8.28 (2H, d), 8.11-8.00 |
| | (6H, m), 7.86-7.79 (5H, m), 7.58-7.41 (14H, m) |
| 1-155 | δ = 8.55 (1H, d), 8.41 (1H, d), 8.28 (2H, d), 8.11-8.00 |
| | (7H, m), 7.82-7.79 (4H, m), 7.58-7.41 (14H, m) |
| 1-156 | δ = 8.55 (1H, d), 8.41 (2H, d), 8.28 (2H, d), 8.20 (1H, d), |
| | 8.11-8.08 (3H, m), 8.00 (1H, s), 7.82-7.79 (4H, m), |
| | 7.58-7.41 (15H, m) |
| 1-157 | δ = 8.55 (1H, d), 8.28 (2H, d), 8.18 (1H, d), 7.89-7.79 |
| | (7H, m), 7.71-7.32 (18H, m) |
| 2-3 | δ = 8.55(1H, d), 8.18-8.09(3H, m), 8.00-7.87(3H, m), |
| | 7.77(2H, s), 7.58-7.25(18H, m) |
| 2-4 | δ = 8.55(1H, d), 8.18-8.12(2H, m), 8.00-7.84(3H, m), |
| | 7.79-7.77(4H, m), 7.68-7.25(22H, m) |
| 2-7 | δ = 8.55(1H, d), 8.18-8.09(4H, m), 8.00-7.94(2H, m), |
| | 7.87(1H, m), 7.77(2H, m), 7.69-7.63(2H, m), 7.52-7.25(20H, |
| | m) |
| 2-27 | δ = 8.55(1H, d), 8.18-8.09(4H, m), 8.00-7.94(2H, m), |
| | 7.77(2H, m) |
| 2-28 | δ = 8.55(1H, d), 8.18-8.09(3H, m), 8.00-8.79(2H, m), |
| | 7.87(1H, s), 7.79-7.77(4H, m), 7.69-7.63(4H, m), 7.52-7.25 |
| | (12H, m) |
| 2-29 | δ = 8.55(1H, s), 8.18-8.09(3H, m), 8.00-7.94(2H, m), |
| | 7.87(1H, s), 7.79-7.77(4H, m), 7.69-7.63(4H, m), 7.52-7.25 |
| | (21H, m) |
| 2-32 | δ = 8.55(1H, d), 8.18-8.12(2H, m), 8.00-7.87(3H, m), |
| | 7.79-7.77(6H, M), 7.69-7.63(6H, m), 7.52-7.25(14H, m) |
| 2-34 | δ = 8.55(1H, d), 8.18-8.12(2H, M), 8.00-7.87(3H, M), |
| | 7.79-7.77(6H, M), 7.67-7.63(6H, M), 7.52-7.25(18H, M) |
| 2-38 | δ = 8.55(1H, d), 8.18-8.12(2H, M), 8.05-7.87(6H, M), |
| | 7.79-7.77(4H, M), 7.69-7.63(4H, M), 7.52-7.25(23H, M) |
| 2-46 | δ = 8.55(1H, d), 8.18-8.12(2H, M), 8.00-7.87(3H, M), |
| | 7.77-7.63(4H, M), 7.50(1H, t), 7.33-7.25(3H, M) |
| 2-49 | δ = 8.55(1H, d), 8.18-8.12(2H, m), 8.00-7.87(3H, m), |
| | 7.77-7.63(4H, M), 7.50(1H, t), 7.33-7.25(3H, m) |
| 2-62 | δ = 7.79(4H, M), 7.68(4H. m), 7.52-7.41(10H, m) |
| 2-65 | δ = 7.79(2H, m), 7.70-7.68(3H, m), 7.58-7.41(13H, m) |
| 2-70 | δ = 7.79(4H, m), 7.68(4H, m), 7.52-7.41(10H, m) |
| 2-82 | δ = deuterium content of 100% with no ¹H NMR peak |
| 2-83 | δ = deuterium content of 100% with no ¹H NMR peak |
| 2-85 | δ = deuterium content of 100% with no ¹H NMR peak |
| 2-86 | δ = deuterium content of 100% with no ¹H NMR peak |
| 2-89 | δ = deuterium content of 100% with no ¹H NMR peak |
| 2-90 | δ = deuterium content of 100% with no ¹H NMR peak |

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl (phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using a compound described in the following Table 6 as a host and tris(2-phenylpyridine)iridium (Ir(ppy)3) as a green phosphorescent dopant to dope the host with Ir(ppy)₃ in an amount of 7%. Thereafter, bathocuproine (BCP) as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of the OLED.

### 2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 6.

TABLE 6

| | Light | | | | |
|---|---|---|---|---|---|
| | emitting | Driving | | Color | Service |
| | layer | voltage | Efficiency | coordinate | life |
| | compound | (V) | (cd/A) | (x, y) | (T₉₀) |
| Comparative | A | 5.31 | 70.3 | (0.271, | 85 |
| Example 1 | | | | 0.653) | |
| Comparative | B | 5.02 | 67.9 | (0.262, | 95 |
| Example 2 | | | | 0.661) | |
| Comparative | C | 5.07 | 73.9 | (0.271, | 96 |
| Example 3 | | | | 0.671) | |
| Comparative | D | 5.33 | 73.0 | (0.276, | 96 |
| Example 4 | | | | 0.656) | |
| Comparative | E | 5.28 | 71.0 | (0.266, | 93 |
| Example 5 | | | | 0.659) | |
| Comparative | F | 5.43 | 73.9 | (0.262, | 93 |
| Example 6 | | | | 0.651) | |
| Comparative | G | 5.41 | 69.1 | (0.264, | 92 |
| Example 7 | | | | 0.652) | |
| Example 1 | 1-1 | 3.30 | 125.9 | (0.265, | 277 |
| | | | | 0.683) | |
| Example 2 | 1-4 | 3.35 | 125.9 | (0.269, | 297 |
| | | | | 0.671) | |
| Example 3 | 1-5 | 3.28 | 128.0 | (0.261, | 281 |
| | | | | 0.682) | |
| Example 4 | 1-6 | 3.17 | 128.2 | (0.261, | 293 |
| | | | | 0.672) | |
| Example 5 | 1-12 | 3.43 | 121.9 | (0.272, | 256 |
| | | | | 0.671) | |
| Example 6 | 1-14 | 3.39 | 130.3 | (0.276, | 266 |
| | | | | 0.673) | |
| Example 7 | 1-15 | 3.21 | 130.6 | (0.279, | 273 |
| | | | | 0.651) | |
| Example 8 | 1-17 | 3.30 | 127.5 | (0.268, | 289 |
| | | | | 0.653) | |
| Example 9 | 1-21 | 3.34 | 121.6 | (0.267, | 276 |
| | | | | 0.661) | |
| Example 10 | 1-23 | 3.16 | 125.1 | (0.265, | 258 |
| | | | | 0.683) | |
| Example 11 | 1-25 | 3.42 | 125.0 | (0.265, | 271 |
| | | | | 0.651) | |
| Example 12 | 1-26 | 3.47 | 124.5 | (0.274, | 283 |
| | | | | 0.672) | |
| Example 13 | 1-33 | 4.13 | 95.1 | (0.264, | 188 |
| | | | | 0.683) | |
| Example 14 | 1-35 | 4.49 | 92.5 | (0.281, | 190 |
| | | | | 0.654) | |
| Example 15 | 1-36 | 4.37 | 96.3 | (0.275, | 192 |
| | | | | 0.648) | |
| Example 16 | 1-37 | 4.20 | 91.7 | (0.282, | 189 |
| | | | | 0.652) | |
| Example 17 | 1-41 | 4.25 | 95.6 | (0.271, | 182 |
| | | | | 0.651) | |
| Example 18 | 1-44 | 4.20 | 100.5 | (0.272, | 158 |
| | | | | 0.663) | |
| Example 19 | 1-45 | 4.37 | 96.2 | (0.268, | 171 |
| | | | | 0.663) | |
| Example 20 | 1-46 | 4.01 | 100.6 | (0.264, | 161 |
| | | | | 0.651) | |
| Example 21 | 1-52 | 4.04 | 93.3 | (0.267, | 186 |
| | | | | 0.662) | |
| Example 22 | 1-54 | 4.06 | 100.7 | (0.274, | 164 |
| | | | | 0.684) | |
| Example 23 | 1-56 | 4.37 | 92.8 | (0.282, | 187 |
| | | | | 0.649) | |
| Example 24 | 1-58 | 4.72 | 87.0 | (0.281, | 113 |
| | | | | 0.669) | |
| Example 25 | 1-62 | 4.92 | 86.0 | (0.273, | 102 |
| | | | | 0.671) | |
| Example 26 | 1-63 | 4.57 | 84.2 | (0.282, | 137 |
| | | | | 0.655) | |
| Example 27 | 1-65 | 4.51 | 84.4 | (0.273, | 112 |
| | | | | 0.681) | |
| Example 28 | 1-67 | 4.89 | 90.9 | (0.271, | 112 |
| | | | | 0.681) | |
| Example 29 | 1-71 | 4.76 | 82.9 | (0.271, | 124 |
| | | | | 0.649) | |
| Example 30 | 1-74 | 4.78 | 90.7 | (0.265, | 102 |
| | | | | 0.681) | |
| Example 31 | 1-75 | 4.65 | 83.5 | (0.279, | 110 |
| | | | | 0.679) | |
| Example 32 | 1-77 | 4.91 | 87.4 | (0.281, | 138 |
| | | | | 0.674) | |
| Example 33 | 1-81 | 4.58 | 84.1 | (0.272, | 126 |
| | | | | 0.676) | |
| Example 34 | 1-85 | 4.84 | 81.1 | (0.261, | 109 |
| | | | | 0.663) | |
| Example 35 | 1-87 | 4.83 | 88.4 | (0.278, | 150 |
| | | | | 0.668) | |
| Example 36 | 1-90 | 4.86 | 89.6 | (0.281, | 141 |
| | | | | 0.659) | |
| Example 37 | 1-95 | 4.51 | 90.5 | (0.276, | 108 |
| | | | | 0.653) | |
| Example 38 | 1-96 | 4.91 | 87.6 | (0.266, | 143 |
| | | | | 0.677) | |
| Example 39 | 1-97 | 4.62 | 87.8 | (0.265, | 124 |
| | | | | 0.681) | |
| Example 40 | 1-100 | 3.01 | 125.3 | (0.281, | 278 |
| | | | | 0.671) | |
| Example 41 | 1-101 | 3.15 | 122.7 | (0.268, | 271 |
| | | | | 0.681) | |
| Example 42 | 1-104 | 3.20 | 128.3 | (0.274, | 259 |
| | | | | 0.671) | |
| Example 43 | 1-106 | 3.34 | 123.4 | (0.281, | 252 |
| | | | | 0.649) | |
| Example 44 | 1-107 | 3.82 | 116.0 | (0.269, | 219 |
| | | | | 0.682) | |
| Example 45 | 1-113 | 3.66 | 111.0 | (0.262, | 233 |
| | | | | 0.662) | |
| Example 46 | 1-115 | 3.96 | 107.9 | (0.271, | 224 |
| | | | | 0.672) | |
| Example 47 | 1-116 | 3.97 | 106.4 | (0.273, | 242 |
| | | | | 0.681) | |
| Example 48 | 1-117 | 3.51 | 111.5 | (0.262, | 224 |
| | | | | 0.653) | |
| Example 49 | 1-123 | 3.94 | 112.5 | (0.272, | 247 |
| | | | | 0.674) | |
| Example 50 | 1-125 | 3.86 | 109.2 | (0.281, | 231 |
| | | | | 0.654) | |
| Example 51 | 1-126 | 3.71 | 107.5 | (0.266, | 241 |
| | | | | 0.674) | |
| Example 52 | 1-127 | 3.98 | 113.4 | (0.272, | 234 |
| | | | | 0.678) | |
| Example 53 | 1-131 | 3.64 | 114.2 | (0.265, | 241 |
| | | | | 0.679) | |
| Example 54 | 1-135 | 3.77 | 115.1 | (0.282, | 233 |
| | | | | 0.661) | |
| Example 55 | 1-137 | 3.55 | 112.4 | (0.283, | 221 |
| | | | | 0.659) | |
| Example 56 | 1-139 | 3.77 | 106.8 | (0.272, | 204 |
| | | | | 0.665) | |
| Example 57 | 1-143 | 3.74 | 114.0 | (0.262, | 241 |
| | | | | 0.674) | |
| Example 58 | 1-145 | 3.73 | 109.9 | (0.279, | 236 |
| | | | | 0.664) | |
| Example 59 | 1-146 | 3.84 | 112.1 | (0.279, | 229 |
| | | | | 0.659) | |
| Example 60 | 1-147 | 4.13 | 96.5 | (0.267, | 189 |
| | | | | 0.683) | |
| Example 61 | 1-154 | 4.13 | 98.0 | (0.273, | 159 |
| | | | | 0.651) | |
| Example 62 | 1-155 | 4.17 | 98.7 | (0.271, | 194 |
| | | | | 0.658) | |
| Example 63 | 1-156 | 4.43 | 92.9 | (0.261, | 172 |
| | | | | 0.649) | |
| Example 64 | 1-157 | 4.15 | 98.1 | (0.268, | 184 |
| | | | | 0.649) | |

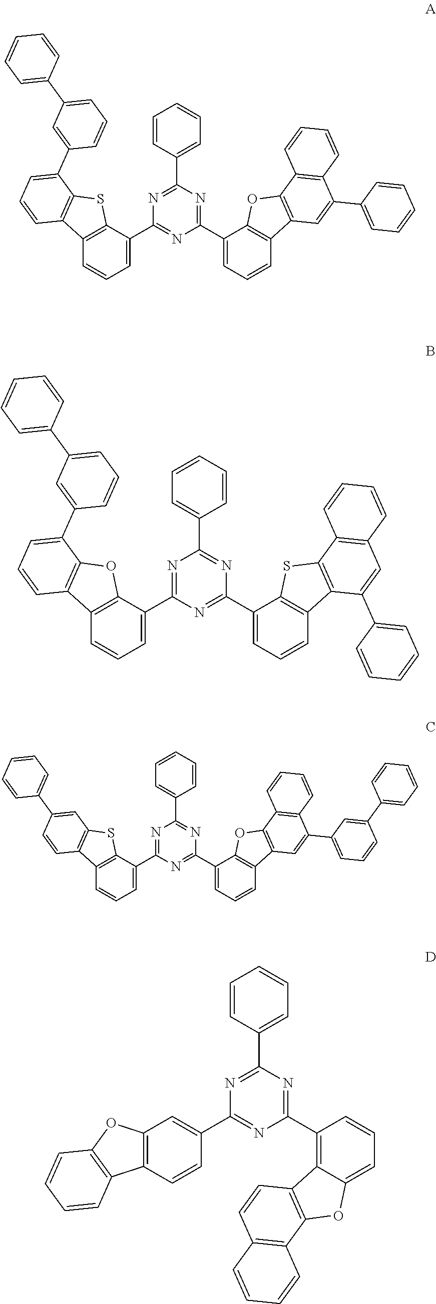 [SMILES: c1ccc(-c2cccc(-c3cccc4c3sc3c(-c5nc(-c6ccccc6)nc(-c6cccc7c6oc6c8ccccc8c(-c8ccccc8)cc76)n5)cccc34)c2)cc1 ; c1ccc(-c2cccc(-c3cccc4c3oc3c(-c5nc(-c6ccccc6)nc(-c6cccc7c6sc6c8ccccc8cc(-c8ccccc8)c76)n5)cccc34)c2)cc1 ; c1ccc(-c2cccc(-c3cc4c5cccc(-c6nc(-c7ccccc7)nc(-c7cccc8c7sc7cc(-c9ccccc9)ccc78)n6)c5oc4c4ccccc34)c2)cc1 ; c1ccc(-c2nc(-c3ccc4c(c3)oc3ccccc34)nc(-c3cccc4oc5c6ccccc6ccc5c34)n2)cc1]

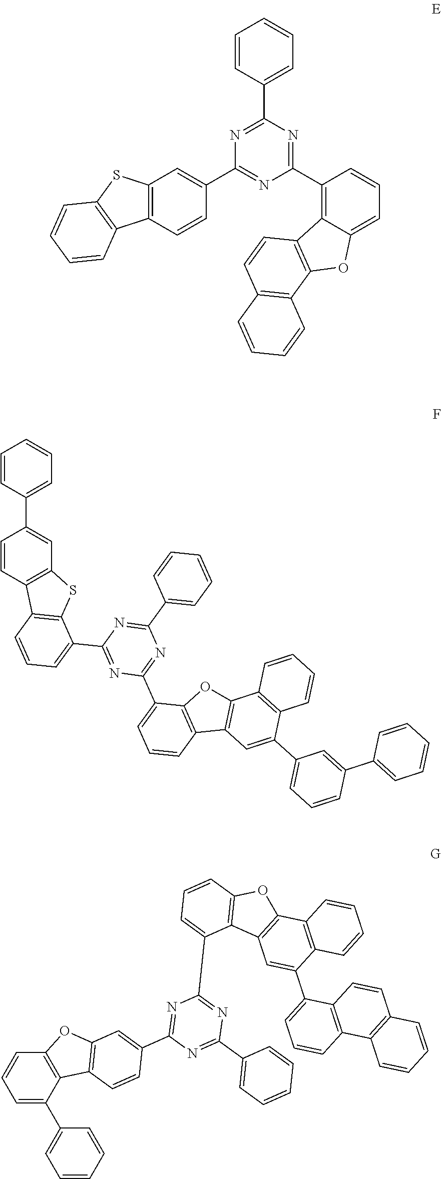 [SMILES: c1ccc(-c2nc(-c3ccc4c(c3)sc3ccccc34)nc(-c3cccc4oc5c6ccccc6ccc5c34)n2)cc1 ; c1ccc(-c2cccc(-c3cc4c5cccc(-c6nc(-c7ccccc7)nc(-c7cccc8c7sc7cc(-c9ccccc9)ccc78)n6)c5oc4c4ccccc34)c2)cc1 ; c1ccc(-c2nc(Cc3cccc4oc5c6ccccc6c(-c6cccc7c6ccc6ccccc67)cc5c34)nc(-c3ccc4c(c3)oc3cccc(-c5ccccc5)c34)n2)cc1]

Referring to the results of Table 6, it can be seen that an organic light emitting device including the heterocyclic compound of the present invention has excellent driving voltage, light emitting efficiency and service life than the Comparative Examples.

### Experimental Example 2

### 1) Manufacture of Organic Light Emitting Device

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å from one supply source after pre-mixing one type of the heterocyclic compound of Chemical Formula 1 and one type of compound of Chemical Formula 2 as hosts, and then was deposited by doping the host with Ir(ppy)₃ as a green phosphorescent dopant in an amount of 7% of the deposition thickness of the light emitting layer. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁸ to 10⁻⁶ torr for each material, and used for the manufacture of the OLED.

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 7.

TABLE 7

| | Light | | | | | |
|---|---|---|---|---|---|---|
| | emitting | | Driving | | Color | Service |
| | layer | | voltage | Efficiency | coordinate | life |
| | compound | Ratio | (V) | (cd/A) | (x, y) | (T90) |
| Comparative | A: 2-27 | 1:1 | 4.51 | 103.8 | (0.230, | 120 |
| Example 8 | | | | | 0.741) | |
| Comparative | | 1:2 | 4.58 | 99.6 | (0.233, | 136 |
| Example 9 | | | | | 0.714) | |
| Comparative | | 1:3 | 4.66 | 88.5 | (0.241, | 112 |
| Example 10 | | | | | 0.702) | |
| Comparative | | 1:1 | 4.61 | 101.6 | (0.230, | 124 |
| Example 11 | | | | | 0.741) | |
| Comparative | C: 2-28 | 1:2 | 4.69 | 98.3 | (0. 233, | 137 |
| Example 12 | | | | | 0.714) | |
| Comparative | | 1:3 | 4.71 | 85.2 | (0.241, | 111 |
| Example 13 | | | | | 0.702) | |
| Example 65 | | 1:1 | 2.32 | 150.3 | (0.247, | 341 |
| | | | | | 0.614) | |
| Example 66 | 1-14: | 1:2 | 2.41 | 143.6 | (0.248, | 384 |
| | 2-27 | | | | 0.659) | |
| Example 67 | | 1:3 | 2.57 | 137.2 | (0.253, | 363 |
| | | | | | 0.702) | |
| Example 68 | 1-14: | 1:1 | 2.31 | 151.6 | (0.252, | 376 |
| | 2-88 | | | | 0.724) | |
| Example 69 | | 1:2 | 2.43 | 142.3 | (0.241, | 437 |
| | | | | | 0.624) | |
| Example 70 | | 1:3 | 2.56 | 137.5 | (0.254, | 402 |
| | | | | | 0.710) | |
| Example 65 | 1-33: | 1:1 | 1.68 | 167.5 | (0.233, | 430 |
| | 2-28 | | | | 0.714) | |
| Example 66 | | 1:2 | 1.72 | 157.2 | (0.241, | 472 |
| | | | | | 0.702) | |
| Example 67 | | 1:3 | 1.81 | 143.8 | (0.231, | 453 |
| | | | | | 0.625) | |
| Example 68 | 1-33: | 1:1 | 1.62 | 169.6 | (0.244, | 462 |
| | | | | | 0.660) | |
| Example 69 | 2-89 | 1:2 | 1.71 | 156.6 | (0.240, | 531 |
| | | | | | 0.647) | |
| Example 70 | | 1:3 | 1.79 | 143.7 | (0.284, | 471 |
| | | | | | 0.679) | |
| Example 71 | 1-62: | 1:1 | 3.46 | 131.1 | (0.240, | 261 |
| | 2-34 | | | | 0.754) | |
| Example 72 | | 1:2 | 3.51 | 120.4 | (0.247, | 304 |
| | | | | | 0.657) | |
| Example 73 | | 1:3 | 3.59 | 113.8 | (0.250, | 275 |
| | | | | | 0.731) | |
| Example 74 | 1-62 : | 1:1 | 3.45 | 131.4 | (0.248, | 293 |
| | 2-75 | | | | 0.705) | |
| Example 75 | | 1:2 | 3.52 | 120.2 | (0.230, | 351 |
| | | | | | 0.741) | |
| Example 76 | | 1:3 | 3,61 | 113.6 | (0.233, | 304 |
| | | | | | 0.714) | |
| Example 77 | 1-75 : | 1:1 | 1.58 | 168.5 | (0.247, | 434 |
| | 2-6 | | | | 0.657) | |
| Example 78 | | 1:2 | 1.63 | 156.2 | (0.250, | 473 |
| | | | | | 0.731) | |
| Example 79 | | 1:3 | 1.71 | 143.8 | (0.248, | 452 |
| | | | | | 0.705) | |
| Example 80 | 1-75: | 1:1 | 1.60 | 170.6 | (0.230, | 466 |
| | 2-86 | | | | 0.741) | |
| Example 81 | | 1:2 | 1.62 | 153.6 | (0.233, | 534 |
| | | | | | 0.714) | |
| Example 82 | | 1:3 | 1.73 | 143.7 | (0.241, | 471 |
| | | | | | 0.702) | |
| Example 83 | 1-85 : | 1:1 | 3.94 | 115.9 | (0.241, | 171 |
| | 2-27 | | | | 0.702) | |
| Example 84 | | 1:2 | 3.99 | 105.2 | (0.231, | 226 |
| | | | | | 0.625) | |
| Example 85 | | 1:3 | 4.01 | 100.1 | (0.252, | 207 |
| | | | | | 0.724) | |
| Example 86 | 1-85: | 1:1 | 3.95 | 116.3 | (0.241, | 209 |
| | 2-48 | | | | 0.624) | |
| Example 87 | | 1:2 | 3.97 | 107.2 | (0.254, | 264 |
| | | | | | 0.710) | |
| Example 88 | | 1:3 | 4.02 | 100.7 | (0.240, | 215 |
| | | | | | 0.754) | |
| Example 89 | 1-97 : | 1:1 | 2.32 | 149.9 | (0.231, | 338 |
| | 2-7 | | | | 0.625) | |
| Example 90 | | 1:2 | 2.39 | 141.2 | (0.248, | 386 |
| | | | | | 0.705) | |
| Example 91 | | 1:3 | 2.41 | 137.2 | (0.230, | 363 |
| | | | | | 0.741) | |
| Example 92 | 1-97 : | 1:1 | 2.31 | 151.6 | (0.233, | 379 |
| | 2-87 | | | | 0.714) | |
| Example 93 | | 1:2 | 2.36 | 142.3 | (0.241, | 427 |
| | | | | | 0.702) | |
| Example 94 | | 1:3 | 2.44 | 137.5 | (0.231, | 404 |
| | | | | | 0.625) | |
| Example 95 | 1-104 : | 1:1 | 3.32 | 132.1 | (0.244, | 270 |
| | 2-33 | | | | 0.660) | |
| Example 96 | | 1:2 | 3.39 | 120.4 | (0.240, | 304 |
| | | | | | 0.647) | |
| Example 97 | | 1:3 | 3.42 | 112.8 | (0.284, | 275 |
| | | | | | 0.679) | |
| Example 98 | 1-104 : | 1:1 | 3.3 | 131.4 | (0.247, | 293 |
| | 2-51 | | | | 0.614) | |
| Example 99 | | 1:2 | 3.39 | 120.8 | (0.248, | 358 |
| | | | | | 0.659) | |
| Example 100 | | 1:3 | 3.41 | 113.6 | (0.253, | 304 |
| | | | | | 0.702) | |
| Example 101 | 1-123 : | 1:1 | 3.81 | 114.9 | (0.252, | 170 |
| | 2-6 | | | | 0.724) | |
| Example 102 | | 1:2 | 3.89 | 105.2 | (0.241, | 227 |
| | | | | | 0.624) | |
| Example 103 | | 1:3 | 3.94 | 100.7 | (0.254, | 207 |
| | | | | | 0.710) | |
| Example 104 | 1-123 : | 1:1 | 3.82 | 115.0 | (0.240, | 209 |
| | | | | | 0.754) | |
| Example 105 | 2-86 | 1:2 | 3.88 | 107.2 | (0.247, | 265 |
| | | | | | 0.657) | |
| Example 106 | | 1:3 | 3.96 | 100.7 | (0.250, | 215 |
| | | | | | 0.731) | |
| Example 107 | 1-137 : | 1:1 | 2.34 | 148.9 | (0.241, | 335 |
| | 2-27 | | | | 0.624) | |
| Example 108 | | 1:2 | 2.37 | 141.2 | (0.254, | 386 |
| | | | | | 0.710) | |
| Example 109 | | 1:3 | 2.41 | 137.2 | (0.240, | 363 |
| | | | | | 0.754) | |
| Example 110 | 1-137 : | 1:1 | 2.33 | 151.6 | (0.247, | 379 |
| | 2-88 | | | | 0.657) | |
| Example 111 | | 1:2 | 2.39 | 142.3 | (0.250, | 427 |
| | | | | | 0.731) | |
| Example 112 | | 1:3 | 2.42 | 137.5 | (0.248, | 404 |
| | | | | | 0.705) | |
| Example 113 | 1-156 : | 1:1 | 1.87 | 168.5 | (0.248, | 436 |
| | 2-34 | | | | 0.705) | |
| Example 114 | | 1:2 | 1.90 | 156.7 | (0.230, | 473 |
| | | | | | 0.741) | |
| Example 115 | | 1:3 | 1.96 | 143.4 | (0.233, | 452 |
| | | | | | 0.714) | |
| Example 116 | 1-156 : | 1:1 | 1.86 | 169.1 | (0.241, | 466 |
| | 2-75 | | | | 0.702) | |
| Example 117 | | 1:2 | 1.91 | 153.6 | (0.231, | 531 |
| | | | | | 0.625) | |
| Example 118 | | 1:3 | 1.99 | 143.7 | (0.252, | 471 |
| | | | | | 0.724) | |

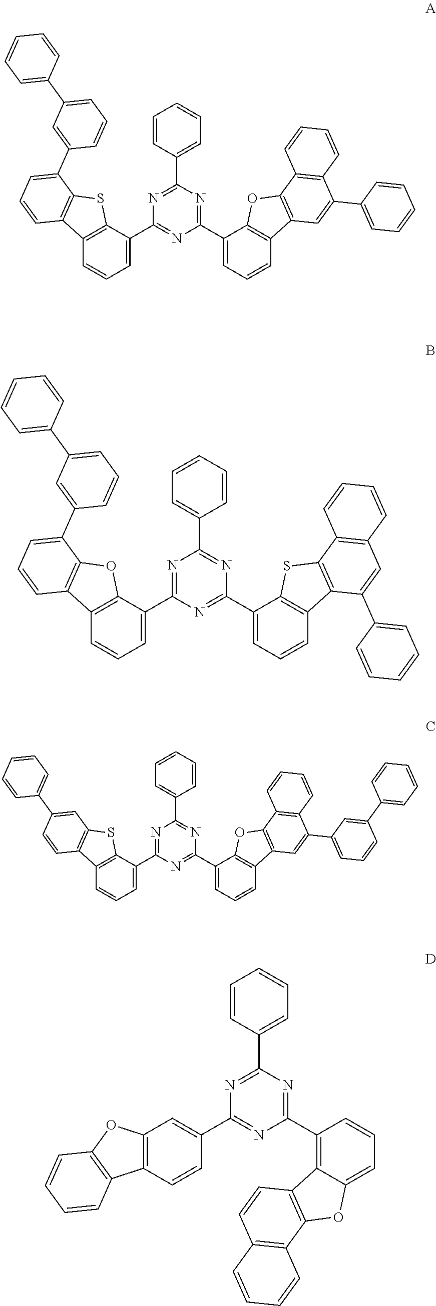 [SMILES: c1ccc(-c2cccc(-c3cccc4c3sc3c(-c5nc(-c6ccccc6)nc(-c6cccc7c6oc6c8ccccc8c(-c8ccccc8)cc76)n5)cccc34)c2)cc1 ; c1ccc(-c2cccc(-c3cccc4c3oc3c(-c5nc(-c6ccccc6)nc(-c6cccc7c6sc6c8ccccc8cc(-c8ccccc8)c76)n5)cccc34)c2)cc1 ; c1ccc(-c2cccc(-c3cc4c5cccc(-c6nc(-c7ccccc7)nc(-c7cccc8c7sc7cc(-c9ccccc9)ccc78)n6)c5oc4c4ccccc34)c2)cc1 ; c1ccc(-c2nc(-c3ccc4c(c3)oc3ccccc34)nc(-c3cccc4oc5c6ccccc6ccc5c34)n2)cc1]

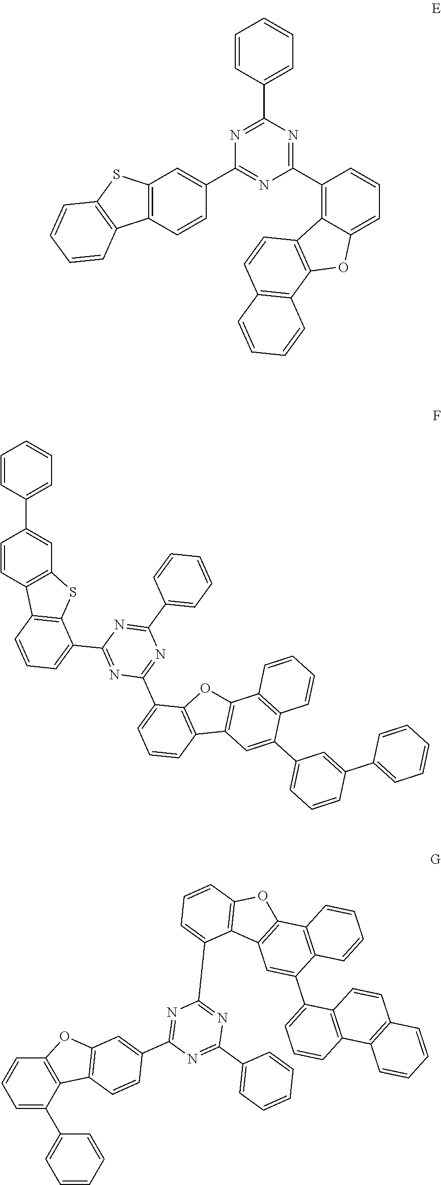 [SMILES: c1ccc(-c2nc(-c3ccc4c(c3)sc3ccccc34)nc(-c3cccc4oc5c6ccccc6ccc5c34)n2)cc1 ; c1ccc(-c2cccc(-c3cc4c5cccc(-c6nc(-c7ccccc7)nc(-c7cccc8c7sc7cc(-c9ccccc9)ccc78)n6)c5oc4c4ccccc34)c2)cc1 ; c1ccc(-c2nc(Cc3cccc4oc5c6ccccc6c(-c6cccc7c6ccc6ccccc67)cc5c34)nc(-c3ccc4c(c3)oc3cccc(-c5ccccc5)c34)n2)cc1]

Referring to the results of Tables 6 and 7, better efficiency and service life effects are exhibited when the compound of Chemical Formula 1 (n-host) and the compound of Chemical Formula 2 (p-host) are simultaneously included. This result is due to exciplex that occurs when the two compounds are simultaneously included.

The exciplex phenomenon is a phenomenon in which energy with a magnitude of the HOMO level of a donor (p-host) and the LUMO level of an acceptor (n-host) is released due to an electron exchange between two molecules. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, it could be confirmed that the compound of Chemical Formula 2 serving as a donor and the compound of Chemical Formula 1 serving as an acceptor exhibit excellent device characteristics when the compounds are used as hosts of the light emitting device.

In particular, it can be confirmed from Tables 6 and 7 that when a chemical species bonded to position 8 or 10 of naphthobenzofuran or naphthobenzothiophene of Chemical Formula 1 is introduced as an n-host, the service life of the device is increased. This is due to the fact that positions 8 and 10 of naphthobenzofuran and naphthobenzothiophene are relatively electron-rich due to the resonance structures of naphthobenzofuran and naphthobenzothiophene.

This interacts with those bonded to position 3 of the electron-deficient dibenzofuran or dibenzothiophene to balance the charge between Chemical Formula 1 (n-host) and Chemical Formula 2 (p-host), thereby improving the performance of the device.

1. A heterocyclic compound of the following Chemical Formula 1:
[Chemical Formula 1] 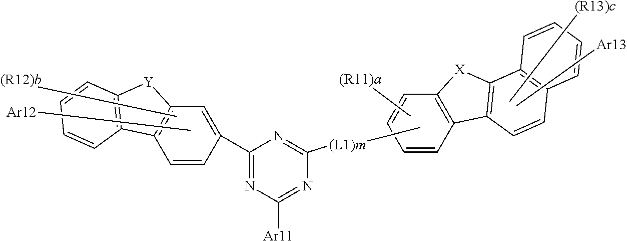
wherein, in Chemical Formula 1,
X and Y are the same as or different from each other, and are each independently O; S; or CRR′,
Ar11 and Ar12 are the same as or different from each other, and are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Ar13 is a substituted or unsubstituted C6 to C10 aryl group; or a substituted or unsubstituted C2 to C60 heteroaryl group,
L1 is a direct bond; a substituted or unsubstituted C6 to C60 arylene group; or a substituted or unsubstituted C2 to C60 heteroarylene group,
R11 to R13 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R and R′ are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
m and c are the same as or different from each other, and are each independently an integer from 0 to 5,
a is an integer from 0 to 3,
b is an integer from 0 to 6, and
when a to c and m are 2 or higher, substituents in the parenthesis are the same as or different from each other.

2. The heterocyclic compound of claim 1, wherein Chemical Formula 1 is represented by any one of the following Chemical Formulae 3 to 6: 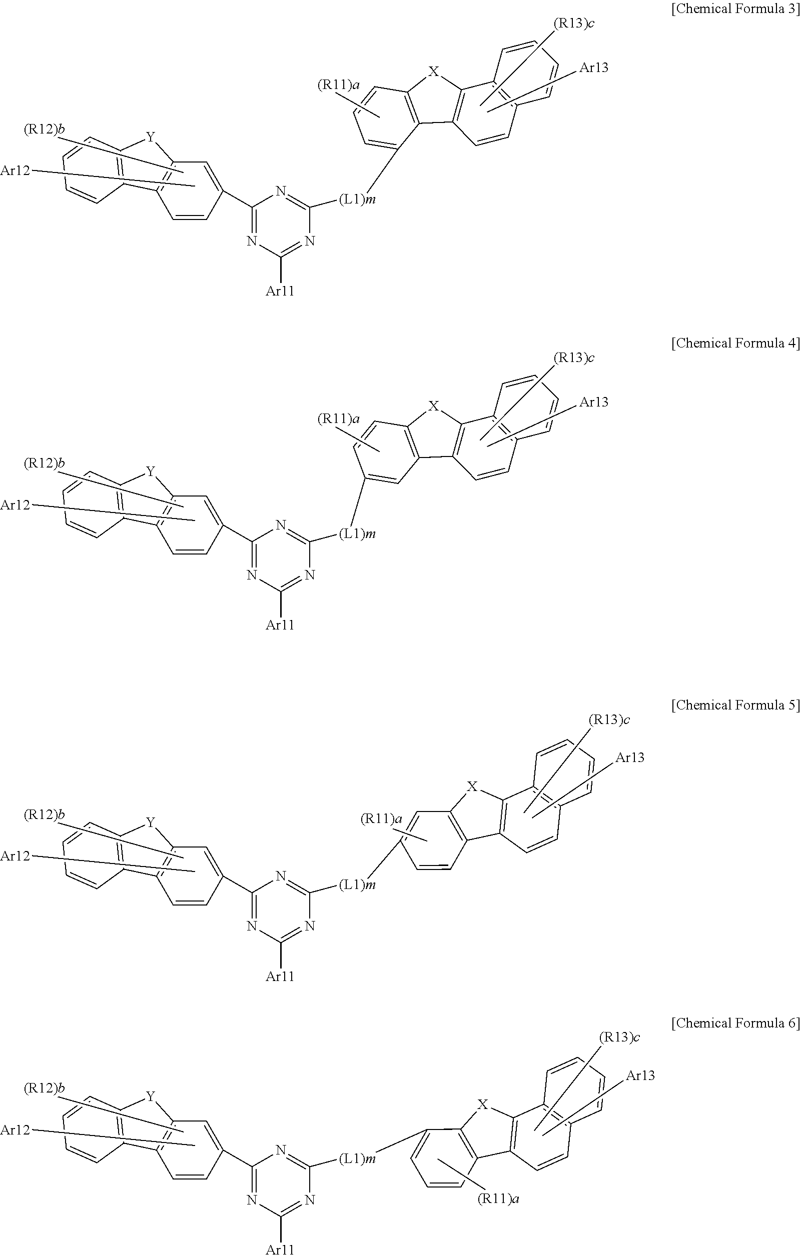
in Chemical Formulae 3 to 6,
the definition of each substituent is the same as the definition in Chemical Formula 1.

3. The heterocyclic compound of claim 1, wherein the 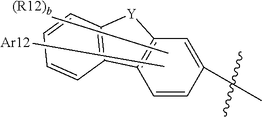
of Chemical Formula 1 is represented by any one of the following Chemical Formulae 1-1 to 1-5: 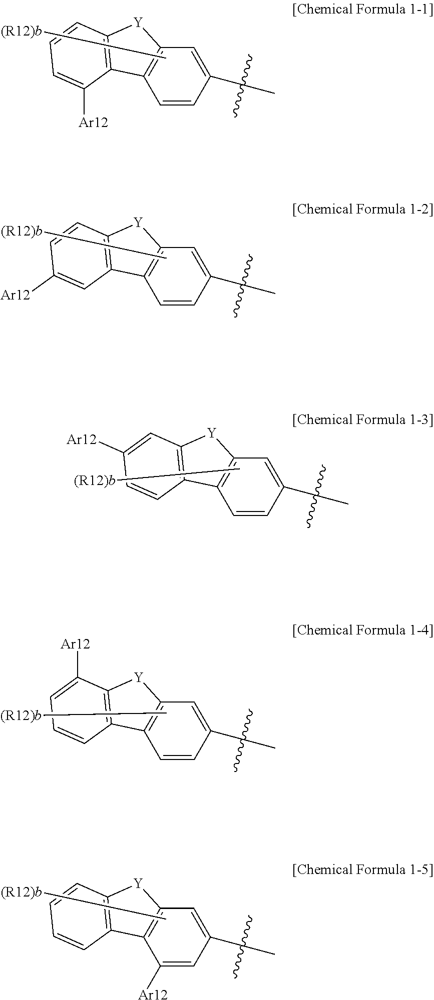
in Chemical Formulae 1-1 to 1-5, 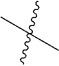
means a position linked to Chemical Formula 1, and
the definition of each substituent is the same as the definition in Chemical Formula 1.

4. The heterocyclic compound of claim 1, wherein the 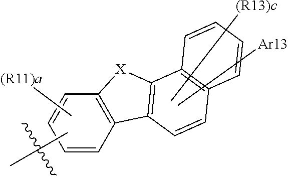
of Chemical Formula 1 is represented by any one of the following Chemical Formulae 2-1 to 2-5: 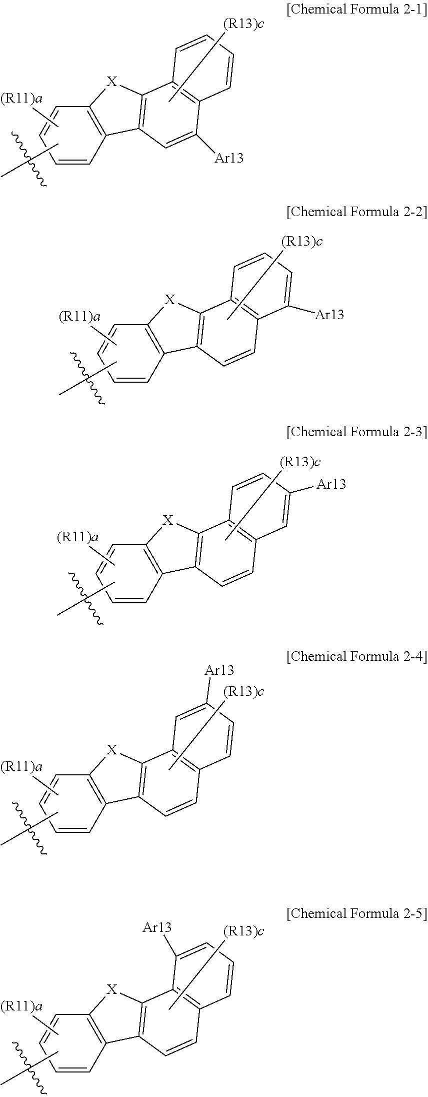
in Chemical Formulae 2-1 to 2-5, 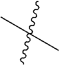
means a position linked to Chemical Formula 1, and
the definition of each substituent is the same as the definition in Chemical Formula 1.

5. The heterocyclic compound of claim 1, wherein R11 to R13 are the same as or different from each other, and are each independently hydrogen; or deuterium.

6. The heterocyclic compound of claim 1, wherein Ar13 is a substituted or unsubstituted phenyl group; or a substituted or unsubstituted naphthyl group.

7. The heterocyclic compound of claim 1, wherein Chemical Formula 1 is represented by any one of the following compounds:

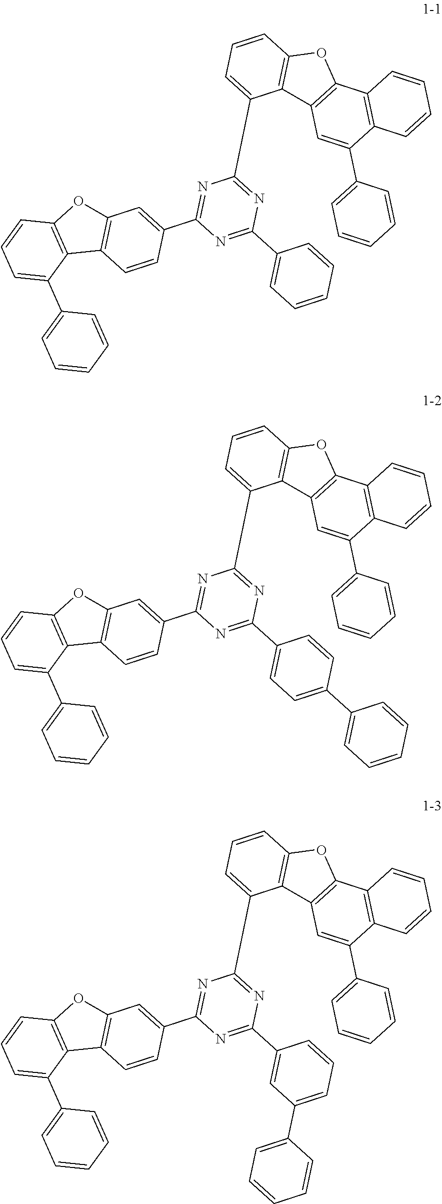 [SMILES: c1ccc(-c2nc(-c3ccc4c(c3)oc3cccc(-c5ccccc5)c34)nc(-c3cccc4oc5c6ccccc6c(-c6ccccc6)cc5c34)n2)cc1 ; c1ccc(-c2ccc(-c3nc(-c4ccc5c(c4)oc4cccc(-c6ccccc6)c45)nc(-c4cccc5oc6c7ccccc7c(-c7ccccc7)cc6c45)n3)cc2)cc1 ; c1ccc(-c2cccc(-c3nc(-c4ccc5c(c4)oc4cccc(-c6ccccc6)c45)nc(-c4cccc5oc6c7ccccc7c(-c7ccccc7)cc6c45)n3)c2)cc1]

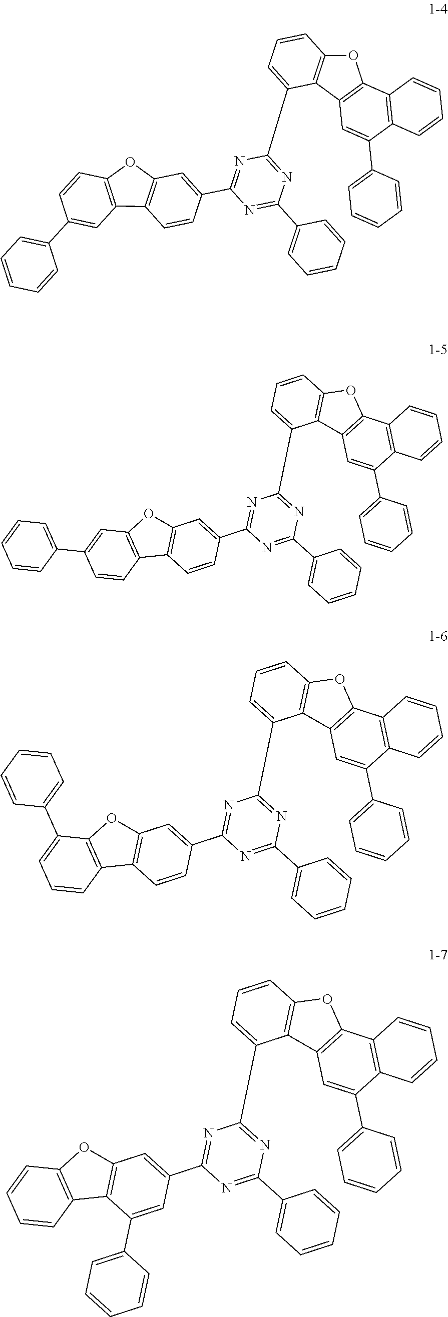 [SMILES: c1ccc(-c2ccc3oc4cc(-c5nc(-c6ccccc6)nc(-c6cccc7oc8c9ccccc9c(-c9ccccc9)cc8c67)n5)ccc4c3c2)cc1 ; c1ccc(-c2ccc3c(c2)oc2cc(-c4nc(-c5ccccc5)nc(-c5cccc6oc7c8ccccc8c(-c8ccccc8)cc7c56)n4)ccc23)cc1 ; c1ccc(-c2nc(-c3ccc4c(c3)oc3c(-c5ccccc5)cccc34)nc(-c3cccc4oc5c6ccccc6c(-c6ccccc6)cc5c34)n2)cc1 ; c1ccc(-c2nc(-c3cc(-c4ccccc4)c4c(c3)oc3ccccc34)nc(-c3cccc4oc5c6ccccc6c(-c6ccccc6)cc5c34)n2)cc1]

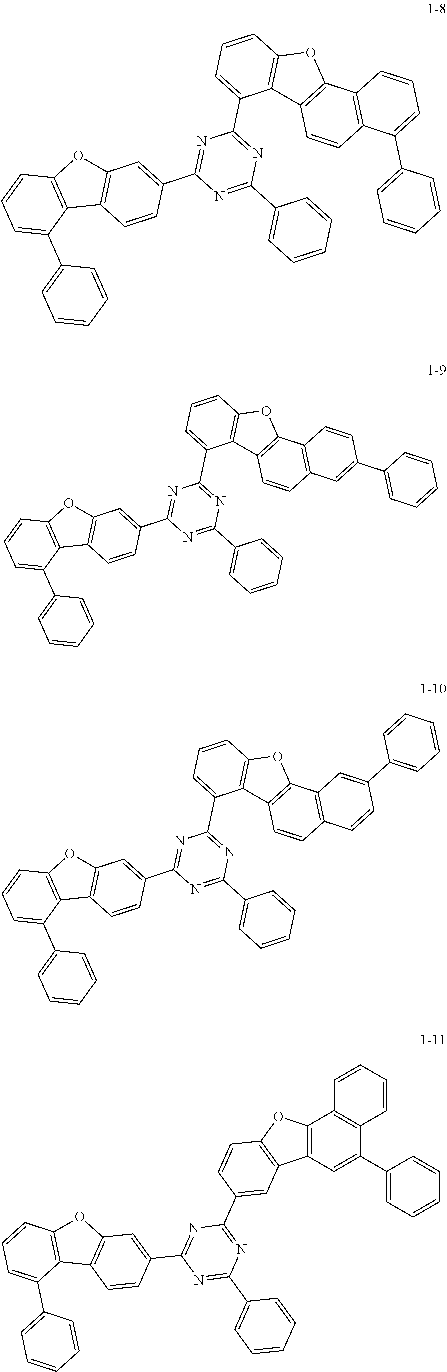 [SMILES: c1ccc(-c2nc(-c3ccc4c(c3)oc3cccc(-c5ccccc5)c34)nc(-c3cccc4oc5c6cccc(-c7ccccc7)c6ccc5c34)n2)cc1 ; c1ccc(-c2ccc3c(ccc4c3oc3cccc(-c5nc(-c6ccccc6)nc(-c6ccc7c(c6)oc6cccc(-c8ccccc8)c67)n5)c34)c2)cc1 ; c1ccc(-c2ccc3ccc4c(oc5cccc(-c6nc(-c7ccccc7)nc(-c7ccc8c(c7)oc7cccc(-c9ccccc9)c78)n6)c54)c3c2)cc1 ; c1ccc(-c2nc(-c3ccc4c(c3)oc3cccc(-c5ccccc5)c34)nc(-c3ccc4oc5c6ccccc6c(-c6ccccc6)cc5c4c3)n2)cc1]

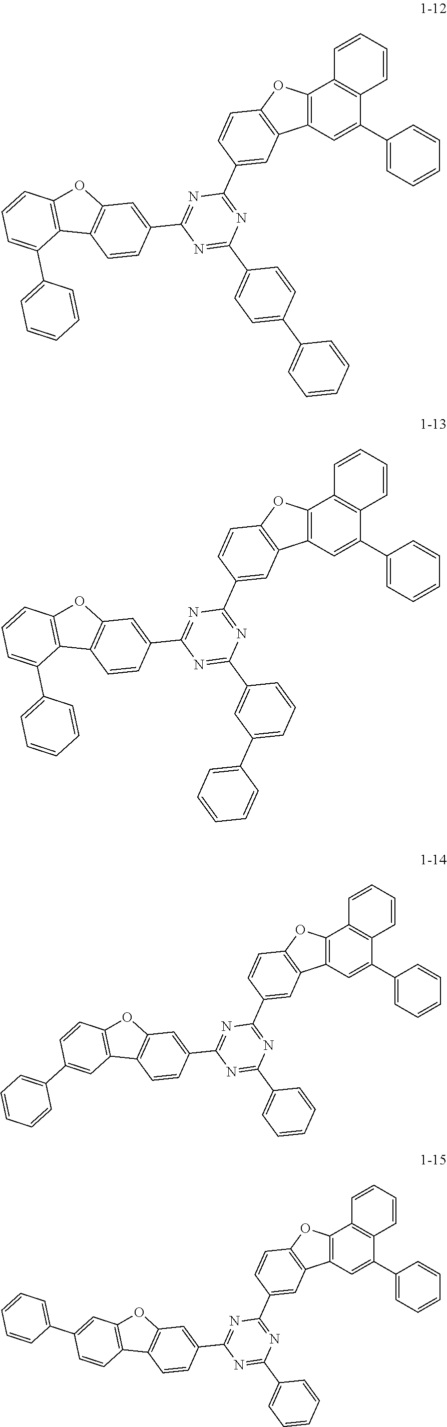 [SMILES: c1ccc(-c2ccc(-c3nc(-c4ccc5c(c4)oc4cccc(-c6ccccc6)c45)nc(-c4ccc5oc6c7ccccc7c(-c7ccccc7)cc6c5c4)n3)cc2)cc1 ; c1ccc(-c2cccc(-c3nc(-c4ccc5c(c4)oc4cccc(-c6ccccc6)c45)nc(-c4ccc5oc6c7ccccc7c(-c7ccccc7)cc6c5c4)n3)c2)cc1 ; c1ccc(-c2ccc3oc4cc(-c5nc(-c6ccccc6)nc(-c6ccc7oc8c9ccccc9c(-c9ccccc9)cc8c7c6)n5)ccc4c3c2)cc1 ; c1ccc(-c2ccc3c(c2)oc2cc(-c4nc(-c5ccccc5)nc(-c5ccc6oc7c8ccccc8c(-c8ccccc8)cc7c6c5)n4)ccc23)cc1]

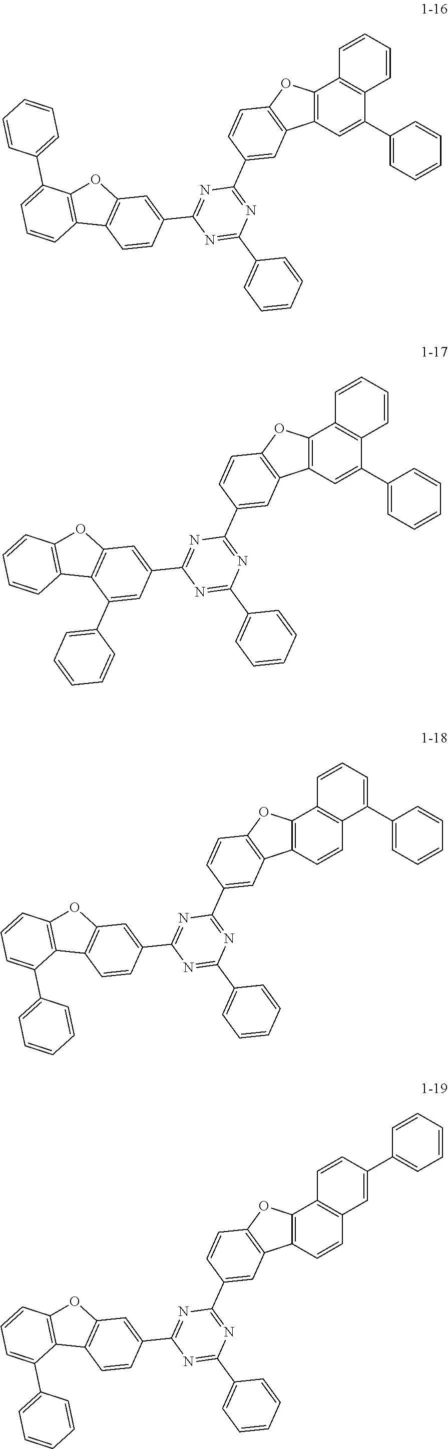 [SMILES: c1ccc(-c2nc(-c3ccc4c(c3)oc3c(-c5ccccc5)cccc34)nc(-c3ccc4oc5c6ccccc6c(-c6ccccc6)cc5c4c3)n2)cc1 ; c1ccc(-c2nc(-c3cc(-c4ccccc4)c4c(c3)oc3ccccc34)nc(-c3ccc4oc5c6ccccc6c(-c6ccccc6)cc5c4c3)n2)cc1 ; c1ccc(-c2nc(-c3ccc4c(c3)oc3cccc(-c5ccccc5)c34)nc(-c3ccc4oc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)oc7cccc(-c9ccccc9)c78)n6)ccc5oc34)c2)cc1]

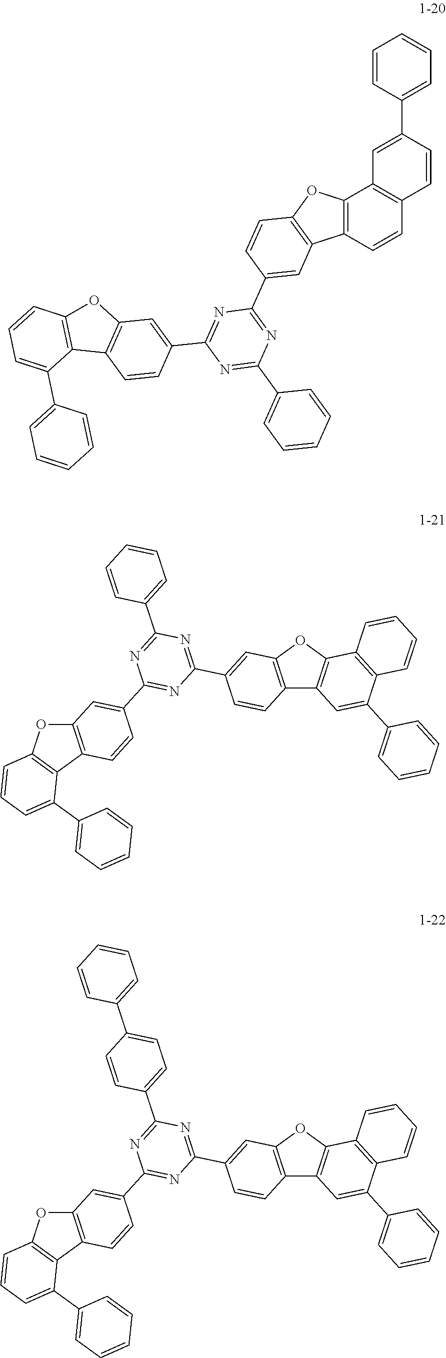 [SMILES: c1ccc(-c2ccc3ccc4c5cc(-c6nc(-c7ccccc7)nc(-c7ccc8c(c7)oc7cccc(-c9ccccc9)c78)n6)ccc5oc4c3c2)cc1 ; c1ccc(-c2nc(-c3ccc4c(c3)oc3c5ccccc5c(-c5ccccc5)cc43)nc(-c3ccc4c(c3)oc3cccc(-c5ccccc5)c34)n2)cc1 ; c1ccc(-c2ccc(-c3nc(-c4ccc5c(c4)oc4c6ccccc6c(-c6ccccc6)cc54)nc(-c4ccc5c(c4)oc4cccc(-c6ccccc6)c45)n3)cc2)cc1]

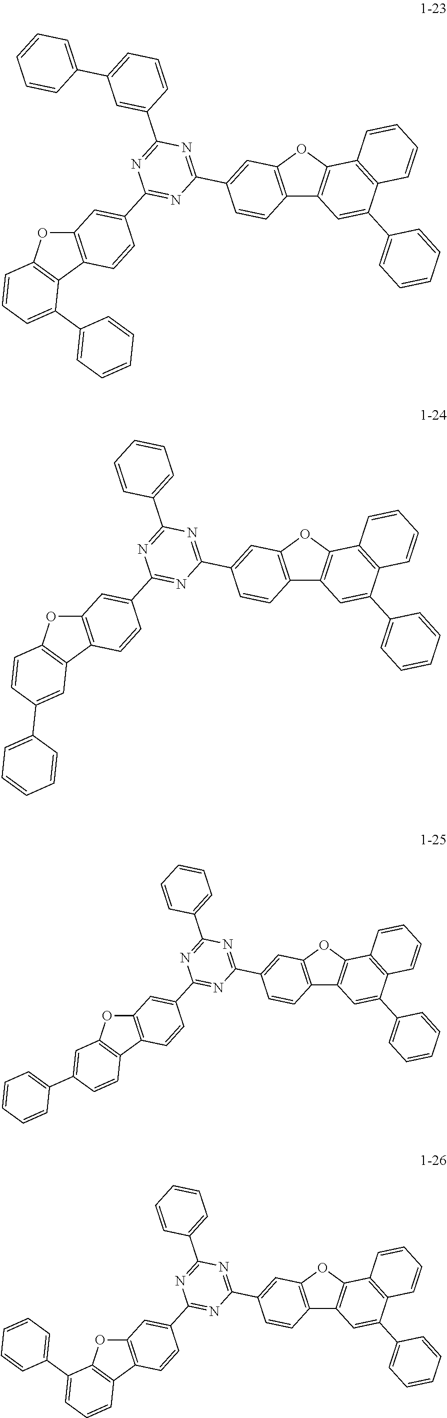 [SMILES: c1ccc(-c2cccc(-c3nc(-c4ccc5c(c4)oc4c6ccccc6c(-c6ccccc6)cc54)nc(-c4ccc5c(c4)oc4cccc(-c6ccccc6)c45)n3)c2)cc1 ; c1ccc(-c2ccc3oc4cc(-c5nc(-c6ccccc6)nc(-c6ccc7c(c6)oc6c8ccccc8c(-c8ccccc8)cc76)n5)ccc4c3c2)cc1 ; c1ccc(-c2ccc3c(c2)oc2cc(-c4nc(-c5ccccc5)nc(-c5ccc6c(c5)oc5c7ccccc7c(-c7ccccc7)cc65)n4)ccc23)cc1 ; c1ccc(-c2nc(-c3ccc4c(c3)oc3c(-c5ccccc5)cccc34)nc(-c3ccc4c(c3)oc3c5ccccc5c(-c5ccccc5)cc43)n2)cc1]

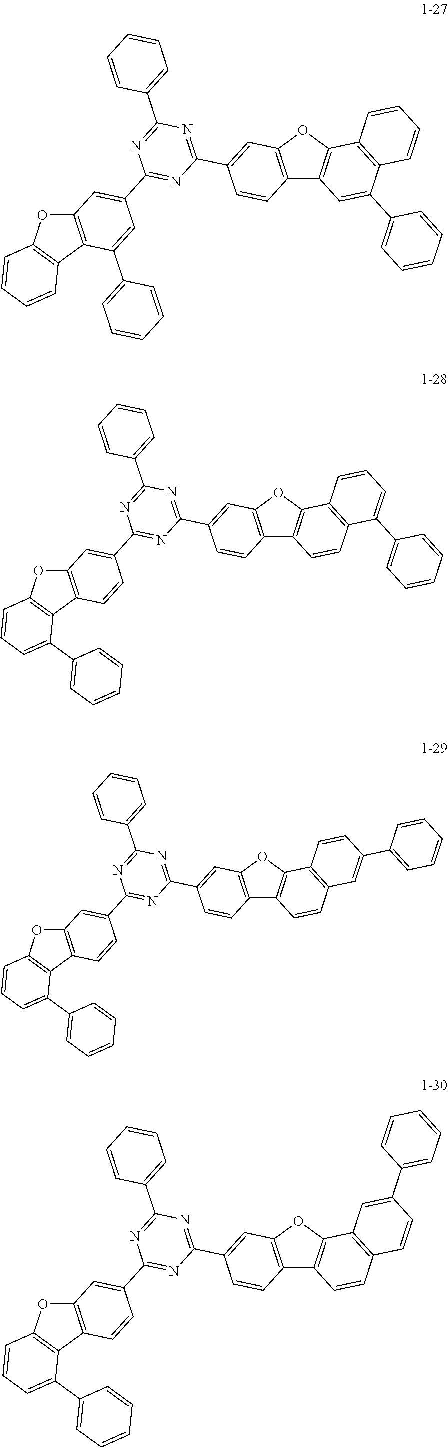 [SMILES: c1ccc(-c2nc(-c3ccc4c(c3)oc3c5ccccc5c(-c5ccccc5)cc43)nc(-c3cc(-c4ccccc4)c4c(c3)oc3ccccc34)n2)cc1 ; c1ccc(-c2nc(-c3ccc4c(c3)oc3c5cccc(-c6ccccc6)c5ccc43)nc(-c3ccc4c(c3)oc3cccc(-c5ccccc5)c34)n2)cc1 ; c1ccc(-c2ccc3c(ccc4c5ccc(-c6nc(-c7ccccc7)nc(-c7ccc8c(c7)oc7cccc(-c9ccccc9)c78)n6)cc5oc34)c2)cc1 ; c1ccc(-c2ccc3ccc4c5ccc(-c6nc(-c7ccccc7)nc(-c7ccc8c(c7)oc7cccc(-c9ccccc9)c78)n6)cc5oc4c3c2)cc1]

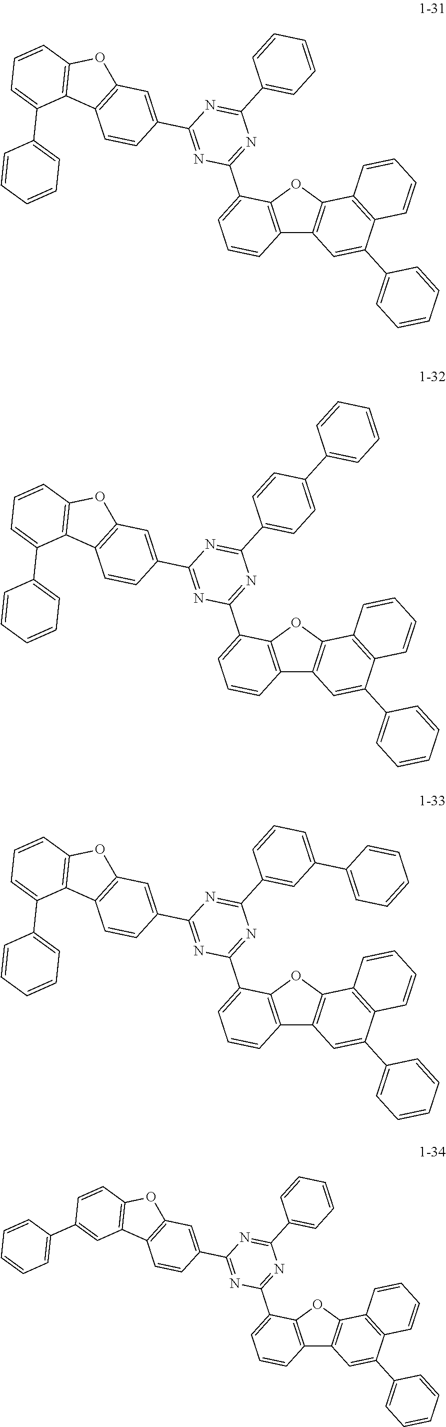 [SMILES: c1ccc(-c2nc(-c3ccc4c(c3)oc3cccc(-c5ccccc5)c34)nc(-c3cccc4c3oc3c5ccccc5c(-c5ccccc5)cc43)n2)cc1 ; c1ccc(-c2ccc(-c3nc(-c4ccc5c(c4)oc4cccc(-c6ccccc6)c45)nc(-c4cccc5c4oc4c6ccccc6c(-c6ccccc6)cc54)n3)cc2)cc1 ; c1ccc(-c2cccc(-c3nc(-c4ccc5c(c4)oc4cccc(-c6ccccc6)c45)nc(-c4cccc5c4oc4c6ccccc6c(-c6ccccc6)cc54)n3)c2)cc1 ; c1ccc(-c2ccc3oc4cc(-c5nc(-c6ccccc6)nc(-c6cccc7c6oc6c8ccccc8c(-c8ccccc8)cc76)n5)ccc4c3c2)cc1]

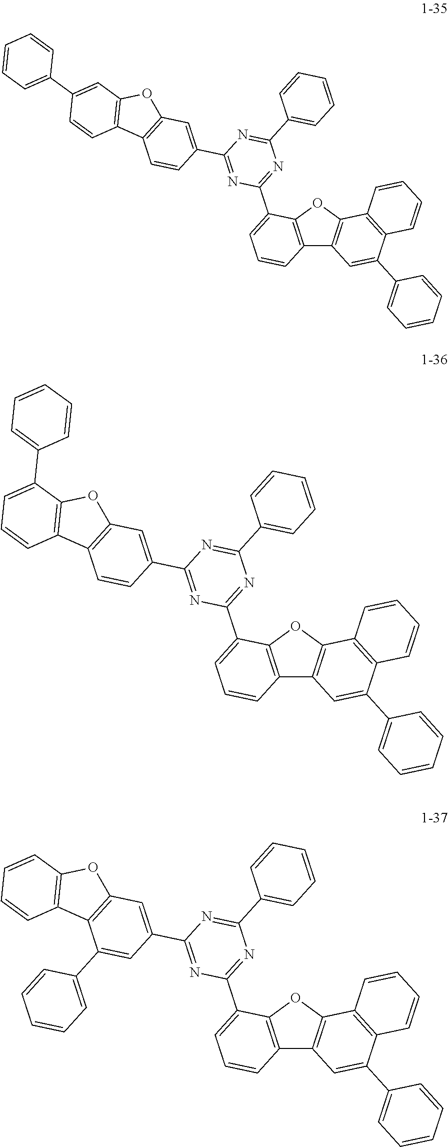 [SMILES: c1ccc(-c2ccc3c(c2)oc2cc(-c4nc(-c5ccccc5)nc(-c5cccc6c5oc5c7ccccc7c(-c7ccccc7)cc65)n4)ccc23)cc1 ; c1ccc(-c2nc(-c3ccc4c(c3)oc3c(-c5ccccc5)cccc34)nc(-c3cccc4c3oc3c5ccccc5c(-c5ccccc5)cc43)n2)cc1 ; c1ccc(-c2nc(-c3cc(-c4ccccc4)c4c(c3)oc3ccccc34)nc(-c3cccc4c3oc3c5ccccc5c(-c5ccccc5)cc43)n2)cc1]

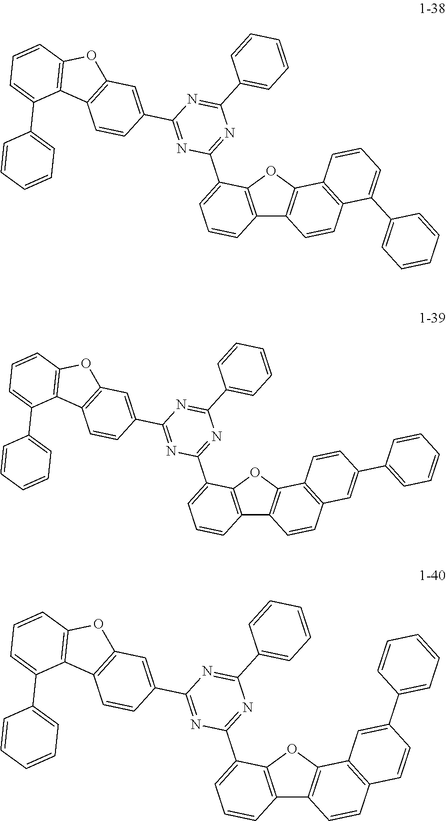 [SMILES: c1ccc(-c2nc(-c3ccc4c(c3)oc3cccc(-c5ccccc5)c34)nc(-c3cccc4c3oc3c5cccc(-c6ccccc6)c5ccc43)n2)cc1 ; c1ccc(-c2ccc3c(ccc4c5cccc(-c6nc(-c7ccccc7)nc(-c7ccc8c(c7)oc7cccc(-c9ccccc9)c78)n6)c5oc34)c2)cc1 ; c1ccc(-c2ccc3ccc4c5cccc(-c6nc(-c7ccccc7)nc(-c7ccc8c(c7)oc7cccc(-c9ccccc9)c78)n6)c5oc4c3c2)cc1]

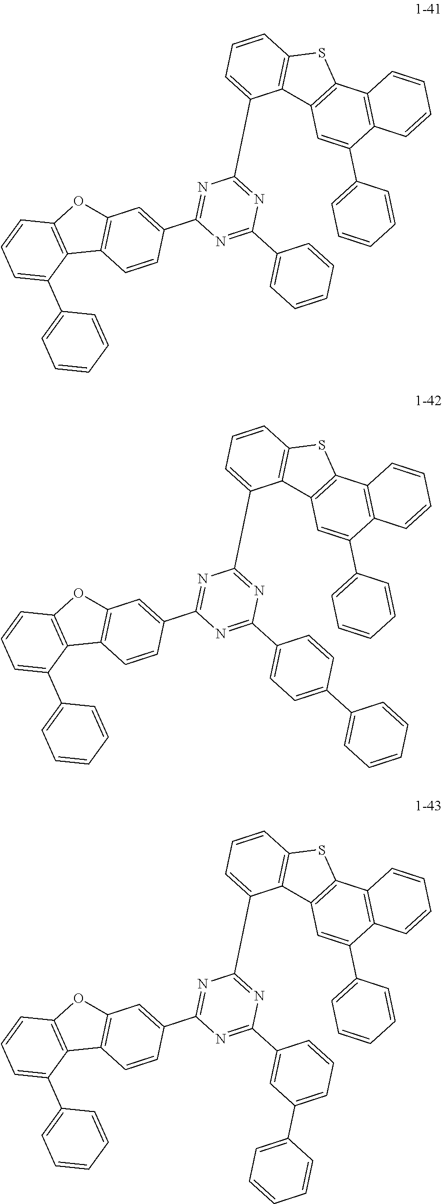 [SMILES: c1ccc(-c2nc(-c3ccc4c(c3)oc3cccc(-c5ccccc5)c34)nc(-c3cccc4sc5c6ccccc6c(-c6ccccc6)cc5c34)n2)cc1 ; c1ccc(-c2ccc(-c3nc(-c4ccc5c(c4)oc4cccc(-c6ccccc6)c45)nc(-c4cccc5sc6c7ccccc7c(-c7ccccc7)cc6c45)n3)cc2)cc1 ; c1ccc(-c2cccc(-c3nc(-c4ccc5c(c4)oc4cccc(-c6ccccc6)c45)nc(-c4cccc5sc6c7ccccc7c(-c7ccccc7)cc6c45)n3)c2)cc1]

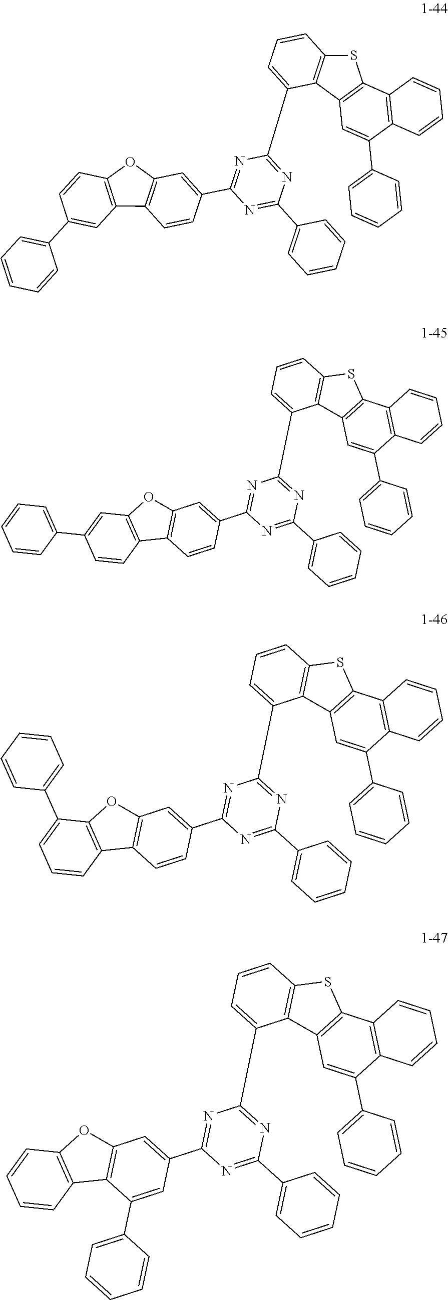 [SMILES: c1ccc(-c2ccc3oc4cc(-c5nc(-c6ccccc6)nc(-c6cccc7sc8c9ccccc9c(-c9ccccc9)cc8c67)n5)ccc4c3c2)cc1 ; c1ccc(-c2ccc3c(c2)oc2cc(-c4nc(-c5ccccc5)nc(-c5cccc6sc7c8ccccc8c(-c8ccccc8)cc7c56)n4)ccc23)cc1 ; c1ccc(-c2nc(-c3ccc4c(c3)oc3c(-c5ccccc5)cccc34)nc(-c3cccc4sc5c6ccccc6c(-c6ccccc6)cc5c34)n2)cc1 ; c1ccc(-c2nc(-c3cc(-c4ccccc4)c4c(c3)oc3ccccc34)nc(-c3cccc4sc5c6ccccc6c(-c6ccccc6)cc5c34)n2)cc1]

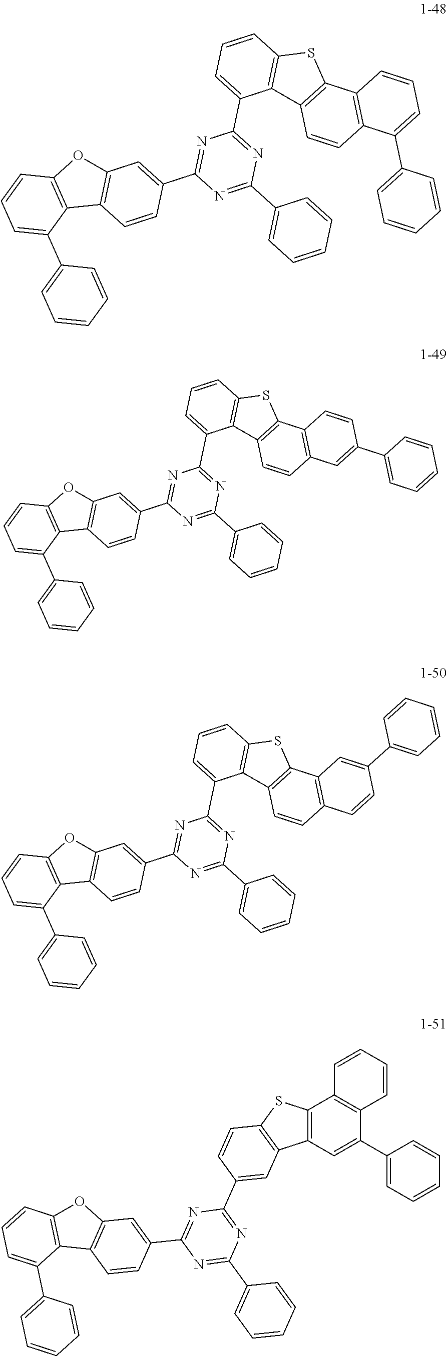 [SMILES: c1ccc(-c2nc(-c3ccc4c(c3)oc3cccc(-c5ccccc5)c34)nc(-c3cccc4sc5c6cccc(-c7ccccc7)c6ccc5c34)n2)cc1 ; c1ccc(-c2ccc3c(ccc4c3sc3cccc(-c5nc(-c6ccccc6)nc(-c6ccc7c(c6)oc6cccc(-c8ccccc8)c67)n5)c34)c2)cc1 ; c1ccc(-c2ccc3ccc4c(sc5cccc(-c6nc(-c7ccccc7)nc(-c7ccc8c(c7)oc7cccc(-c9ccccc9)c78)n6)c54)c3c2)cc1 ; c1ccc(-c2nc(-c3ccc4c(c3)oc3cccc(-c5ccccc5)c34)nc(-c3ccc4sc5c6ccccc6c(-c6ccccc6)cc5c4c3)n2)cc1]

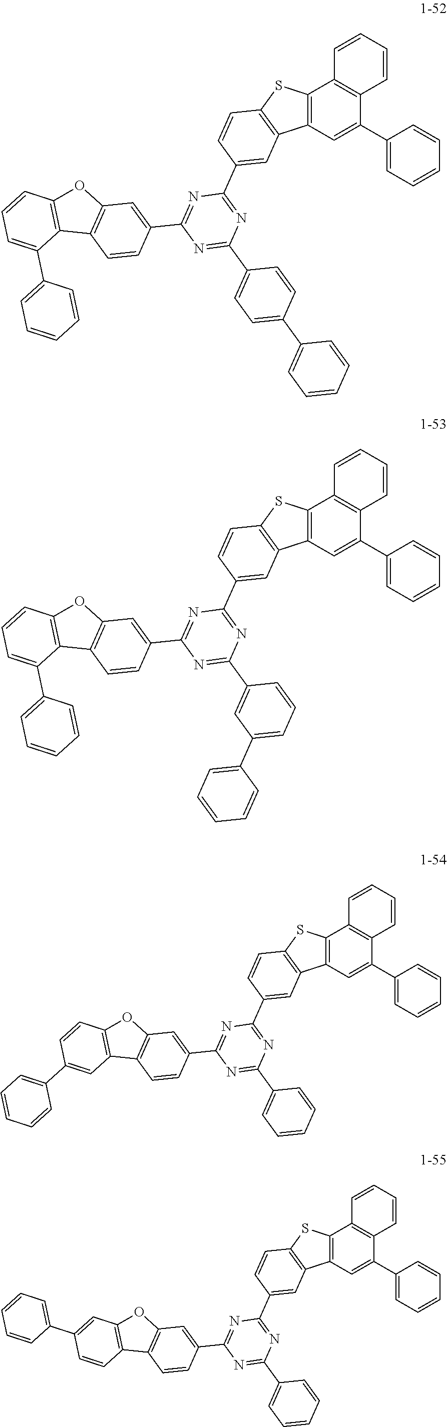 [SMILES: c1ccc(-c2ccc(-c3nc(-c4ccc5c(c4)oc4cccc(-c6ccccc6)c45)nc(-c4ccc5sc6c7ccccc7c(-c7ccccc7)cc6c5c4)n3)cc2)cc1 ; c1ccc(-c2cccc(-c3nc(-c4ccc5c(c4)oc4cccc(-c6ccccc6)c45)nc(-c4ccc5sc6c7ccccc7c(-c7ccccc7)cc6c5c4)n3)c2)cc1 ; c1ccc(-c2ccc3oc4cc(-c5nc(-c6ccccc6)nc(-c6ccc7sc8c9ccccc9c(-c9ccccc9)cc8c7c6)n5)ccc4c3c2)cc1 ; c1ccc(-c2ccc3c(c2)oc2cc(-c4nc(-c5ccccc5)nc(-c5ccc6sc7c8ccccc8c(-c8ccccc8)cc7c6c5)n4)ccc23)cc1]

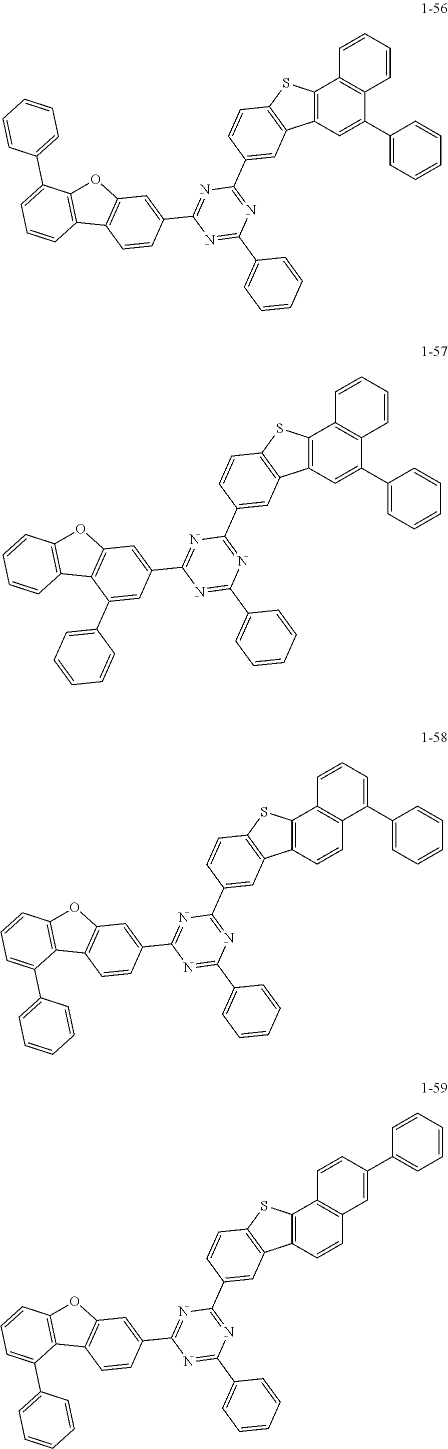 [SMILES: c1ccc(-c2nc(-c3ccc4c(c3)oc3c(-c5ccccc5)cccc34)nc(-c3ccc4sc5c6ccccc6c(-c6ccccc6)cc5c4c3)n2)cc1 ; c1ccc(-c2nc(-c3cc(-c4ccccc4)c4c(c3)oc3ccccc34)nc(-c3ccc4sc5c6ccccc6c(-c6ccccc6)cc5c4c3)n2)cc1 ; c1ccc(-c2nc(-c3ccc4c(c3)oc3cccc(-c5ccccc5)c34)nc(-c3ccc4sc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)oc7cccc(-c9ccccc9)c78)n6)ccc5sc34)c2)cc1]

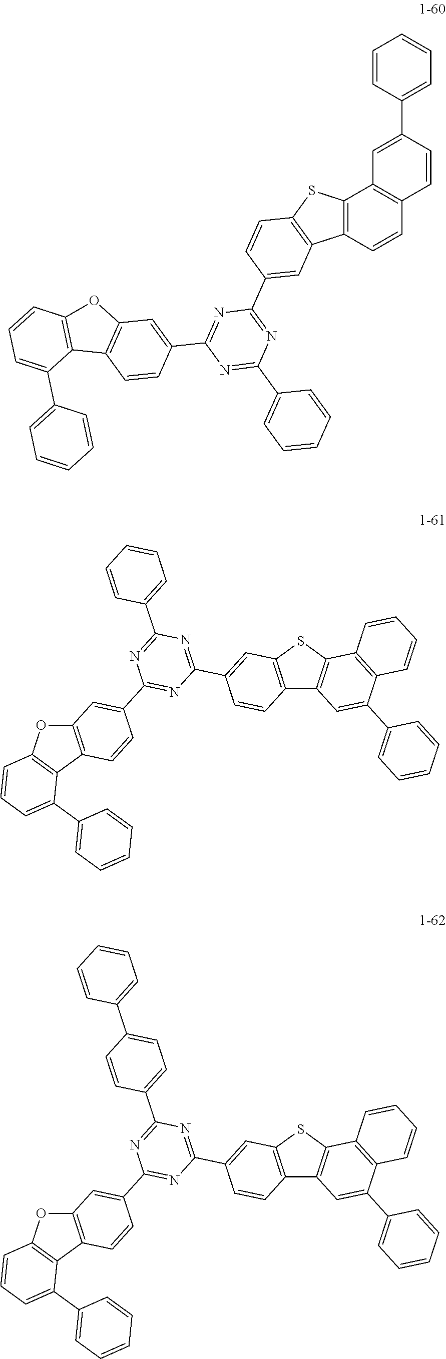 [SMILES: c1ccc(-c2ccc3ccc4c5cc(-c6nc(-c7ccccc7)nc(-c7ccc8c(c7)oc7cccc(-c9ccccc9)c78)n6)ccc5sc4c3c2)cc1 ; c1ccc(-c2nc(-c3ccc4c(c3)oc3cccc(-c5ccccc5)c34)nc(-c3ccc4c(c3)sc3c5ccccc5c(-c5ccccc5)cc43)n2)cc1 ; c1ccc(-c2ccc(-c3nc(-c4ccc5c(c4)oc4cccc(-c6ccccc6)c45)nc(-c4ccc5c(c4)sc4c6ccccc6c(-c6ccccc6)cc54)n3)cc2)cc1]

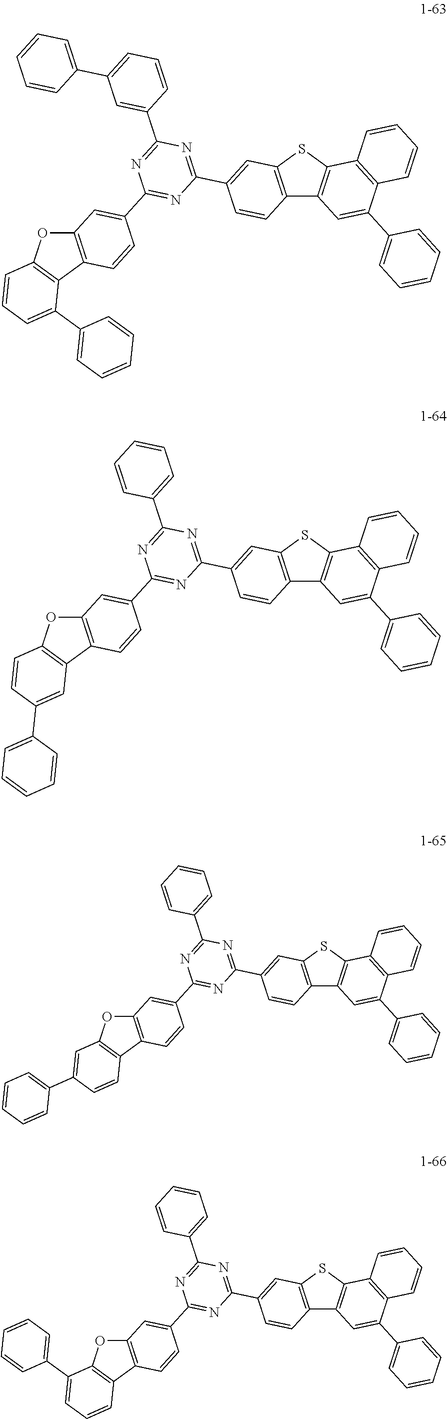 [SMILES: c1ccc(-c2cccc(-c3nc(-c4ccc5c(c4)oc4cccc(-c6ccccc6)c45)nc(-c4ccc5c(c4)sc4c6ccccc6c(-c6ccccc6)cc54)n3)c2)cc1 ; c1ccc(-c2ccc3oc4cc(-c5nc(-c6ccccc6)nc(-c6ccc7c(c6)sc6c8ccccc8c(-c8ccccc8)cc76)n5)ccc4c3c2)cc1 ; c1ccc(-c2ccc3c(c2)oc2cc(-c4nc(-c5ccccc5)nc(-c5ccc6c(c5)sc5c7ccccc7c(-c7ccccc7)cc65)n4)ccc23)cc1 ; c1ccc(-c2nc(-c3ccc4c(c3)oc3c(-c5ccccc5)cccc34)nc(-c3ccc4c(c3)sc3c5ccccc5c(-c5ccccc5)cc43)n2)cc1]

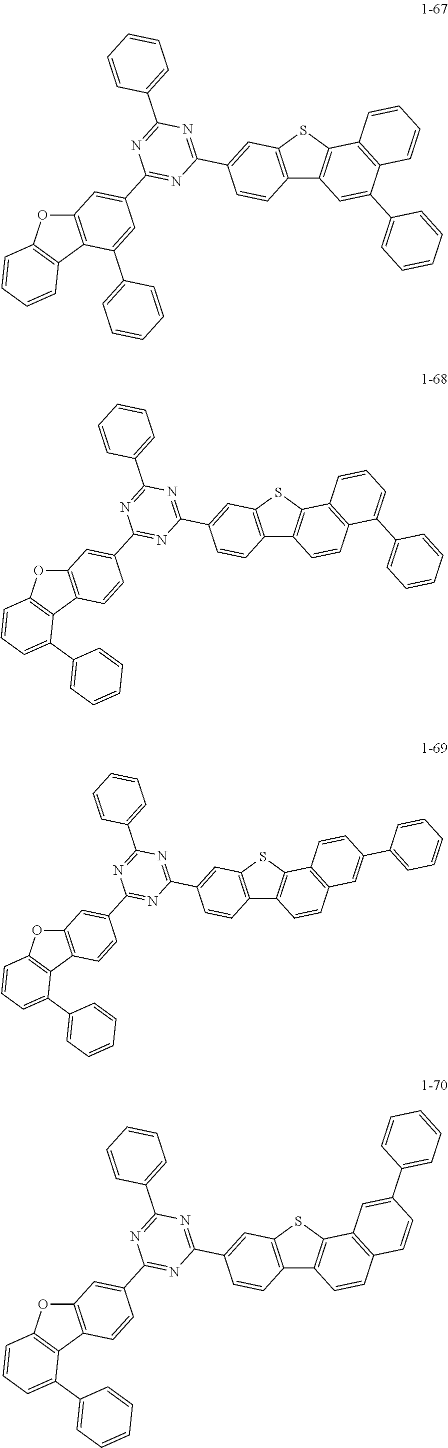 [SMILES: c1ccc(-c2nc(-c3cc(-c4ccccc4)c4c(c3)oc3ccccc34)nc(-c3ccc4c(c3)sc3c5ccccc5c(-c5ccccc5)cc43)n2)cc1 ; c1ccc(-c2nc(-c3ccc4c(c3)oc3cccc(-c5ccccc5)c34)nc(-c3ccc4c(c3)sc3c5cccc(-c6ccccc6)c5ccc43)n2)cc1 ; c1ccc(-c2ccc3c(ccc4c5ccc(-c6nc(-c7ccccc7)nc(-c7ccc8c(c7)oc7cccc(-c9ccccc9)c78)n6)cc5sc34)c2)cc1 ; c1ccc(-c2ccc3ccc4c5ccc(-c6nc(-c7ccccc7)nc(-c7ccc8c(c7)oc7cccc(-c9ccccc9)c78)n6)cc5sc4c3c2)cc1]

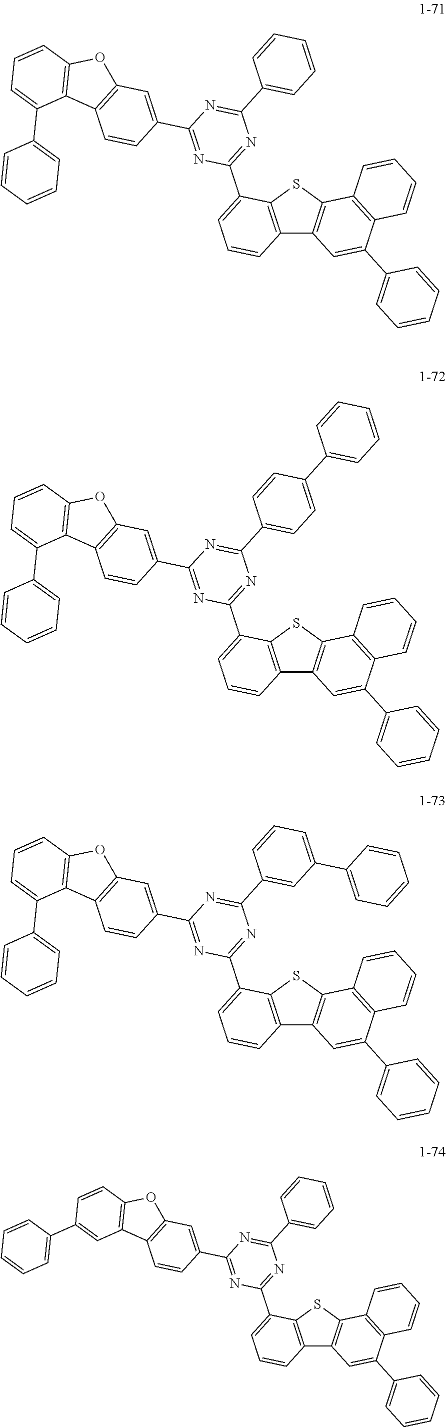 [SMILES: c1ccc(-c2nc(-c3ccc4c(c3)oc3cccc(-c5ccccc5)c34)nc(-c3cccc4c3sc3c5ccccc5c(-c5ccccc5)cc43)n2)cc1 ; c1ccc(-c2ccc(-c3nc(-c4ccc5c(c4)oc4cccc(-c6ccccc6)c45)nc(-c4cccc5c4sc4c6ccccc6c(-c6ccccc6)cc54)n3)cc2)cc1 ; c1ccc(-c2cccc(-c3nc(-c4ccc5c(c4)oc4cccc(-c6ccccc6)c45)nc(-c4cccc5c4sc4c6ccccc6c(-c6ccccc6)cc54)n3)c2)cc1 ; c1ccc(-c2ccc3oc4cc(-c5nc(-c6ccccc6)nc(-c6cccc7c6sc6c8ccccc8c(-c8ccccc8)cc76)n5)ccc4c3c2)cc1]

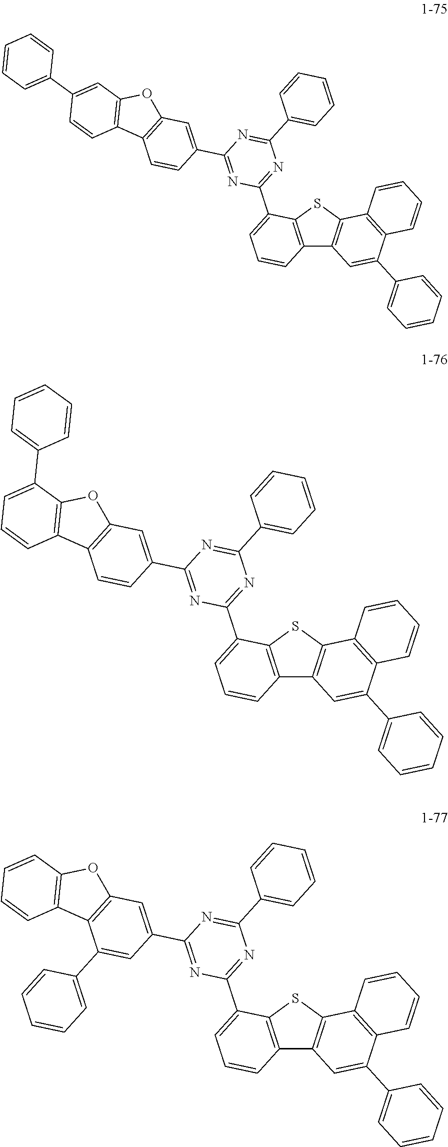 [SMILES: c1ccc(-c2ccc3c(c2)oc2cc(-c4nc(-c5ccccc5)nc(-c5cccc6c5sc5c7ccccc7c(-c7ccccc7)cc65)n4)ccc23)cc1 ; c1ccc(-c2nc(-c3ccc4c(c3)oc3c(-c5ccccc5)cccc34)nc(-c3cccc4c3sc3c5ccccc5c(-c5ccccc5)cc43)n2)cc1 ; c1ccc(-c2nc(-c3cc(-c4ccccc4)c4c(c3)oc3ccccc34)nc(-c3cccc4c3sc3c5ccccc5c(-c5ccccc5)cc43)n2)cc1]

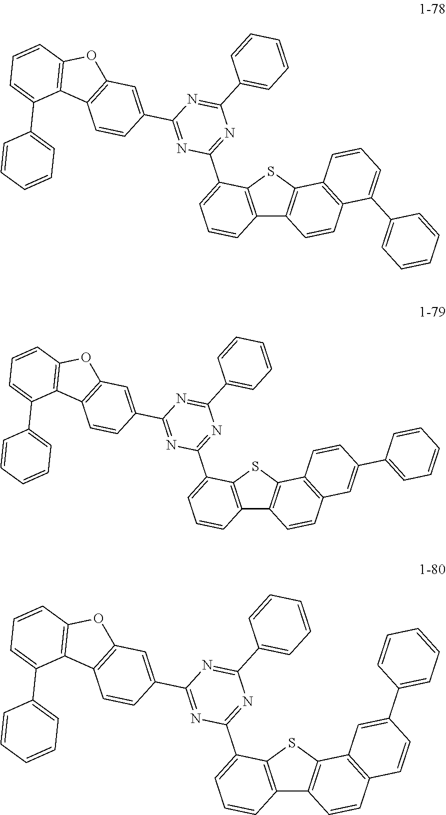 [SMILES: c1ccc(-c2nc(-c3ccc4c(c3)oc3cccc(-c5ccccc5)c34)nc(-c3cccc4c3sc3c5cccc(-c6ccccc6)c5ccc43)n2)cc1 ; c1ccc(-c2ccc3c(ccc4c5cccc(-c6nc(-c7ccccc7)nc(-c7ccc8c(c7)oc7cccc(-c9ccccc9)c78)n6)c5sc34)c2)cc1 ; c1ccc(-c2ccc3ccc4c5cccc(-c6nc(-c7ccccc7)nc(-c7ccc8c(c7)oc7cccc(-c9ccccc9)c78)n6)c5sc4c3c2)cc1]

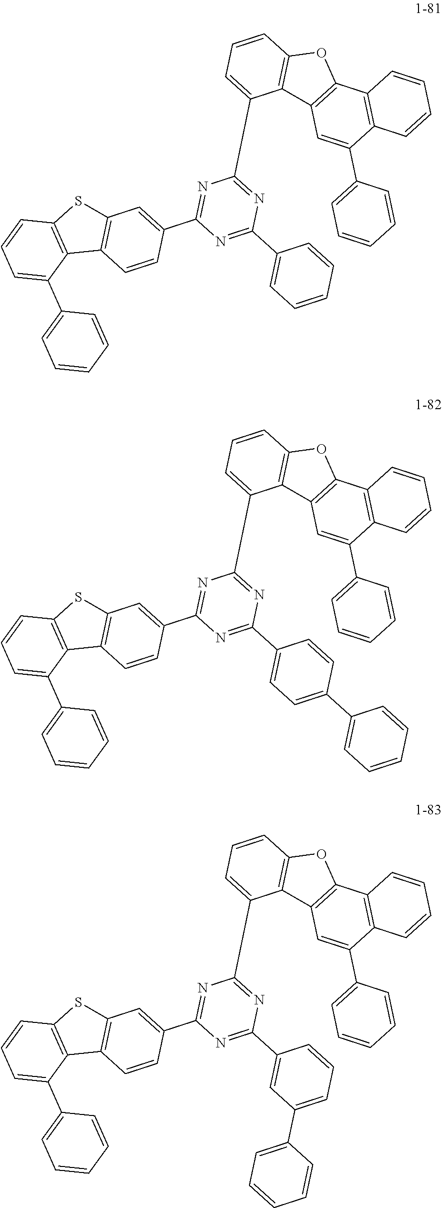 [SMILES: c1ccc(-c2nc(-c3ccc4c(c3)sc3cccc(-c5ccccc5)c34)nc(-c3cccc4oc5c6ccccc6c(-c6ccccc6)cc5c34)n2)cc1 ; c1ccc(-c2ccc(-c3nc(-c4ccc5c(c4)sc4cccc(-c6ccccc6)c45)nc(-c4cccc5oc6c7ccccc7c(-c7ccccc7)cc6c45)n3)cc2)cc1 ; c1ccc(-c2cccc(-c3nc(-c4ccc5c(c4)sc4cccc(-c6ccccc6)c45)nc(-c4cccc5oc6c7ccccc7c(-c7ccccc7)cc6c45)n3)c2)cc1]

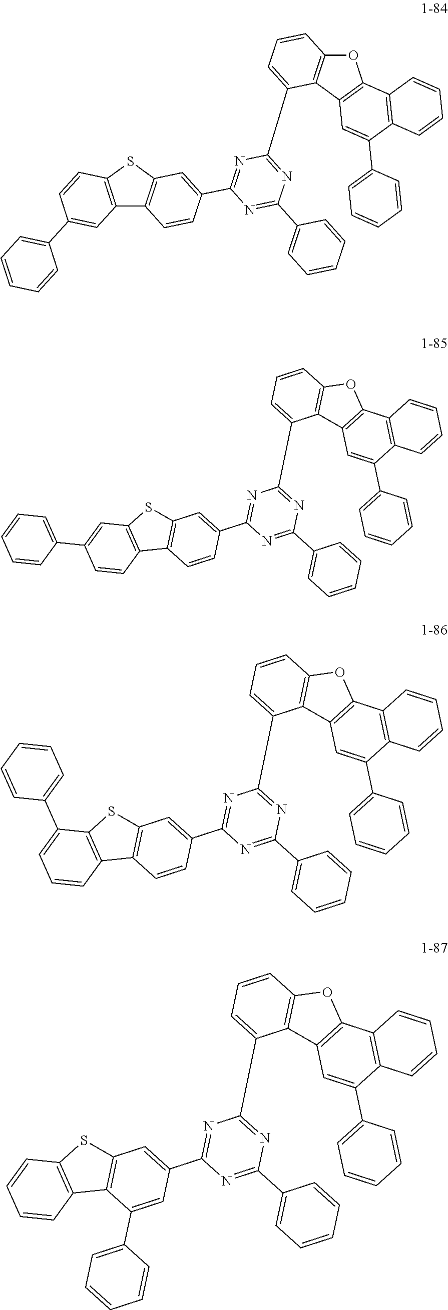 [SMILES: c1ccc(-c2ccc3sc4cc(-c5nc(-c6ccccc6)nc(-c6cccc7oc8c9ccccc9c(-c9ccccc9)cc8c67)n5)ccc4c3c2)cc1 ; c1ccc(-c2ccc3c(c2)sc2cc(-c4nc(-c5ccccc5)nc(-c5cccc6oc7c8ccccc8c(-c8ccccc8)cc7c56)n4)ccc23)cc1 ; c1ccc(-c2nc(-c3ccc4c(c3)sc3c(-c5ccccc5)cccc34)nc(-c3cccc4oc5c6ccccc6c(-c6ccccc6)cc5c34)n2)cc1 ; c1ccc(-c2nc(-c3cc(-c4ccccc4)c4c(c3)sc3ccccc34)nc(-c3cccc4oc5c6ccccc6c(-c6ccccc6)cc5c34)n2)cc1]

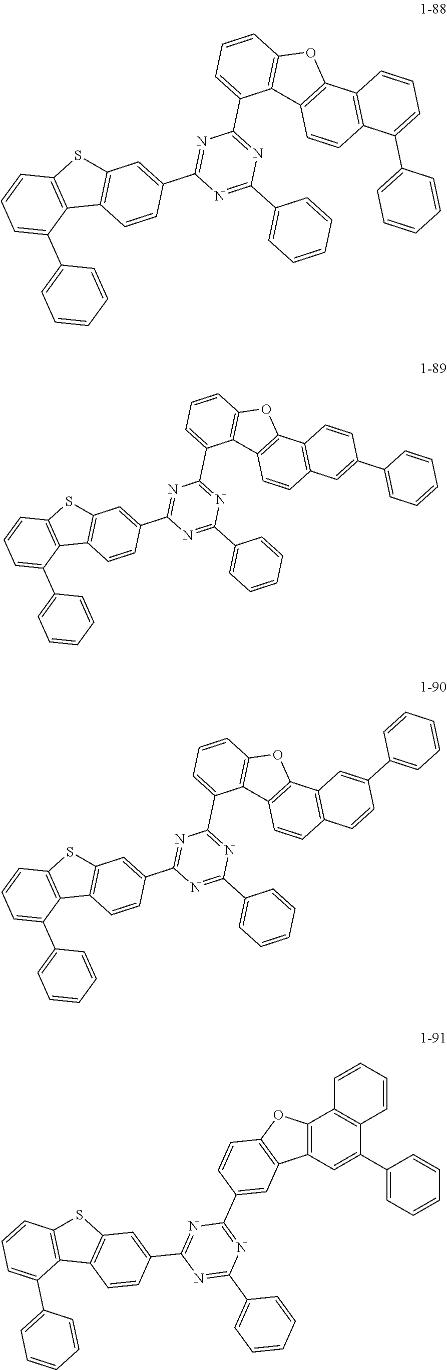 [SMILES: c1ccc(-c2nc(-c3ccc4c(c3)sc3cccc(-c5ccccc5)c34)nc(-c3cccc4oc5c6cccc(-c7ccccc7)c6ccc5c34)n2)cc1 ; c1ccc(-c2ccc3c(ccc4c3oc3cccc(-c5nc(-c6ccccc6)nc(-c6ccc7c(c6)sc6cccc(-c8ccccc8)c67)n5)c34)c2)cc1 ; c1ccc(-c2ccc3ccc4c(oc5cccc(-c6nc(-c7ccccc7)nc(-c7ccc8c(c7)sc7cccc(-c9ccccc9)c78)n6)c54)c3c2)cc1 ; c1ccc(-c2nc(-c3ccc4c(c3)sc3cccc(-c5ccccc5)c34)nc(-c3ccc4oc5c6ccccc6c(-c6ccccc6)cc5c4c3)n2)cc1]

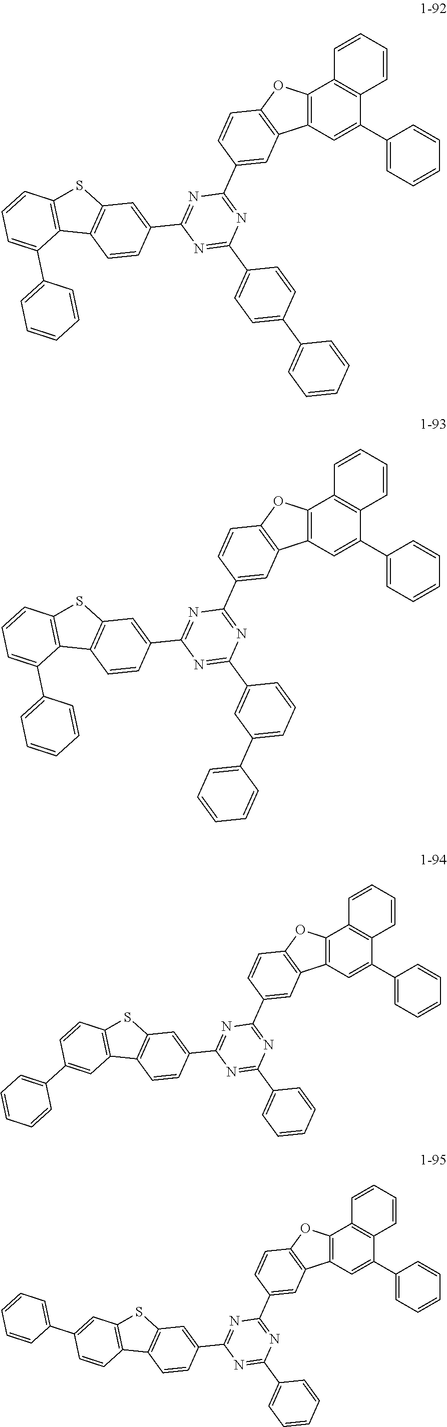 [SMILES: c1ccc(-c2ccc(-c3nc(-c4ccc5c(c4)sc4cccc(-c6ccccc6)c45)nc(-c4ccc5oc6c7ccccc7c(-c7ccccc7)cc6c5c4)n3)cc2)cc1 ; c1ccc(-c2cccc(-c3nc(-c4ccc5c(c4)sc4cccc(-c6ccccc6)c45)nc(-c4ccc5oc6c7ccccc7c(-c7ccccc7)cc6c5c4)n3)c2)cc1 ; c1ccc(-c2ccc3sc4cc(-c5nc(-c6ccccc6)nc(-c6ccc7oc8c9ccccc9c(-c9ccccc9)cc8c7c6)n5)ccc4c3c2)cc1 ; c1ccc(-c2ccc3c(c2)sc2cc(-c4nc(-c5ccccc5)nc(-c5ccc6oc7c8ccccc8c(-c8ccccc8)cc7c6c5)n4)ccc23)cc1]

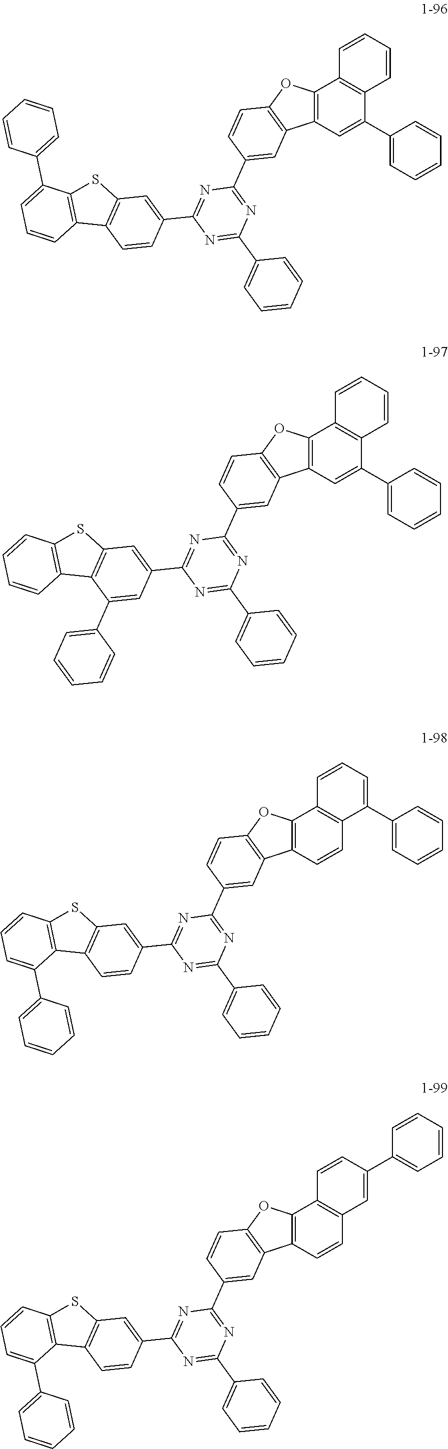 [SMILES: c1ccc(-c2nc(-c3ccc4c(c3)sc3c(-c5ccccc5)cccc34)nc(-c3ccc4oc5c6ccccc6c(-c6ccccc6)cc5c4c3)n2)cc1 ; c1ccc(-c2nc(-c3cc(-c4ccccc4)c4c(c3)sc3ccccc34)nc(-c3ccc4oc5c6ccccc6c(-c6ccccc6)cc5c4c3)n2)cc1 ; c1ccc(-c2nc(-c3ccc4c(c3)sc3cccc(-c5ccccc5)c34)nc(-c3ccc4oc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)sc7cccc(-c9ccccc9)c78)n6)ccc5oc34)c2)cc1]

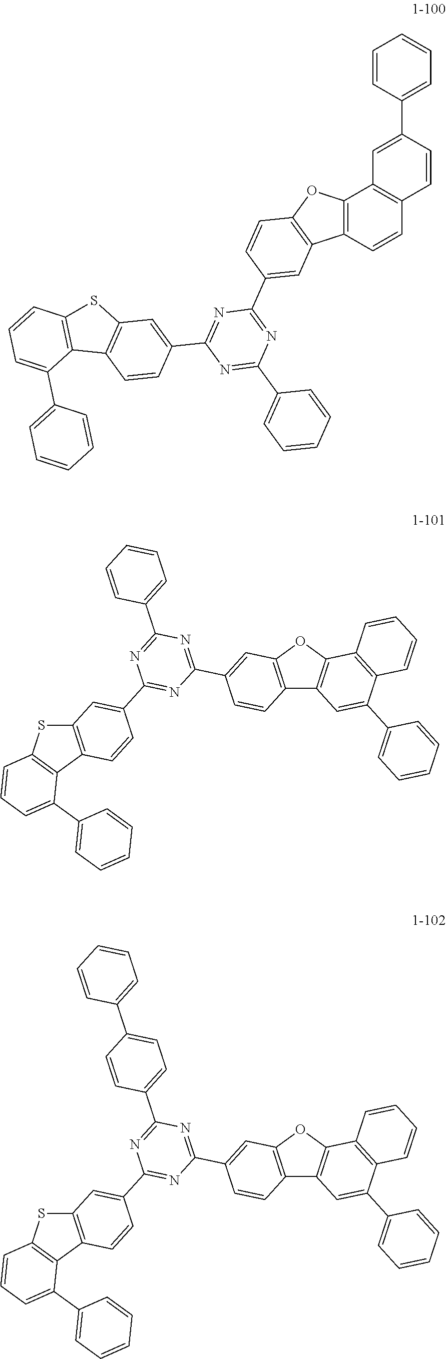 [SMILES: c1ccc(-c2ccc3ccc4c5cc(-c6nc(-c7ccccc7)nc(-c7ccc8c(c7)sc7cccc(-c9ccccc9)c78)n6)ccc5oc4c3c2)cc1 ; c1ccc(-c2nc(-c3ccc4c(c3)oc3c5ccccc5c(-c5ccccc5)cc43)nc(-c3ccc4c(c3)sc3cccc(-c5ccccc5)c34)n2)cc1 ; c1ccc(-c2ccc(-c3nc(-c4ccc5c(c4)oc4c6ccccc6c(-c6ccccc6)cc54)nc(-c4ccc5c(c4)sc4cccc(-c6ccccc6)c45)n3)cc2)cc1]

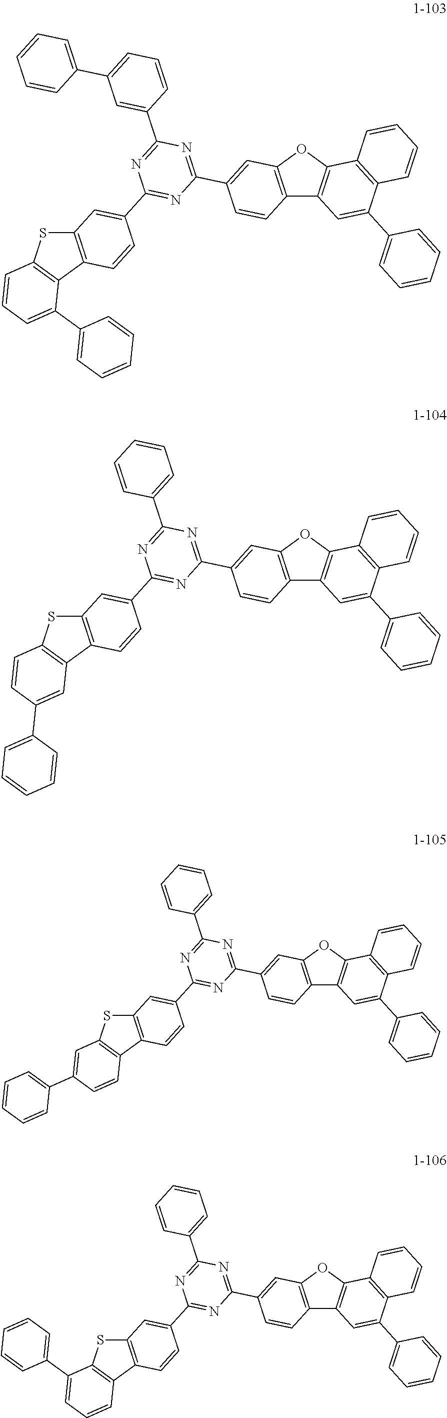 [SMILES: c1ccc(-c2cccc(-c3nc(-c4ccc5c(c4)oc4c6ccccc6c(-c6ccccc6)cc54)nc(-c4ccc5c(c4)sc4cccc(-c6ccccc6)c45)n3)c2)cc1 ; c1ccc(-c2ccc3sc4cc(-c5nc(-c6ccccc6)nc(-c6ccc7c(c6)oc6c8ccccc8c(-c8ccccc8)cc76)n5)ccc4c3c2)cc1 ; c1ccc(-c2ccc3c(c2)sc2cc(-c4nc(-c5ccccc5)nc(-c5ccc6c(c5)oc5c7ccccc7c(-c7ccccc7)cc65)n4)ccc23)cc1 ; c1ccc(-c2nc(-c3ccc4c(c3)oc3c5ccccc5c(-c5ccccc5)cc43)nc(-c3ccc4c(c3)sc3c(-c5ccccc5)cccc34)n2)cc1]

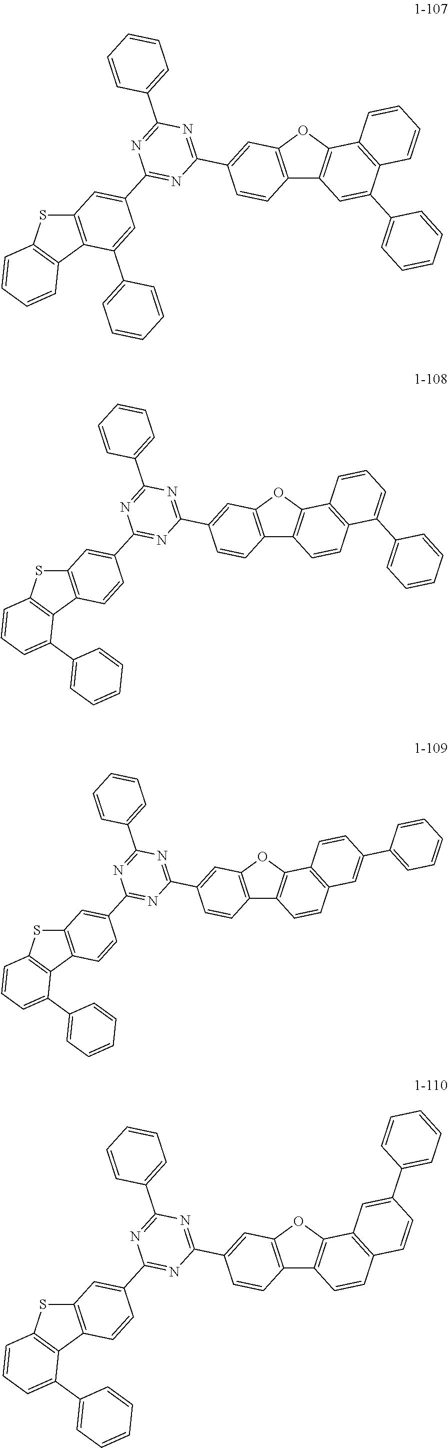 [SMILES: c1ccc(-c2nc(-c3ccc4c(c3)oc3c5ccccc5c(-c5ccccc5)cc43)nc(-c3cc(-c4ccccc4)c4c(c3)sc3ccccc34)n2)cc1 ; c1ccc(-c2nc(-c3ccc4c(c3)oc3c5cccc(-c6ccccc6)c5ccc43)nc(-c3ccc4c(c3)sc3cccc(-c5ccccc5)c34)n2)cc1 ; c1ccc(-c2ccc3c(ccc4c5ccc(-c6nc(-c7ccccc7)nc(-c7ccc8c(c7)sc7cccc(-c9ccccc9)c78)n6)cc5oc34)c2)cc1 ; c1ccc(-c2ccc3ccc4c5ccc(-c6nc(-c7ccccc7)nc(-c7ccc8c(c7)sc7cccc(-c9ccccc9)c78)n6)cc5oc4c3c2)cc1]

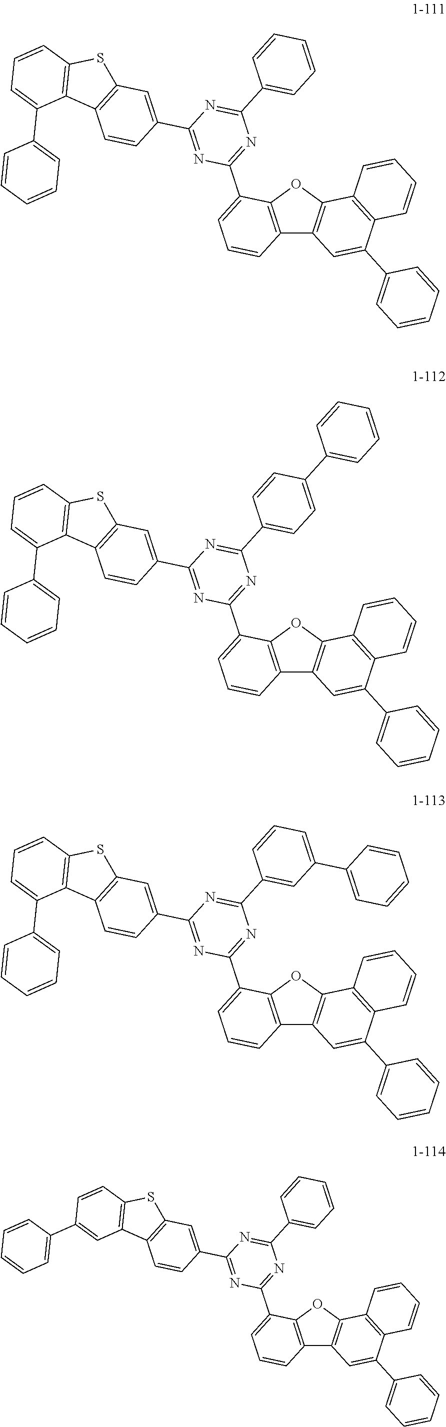 [SMILES: c1ccc(-c2nc(-c3ccc4c(c3)sc3cccc(-c5ccccc5)c34)nc(-c3cccc4c3oc3c5ccccc5c(-c5ccccc5)cc43)n2)cc1 ; c1ccc(-c2ccc(-c3nc(-c4ccc5c(c4)sc4cccc(-c6ccccc6)c45)nc(-c4cccc5c4oc4c6ccccc6c(-c6ccccc6)cc54)n3)cc2)cc1 ; c1ccc(-c2cccc(-c3nc(-c4ccc5c(c4)sc4cccc(-c6ccccc6)c45)nc(-c4cccc5c4oc4c6ccccc6c(-c6ccccc6)cc54)n3)c2)cc1 ; c1ccc(-c2ccc3sc4cc(-c5nc(-c6ccccc6)nc(-c6cccc7c6oc6c8ccccc8c(-c8ccccc8)cc76)n5)ccc4c3c2)cc1]

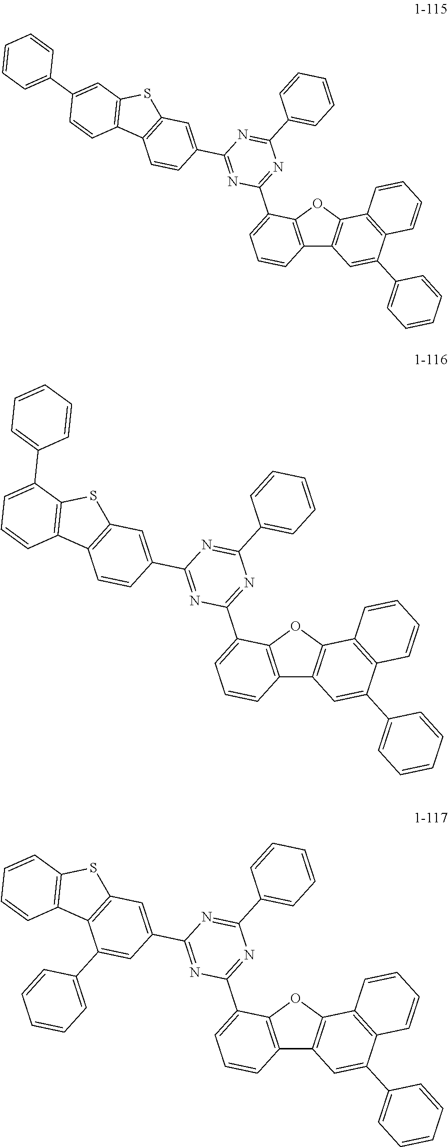 [SMILES: c1ccc(-c2ccc3c(c2)sc2cc(-c4nc(-c5ccccc5)nc(-c5cccc6c5oc5c7ccccc7c(-c7ccccc7)cc65)n4)ccc23)cc1 ; c1ccc(-c2nc(-c3ccc4c(c3)sc3c(-c5ccccc5)cccc34)nc(-c3cccc4c3oc3c5ccccc5c(-c5ccccc5)cc43)n2)cc1 ; c1ccc(-c2nc(-c3cc(-c4ccccc4)c4c(c3)sc3ccccc34)nc(-c3cccc4c3oc3c5ccccc5c(-c5ccccc5)cc43)n2)cc1]

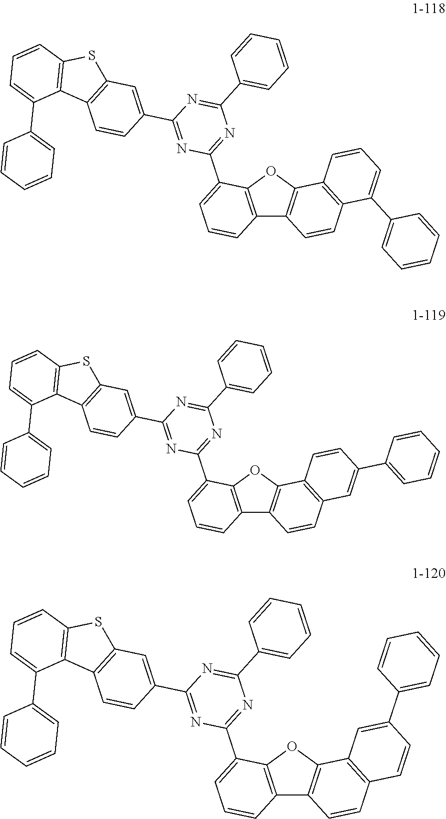 [SMILES: c1ccc(-c2nc(-c3ccc4c(c3)sc3cccc(-c5ccccc5)c34)nc(-c3cccc4c3oc3c5cccc(-c6ccccc6)c5ccc43)n2)cc1 ; c1ccc(-c2ccc3c(ccc4c5cccc(-c6nc(-c7ccccc7)nc(-c7ccc8c(c7)sc7cccc(-c9ccccc9)c78)n6)c5oc34)c2)cc1 ; c1ccc(-c2ccc3ccc4c5cccc(-c6nc(-c7ccccc7)nc(-c7ccc8c(c7)sc7cccc(-c9ccccc9)c78)n6)c5oc4c3c2)cc1]

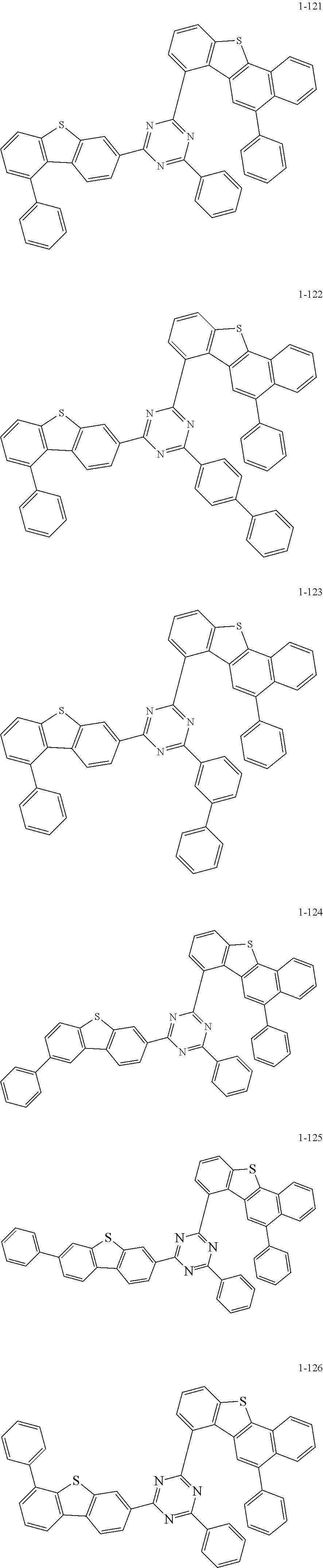

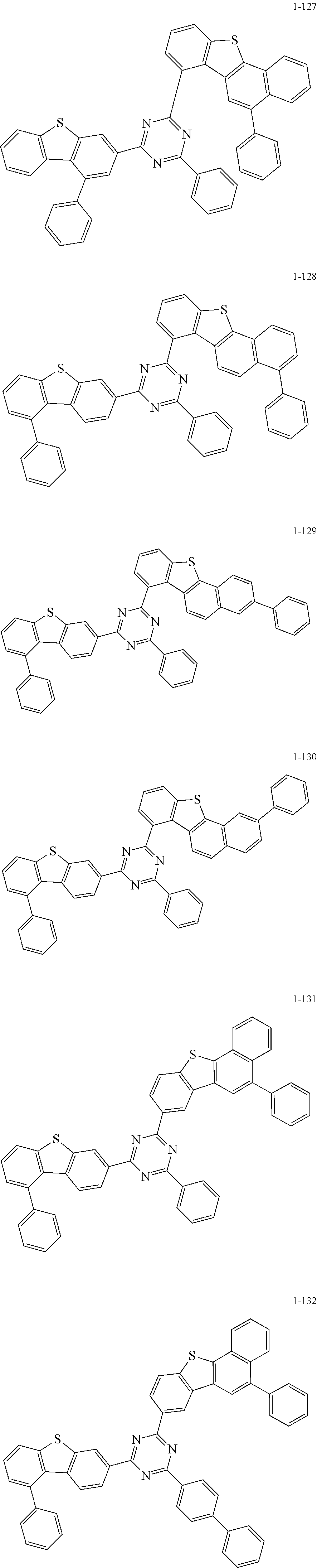

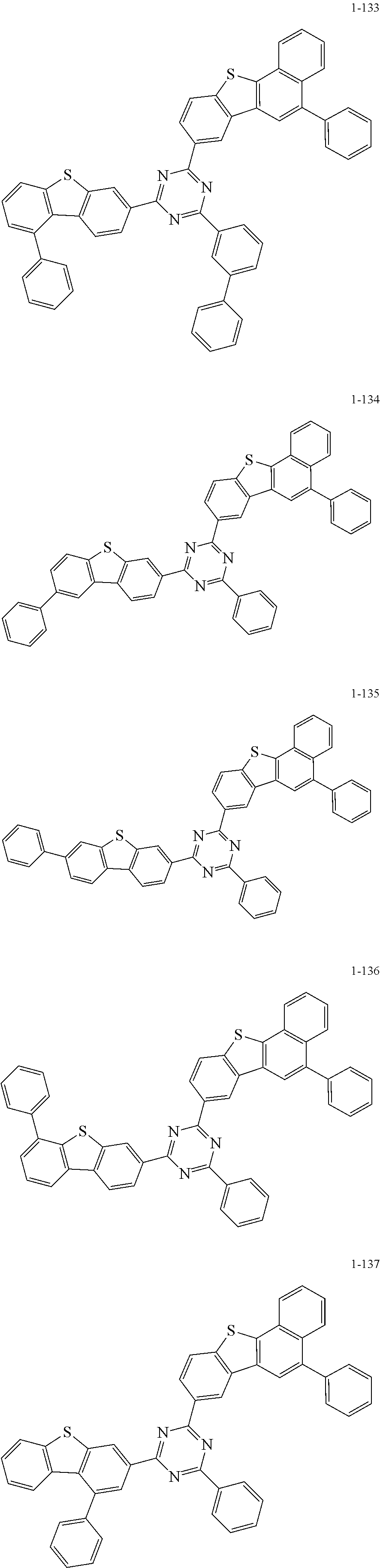 [SMILES: c1ccc(-c2cccc(-c3nc(-c4ccc5c(c4)sc4cccc(-c6ccccc6)c45)nc(-c4ccc5sc6c7ccccc7c(-c7ccccc7)cc6c5c4)n3)c2)cc1 ; c1ccc(-c2ccc3sc4cc(-c5nc(-c6ccccc6)nc(-c6ccc7sc8c9ccccc9c(-c9ccccc9)cc8c7c6)n5)ccc4c3c2)cc1 ; c1ccc(-c2ccc3c(c2)sc2cc(-c4nc(-c5ccccc5)nc(-c5ccc6sc7c8ccccc8c(-c8ccccc8)cc7c6c5)n4)ccc23)cc1 ; c1ccc(-c2nc(-c3ccc4c(c3)sc3c(-c5ccccc5)cccc34)nc(-c3ccc4sc5c6ccccc6c(-c6ccccc6)cc5c4c3)n2)cc1 ; c1ccc(-c2nc(-c3cc(-c4ccccc4)c4c(c3)sc3ccccc34)nc(-c3ccc4sc5c6ccccc6c(-c6ccccc6)cc5c4c3)n2)cc1]

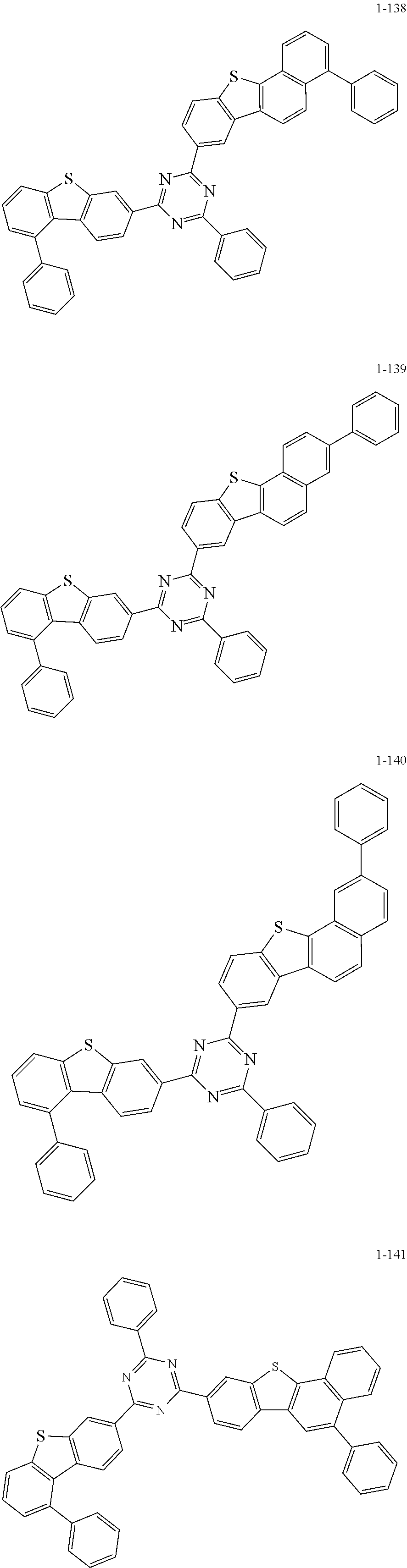 [SMILES: c1ccc(-c2nc(-c3ccc4c(c3)sc3cccc(-c5ccccc5)c34)nc(-c3ccc4sc5c6cccc(-c7ccccc7)c6ccc5c4c3)n2)cc1 ; c1ccc(-c2ccc3c(ccc4c5cc(-c6nc(-c7ccccc7)nc(-c7ccc8c(c7)sc7cccc(-c9ccccc9)c78)n6)ccc5sc34)c2)cc1 ; c1ccc(-c2ccc3ccc4c5cc(-c6nc(-c7ccccc7)nc(-c7ccc8c(c7)sc7cccc(-c9ccccc9)c78)n6)ccc5sc4c3c2)cc1 ; c1ccc(-c2nc(-c3ccc4c(c3)sc3c5ccccc5c(-c5ccccc5)cc43)nc(-c3ccc4c(c3)sc3cccc(-c5ccccc5)c34)n2)cc1]

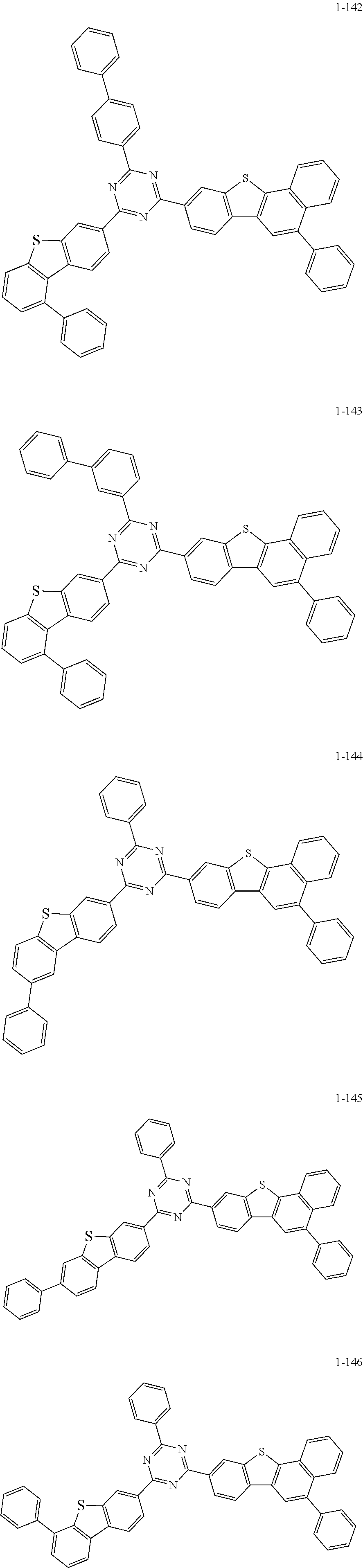

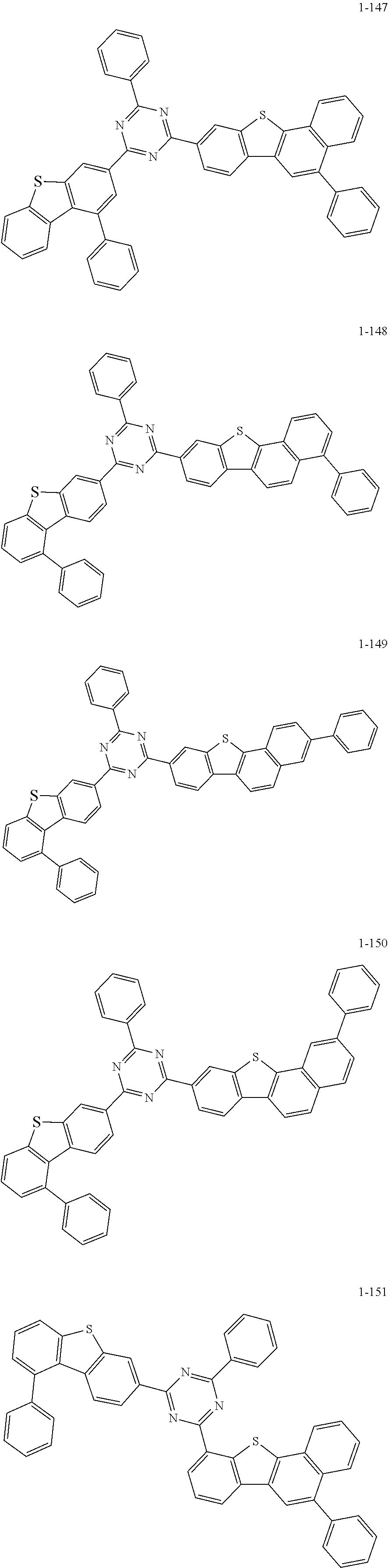 [SMILES: c1ccc(-c2nc(-c3ccc4c(c3)sc3c5ccccc5c(-c5ccccc5)cc43)nc(-c3cc(-c4ccccc4)c4c(c3)sc3ccccc34)n2)cc1 ; c1ccc(-c2nc(-c3ccc4c(c3)sc3c5cccc(-c6ccccc6)c5ccc43)nc(-c3ccc4c(c3)sc3cccc(-c5ccccc5)c34)n2)cc1 ; c1ccc(-c2ccc3c(ccc4c5ccc(-c6nc(-c7ccccc7)nc(-c7ccc8c(c7)sc7cccc(-c9ccccc9)c78)n6)cc5sc34)c2)cc1 ; c1ccc(-c2ccc3ccc4c5ccc(-c6nc(-c7ccccc7)nc(-c7ccc8c(c7)sc7cccc(-c9ccccc9)c78)n6)cc5sc4c3c2)cc1 ; c1ccc(-c2nc(-c3ccc4c(c3)sc3cccc(-c5ccccc5)c34)nc(-c3cccc4c3sc3c5ccccc5c(-c5ccccc5)cc43)n2)cc1]

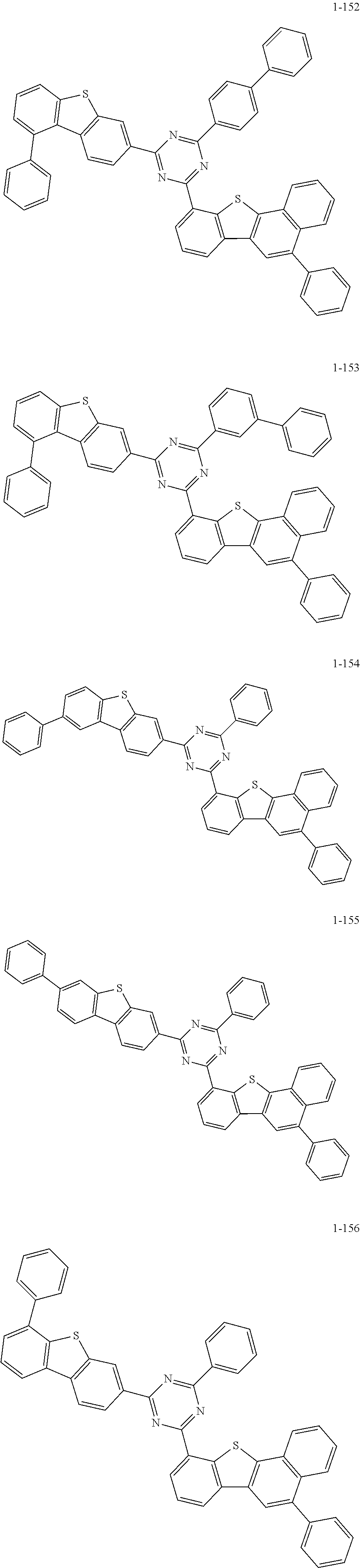 [SMILES: c1ccc(-c2ccc(-c3nc(-c4ccc5c(c4)sc4cccc(-c6ccccc6)c45)nc(-c4cccc5c4sc4c6ccccc6c(-c6ccccc6)cc54)n3)cc2)cc1 ; c1ccc(-c2cccc(-c3nc(-c4ccc5c(c4)sc4cccc(-c6ccccc6)c45)nc(-c4cccc5c4sc4c6ccccc6c(-c6ccccc6)cc54)n3)c2)cc1 ; c1ccc(-c2ccc3sc4cc(-c5nc(-c6ccccc6)nc(-c6cccc7c6sc6c8ccccc8c(-c8ccccc8)cc76)n5)ccc4c3c2)cc1 ; c1ccc(-c2ccc3c(c2)sc2cc(-c4nc(-c5ccccc5)nc(-c5cccc6c5sc5c7ccccc7c(-c7ccccc7)cc65)n4)ccc23)cc1 ; c1ccc(-c2nc(-c3ccc4c(c3)sc3c(-c5ccccc5)cccc34)nc(-c3cccc4c3sc3c5ccccc5c(-c5ccccc5)cc43)n2)cc1]

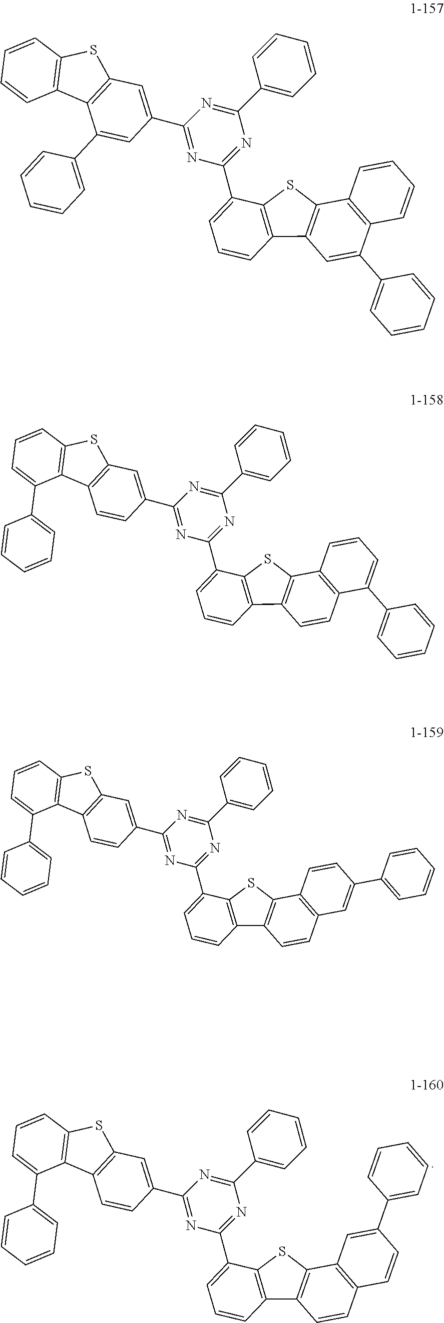 [SMILES: c1ccc(-c2nc(-c3cc(-c4ccccc4)c4c(c3)sc3ccccc34)nc(-c3cccc4c3sc3c5ccccc5c(-c5ccccc5)cc43)n2)cc1 ; c1ccc(-c2nc(-c3ccc4c(c3)sc3cccc(-c5ccccc5)c34)nc(-c3cccc4c3sc3c5cccc(-c6ccccc6)c5ccc43)n2)cc1 ; c1ccc(-c2ccc3c(ccc4c5cccc(-c6nc(-c7ccccc7)nc(-c7ccc8c(c7)sc7cccc(-c9ccccc9)c78)n6)c5sc34)c2)cc1 ; c1ccc(-c2ccc3ccc4c5cccc(-c6nc(-c7ccccc7)nc(-c7ccc8c(c7)sc7cccc(-c9ccccc9)c78)n6)c5sc4c3c2)cc1]

8. An organic light emitting device comprising:
a first electrode;
a second electrode; and
an organic material layer having one or more layers provided between the first electrode and the second electrode,
wherein one or more layers of the organic material layer comprise the heterocyclic compound of claim 1.

9. The organic light emitting device of claim 8, wherein the organic material layer comprising the heterocyclic compound further comprises a compound of the following Chemical Formula 2: 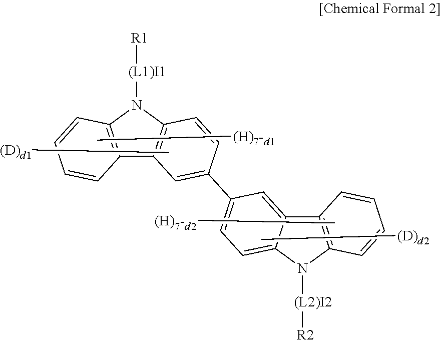
in Chemical Formula 2,
L2 and L3 are each independently a direct bond; or a substituted or unsubstituted arylene group having 6 to 60 carbon atoms,
l1 and l2 are each an integer from 1 to 3, and when l1 and l2 are each 2 or higher, substituents in the parenthesis are the same as or different from each other,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms,
H is hydrogen and D is deuterium, and
d1 and d2 are each independently an integer from 0 to 7.

10. The organic light emitting device of claim 9, wherein the deuterium content of Chemical Formula 2 is 0% or 10% to 100%.

11. The organic light emitting device of claim 9, wherein Chemical Formula 2 is represented by any one of the following compounds:

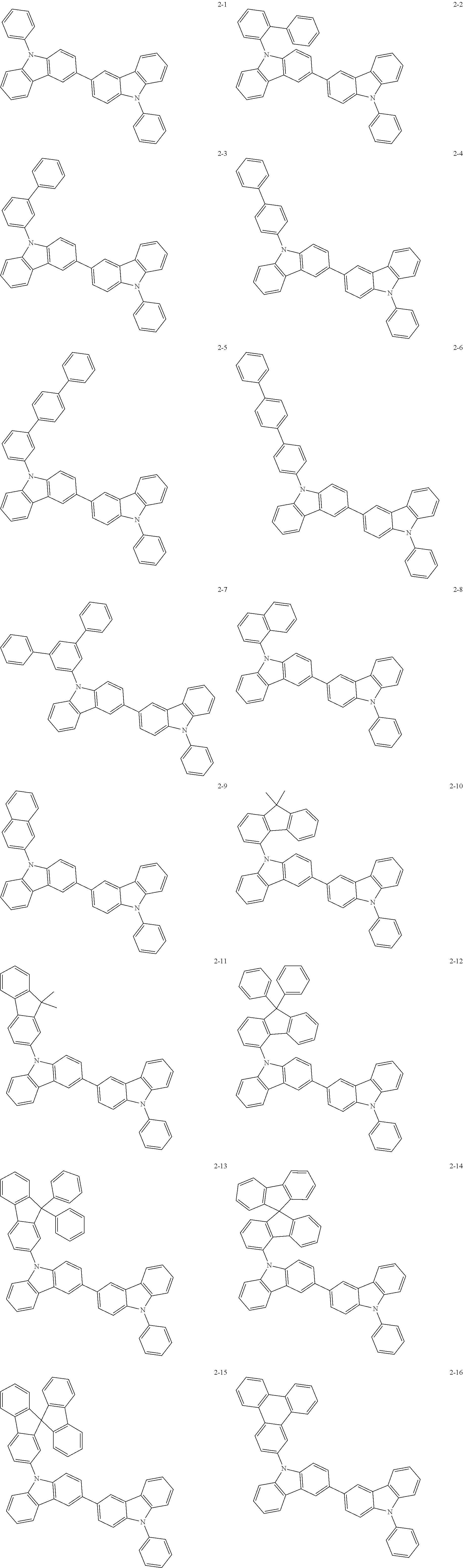

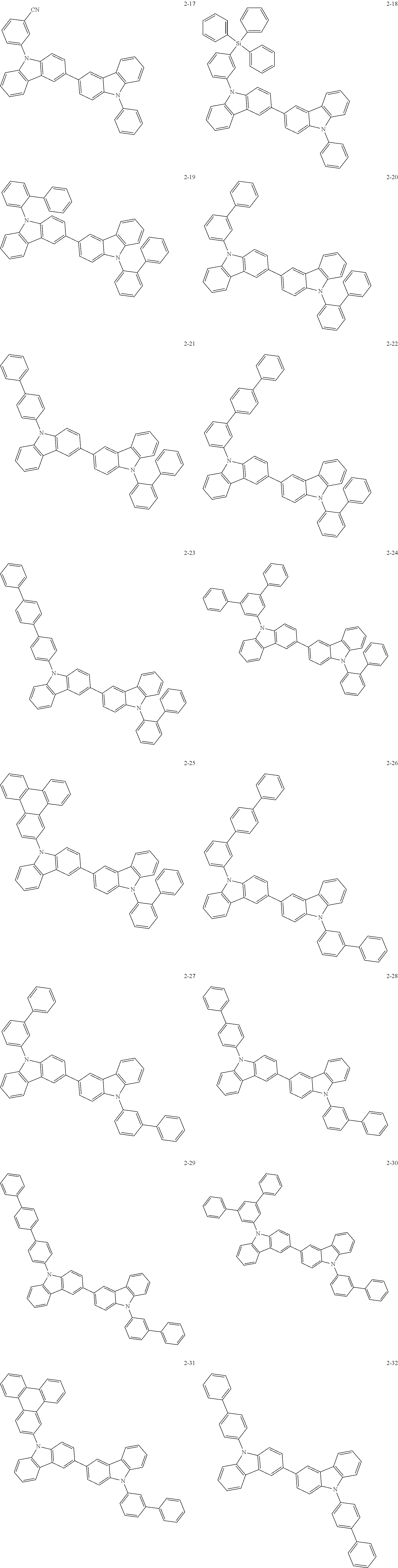

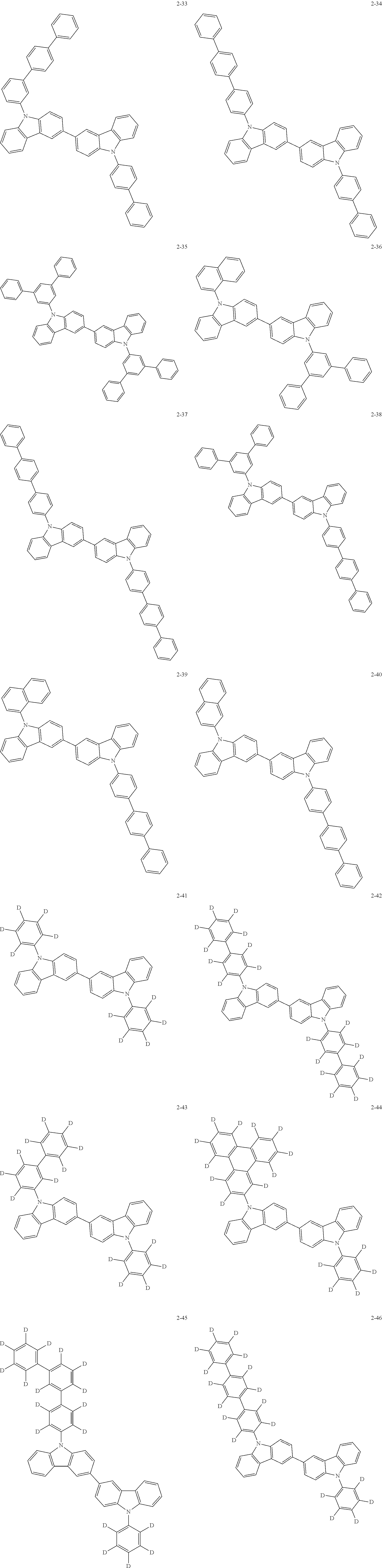

[chemical structure]

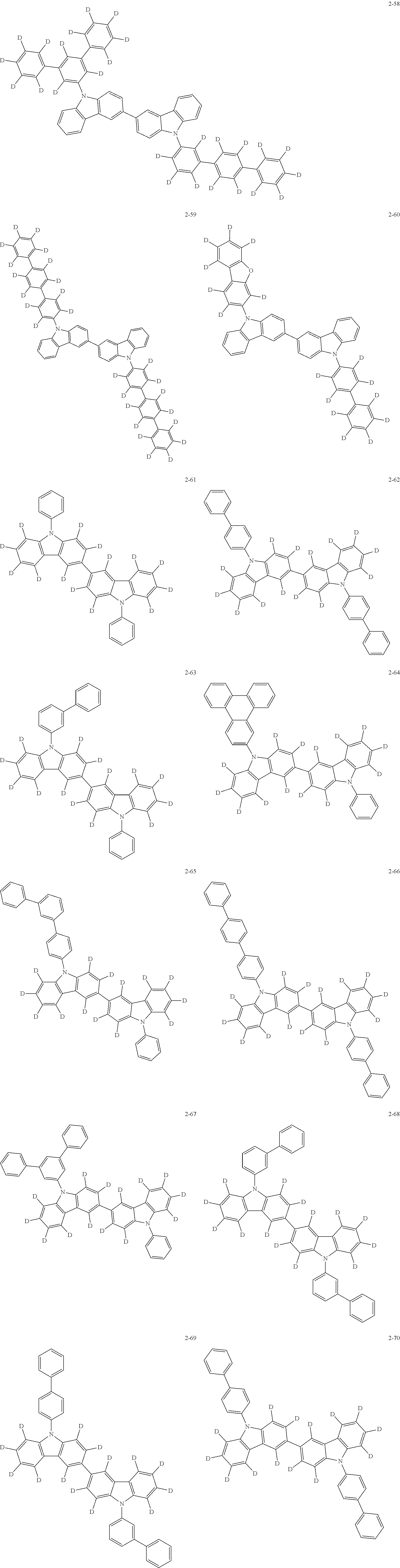

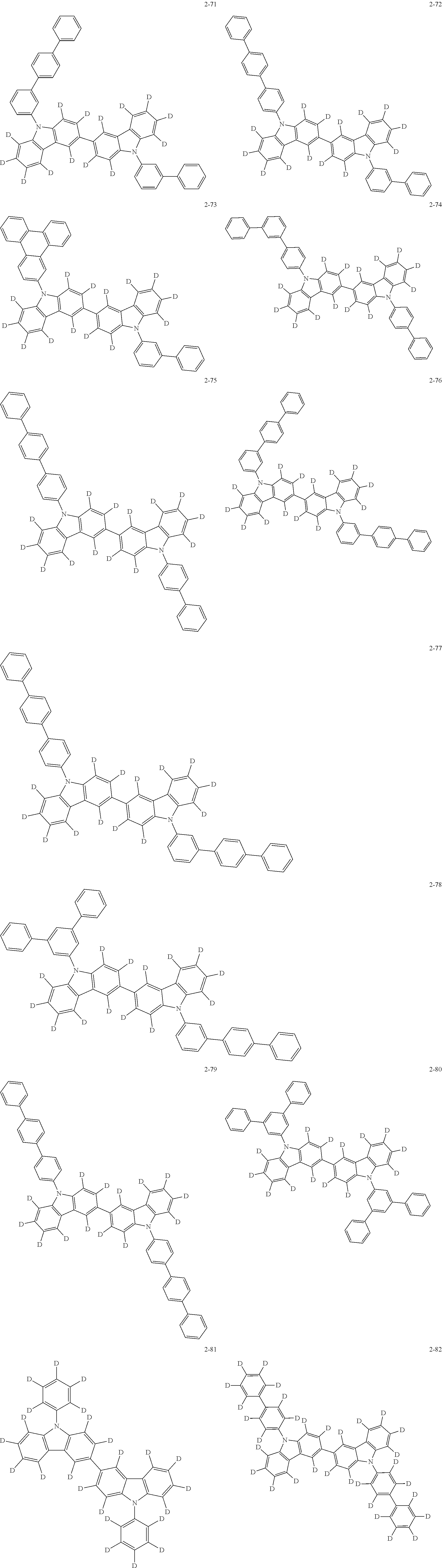

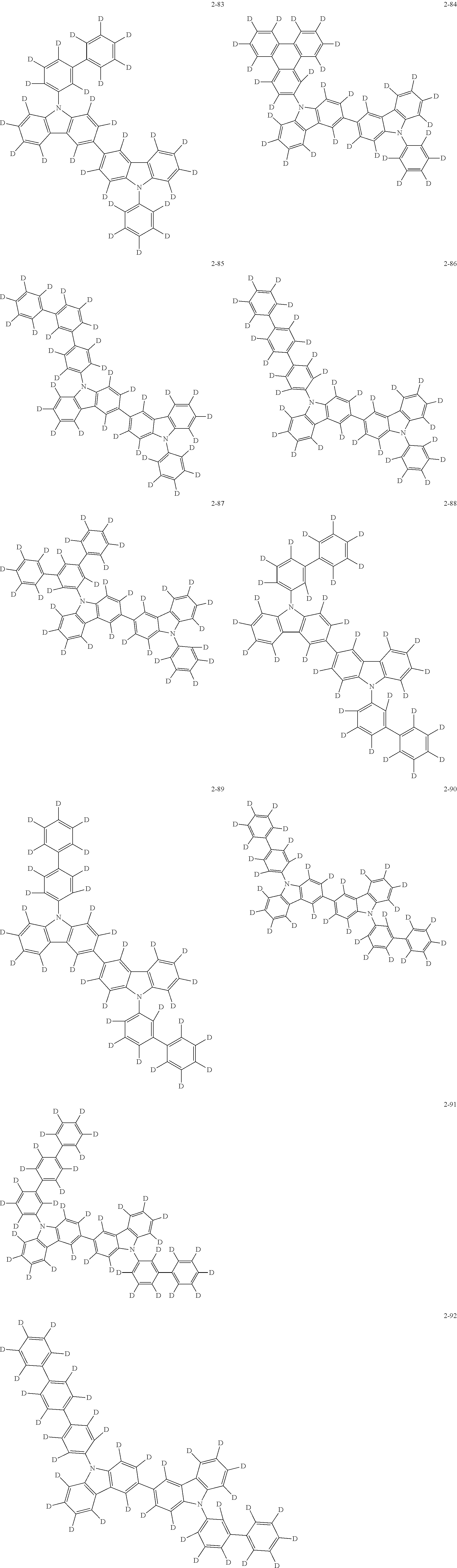

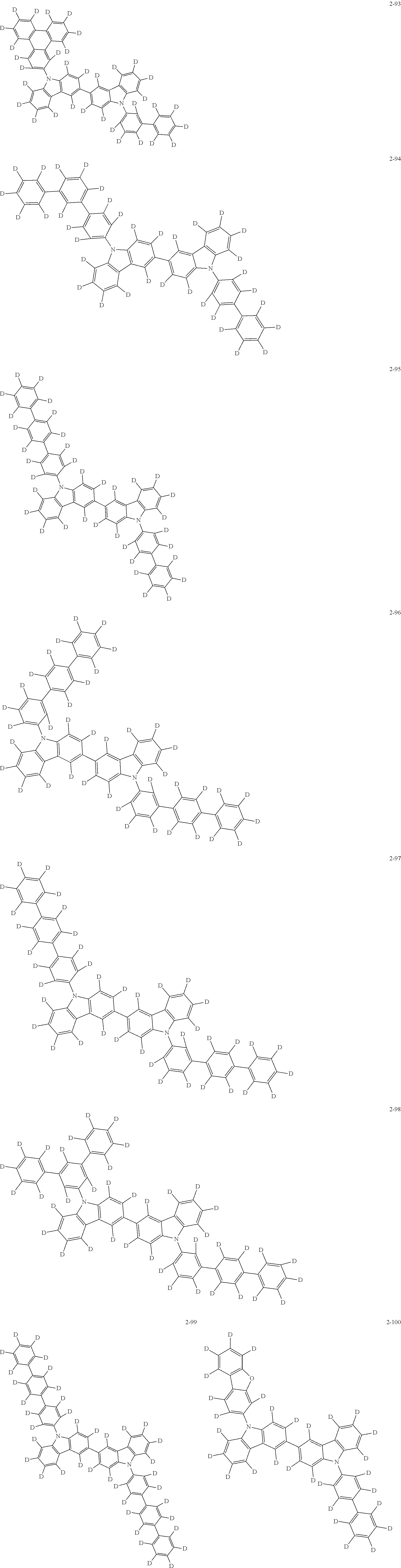

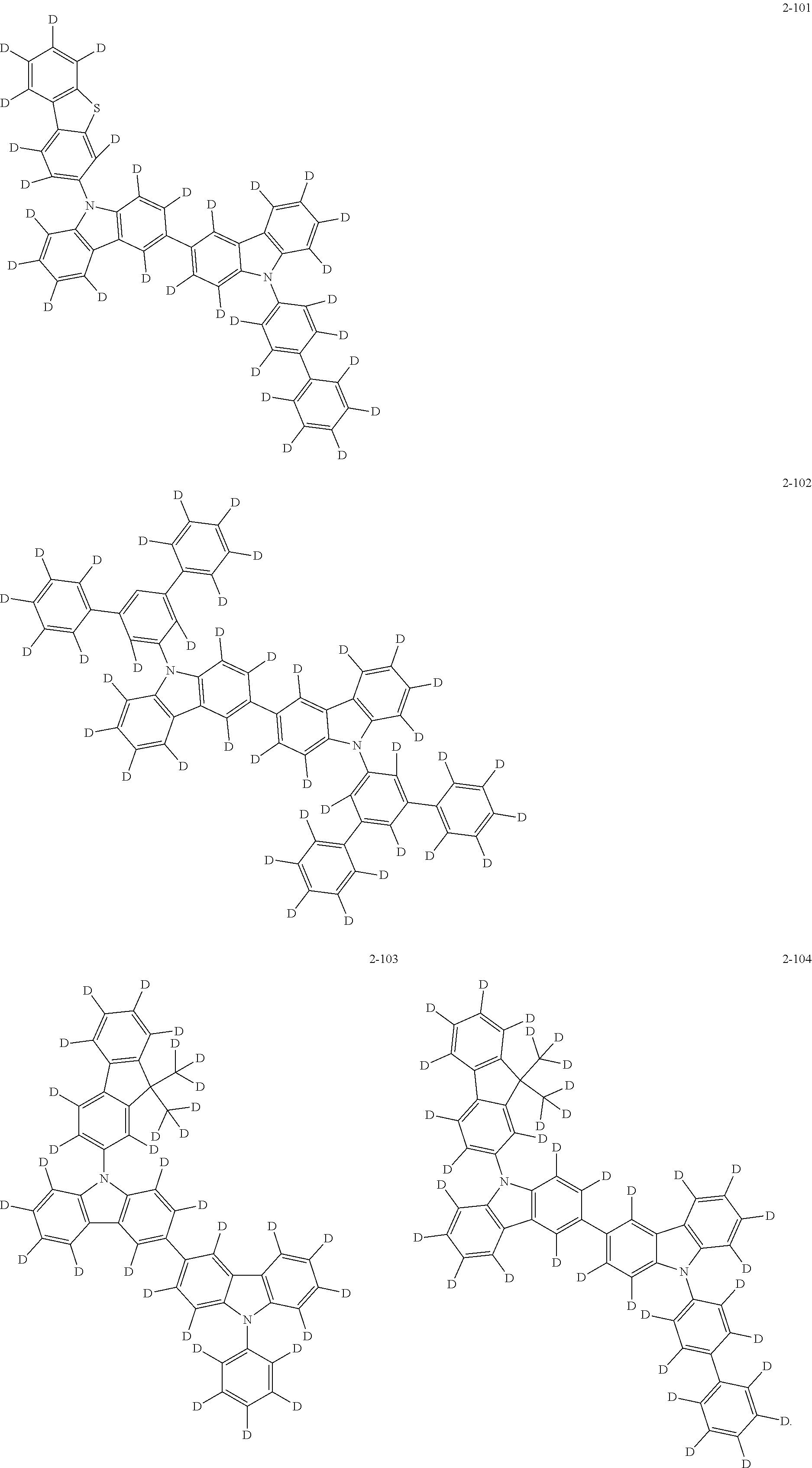

12. The organic light emitting device of claim 8, wherein the organic mate layer comprises a light emitting layer, and
the light emitting layer comprises the heterocyclic compound of Chemical Formula 1.

13. The organic light emitting device of claim 9, wherein the organic material layer comprises a light emitting layer, and
the light emitting layer comprises the heterocyclic compound of Chemical Formula 1 and the compound of Chemical Formula 2.

14. The organic light emitting device of claim 8, further comprising one or more layers selected from the group consisting of a light emitting layer, a hole injection layer, a hole transport layer, an electron injection layer, an electron transport layer, an electron blocking layer and a hole blocking layer.

15. A composition for an organic material layer of an organic light emitting device, comprising the heterocyclic compound of claim 1.

16. The composition of claim 15, further comprising a compound of the following Chemical Formula 2: 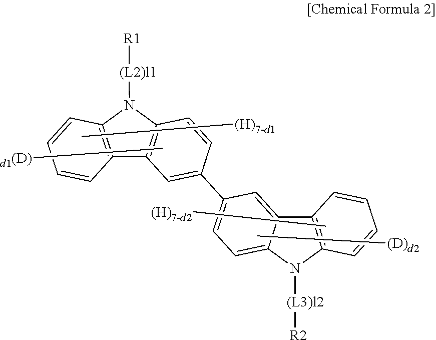
in Chemical Formula 2,
L2 and L3 are each independently a direct bond; or a substituted or unsubstituted arylene group having 6 to 60 carbon atoms,
l1 and l2 are each an integer from 1 to 3, and when l1 and l2 are each 2 or higher, substituents in the parenthesis are the same as or different from each other,
R1 and R2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms,
H is hydrogen and D is deuterium, and
d1 and d2 are each independently an integer from 0 to 7.

17. The composition of claim 16, wherein a weight ratio of the heterocyclic compound of Chemical Formula 1 and the compound of Chemical Formula 2 in the composition is 1:10 to 10:1.
